(19) United States
(12) Patent Application Publication
(10) Pub. No.: US 2026/0069535 A1
(43) Pub. Date: Mar. 12, 2026

(54) NEBULIZER KITS AND USES THEREOF

(71) Applicant: Pharmosa Biopharm Inc., Taipei City (TW)

(72) Inventors: Pei Kan, Taipei City (TW); Ko-Chieh Chen, Taipei City (TW); Hsiang-Fa Liang, Taipei City (TW)

(21) Appl. No.: 19/243,441

(22) Filed: Jun. 19, 2025

Related U.S. Application Data
(60) Provisional application No. 63/661,881, filed on Jun. 19, 2024.
(60) Provisional application No. 63/687,788, filed on Aug. 28, 2024.
(60) Provisional application No. 63/768,176, filed on Mar. 7, 2025.
(60) Provisional application No. 63/769,164, filed on Mar. 10, 2025.

(51) Int. Cl. A61K 9/00; A61K 9/1271; A61K 9/1272; A61K 31/5575; A61K 31/5578

(52) U.S. Cl. CPC A61K 9/0078; A61K 9/1271; A61K 9/1272; A61K 31/5575; A61K 31/5578

(57) ABSTRACT

The present invention relates to a nebulizer kit, comprising a pharmaceutical composition including a prostaglandin, prostacyclin and the analogue thereof and a nebulizer to deliver a prostaglandin, prostaglandin analogue, prostacyclin or prostacyclin analogue with a higher drug delivery efficiency. Also disclosed are methods for treating a respiratory disease and heart failure, using the nebulizer kit disclosed herein.

### CROSS-REFERENCE TO RELATED APPLICATIONS

This application claims the benefit of U.S. Application No. 63/661,881, filed on 19 Jun. 2024, U.S. Application No. 63/687,788, filed on 28 Aug. 2024, U.S. Application No. 63/768,176, filed on 7 Mar. 2025 and U.S. 63/769,164, filed on 10 Mar. 2025, and the entire disclosures of which are incorporated herein by reference.

### TECHNOLOGY FIELD

The present invention relates to a nebulizer kit to deliver about 5 to about 600 μg of a prostaglandin, prostaglandin analogue, prostacyclin or prostacyclin analogue, with reduced inhalation time and increased drug delivery efficiency, as well as the use of the nebulizer kit to treat a respiratory disease or heart failure.

### BACKGROUND OF THE INVENTION

Aerosols can be produced by different techniques, classified into three main categories: nebulizers (jet, ultrasonic, non-ultrasonic, and vibrating mesh), metered dose inhalers and dry powder inhalers. The inhalation of these aerosols provides an effective, but often inefficient method to deliver medications directly to the respiratory track to treat diseases, most commonly respiratory diseases.

Compared to metered dose inhalers and dry powder inhalers, nebulizers deliver high-drug doses with an aerosol deposition in the pulmonary region. However, one of the major disadvantages of nebulizers is long delivery times for high-dose medications.

Increasing drug delivery efficiency to decrease nebulization time while maintaining treatment dosage would be of considerable value for aerosol therapy, as it improves treatment convenience and improves long-term compliance.

There is still an unmet need for aerosol therapy to deliver the medication with a higher drug delivery efficiency and a shorter delivery time. The present invention addresses this need and other needs.

### BRIEF SUMMARY OF THE INVENTION

In one embodiment, a nebulizer kit, comprising (a) about 0.01 mL to about 2 mL of a pharmaceutical composition, said pharmaceutical composition comprising about 100 to about 2000 μg/mL of a prostaglandin, prostaglandin analogue, prostacyclin or prostacyclin analogue; and (b) a nebulizer, comprising a mesh, a breath-activated sensor, and a reservoir that is configured to hold about 0.01 mL to about 2 mL of the pharmaceutical composition is disclosed. The nebulizer is configured to deliver about 5 to about 600 μg of the prostaglandin, prostacyclin or the analogue thereof in less than 3 minutes.

In another embodiment, a nebulizer kit, comprising (a) about 0.01 mL to about 2 mL of a pharmaceutical composition, said pharmaceutical composition comprising a plurality of liposomes, said liposomes encapsulate about 100 to 2000 μg/mL of a prostaglandin, prostacyclin or the analogue thereof; and (b) a nebulizer, comprising a mesh, a breath-activated sensor, and a reservoir that is configured to hold about 0.01 mL to about 2 mL of the pharmaceutical composition is disclosed. The nebulizer is configured to deliver about 5 to about 600 μg of the prostaglandin, prostacyclin or the analogues thereof with a drug delivery efficiency higher than about 50%.

Also provided are methods for treating and/or prophylactic treating a respiratory disease or heart failure, by administering about 0.01 mL to about 2 mL of a pharmaceutical composition using the kit disclosed herein. In an exemplary embodiment, the pharmaceutical composition comprises about 100 to about 2000 μg/mL of a prostaglandin, prostacyclin or the analogue thereof. In another embodiment, the pharmaceutical composition comprises a plurality of liposomes, said liposomes encapsulate about 100 to about 2000 μg/mL of a prostaglandin, prostacyclin or the analogue thereof. In an exemplary embodiment, when the prostacyclin or its analogue is iloprost, the treatment or prophylactic treatment of systemic sclerosis and frost bite is also provided by the kit disclosed herein.

Also provided is the use of the kit for treating and/or prophylactic treatment of a respiratory disease or heart failure. When the prostacyclin or its analogue is iloprost, the use of the kit for treating systemic sclerosis and frost bite is also provided.

The invention also includes the use of the pharmaceutical composition and the nebulizer disclosed herein for the manufacture of a kit for the treatment and/or prophylactic treatment of a respiratory disease or heart failure. When the prostacyclin or its analogue is iloprost, the use of the pharmaceutical composition and the nebulizer disclosed herein for the manufacture of a kit for treating systemic sclerosis and frost bite is also provided.

The details of one or more embodiments of the invention are set forth in the description below. Other features or advantages of the present invention will be apparent from the following detailed description of several embodiments, and also from the appending claims. Statements containing these terms should be understood not to limit the subject matter described herein or to limit the meaning or scope of the patent claims below. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

### BRIEF DESCRIPTION OF THE DRAWINGS

Illustrative embodiments of the present invention are described in detail below with reference to the following figures.

FIG. 1 and FIG. 2 are perspective views of an embodiment of the non-ultrasonic nebulizer of the present invention. Both figures illustrate a main body 1, a reservoir 2, a mesh 3, a breath actuated sensor 4 and a mouth piece 5. The mesh 3 and the sensor 4 are connected by an electrode pin (not shown).

FIG. 3 is a perspective view of an embodiment of the non-ultrasonic nebulizer of the present invention, with the mouth piece 5 connected to the main body 1 for dispensing the aerosols.

FIG. 4A and FIG. 4B are cross section views of the non-ultrasonic nebulizer of FIG. 1 to FIG. 3, showing a reservoir 2, a mesh 3, the depth of the pharmaceutical composition D, and the angle A between the two opposite walls of the reservoir.

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “comprises” or “comprising” is generally used in the sense of include/including which means permitting the presence of one or more features, ingredients or components. The term “comprises” or “comprising” encompasses the term “consists” or “consisting of.”

All numbers are modified by the term “about”. As used herein, the term “about” refers to a range of ±10% of a specified value.

The term “subject” can refer to a vertebrate having a respiratory disease or heart failure or to a vertebrate deemed to be in need of treatment for a respiratory disease or heart failure. Subjects include warm-blooded animals, such as mammals, such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). Thus, veterinary uses and medical formulations are contemplated herein.

As used herein, “substantially free” means that pharmaceutical composition contains less than 5%, 4%, 3%, 2% or 1% of a specific substance or does not contain the specific substance.

### Nebulizer Kit

The present invention relates to a nebulizer kit, comprising:
(i) about 0.01 mL to about 2 mL of a pharmaceutical composition, said pharmaceutical composition comprising about 100 to about 2000 μg/mL of a prostaglandin, prostaglandin analogue, prostacyclin or prostacyclin analogue; and
(ii) a nebulizer, comprising a mesh, a breath-activated sensor and a reservoir, said reservoir configured to hold about 0.01 mL to about 2 mL of the pharmaceutical composition.

In some embodiments, the nebulizer is configured to deliver about 5 to about 600 μg of the prostaglandin, prostaglandin analogue, prostacyclin or prostacyclin analogue in less than 5 minutes. In other embodiments, the nebulizer is configured to deliver about 5 to about 600 μg of the prostaglandin, prostaglandin analogue, prostacyclin or prostacyclin analogue with a drug delivery efficiency higher than about 50, 55, 60, 65 or 70%.

An instruction to administer the pharmaceutical composition 2 times, 3 times, 4 times or 2-4 times within 24 hours is optionally included in the kit.

### Pharmaceutical Composition

In one embodiment, about 0.01 mL to about 0.3 mL of the pharmaceutical composition is in one or more ampules, said pharmaceutical composition comprising about 100 to about 2000 μg/mL or about 5 to about 600 μg of a prostaglandin, prostaglandin analogue, prostacyclin or prostacyclin analogue.

The pharmaceutical composition is transferable from one or more ampules to the reservoir of the nebulizer by a subject or user. In one embodiment, about 0.01 mL to 2 mL, about 0.05 mL to less than 1 mL, about 0.05 mL to less than 0.5 mL or about 0.01 mL to about 0.5 mL of a pharmaceutical composition is loaded in the reservoir of the nebulizer. In one embodiment, about 5 to about 600 μg of a prostaglandin, prostaglandin analogue, prostacyclin or prostacyclin analogue can be delivered in less than 3, 2, 1.5 or 1 minute and/or with a drug delivery efficiency higher than 50, 55, 60, 65 or 70%.

In some exemplary embodiments, said pharmaceutical composition and/or the nebulizer can deliver in less than 3, 2 1.5 or 1 minute, about 100 to about 1750 μg/mL, about 100 to about 1500 μg/mL, about 100 to about 1250 μg/mL, about 100 to about 1000 μg/mL, about 100 to about 750 μg/mL about 100 to about 500 μg/mL, about 100 to about 250 μg/mL, about 250 to about 2000 μg/mL, about 250 to about 1750 μg/mL, about 250 to about 1500 μg/mL, about 250 to about 1250 μg/mL, about 250 to about 1000 μg/mL, about 250 to about 750 μg/mL, or about 250 to about 500 μg/mL, about 500 to about 2000 μg/mL, about 500 to about 1750 μg/mL, about 500 to about 1500 μg/mL, about 500 to about 1250 μg/mL, about 500 to about 1000 g/mL, or about 500 to about 750 μg/mL, about 750 to about 2000 g/mL, about 750 to about 1750 μg/mL, about 750 to about 1500 μg/mL, about 750 to about 1250 μg/mL, or about 750 to about 1000 μg/mL, about 1000 to about 2000 μg/mL, about 1000 to about 1750 μg/mL, about 1000 to about 1500 μg/mL, or about 1000 to about 1250 μg/mL, about 1250 to about 2000 μg/mL, about 1250 to about 1750 μg/mL, about 1250 to about 1500 μg/mL, about 1500 to about 2000 μg/mL, about 1500 to about 1750 μg/mL, or about 1750 to about 2000 μg/mL of a prostaglandin, prostaglandin analogue, prostacyclin or prostacyclin analogue.

In some exemplary embodiments, said pharmaceutical composition and/or the nebulizer can deliver about 100 to about 1750 μg/mL of a prostaglandin, prostaglandin analogue, prostacyclin or prostacyclin analogue in less than 3 minutes, about 100 to about 1500 μg/mL of a prostaglandin, prostaglandin analogue, prostacyclin or prostacyclin analogue in less than 3 minutes, about 100 to about 1250 μg/mL of a prostaglandin, prostaglandin analogue, prostacyclin or prostacyclin analogue in less than 3 minutes, about 100 to about 1000 μg/mL of a prostaglandin, prostaglandin analogue, prostacyclin or prostacyclin analogue in less than 3 minutes, about 100 to about 750 μg/ml of a prostaglandin, prostaglandin analogue, prostacyclin or prostacyclin analogue in less than 3 minutes, about 100 to about 500 μg/mL of a prostaglandin, prostaglandin analogue, prostacyclin or prostacyclin analogue in less than 3 minutes, about 100 to about 250 μg/mL of a prostaglandin, prostaglandin analogue, prostacyclin or prostacyclin analogue in less than 3 minutes, about 250 to about 2000 μg/mL of a prostaglandin, prostaglandin analogue, prostacyclin or prostacyclin analogue in less than 3 minutes, about 250 to about 1750 μg/mL of a prostaglandin, prostaglandin analogue, prostacyclin or prostacyclin analogue in less than 3 minutes, about 250 to about 1500 μg/mL of a prostaglandin, prostaglandin analogue, prostacyclin or prostacyclin analogue in less than 3 minutes, about 250 to about 1250 μg/mL of a prostaglandin, prostaglandin analogue, prostacyclin or prostacyclin analogue in less than 3 minutes, about 250 to about 1000 μg/mL of a prostaglandin, prostaglandin analogue, prostacyclin or prostacyclin analogue in less than 3 minutes, about 250 to about 750 μg/mL in less than 3 minutes, about 250 to about 500 μg/mL of a prostaglandin, prostaglandin analogue, prostacyclin or prostacyclin analogue in less than 3 minutes, about 500 to about 2000 μg/mL of a prostaglandin, prostaglandin analogue, prostacyclin or prostacyclin analogue in less than 3 minutes, about 500 to about 1750 μg/mL of a prostaglandin, prostaglandin analogue, prostacyclin or prostacyclin analogue in less than 3 minutes, about 500 to about 1500 μg/mL of a prostaglandin, prostaglandin analogue, prostacyclin or prostacyclin analogue in less than 3 minutes, about 500 to about 1250 μg/mL of a prostaglandin, prostaglandin analogue, prostacyclin or prostacyclin analogue in less than 3 minutes, about 500 to about 1000 μg/mL of a prostaglandin, prostaglandin analogue, prostacyclin or prostacyclin analogue in less than 3 minutes, or about 500 to about 750 μg/mL of a prostaglandin, prostaglandin analogue, prostacyclin or prostacyclin analogue in less than 3 minutes, about 750 to about 2000 μg/mL of a prostaglandin, prostaglandin analogue, prostacyclin or prostacyclin analogue in less than 3 minutes, about 750 to about 1750 μg/mL of a prostaglandin, prostaglandin analogue, prostacyclin or prostacyclin analogue in less than 3 minutes, about 750 to about 1500 μg/mL of a prostaglandin, prostaglandin analogue, prostacyclin or prostacyclin analogue in less than 3 minutes, about 750 to about 1250 μg/mL of a prostaglandin, prostaglandin analogue, prostacyclin or prostacyclin analogue in less than 3 minutes, about 750 to about 1000 μg/ml of a prostaglandin, prostaglandin analogue, prostacyclin or prostacyclin analogue in less than 3 minutes, about 1000 to about 2000 μg/mL of a prostaglandin, prostaglandin analogue, prostacyclin or prostacyclin analogue in less than 3 minutes, about 1000 to about 1750 μg/mL of a prostaglandin, prostaglandin analogue, prostacyclin or prostacyclin analogue in less than 3 minutes, about 1000 to about 1500 μg/mL of a prostaglandin, prostaglandin analogue, prostacyclin or prostacyclin analogue in less than 3 minutes, about 1000 to about 1250 μg/mL of a prostaglandin, prostaglandin analogue, prostacyclin or prostacyclin analogue in less than 3 minutes, about 1250 to about 2000 μg/mL of a prostaglandin, prostaglandin analogue, prostacyclin or prostacyclin analogue in less than 3 minutes, about 1250 to about 1750 μg/mL of a prostaglandin, prostaglandin analogue, prostacyclin or prostacyclin analogue in less than 3 minutes, about 1250 to about 1500 μg/mL of a prostaglandin, prostaglandin analogue, prostacyclin or prostacyclin analogue in less than 3 minutes, about 1500 to about 2000 μg/mL of a prostaglandin, prostaglandin analogue, prostacyclin or prostacyclin analogue in less than 3 minutes, about 1500 to about 1750 μg/mL of a prostaglandin, prostaglandin analogue, prostacyclin or prostacyclin analogue in less than 3 minutes, or about 1750 to about 2000 μg/mL of a prostaglandin, prostaglandin analogue, prostacyclin or prostacyclin analogue in less than 3 minutes.

In some exemplary embodiments, said pharmaceutical composition and/or the nebulizer can deliver about 100 to about 1750 μg/mL of a prostaglandin, prostaglandin analogue, prostacyclin or prostacyclin analogue in less than 2 minutes, about 100 to about 1500 μg/mL of a prostaglandin, prostaglandin analogue, prostacyclin or prostacyclin analogue in less than 2 minutes, about 100 to about 1250 μg/mL of a prostaglandin, prostaglandin analogue, prostacyclin or prostacyclin analogue in less than 2 minutes, about 100 to about 1000 μg/mL of a prostaglandin, prostaglandin analogue, prostacyclin or prostacyclin analogue in less than 2 minutes, about 100 to about 750 μg/mL of a prostaglandin, prostaglandin analogue, prostacyclin or prostacyclin analogue in less than 2 minutes, about 100 to about 500 μg/mL of a prostaglandin, prostaglandin analogue, prostacyclin or prostacyclin analogue in less than 2 minutes, about 100 to about 250 μg/mL of a prostaglandin, prostaglandin analogue, prostacyclin or prostacyclin analogue in less than 2 minutes, about 250 to about 2000 μg/mL of a prostaglandin, prostaglandin analogue, prostacyclin or prostacyclin analogue in less than 2 minutes, about 250 to about 1750 μg/mL in less than 2 minutes, about 250 to about 1500 μg/mL of a prostaglandin, prostaglandin analogue, prostacyclin or prostacyclin analogue in less than 2 minutes, about 250 to about 1250 μg/mL of a prostaglandin, prostaglandin analogue, prostacyclin or prostacyclin analogue in less than 2 minutes, about 250 to about 1000 μg/mL of a prostaglandin, prostaglandin analogue, prostacyclin or prostacyclin analogue in less than 2 minutes, about 250 to about 750 μg/mL of a prostaglandin, prostaglandin analogue, prostacyclin or prostacyclin analogue in less than 2 minutes, about 250 to about 500 μg/mL of a prostaglandin, prostaglandin analogue, prostacyclin or prostacyclin analogue in less than 2 minutes, about 500 to about 2000 μg/mL of a prostaglandin, prostaglandin analogue, prostacyclin or prostacyclin analogue in less than 2 minutes, about 500 to about 1750 μg/mL of a prostaglandin, prostaglandin analogue, prostacyclin or prostacyclin analogue in less than 2 minutes, about 500 to about 1500 μg/mL of a prostaglandin, prostaglandin analogue, prostacyclin or prostacyclin analogue in less than 2 minutes, about 500 to about 1250 μg/mL of a prostaglandin, prostaglandin analogue, prostacyclin or prostacyclin analogue in less than 2 minutes, about 500 to about 1000 μg/mL of a prostaglandin, prostaglandin analogue, prostacyclin or prostacyclin analogue in less than 2 minutes, or about 500 to about 750 μg/mL of a prostaglandin, prostaglandin analogue, prostacyclin or prostacyclin analogue in less than 2 minutes, about 750 to about 2000 μg/mL of a prostaglandin, prostaglandin analogue, prostacyclin or prostacyclin analogue in less than 2 minutes, about 750 to about 1750 μg/mL of a prostaglandin, prostaglandin analogue, prostacyclin or prostacyclin analogue in less than 2 minutes, about 750 to about 1500 μg/mL of a prostaglandin, prostaglandin analogue, prostacyclin or prostacyclin analogue in less than 2 minutes, about 750 to about 1250 μg/mL of a prostaglandin, prostaglandin analogue, prostacyclin or prostacyclin analogue in less than 2 minutes, about 750 to about 1000 μg/mL of a prostaglandin, prostaglandin analogue, prostacyclin or prostacyclin analogue in less than 2 minutes, about 1000 to about 2000 μg/mL of a prostaglandin, prostaglandin analogue, prostacyclin or prostacyclin analogue in less than 2 minutes, about 1000 to about 1750 μg/mL of a prostaglandin, prostaglandin analogue, prostacyclin or prostacyclin analogue in less than 2 minutes, about 1000 to about 1500 μg/mL of a prostaglandin, prostaglandin analogue, prostacyclin or prostacyclin analogue in less than 2 minutes, about 1000 to about 1250 μg/mL of a prostaglandin, prostaglandin analogue, prostacyclin or prostacyclin analogue in less than 2 minutes, about 1250 to about 2000 μg/mL of a prostaglandin, prostaglandin analogue, prostacyclin or prostacyclin analogue in less than 2 minutes, about 1250 to about 1750 μg/mL of a prostaglandin, prostaglandin analogue, prostacyclin or prostacyclin analogue in less than 2 minutes, about 1250 to about 1500 μg/mL of a prostaglandin, prostaglandin analogue, prostacyclin or prostacyclin analogue in less than 2 minutes, about 1500 to about 2000 μg/mL of a prostaglandin, prostaglandin analogue, prostacyclin or prostacyclin analogue in less than 2 minutes, about 1500 to about 1750 μg/mL of a prostaglandin, prostaglandin analogue, prostacyclin or prostacyclin analogue in less than 2 minutes, or about 1750 to about 2000 μg/mL of a prostaglandin, prostaglandin analogue, prostacyclin or prostacyclin analogue in less than 2 minutes.

In some exemplary embodiments, said pharmaceutical composition and/or the nebulizer can deliver about 100 to about 1750 μg/mL of a prostaglandin, prostaglandin analogue, prostacyclin or prostacyclin analogue in less than 1.5 minutes, about 100 to about 1500 μg/mL of a prostaglandin, prostaglandin analogue, prostacyclin or prostacyclin analogue in less than 1.5 minutes, about 100 to about 1250 μg/mL of a prostaglandin, prostaglandin analogue, prostacyclin or prostacyclin analogue in less than 1.5 minutes, about 100 to about 1000 μg/mL of a prostaglandin, prostaglandin analogue, prostacyclin or prostacyclin analogue in less than 1.5 minutes, about 100 to about 750 μg/mL of a prostaglandin, prostaglandin analogue, prostacyclin or prostacyclin analogue in less than 1.5 minutes, about 100 to about 500 μg/mL of a prostaglandin, prostaglandin analogue, prostacyclin or prostacyclin analogue in less than 1.5 minutes, about 100 to about 250 μg/mL of a prostaglandin, prostaglandin analogue, prostacyclin or prostacyclin analogue in less than 1.5 minutes, about 250 to about 2000 μg/mL of a prostaglandin, prostaglandin analogue, prostacyclin or prostacyclin analogue in less than 1.5 minutes, about 250 to about 1750 μg/mL of a prostaglandin, prostaglandin analogue, prostacyclin or prostacyclin analogue in less than 1.5 minutes, about 250 to about 1500 μg/mL of a prostaglandin, prostaglandin analogue, prostacyclin or prostacyclin analogue in less than 1.5 minutes, about 250 to about 1250 μg/mL of a prostaglandin, prostaglandin analogue, prostacyclin or prostacyclin analogue in less than 1.5 minutes, about 250 to about 1000 μg/mL of a prostaglandin, prostaglandin analogue, prostacyclin or prostacyclin analogue in less than 1.5 minutes, about 250 to about 750 μg/mL of a prostaglandin, prostaglandin analogue, prostacyclin or prostacyclin analogue in less than 1.5 minutes, about 250 to about 500 μg/mL of a prostaglandin, prostaglandin analogue, prostacyclin or prostacyclin analogue in less than 1.5 minutes, about 500 to about 2000 μg/mL of a prostaglandin, prostaglandin analogue, prostacyclin or prostacyclin analogue in less than 1.5 minutes, about 500 to about 1750 μg/mL of a prostaglandin, prostaglandin analogue, prostacyclin or prostacyclin analogue in less than 1.5 minutes, about 500 to about 1500 μg/mL of a prostaglandin, prostaglandin analogue, prostacyclin or prostacyclin analogue in less than 1.5 minutes, about 500 to about 1250 μg/mL of a prostaglandin, prostaglandin analogue, prostacyclin or prostacyclin analogue in less than 1.5 minutes, about 500 to about 1000 μg/mL of a prostaglandin, prostaglandin analogue, prostacyclin or prostacyclin analogue in less than 1.5 minutes, or about 500 to about 750 μg/mL of a prostaglandin, prostaglandin analogue, prostacyclin or prostacyclin analogue in less than 1.5 minutes, about 750 to about 2000 μg/mL of a prostaglandin, prostaglandin analogue, prostacyclin or prostacyclin analogue in less than 1.5 minutes, about 750 to about 1750 μg/mL of a prostaglandin, prostaglandin analogue, prostacyclin or prostacyclin analogue in less than 1.5 minutes, about 750 to about 1500 μg/mL of a prostaglandin, prostaglandin analogue, prostacyclin or prostacyclin analogue in less than 1.5 minutes, about 750 to about 1250 μg/mL of a prostaglandin, prostaglandin analogue, prostacyclin or prostacyclin analogue in less than 1.5 minutes, about 750 to about 1000 μg/mL of a prostaglandin, prostaglandin analogue, prostacyclin or prostacyclin analogue in less than 1.5 minutes, about 1000 to about 2000 μg/mL of a prostaglandin, prostaglandin analogue, prostacyclin or prostacyclin analogue in less than 1.5 minutes, about 1000 to about 1750 μg/mL of a prostaglandin, prostaglandin analogue, prostacyclin or prostacyclin analogue in less than 1.5 minutes, about 1000 to about 1500 μg/mL of a prostaglandin, prostaglandin analogue, prostacyclin or prostacyclin analogue in less than 1.5 minutes, about 1000 to about 1250 μg/mL of a prostaglandin, prostaglandin analogue, prostacyclin or prostacyclin analogue in less than 1.5 minutes, about 1250 to about 2000 μg/mL of a prostaglandin, prostaglandin analogue, prostacyclin or prostacyclin analogue in less than 1.5 minutes, about 1250 to about 1750 μg/mL of a prostaglandin, prostaglandin analogue, prostacyclin or prostacyclin analogue in less than 1.5 minutes, about 1250 to about 1500 μg/mL of a prostaglandin, prostaglandin analogue, prostacyclin or prostacyclin analogue in less than 1.5 minutes, about 1500 to about 2000 μg/mL of a prostaglandin, prostaglandin analogue, prostacyclin or prostacyclin analogue in less than 1.5 minutes, about 1500 to about 1750 μg/mL of a prostaglandin, prostaglandin analogue, prostacyclin or prostacyclin analogue in less than 1.5 minutes, or about 1750 to about 2000 μg/mL of a prostaglandin, prostaglandin analogue, prostacyclin or prostacyclin analogue in less than 1.5 minutes.

In some exemplary embodiments, the volume of the pharmaceutical composition loaded in the reservoir of the nebulizer is about 0.01 mL to about 1.75 mL, about 0.01 mL to about 1.5 mL, about 0.01 mL to about 1.25 mL, about 0.01 mL to about 1 mL, about 0.01 mL to about 0.95 mL, about 0.01 mL to about 0.9 mL, about 0.01 mL to about 0.85 mL, about 0.01 mL to about 0.8 mL, about 0.01 mL to about 0.75 mL, about 0.01 mL to about 0.7 mL, about 0.01 mL to about 0.65 mL, about 0.01 mL to about 0.6 mL, about 0.01 mL to about 0.55 mL, about 0.01 mL to about 0.5 mL, about 0.01 mL to about 0.45 mL, about 0.01 mL to about 0.4 mL, about 0.01 mL to about 0.35 mL, about 0.01 mL to about 0.3 mL, about 0.01 mL to about 0.25 mL, about 0.01 mL to about 0.2 mL, about 0.01 mL to about 0.15 mL, about 0.01 mL to about 0.1 mL, about 0.05 mL to about 2 mL, about 0.05 mL to about 1.75 mL, about 0.05 mL to about 1.5 mL, about 0.05 mL to about 1.25 mL, about 0.05 mL to about 1 mL, about 0.05 mL to about 0.95 mL, about 0.05 mL to about 0.9 mL, about 0.05 mL to about 0.85 mL, about 0.05 mL to about 0.8 mL, about 0.05 mL to about 0.75 mL, about 0.05 mL to about 0.7 mL, about 0.05 mL to about 0.65 mL, about 0.05 mL to about 0.6 mL, about 0.05 mL to about 0.55 mL, about 0.05 mL to about 0.5 mL, about 0.05 mL to about 0.45 mL, about 0.05 mL to about 0.4 mL, about 0.05 mL to about 0.35 mL, about 0.05 mL to about 0.3 mL, about 0.05 mL to about 0.25 mL, about 0.05 mL to about 0.2 mL, about 0.05 mL to about 0.15 mL, about 0.05 mL to about 0.1 mL, about 0.08 mL to about 2 mL, about 0.08 mL to about 1.75 mL, about 0.08 mL to about 1.5 mL, about 0.08 mL to about 1.25 mL, about 0.08 mL to about 1 mL, about 0.08 mL to about 0.95 mL, about 0.08 mL to about 0.9 mL, about 0.08 mL to about 0.85 mL, about 0.08 mL to about 0.8 mL, about 0.08 mL to about 0.75 mL, about 0.08 mL to about 0.7 mL, about 0.08 mL to about 0.65 mL, about 0.08 mL to about 0.6 mL, about 0.08 mL to about 0.55 mL, about 0.08 mL to about 0.5 mL, about 0.08 mL to about 0.45 mL, about 0.08 mL to about 0.4 mL, about 0.08 mL to about 0.35 mL, about 0.08 mL to about 0.3 mL, about 0.08 mL to about 0.25 mL, about 0.08 mL to about 0.2 mL, about 0.08 mL to about 0.15 mL, about 0.08 mL to about 0.1 mL, about 0.1 mL to about 2 mL, about 0.1 mL to about 1.75 mL, about 0.1 mL to about 1.5 mL, about 0.1 mL to about 1.25 mL, about 0.1 mL to about 1 mL, about 0.1 mL to about 0.95 mL, about 0.1 mL to about 0.9 mL, about 0.1 mL to about 0.85 mL, about 0.1 mL to about 0.8 mL, about 0.1 mL to about 0.75 mL, about 0.1 mL to about 0.7 mL, about 0.1 mL to about 0.65 mL, about 0.1 mL to about 0.6 mL, about 0.1 mL to about 0.55 mL, about 0.1 mL to about 0.5 mL, about 0.1 mL to about 0.45 mL, about 0.1 mL to about 0.4 mL, about 0.1 mL to about 0.35 mL, about 0.1 mL to about 0.3 mL, about 0.1 mL to about 0.25 mL, about 0.1 mL to about 0.2 mL, about 0.1 mL to about 0.15 mL, about 0.15 mL to about 1 mL, about 0.15 mL to about 0.95 mL, about 0.15 mL to about 0.9 mL, about 0.15 mL to about 0.85 mL, about 0.15 mL to about 0.8 mL, about 0.15 mL to about 0.75 mL, about 0.15 mL to about 0.7 mL, about 0.15 mL to about 0.65 mL, about 0.15 mL to about 0.6 mL, about 0.15 mL to about 0.55 mL, about 0.15 mL to about 0.5 mL, about 0.15 mL to about 0.45 mL, about 0.15 mL to about 0.4 mL, about 0.15 mL to about 0.35 mL, about 0.15 mL to about 0.3 mL, about 0.15 mL to about 0.25 mL, about 0.15 mL to about 0.2 mL, about 0.2 mL to about 1 mL, about 0.2 mL to about 0.95 mL, about 0.2 mL to about 0.9 mL, about 0.2 mL to about 0.85 mL, about 0.2 mL to about 0.8 mL, about 0.2 mL to about 0.75 mL, about 0.2 mL to about 0.7 mL, about 0.2 mL to about 0.65 mL, about 0.2 mL to about 0.6 mL, about 0.2 mL to about 0.55 mL, about 0.2 mL to about 0.5 mL, about 0.2 mL to about 0.45 mL, about 0.2 mL to about 0.4 mL, about 0.2 mL to about 0.35 mL, about 0.2 mL to about 0.3 mL, about 0.2 mL to about 0.25 mL, about 0.25 mL to about 1 mL, about 0.25 mL to about 0.95 mL, about 0.25 mL to about 0.9 mL, about 0.25 mL to about 0.85 mL, about 0.25 mL to about 0.8 mL, about 0.25 mL to about 0.75 mL, about 0.25 mL to about 0.7 mL, about 0.25 mL to about 0.65 mL, about 0.25 mL to about 0.6 mL, about 0.25 mL to about 0.55 mL, about 0.25 mL to about 0.5 mL, about 0.25 mL to about 0.45 mL, about 0.25 mL to about 0.4 mL, about 0.25 mL to about 0.35 mL, about 0.25 mL to about 0.3 mL, about 0.3 mL to about 1 mL, about 0.3 mL to about 0.95 mL, about 0.3 mL to about 0.9 mL, about 0.3 mL to about 0.85 mL, about 0.3 mL to about 0.8 mL, about 0.3 mL to about 0.75 mL, about 0.3 mL to about 0.7 mL, about 0.3 mL to about 0.65 mL, about 0.3 mL to about 0.6 mL, about 0.3 mL to about 0.55 mL, about 0.3 mL to about 0.5 mL, about 0.3 mL to about 0.45 mL, about 0.3 mL to about 0.4 mL, about 0.3 mL to about 0.35 mL, about 0.35 mL to about 1 mL, about 0.35 mL to about 0.95 mL, about 0.35 mL to about 0.9 mL, about 0.35 mL to about 0.85 mL, about 0.35 mL to about 0.8 mL, about 0.35 mL to about 0.75 mL, about 0.35 mL to about 0.7 mL, about 0.35 mL to about 0.65 mL, about 0.35 mL to about 0.6 mL, about 0.35 mL to about 0.55 mL, about 0.35 mL to about 0.5 mL, about 0.35 mL to about 0.45 mL, about 0.35 mL to about 0.4 mL, about 0.4 mL to about 1 mL, about 0.4 mL to about 0.95 mL, about 0.4 mL to about 0.9 mL, about 0.4 mL to about 0.85 mL, about 0.4 mL to about 0.8 mL, about 0.4 mL to about 0.75 mL, about 0.4 mL to about 0.7 mL, about 0.4 mL to about 0.65 mL, about 0.4 mL to about 0.6 mL, about 0.4 mL to about 0.55 mL, about 0.4 mL to about 0.5 mL, about 0.4 mL to about 0.45 mL, about 0.45 mL to about 1 mL, about 0.45 mL to about 0.95 mL, about 0.45 mL to about 0.9 mL, about 0.45 mL to about 0.85 mL, about 0.45 mL to about 0.8 mL, about 0.45 mL to about 0.75 mL, about 0.45 mL to about 0.7 mL, about 0.45 mL to about 0.65 mL, about 0.45 mL to about 0.6 mL, about 0.45 mL to about 0.55 mL, about 0.45 mL to about 0.5 mL, about 0.5 mL to about 1 mL, about 0.5 mL to about 0.95 mL, about 0.5 mL to about 0.9 mL, about 0.5 mL to about 0.85 mL, about 0.5 mL to about 0.8 mL, about 0.5 mL to about 0.75 mL, about 0.5 mL to about 0.7 mL, about 0.5 mL to about 0.65 mL, about 0.5 mL to about 0.6 mL, or about 0.5 mL to about 0.55 mL, or any range of volumes therebetween (e.g., 0.08 to 0.5 mL, 0.05 mL to 1.75 mL, 0.05 to 1.85 mL, 0.05 mL to 1.85 mL, 0.05 mL to 1.75 mL).

In some embodiments, each of the one or more ampules or the pharmaceutical composition in the reservoir of the nebulizer contains a prostaglandin, prostaglandin analogue, prostacyclin or prostacyclin analogue in an amount of about 5 μg to about 575 μg in about 0.01 mL to about 0.30 mL, about 5 μg to about 550 μg in about 0.01 mL to about 0.30 mL, about 5 μg to about 525 μg in about 0.01 mL to about 0.3 mL, about 5 μg to about 500 μg in about 0.01 mL to about 0.30 mL, about 5 μg to about 475 μg in about 0.01 mL to about 0.30 mL, about 5 μg to about 450 μg in about 0.01 mL to about 0.30 mL, about 5 μg to about 425 μg in about 0.01 mL to about 0.30 mL, about 5 μg to about 400 μg in about 0.01 mL to about 0.30 mL, about 5 μg to about 375 μg in about 0.01 mL to about 0.30 mL, about 5 μg to about 350 μg in about 0.01 mL to about 0.30 mL, about 5 μg to about 325 μg in about 0.01 mL to about 0.30 mL, about 5 μg to about 300 μg in about 0.01 mL to about 0.3 mL, about 5 μg to about 275 μg in about 0.01 mL to about 0.30 mL, about 5 μg to about 250 μg in about 0.01 mL to about 0.30 mL, about 5 μg to about 225 μg in about 0.01 mL to about 0.30 mL, about 5 μg to about 200 μg in about 0.01 mL to about 0.30 mL, about 5 μg to about 175 μg in about 0.01 mL to about 0.30 mL, about 5 μg to about 150 μg in about 0.10 mL to about 0.30 mL, about 5 μg to about 125 μg in about 0.10 mL to about 0.30 mL, about 5 μg to about 100 μg in about 0.10 mL to about 0.30 mL, about 5 μg to about 75 μg in about 0.10 mL to about 0.30 mL, about 5 μg to about 50 μg in about 0.10 mL to about 0.30 mL, about 5 μg to about 25 μg in about 0.10 mL to about 0.30 mL, about 5 μg to about 20 μg in about 0.10 mL to about 0.30 mL, about 5 μg to about 15 μg in about 0.10 mL to about 0.30 mL, about 5 μg to about 10 μg in about 0.10 mL to about 0.30 mL, about 5 μg to about 600 μg in about 0.10 mL to about 0.25 mL, about 5 μg to about 575 μg in about 0.10 mL to about 0.25 mL, about 5 μg to about 550 μg in about 0.10 mL to about 0.25 mL, about 5 μg to about 525 μg in about 0.10 mL to about 0.25 mL, about 5 μg to about 500 μg in about 0.10 mL to about 0.25 mL, about 5 μg to about 475 μg in about 0.10 mL to about 0.25 mL, about 5 μg to about 450 μg in about 0.10 mL to about 0.25 mL, about 5 μg to about 425 μg in about 0.10 mL to about 0.25 mL, about 5 μg to about 400 μg in about 0.10 mL to about 0.25 mL, about 5 μg to about 375 μg in about 0.10 mL to about 0.25 mL, about 5 μg to about 350 μg in about 0.10 mL to about 0.25 mL, about 5 μg to about 325 μg in about 0.10 mL to about 0.25 mL, about 5 μg to about 300 μg in about 0.10 mL to about 0.25 mL, about 5 μg to about 275 μg in about 0.10 mL to about 0.25 mL, about 5 μg to about 250 μg in about 0.10 mL to about 0.25 mL, about 5 μg to about 225 μg in about 0.10 mL to about 0.25 mL, about 5 μg to about 200 μg in about 0.10 mL to about 0.25 mL, about 5 μg to about 175 μg in about 0.10 mL to about 0.25 mL, about 5 μg to about 150 μg in about 0.10 mL to about 0.25 mL, about 5 μg to about 125 μg in about 0.10 mL to about 0.25 mL, about 5 μg to about 100 μg in about 0.10 mL to about 0.25 mL, about 5 μg to about 75 μg in about 0.10 mL to about 0.25 mL, about 5 μg to about 50 μg in about 0.10 mL to about 0.25 mL, about 5 μg to about 25 μg in about 0.10 mL to about 0.25 mL, about 5 μg to about 20 μg in about 0.10 mL to about 0.25 mL, about 5 μg to about 15 μg in about 0.10 mL to about 0.25 mL, about 5 μg to about 575 μg in about 0.10 mL to about 0.20 mL, about 5 μg to about 550 μg in about 0.10 mL to about 0.20 mL, about 5 μg to about 525 μg in about 0.10 mL to about 0.20 mL, about 5 μg to about 500 μg in about 0.10 mL to about 0.20 mL, about 5 μg to about 475 μg in about 0.10 mL to about 0.20 mL, about 5 μg to about 450 μg in about 0.10 mL to about 0.20 mL, about 5 μg to about 425 μg in about 0.10 mL to about 0.20 mL, about 5 μg to about 400 μg in about 0.10 mL to about 0.20 mL, about 5 μg to about 375 μg in about 0.10 mL to about 0.20 mL, about 5 μg to about 350 μg in about 0.10 mL to about 0.20 mL, about 5 μg to about 325 μg in about 0.10 mL to about 0.20 mL, about 5 μg to about 300 μg in about 0.50 mL, about 5 μg to about 275 μg in about 0.10 mL to about 0.20 mL, about 5 μg to about 250 μg in about 0.10 mL to about 0.20 mL, about 5 μg to about 225 μg in about 0.10 mL to about 0.20 mL, about 5 μg to about 200 μg in about 0.10 mL to about 0.20 mL, about 5 μg to about 175 μg in about 0.10 mL to about 0.20 mL, about 5 μg to about 150 μg in about 0.10 mL to about 0.20 mL, about 5 μg to about 125 μg in about 0.10 mL to about 0.20 mL, about 5 μg to about 100 μg in about 0.10 mL to about 0.20 mL, about 5 μg to about 75 μg in about 0.10 mL to about 0.20 mL, about 5 μg to about 50 μg in about 0.10 mL to about 0.20 mL, about 5 μg to about 25 μg in about 0.10 mL to about 0.20 mL, about 5 μg to about 20 μg in about 0.10 mL to about 0.20 mL, about 5 μg to about 15 μg in about 0.10 mL to about 0.20 mL, about 5 μg to about 10 μg in about 0.10 mL to about 0.20 mL, about 10 μg to about 600 μg in about 0.10 mL to about 0.20 mL, about 10 μg to about 575 μg in about 0.10 mL to about 0.20 mL, about 10 μg to about 550 μg in about 0.10 mL to about 0.20 mL, about 10 μg to about 525 μg in about 0.10 mL to about 0.20 mL, about 10 μg to about 500 μg in about 0.10 mL to about 0.20 mL, about 10 μg to about 475 μg in about 0.10 mL to about 0.20 mL, about 10 μg to about 450 μg in about 0.10 mL to about 0.20 mL, about 10 μg to about 425 μg in about 0.10 mL to about 0.20 mL, about 10 μg to about 400 μg in about 0.10 mL to about 0.20 mL, about 10 μg to about 375 μg in about 0.10 mL to about 0.20 mL, about 10 μg to about 350 μg in about 0.10 mL to about 0.20 mL, about 10 μg to about 325 μg in about 0.10 mL to about 0.20 mL, about 10 μg to about 300 μg in about 0.10 mL to about 0.20 mL, about 10 μg to about 275 μg in about 0.10 mL to about 0.20 mL, about 10 μg to about 250 μg in about 0.10 mL to about 0.20 mL, about 10 μg to about 225 μg in about 0.10 mL to about 0.20 mL, about 10 μg to about 200 μg in about 0.10 mL to about 0.20 mL, about 10 μg to about 175 μg in about 0.10 mL to about 0.20 mL, about 10 μg to about 150 μg in about 0.10 mL to about 0.20 mL, about 10 μg to about 125 μg in about 0.10 mL to about 0.20 mL, about 10 μg to about 100 μg in about 0.10 mL to about 0.20 mL, about 10 μg to about 75 μg in about 0.10 mL to about 0.20 mL, about 10 μg to about 50 μg in about 0.10 mL to about 0.20 mL, about 10 μg to about 25 μg in about 0.10 mL to about 0.20 mL, about 10 μg to about 20 μg in about 0.10 mL to about 0.20 mL, about 10 μg to about 15 μg in about 0.10 mL to about 0.20 mL, about 15 μg to about 600 μg in about 0.10 mL to about 0.20 mL, about 15 μg to about 575 μg in about 0.10 mL to about 0.20 mL, about 15 μg to about 550 μg in about 0.10 mL to about 0.20 mL, about 15 μg to about 525 μg in about 0.10 mL to about 0.20 mL, about 15 μg to about 500 μg in about 0.10 mL to about 0.20 mL, about 15 μg to about 475 μg in about 0.10 mL to about 0.20 mL, about 15 μg to about 450 μg in about 0.10 mL to about 0.20 mL, about 15 μg to about 425 μg in about 0.10 mL to about 0.20 mL, about 15 μg to about 400 μg in about 0.10 mL to about 0.20 mL, about 15 μg to about 375 μg in about 0.10 mL to about 0.20 mL, about 15 μg to about 350 μg in about 0.10 mL to about 0.20 mL, about 15 μg to about 325 μg in about 0.10 mL to about 0.20 mL, about 15 μg to about 300 μg in about 0.10 mL to about 0.20 mL, about 15 μg to about 275 μg in about 0.10 mL to about 0.20 mL, about 15 μg to about 250 μg in about 0.10 mL to about 0.20 mL, about 15 μg to about 225 μg in about 0.10 mL to about 0.20 mL, about 15 μg to about 200 μg in about 0.10 mL to about 0.20 mL, about 15 μg to about 175 μg in about 0.10 mL to about 0.20 mL, about 15 μg to about 150 μg in about 0.10 mL to about 0.20 mL, about 15 μg to about 125 μg in about 0.10 mL to about 0.20 mL, about 15 μg to about 100 μg in about 0.10 mL to about 0.20 mL, about 15 μg to about 75 μg in about 0.10 mL to about 0.20 mL, about 15 μg to about 50 μg in about 0.10 mL to about 0.20 mL, about 15 μg to about 25 μg in about 0.10 mL to about 0.20 mL, about 15 μg to about 20 μg in about 0.10 mL to about 0.20 mL, about 20 μg to about 600 μg in about 0.10 mL to about 0.20 mL, about 20 μg to about 575 μg in about 0.10 mL to about 0.20 mL, about 20 μg to about 550 μg in about 0.10 mL to about 0.20 mL, about 20 μg to about 525 μg in about 0.10 mL to about 0.20 mL, about 20 μg to about 500 μg in about 0.10 mL to about 0.20 mL, about 20 μg to about 475 μg in about 0.10 mL to about 0.20 mL, about 20 μg to about 450 μg in about 0.10 mL to about 0.20 mL, about 20 μg to about 425 μg in about 0.10 mL to about 0.20 mL, about 20 μg to about 400 μg in about 0.10 mL to about 0.20 mL, about 20 μg to about 375 μg in about 0.10 mL to about 0.20 mL, about 20 μg to about 350 μg in about 0.10 mL to about 0.20 mL, about 20 μg to about 325 μg in about 0.10 mL to about 0.20 mL, about 20 μg to about 300 μg in about 0.10 mL to about 0.20 mL, about 20 μg to about 275 μg in about 0.10 mL to about 0.20 mL, about 20 μg to about 250 μg in about 0.10 mL to about 0.20 mL, about 20 μg to about 225 μg in about 0.10 mL to about 0.20 mL, about 20 μg to about 200 μg in about 0.10 mL to about 0.20 mL, about 20 μg to about 175 μg in about 0.10 mL to about 0.20 mL, about 20 μg to about 150 μg in about 0.10 mL to about 0.20 mL, about 20 μg to about 125 μg in about 0.10 mL to about 0.20 mL, about 20 μg to about 100 μg in about 0.10 mL to about 0.20 mL, about 20 μg to about 75 μg in about 0.10 mL to about 0.20 mL, about 20 μg to about 50 μg in about 0.10 mL to about 0.20 mL, about 20 μg to about 25 μg in about 0.10 mL to about 0.20 mL, about 50 μg to about 600 μg in about 0.10 mL to about 0.20 mL, about 50 μg to about 575 μg in about 0.10 mL to about 0.20 mL, about 50 μg to about 550 μg in about 0.10 mL to about 0.20 mL, about 50 μg to about 525 μg in about 0.10 mL to about 0.20 mL, about 50 μg to about 500 μg in about 0.10 mL to about 0.20 mL, about 50 μg to about 475 μg in about 0.10 mL to about 0.20 mL, about 50 μg to about 450 μg in about 0.10 mL to about 0.20 mL, about 50 μg to about 425 μg in about 0.10 mL to about 0.20 mL, about 50 μg to about 400 μg in about 0.10 mL to about 0.20 mL, about 50 μg to about 375 μg in about 0.10 mL to about 0.20 mL, about 50 μg to about 350 μg in about 0.10 mL to about 0.20 mL, about 50 μg to about 325 μg in about 0.10 mL to about 0.20 mL, about 50 μg to about 300 μg in about 0.10 mL to about 0.20 mL, about 50 μg to about 275 μg in about 0.10 mL to about 0.20 mL, about 50 μg to about 250 μg in about 0.10 mL to about 0.20 mL, about 50 μg to about 225 μg in about 0.10 mL to about 0.20 mL, about 50 μg to about 200 μg in about 0.10 mL to about 0.20 mL, about 50 μg to about 175 μg in about 0.10 mL to about 0.20 mL, about 50 μg to about 150 μg in about 0.10 mL to about 0.20 mL, about 50 μg to about 125 μg in about 0.10 mL to about 0.20 mL, about 50 μg to about 100 μg in about 0.10 mL to about 0.20 mL, about 50 μg to about 75 μg in about 0.10 mL to about 0.20 mL, about 75 μg to about 600 μg in about 0.10 mL to about 0.20 mL, about 75 μg to about 575 μg in about 0.10 mL to about 0.20 mL, about 75 μg to about 550 μg in about 0.10 mL to about 0.20 mL, about 75 μg to about 525 μg in about 0.10 mL to about 0.20 mL, about 75 μg to about 500 μg in about 0.10 mL to about 0.20 mL, about 75 μg to about 475 μg in about 0.10 mL to about 0.20 mL, about 75 μg to about 450 μg in about 0.10 mL to about 0.20 mL, about 75 μg to about 425 μg in about 0.10 mL to about 0.20 mL, about 75 μg to about 400 μg in about 0.10 mL to about 0.20 mL, about 75 μg to about 375 μg in about 0.10 mL to about 0.20 mL, about 75 μg to about 350 μg in about 0.10 mL to about 0.20 mL, about 75 μg to about 325 μg in about 0.10 mL to about 0.20 mL, about 75 μg to about 300 μg in about 0.10 mL to about 0.20 mL, about 75 μg to about 275 μg in about 0.10 mL to about 0.20 mL, about 75 μg to about 250 μg in about 0.10 mL to about 0.20 mL, about 75 μg to about 225 μg in about 0.10 mL to about 0.20 mL, about 75 μg to about 200 μg in about 0.10 mL to about 0.20 mL, about 75 μg to about 175 μg in about 0.10 mL to about 0.20 mL, about 75 μg to about 150 μg in about 0.10 mL to about 0.20 mL, about 75 μg to about 125 μg in about 0.10 mL to about 0.20 mL, about 75 μg to about 100 μg in about 0.10 mL to about 0.20 mL, about 100 μg to about 600 μg in about 0.10 mL to about 0.20 mL, about 100 μg to about 575 μg in about 0.10 mL to about 0.20 mL, about 100 μg to about 550 μg in about 0.10 mL to about 0.20 mL, about 100 μg to about 525 μg in about 0.10 mL to about 0.20 mL, about 100 μg to about 500 μg in about 0.10 mL to about 0.20 mL, about 100 μg to about 475 μg in about 0.10 mL to about 0.20 mL, about 100 μg to about 450 μg in about 0.10 mL to about 0.20 mL, about 100 μg to about 425 μg in about 0.10 mL to about 0.20 mL, about 100 μg to about 400 μg in about 0.10 mL to about 0.20 mL, about 100 μg to about 375 μg in about 0.10 mL to about 0.20 mL, about 100 μg to about 350 μg in about 0.10 mL to about 0.20 mL, about 100 μg to about 325 μg in about 0.10 mL to about 0.20 mL, about 100 μg to about 300 μg in about 0.10 mL to about 0.20 mL, about 100 μg to about 275 μg in about 0.10 mL to about 0.20 mL, about 100 μg to about 250 μg in about 0.10 mL to about 0.20 mL, about 100 μg to about 225 μg in about 0.10 mL to about 0.20 mL, about 100 μg to about 200 μg in about 0.10 mL to about 0.20 mL, about 100 μg to about 175 μg in about 0.10 mL to about 0.20 mL, about 100 μg to about 150 μg in about 0.10 mL to about 0.20 mL, about 100 μg to about 125 μg in about 0.10 mL to about 0.20 mL, about 125 μg to about 600 μg in about 0.10 mL to about 0.20 mL, about 125 μg to about 575 μg in about 0.10 mL to about 0.20 mL, about 125 μg to about 550 μg in about 0.10 mL to about 0.20 mL, about 125 μg to about 525 μg in about 0.10 mL to about 0.20 mL, about 125 μg to about 500 μg in about 0.10 mL to about 0.20 mL, about 125 μg to about 475 μg in about 0.10 mL to about 0.20 mL, about 125 μg to about 450 μg in about 0.10 mL to about 0.20 mL, about 125 μg to about 425 μg in about 0.10 mL to about 0.20 mL, about 125 μg to about 400 μg in about 0.10 mL to about 0.20 mL, about 125 μg to about 375 μg in about 0.10 mL to about 0.20 mL, about 125 μg to about 350 μg in about 0.10 mL to about 0.20 mL, about 125 μg to about 325 μg in about 0.10 mL to about 0.20 mL, about 125 μg to about 300 μg in about 0.10 mL to about 0.20 mL, about 125 μg to about 275 μg in about 0.10 mL to about 0.20 mL, about 125 μg to about 250 μg in about 0.10 mL to about 0.20 mL, about 125 μg to about 225 μg in about 0.10 mL to about 0.20 mL, about 125 μg to about 200 μg in about 0.10 mL to about 0.20 mL, about 125 μg to about 175 μg in about 0.10 mL to about 0.20 mL, about 125 μg to about 150 μg in about 0.10 mL to about 0.20 mL, about 150 μg to about 600 μg in about 0.10 mL to about 0.20 mL, about 150 μg to about 575 μg in about 0.10 mL to about 0.20 mL, about 150 μg to about 550 μg in about 0.10 mL to about 0.20 mL, about 150 μg to about 525 μg in about 0.10 mL to about 0.20 mL, about 150 μg to about 500 μg in about 0.10 mL to about 0.20 mL, about 150 μg to about 475 μg in about 0.10 mL to about 0.20 mL, about 150 μg to about 450 μg in about 0.10 mL to about 0.20 mL, about 150 μg to about 425 μg in about 0.10 mL to about 0.20 mL, about 150 μg to about 400 μg in about 0.10 mL to about 0.20 mL, about 150 μg to about 375 μg in about 0.10 mL to about 0.20 mL, about 150 μg to about 350 μg in about 0.10 mL to about 0.20 mL, about 150 μg to about 325 μg in about 0.10 mL to about 0.20 mL, about 150 μg to about 300 μg in about 0.10 mL to about 0.20 mL, about 150 μg to about 275 μg in about 0.10 mL to about 0.20 mL, about 150 μg to about 250 μg in about 0.10 mL to about 0.20 mL, about 150 μg to about 225 μg in about 0.10 mL to about 0.20 mL, about 150 μg to about 200 μg in about 0.10 mL to about 0.20 mL, about 150 μg to about 175 μg in about 0.10 mL to about 0.20 mL, about 175 μg to about 600 μg in about 0.10 mL to about 0.20 mL, about 175 μg to about 575 μg in about 0.10 mL to about 0.20 mL, about 175 μg to about 550 μg in about 0.10 mL to about 0.20 mL, about 175 μg to about 525 μg in about 0.10 mL to about 0.20 mL, about 175 μg to about 500 μg in about 0.10 mL to about 0.20 mL, about 175 μg to about 475 μg in about 0.10 mL to about 0.20 mL, about 175 μg to about 450 μg in about 0.10 mL to about 0.20 mL, about 175 μg to about 425 μg in about 0.10 mL to about 0.20 mL, about 175 μg to about 400 μg in about 0.10 mL to about 0.20 mL, about 175 μg to about 375 μg in about 0.10 mL to about 0.20 mL, about 175 μg to about 350 μg in about 0.10 mL to about 0.20 mL, about 175 μg to about 325 μg in about 0.10 mL to about 0.20 mL, about 175 μg to about 300 μg in about 0.10 mL to about 0.20 mL, about 175 μg to about 275 μg in about 0.10 mL to about 0.20 mL, about 175 μg to about 250 μg in about 0.10 mL to about 0.20 mL, about 175 μg to about 225 μg in about 0.10 mL to about 0.20 mL, about 175 μg to about 200 μg in about 0.10 mL to about 0.20 mL, about 200 μg to about 600 μg in about 0.10 mL to about 0.20 mL, about 200 μg to about 575 μg in about 0.10 mL to about 0.20 mL, about 200 μg to about 550 μg in about 0.10 mL to about 0.20 mL, about 200 μg to about 525 μg in about 0.10 mL to about 0.20 mL, about 200 μg to about 500 μg in about 0.10 mL to about 0.20 mL, about 200 μg to about 475 μg in about 0.10 mL to about 0.20 mL, about 200 μg to about 450 μg in about 0.10 mL to about 0.20 mL, about 200 μg to about 425 μg in about 0.10 mL to about 0.20 mL, about 200 μg to about 400 μg in about 0.10 mL to about 0.20 mL, about 200 μg to about 375 μg in about 0.10 mL to about 0.20 mL, about 200 μg to about 350 μg in about 0.10 mL to about 0.20 mL, about 200 μg to about 325 μg in about 0.10 mL to about 0.20 mL, about 200 μg to about 300 μg in about 0.10 mL to about 0.20 mL, about 200 μg to about 275 μg in about 0.10 mL to about 0.20 mL, about 200 μg to about 250 μg in about 0.10 mL to about 0.20 mL, about 200 μg to about 225 μg in about 0.10 mL to about 0.20 mL, about 225 μg to about 600 μg in about 0.10 ml to about 0.20 mL, about 225 μg to about 575 μg in about 0.10 mL to about 0.20 mL, about 225 μg to about 550 μg in about 0.10 mL to about 0.20 mL, about 225 μg to about 525 μg in about 0.10 mL to about 0.20 mL, about 225 μg to about 500 μg in about 0.10 mL to about 0.20 mL, about 225 μg to about 475 μg in about 0.10 mL to about 0.20 mL, about 225 μg to about 450 μg in about 0.10 mL to about 0.20 mL, about 225 μg to about 425 μg in about 0.10 mL to about 0.20 mL, about 225 μg to about 400 μg in about 0.10 mL to about 0.20 mL, about 225 μg to about 375 μg in about 0.10 mL to about 0.20 mL, about 225 μg to about 350 μg in about 0.10 mL to about 0.20 mL, about 225 μg to about 325 μg in about 0.10 mL to about 0.20 mL, about 225 μg to about 300 μg in about 0.10 mL to about 0.20 mL, about 225 μg to about 275 μg in about 0.10 mL to about 0.20 mL, or about 225 μg to about 250 μg in about 0.10 mL to about 0.20 mL. In some embodiments, each of the one or more ampules contains about 5 μg to about 600 μg prostaglandin, prostaglandin analogue, prostacyclin or prostacyclin analogue in about 0.20 mL to about 0.30 mL, about 5 μg to about 575 μg in about 0.20 mL to about 0.30 mL, about 5 μg to about 550 μg in about 0.20 mL to about 0.30 mL, about 5 μg to about 525 μg in about 0.20 mL to about 0.30 mL, about 5 μg to about 500 μg in about 0.20 mL to about 0.30 mL, about 5 μg to about 475 μg in about 0.20 mL to about 0.30 mL, about 5 μg to about 450 μg in about 0.20 mL to about 0.30 mL, about 5 μg to about 425 μg in about 0.20 mL to about 0.30 mL, about 5 μg to about 400 μg in about 0.20 mL to about 0.30 mL, about 5 μg to about 375 μg in about 0.20 mL to about 0.30 mL, about 5 μg to about 350 μg in about 0.20 mL to about 0.30 mL, about 5 μg to about 325 μg in about 0.20 mL to about 0.30 mL, about 5 μg to about 300 μg in about 0.50 mL, about 5 μg to about 275 μg in about 0.20 mL to about 0.30 mL, about 5 μg to about 250 μg in about 0.20 mL to about 0.30 mL, about 5 μg to about 225 μg in about 0.20 mL to about 0.30 mL, about 5 μg to about 200 μg in about 0.20 mL to about 0.30 mL, about 5 μg to about 175 μg in about 0.20 mL to about 0.30 mL, about 5 μg to about 150 μg in about 0.20 mL to about 0.30 mL, about 5 μg to about 125 μg in about 0.20 mL to about 0.30 mL, about 5 μg to about 100 μg in about 0.20 mL to about 0.30 mL, about 5 μg to about 75 μg in about 0.20 mL to about 0.30 mL, about 5 μg to about 50 μg in about 0.20 mL to about 0.30 mL, about 5 μg to about 25 μg in about 0.20 mL to about 0.30 mL, about 5 μg to about 20 μg in about 0.20 mL to about 0.30 mL, about 5 μg to about 15 μg in about 0.20 mL to about 0.30 mL, about 5 μg to about 10 μg in about 0.20 mL to about 0.30 mL, about 10 μg to about 600 μg in about 0.20 mL to about 0.30 mL, about 10 μg to about 575 μg in about 0.20 mL to about 0.30 mL, about 10 μg to about 550 μg in about 0.20 mL to about 0.30 mL, about 10 μg to about 525 μg in about 0.20 mL to about 0.30 mL, about 10 μg to about 500 μg in about 0.20 mL to about 0.30 mL, about 10 μg to about 475 μg in about 0.20 mL to about 0.30 mL, about 10 μg to about 450 μg in about 0.20 mL to about 0.30 mL, about 10 μg to about 425 μg in about 0.20 mL to about 0.30 mL, about 10 μg to about 400 μg in about 0.20 mL to about 0.30 mL, about 10 μg to about 375 μg in about 0.20 mL to about 0.30 mL, about 10 μg to about 350 μg in about 0.20 mL to about 0.30 mL, about 10 μg to about 325 μg in about 0.20 mL to about 0.30 mL, about 10 μg to about 300 μg in about 0.20 mL to about 0.30 mL, about 10 μg to about 275 μg in about 0.20 mL to about 0.30 mL, about 10 μg to about 250 μg in about 0.20 mL to about 0.30 mL, about 10 μg to about 225 μg in about 0.20 mL to about 0.30 mL, about 10 μg to about 200 μg in about 0.20 mL to about 0.30 mL, about 10 μg to about 175 μg in about 0.20 mL to about 0.30 mL, about 10 μg to about 150 μg in about 0.20 mL to about 0.30 mL, about 10 μg to about 125 μg in about 0.20 mL to about 0.30 mL, about 10 μg to about 100 μg in about 0.20 mL to about 0.30 mL, about 10 μg to about 75 μg in about 0.20 mL to about 0.30 mL, about 10 μg to about 50 μg in about 0.20 mL to about 0.30 mL, about 10 μg to about 25 μg in about 0.20 mL to about 0.30 mL, about 10 μg to about 20 μg in about 0.20 mL to about 0.30 mL, about 10 μg to about 15 μg in about 0.20 mL to about 0.30 mL, about 15 μg to about 600 μg in about 0.20 mL to about 0.30 mL, about 15 μg to about 575 μg in about 0.20 mL to about 0.30 mL, about 15 μg to about 550 μg in about 0.20 mL to about 0.30 mL, about 15 μg to about 525 μg in about 0.20 mL to about 0.30 mL, about 15 μg to about 500 μg in about 0.20 mL to about 0.30 mL, about 15 μg to about 475 μg in about 0.20 mL to about 0.30 mL, about 15 μg to about 450 μg in about 0.20 mL to about 0.30 mL, about 15 μg to about 425 μg in about 0.20 mL to about 0.30 mL, about 15 μg to about 400 μg in about 0.20 mL to about 0.30 mL, about 15 μg to about 375 μg in about 0.20 mL to about 0.30 mL, about 15 μg to about 350 μg in about 0.20 mL to about 0.30 mL, about 15 μg to about 325 μg in about 0.20 mL to about 0.30 mL, about 15 μg to about 300 μg in about 0.20 mL to about 0.30 mL, about 15 μg to about 275 μg in about 0.20 mL to about 0.30 mL, about 15 μg to about 250 μg in about 0.20 mL to about 0.30 mL, about 15 μg to about 225 μg in about 0.20 mL to about 0.30 mL, about 15 μg to about 200 μg in about 0.20 mL to about 0.30 mL, about 15 μg to about 175 μg in about 0.20 mL to about 0.30 mL, about 15 μg to about 150 μg in about 0.20 mL to about 0.30 mL, about 15 μg to about 125 μg in about 0.20 mL to about 0.30 mL, about 15 μg to about 100 μg in about 0.20 mL to about 0.30 mL, about 15 μg to about 75 μg in about 0.20 mL to about 0.30 mL, about 15 μg to about 50 μg in about 0.20 mL to about 0.30 mL, about 15 μg to about 25 μg in about 0.20 mL to about 0.30 mL, about 15 μg to about 20 μg in about 0.20 mL to about 0.30 mL, about 20 μg to about 600 μg in about 0.20 mL to about 0.30 mL, about 20 μg to about 575 μg in about 0.20 mL to about 0.30 mL, about 20 μg to about 550 μg in about 0.20 mL to about 0.30 mL, about 20 μg to about 525 μg in about 0.20 mL to about 0.30 mL, about 20 μg to about 500 μg in about 0.20 mL to about 0.30 mL, about 20 μg to about 475 μg in about 0.20 mL to about 0.30 mL, about 20 μg to about 450 μg in about 0.20 mL to about 0.30 mL, about 20 μg to about 425 μg in about 0.20 mL to about 0.30 mL, about 20 μg to about 400 μg in about 0.20 mL to about 0.30 mL, about 20 μg to about 375 μg in about 0.20 mL to about 0.30 mL, about 20 μg to about 350 μg in about 0.20 mL to about 0.30 mL, about 20 μg to about 325 μg in about 0.20 mL to about 0.30 mL, about 20 μg to about 300 μg in about 0.20 mL to about 0.30 mL, about 20 μg to about 275 μg in about 0.20 mL to about 0.30 mL, about 20 μg to about 250 μg in about 0.20 mL to about 0.30 mL, about 20 μg to about 225 μg in about 0.20 mL to about 0.30 mL, about 20 μg to about 200 μg in about 0.20 mL to about 0.30 mL, about 20 μg to about 175 μg in about 0.20 mL to about 0.30 mL, about 20 μg to about 150 μg in about 0.20 mL to about 0.30 mL, about 20 μg to about 125 μg in about 0.20 mL to about 0.30 mL, about 20 μg to about 100 μg in about 0.20 mL to about 0.30 mL, about 20 μg to about 75 μg in about 0.20 mL to about 0.30 mL, about 20 μg to about 50 μg in about 0.20 mL to about 0.30 mL, about 20 μg to about 25 μg in about 0.20 mL to about 0.30 mL, about 50 μg to about 600 μg in about 0.20 mL to about 0.30 mL, about 50 μg to about 575 μg in about 0.20 mL to about 0.30 mL, about 50 μg to about 550 μg in about 0.20 mL to about 0.30 mL, about 50 μg to about 525 μg in about 0.20 mL to about 0.30 mL, about 50 μg to about 500 μg in about 0.20 mL to about 0.30 mL, about 50 μg to about 475 μg in about 0.20 mL to about 0.30 mL, about 50 μg to about 450 μg in about 0.20 mL to about 0.30 mL, about 50 μg to about 425 μg in about 0.20 mL to about 0.30 mL, about 50 μg to about 400 μg in about 0.20 mL to about 0.30 mL, about 50 μg to about 375 μg in about 0.20 mL to about 0.30 mL, about 50 μg to about 350 μg in about 0.20 mL to about 0.30 mL, about 50 μg to about 325 μg in about 0.20 mL to about 0.30 mL, about 50 μg to about 300 μg in about 0.20 mL to about 0.30 mL, about 50 μg to about 275 μg in about 0.20 mL to about 0.30 mL, about 50 μg to about 250 μg in about 0.20 mL to about 0.30 mL, about 50 μg to about 225 μg in about 0.20 mL to about 0.30 mL, about 50 μg to about 200 μg in about 0.20 mL to about 0.30 mL, about 50 μg to about 175 μg in about 0.20 mL to about 0.30 mL, about 50 μg to about 150 μg in about 0.20 mL to about 0.30 mL, about 50 μg to about 125 μg in about 0.20 mL to about 0.30 mL, about 50 μg to about 100 μg in about 0.20 mL to about 0.30 mL, about 50 μg to about 75 μg in about 0.20 mL to about 0.30 mL, about 75 μg to about 600 μg in about 0.20 mL to about 0.30 mL, about 75 μg to about 575 μg in about 0.20 mL to about 0.30 mL, about 75 μg to about 550 μg in about 0.20 mL to about 0.30 mL, about 75 μg to about 525 μg in about 0.20 mL to about 0.30 mL, about 75 μg to about 500 μg in about 0.20 mL to about 0.30 mL, about 75 μg to about 475 μg in about 0.20 mL to about 0.30 mL, about 75 μg to about 450 μg in about 0.20 mL to about 0.30 mL, about 75 μg to about 425 μg in about 0.20 mL to about 0.30 mL, about 75 μg to about 400 μg in about 0.20 mL to about 0.30 mL, about 75 μg to about 375 μg in about 0.20 mL to about 0.30 mL, about 75 μg to about 350 μg in about 0.20 mL to about 0.30 mL, about 75 μg to about 325 μg in about 0.20 mL to about 0.30 mL, about 75 μg to about 300 μg in about 0.20 mL to about 0.30 mL, about 75 μg to about 275 μg in about 0.20 mL to about 0.30 mL, about 75 μg to about 250 μg in about 0.20 mL to about 0.30 mL, about 75 μg to about 225 μg in about 0.20 mL to about 0.30 mL, about 75 μg to about 200 μg in about 0.20 mL to about 0.30 mL, about 75 μg to about 175 μg in about 0.20 mL to about 0.30 mL, about 75 μg to about 150 μg in about 0.20 mL to about 0.30 mL, about 75 μg to about 125 μg in about 0.20 mL to about 0.30 mL, about 75 μg to about 100 μg in about 0.20 mL to about 0.30 mL, about 100 μg to about 600 μg in about 0.20 mL to about 0.30 mL, about 100 μg to about 575 μg in about 0.20 mL to about 0.30 mL, about 100 μg to about 550 μg in about 0.20 mL to about 0.30 mL, about 100 μg to about 525 μg in about 0.20 mL to about 0.30 mL, about 100 μg to about 500 μg in about 0.20 mL to about 0.30 mL, about 100 μg to about 475 μg in about 0.20 mL to about 0.30 mL, about 100 μg to about 450 μg in about 0.20 mL to about 0.30 mL, about 100 μg to about 425 μg in about 0.20 mL to about 0.30 mL, about 100 μg to about 400 μg in about 0.20 mL to about 0.30 mL, about 100 μg to about 375 μg in about 0.20 mL to about 0.30 mL, about 100 μg to about 350 μg in about 0.20 mL to about 0.30 mL, about 100 μg to about 325 μg in about 0.20 mL to about 0.30 mL, about 100 μg to about 300 μg in about 0.20 mL to about 0.30 mL, about 100 μg to about 275 μg in about 0.20 mL to about 0.30 mL, about 100 μg to about 250 μg in about 0.20 mL to about 0.30 mL, about 100 μg to about 225 μg in about 0.20 mL to about 0.30 mL, about 100 μg to about 200 μg in about 0.20 mL to about 0.30 mL, about 100 μg to about 175 μg in about 0.20 mL to about 0.30 mL, about 100 μg to about 150 μg in about 0.20 mL to about 0.30 mL, about 100 μg to about 125 μg in about 0.20 mL to about 0.30 mL, about 125 μg to about 600 μg in about 0.20 mL to about 0.30 mL, about 125 μg to about 575 μg in about 0.20 mL to about 0.30 mL, about 125 μg to about 550 μg in about 0.20 mL to about 0.30 mL, about 125 μg to about 525 μg in about 0.20 mL to about 0.30 mL, about 125 μg to about 500 μg in about 0.20 mL to about 0.30 mL, about 125 μg to about 475 μg in about 0.20 mL to about 0.30 mL, about 125 μg to about 450 μg in about 0.20 mL to about 0.30 mL, about 125 μg to about 425 μg in about 0.20 mL to about 0.30 mL, about 125 μg to about 400 μg in about 0.20 mL to about 0.30 mL, about 125 μg to about 375 μg in about 0.20 mL to about 0.30 mL, about 125 μg to about 350 μg in about 0.20 mL to about 0.30 mL, about 125 μg to about 325 μg in about 0.20 mL to about 0.30 mL, about 125 μg to about 300 μg in about 0.20 mL to about 0.30 mL, about 125 μg to about 275 μg in about 0.20 mL to about 0.30 mL, about 125 μg to about 250 μg in about 0.20 mL to about 0.30 mL, about 125 μg to about 225 μg in about 0.20 mL to about 0.30 mL, about 125 μg to about 200 μg in about 0.20 mL to about 0.30 mL, about 125 μg to about 175 μg in about 0.20 mL to about 0.30 mL, about 125 μg to about 150 μg in about 0.20 mL to about 0.30 mL, about 150 μg to about 600 μg in about 0.20 mL to about 0.30 mL, about 150 μg to about 575 μg in about 0.20 mL to about 0.30 mL, about 150 μg to about 550 μg in about 0.20 mL to about 0.30 mL, about 150 μg to about 525 μg in about 0.20 mL to about 0.30 mL, about 150 μg to about 500 μg in about 0.20 mL to about 0.30 mL, about 150 μg to about 475 μg in about 0.20 mL to about 0.30 mL, about 150 μg to about 450 μg in about 0.20 mL to about 0.30 mL, about 150 μg to about 425 μg in about 0.20 mL to about 0.30 mL, about 150 μg to about 400 μg in about 0.20 mL to about 0.30 mL, about 150 μg to about 375 μg in about 0.20 mL to about 0.30 mL, about 150 μg to about 350 μg in about 0.20 mL to about 0.30 mL, about 150 μg to about 325 μg in about 0.20 mL to about 0.30 mL, about 150 μg to about 300 μg in about 0.20 mL to about 0.30 mL, about 150 μg to about 275 μg in about 0.20 mL to about 0.30 mL, about 150 μg to about 250 μg in about 0.20 mL to about 0.30 mL, about 150 μg to about 225 μg in about 0.20 mL to about 0.30 mL, about 150 μg to about 200 μg in about 0.20 mL to about 0.30 mL, about 150 μg to about 175 μg in about 0.20 mL to about 0.30 mL, about 175 μg to about 600 μg in about 0.20 mL to about 0.30 mL, about 175 μg to about 575 μg in about 0.20 mL to about 0.30 mL, about 175 μg to about 550 μg in about 0.20 mL to about 0.30 mL, about 175 μg to about 525 μg in about 0.20 mL to about 0.30 mL, about 175 μg to about 500 μg in about 0.20 mL to about 0.30 mL, about 175 μg to about 475 μg in about 0.20 mL to about 0.30 mL, about 175 μg to about 450 μg in about 0.20 mL to about 0.30 mL, about 175 μg to about 425 μg in about 0.20 mL to about 0.30 mL, about 175 μg to about 400 μg in about 0.20 mL to about 0.30 mL, about 175 μg to about 375 μg in about 0.20 mL to about 0.30 mL, about 175 μg to about 350 μg in about 0.20 mL to about 0.30 mL, about 175 μg to about 325 μg in about 0.20 mL to about 0.30 mL, about 175 μg to about 300 μg in about 0.20 mL to about 0.30 mL, about 175 μg to about 275 μg in about 0.20 mL to about 0.30 mL, about 175 μg to about 250 μg in about 0.20 mL to about 0.30 mL, about 175 μg to about 225 μg in about 0.20 mL to about 0.30 mL, about 175 μg to about 200 μg in about 0.20 mL to about 0.30 mL, about 200 μg to about 600 μg in about 0.20 mL to about 0.30 mL, about 200 μg to about 575 μg in about 0.20 mL to about 0.30 mL, about 200 μg to about 550 μg in about 0.20 mL to about 0.30 mL, about 200 μg to about 525 μg in about 0.20 mL to about 0.30 mL, about 200 μg to about 500 μg in about 0.20 mL to about 0.30 mL, about 200 μg to about 475 μg in about 0.20 mL to about 0.30 mL, about 200 μg to about 450 μg in about 0.20 mL to about 0.30 mL, about 200 μg to about 425 μg in about 0.20 mL to about 0.30 mL, about 200 μg to about 400 μg in about 0.20 mL to about 0.30 mL, about 200 μg to about 375 μg in about 0.20 mL to about 0.30 mL, about 200 μg to about 350 μg in about 0.20 mL to about 0.30 mL, about 200 μg to about 325 μg in about 0.20 mL to about 0.30 mL, about 200 μg to about 300 μg in about 0.20 mL to about 0.30 mL, about 200 μg to about 275 μg in about 0.20 mL to about 0.30 mL, about 200 μg to about 250 μg in about 0.20 mL to about 0.30 mL, about 200 μg to about 225 μg in about 0.20 mL to about 0.30 mL, about 225 μg to about 600 μg in about 0.20 mL to about 0.30 mL, about 225 μg to about 575 μg in about 0.20 mL to about 0.30 mL, about 225 μg to about 550 μg in about 0.20 mL to about 0.30 mL, about 225 μg to about 525 μg in about 0.20 mL to about 0.30 mL, about 225 μg to about 500 μg in about 0.20 mL to about 0.30 mL, about 225 μg to about 475 μg in about 0.20 mL to about 0.30 mL, about 225 μg to about 450 μg in about 0.20 mL to about 0.30 mL, about 225 μg to about 425 μg in about 0.20 mL to about 0.30 mL, about 225 μg to about 400 μg in about 0.20 mL to about 0.30 mL, about 225 μg to about 375 μg in about 0.20 mL to about 0.30 mL, about 225 μg to about 350 μg in about 0.20 mL to about 0.30 mL, about 225 μg to about 325 μg in about 0.20 mL to about 0.30 mL, about 225 μg to about 300 μg in about 0.20 mL to about 0.30 mL, about 225 μg to about 275 μg in about 0.20 mL to about 0.30 mL, or about 225 μg to about 250 μg in about 0.20 mL to about 0.30 mL.

In some embodiments, the reservoir of the nebulizer contains prostaglandin, prostaglandin analogue, prostacyclin or prostacyclin analogue in an amount of about 5 μg to about 600 μg in about 0.30 mL to about 0.40 mL, about 5 μg to about 575 μg in about 0.30 mL to about 0.40 mL, about 5 μg to about 550 μg in about 0.30 mL to about 0.40 mL, about 5 μg to about 525 μg in about 0.30 mL to about 0.40 mL, about 5 μg to about 500 μg in about 0.30 mL to about 0.40 mL, about 5 μg to about 475 μg in about 0.30 mL to about 0.40 mL, about 5 μg to about 450 μg in about 0.30 mL to about 0.40 mL, about 5 μg to about 425 μg in about 0.30 mL to about 0.40 mL, about 5 μg to about 400 μg in about 0.30 mL to about 0.40 mL, about 5 μg to about 375 μg in about 0.30 mL to about 0.40 mL, about 5 μg to about 350 μg in about 0.30 mL to about 0.40 mL, about 5 μg to about 325 μg in about 0.30 mL to about 0.40 mL, about 5 μg to about 300 μg in about 0.50 mL, about 5 μg to about 275 μg in about 0.30 mL to about 0.40 mL, about 5 μg to about 250 μg in about 0.30 mL to about 0.40 mL, about 5 μg to about 225 μg in about 0.30 mL to about 0.40 mL, about 5 μg to about 200 μg in about 0.30 mL to about 0.40 mL, about 5 μg to about 175 μg in about 0.30 mL to about 0.40 mL, about 5 μg to about 150 μg in about 0.30 mL to about 0.40 mL, about 5 μg to about 125 μg in about 0.30 mL to about 0.40 mL, about 5 μg to about 100 μg in about 0.30 mL to about 0.40 mL, about 5 μg to about 75 μg in about 0.30 mL to about 0.40 mL, about 5 μg to about 50 μg in about 0.30 mL to about 0.40 mL, about 5 μg to about 25 μg in about 0.30 mL to about 0.40 mL, about 5 μg to about 20 μg in about 0.30 mL to about 0.40 mL, about 5 μg to about 15 μg in about 0.30 mL to about 0.40 mL, about 5 μg to about 10 μg in about 0.30 mL to about 0.40 mL, about 10 μg to about 600 μg in about 0.30 mL to about 0.40 mL, about 10 μg to about 575 μg in about 0.30 mL to about 0.40 mL, about 10 μg to about 550 μg in about 0.30 mL to about 0.40 mL, about 10 μg to about 525 μg in about 0.30 mL to about 0.40 mL, about 10 μg to about 500 μg in about 0.30 mL to about 0.40 mL, about 10 μg to about 475 μg in about 0.30 mL to about 0.40 mL, about 10 μg to about 450 μg in about 0.30 mL to about 0.40 mL, about 10 μg to about 425 μg in about 0.30 mL to about 0.40 mL, about 10 μg to about 400 μg in about 0.30 mL to about 0.40 mL, about 10 μg to about 375 μg in about 0.30 mL to about 0.40 mL, about 10 μg to about 350 μg in about 0.30 mL to about 0.40 mL, about 10 μg to about 325 μg in about 0.30 mL to about 0.40 mL, about 10 μg to about 300 μg in about 0.30 mL to about 0.40 mL, about 10 μg to about 275 μg in about 0.30 mL to about 0.40 mL, about 10 μg to about 250 μg in about 0.30 mL to about 0.40 mL, about 10 μg to about 225 μg in about 0.30 mL to about 0.40 mL, about 10 μg to about 200 μg in about 0.30 mL to about 0.40 mL, about 10 μg to about 175 μg in about 0.30 mL to about 0.40 mL, about 10 μg to about 150 μg in about 0.30 mL to about 0.40 mL, about 10 μg to about 125 μg in about 0.30 mL to about 0.40 mL, about 10 μg to about 100 μg in about 0.30 mL to about 0.40 mL, about 10 μg to about 75 μg in about 0.30 mL to about 0.40 mL, about 10 μg to about 50 μg in about 0.30 mL to about 0.40 mL, about 10 μg to about 25 μg in about 0.30 mL to about 0.40 mL, about 10 μg to about 20 μg in about 0.30 mL to about 0.40 mL, about 10 μg to about 15 μg in about 0.30 mL to about 0.40 mL, about 15 μg to about 600 μg in about 0.30 mL to about 0.40 mL, about 15 μg to about 575 μg in about 0.30 mL to about 0.40 mL, about 15 μg to about 550 μg in about 0.30 mL to about 0.40 mL, about 15 μg to about 525 μg in about 0.30 mL to about 0.40 mL, about 15 μg to about 500 μg in about 0.30 mL to about 0.40 mL, about 15 μg to about 475 μg in about 0.30 mL to about 0.40 mL, about 15 μg to about 450 μg in about 0.30 mL to about 0.40 mL, about 15 μg to about 425 μg in about 0.30 mL to about 0.40 mL, about 15 μg to about 400 μg in about 0.30 mL to about 0.40 mL, about 15 μg to about 375 μg in about 0.30 mL to about 0.40 mL, about 15 μg to about 350 μg in about 0.30 mL to about 0.40 mL, about 15 μg to about 325 μg in about 0.30 mL to about 0.40 mL, about 15 μg to about 300 μg in about 0.30 mL to about 0.40 mL, about 15 μg to about 275 μg in about 0.30 mL to about 0.40 mL, about 15 μg to about 250 μg in about 0.30 mL to about 0.40 mL, about 15 μg to about 225 μg in about 0.30 mL to about 0.40 mL, about 15 μg to about 200 μg in about 0.30 mL to about 0.40 mL, about 15 μg to about 175 μg in about 0.30 mL to about 0.40 mL, about 15 μg to about 150 μg in about 0.30 mL to about 0.40 mL, about 15 μg to about 125 μg in about 0.30 mL to about 0.40 mL, about 15 μg to about 100 μg in about 0.30 mL to about 0.40 mL, about 15 μg to about 75 μg in about 0.30 mL to about 0.40 mL, about 15 μg to about 50 μg in about 0.30 mL to about 0.40 mL, about 15 μg to about 25 μg in about 0.30 mL to about 0.40 mL, about 15 μg to about 20 μg in about 0.30 mL to about 0.40 mL, about 20 μg to about 600 μg in about 0.30 mL to about 0.40 mL, about 20 μg to about 575 μg in about 0.30 mL to about 0.40 mL, about 20 μg to about 550 μg in about 0.30 mL to about 0.40 mL, about 20 μg to about 525 μg in about 0.30 mL to about 0.40 mL, about 20 μg to about 500 μg in about 0.30 mL to about 0.40 mL, about 20 μg to about 475 μg in about 0.30 mL to about 0.40 mL, about 20 μg to about 450 μg in about 0.30 mL to about 0.40 mL, about 20 μg to about 425 μg in about 0.30 mL to about 0.40 mL, about 20 μg to about 400 μg in about 0.30 mL to about 0.40 mL, about 20 μg to about 375 μg in about 0.30 mL to about 0.40 mL, about 20 μg to about 350 μg in about 0.30 mL to about 0.40 mL, about 20 μg to about 325 μg in about 0.30 mL to about 0.40 mL, about 20 μg to about 300 μg in about 0.30 mL to about 0.40 mL, about 20 μg to about 275 μg in about 0.30 mL to about 0.40 mL, about 20 μg to about 250 μg in about 0.30 mL to about 0.40 mL, about 20 μg to about 225 μg in about 0.30 mL to about 0.40 mL, about 20 μg to about 200 μg in about 0.30 mL to about 0.40 mL, about 20 μg to about 175 μg in about 0.30 mL to about 0.40 mL, about 20 μg to about 150 μg in about 0.30 mL to about 0.40 mL, about 20 μg to about 125 μg in about 0.30 mL to about 0.40 mL, about 20 μg to about 100 μg in about 0.30 mL to about 0.40 mL, about 20 μg to about 75 μg in about 0.30 mL to about 0.40 mL, about 20 μg to about 50 μg in about 0.30 mL to about 0.40 mL, about 20 μg to about 25 μg in about 0.30 mL to about 0.40 mL, about 50 μg to about 600 μg in about 0.30 mL to about 0.40 mL, about 50 μg to about 575 μg in about 0.30 mL to about 0.40 mL, about 50 μg to about 550 μg in about 0.30 mL to about 0.40 mL, about 50 μg to about 525 μg in about 0.30 mL to about 0.40 mL, about 50 μg to about 500 μg in about 0.30 mL to about 0.40 mL, about 50 μg to about 475 μg in about 0.30 mL to about 0.40 mL, about 50 μg to about 450 μg in about 0.30 mL to about 0.40 mL, about 50 μg to about 425 μg in about 0.30 mL to about 0.40 mL, about 50 μg to about 400 μg in about 0.30 mL to about 0.40 mL, about 50 μg to about 375 μg in about 0.30 mL to about 0.40 mL, about 50 μg to about 350 μg in about 0.30 mL to about 0.40 mL, about 50 μg to about 325 μg in about 0.30 mL to about 0.40 mL, about 50 μg to about 300 μg in about 0.30 mL to about 0.40 mL, about 50 μg to about 275 μg in about 0.30 mL to about 0.40 mL, about 50 μg to about 250 μg in about 0.30 mL to about 0.40 mL, about 50 μg to about 225 μg in about 0.30 mL to about 0.40 mL, about 50 μg to about 200 μg in about 0.30 mL to about 0.40 mL, about 50 μg to about 175 μg in about 0.30 mL to about 0.40 mL, about 50 μg to about 150 μg in about 0.30 mL to about 0.40 mL, about 50 μg to about 125 μg in about 0.30 mL to about 0.40 mL, about 50 μg to about 100 μg in about 0.30 mL to about 0.40 mL, about 50 μg to about 75 μg in about 0.30 mL to about 0.40 mL, about 75 μg to about 600 μg in about 0.30 mL to about 0.40 mL, about 75 μg to about 575 μg in about 0.30 mL to about 0.40 mL, about 75 μg to about 550 μg in about 0.30 mL to about 0.40 mL, about 75 μg to about 525 μg in about 0.30 mL to about 0.40 mL, about 75 μg to about 500 μg in about 0.30 mL to about 0.40 mL, about 75 μg to about 475 μg in about 0.30 mL to about 0.40 mL, about 75 μg to about 450 μg in about 0.30 mL to about 0.40 mL, about 75 μg to about 425 μg in about 0.30 mL to about 0.40 mL, about 75 μg to about 400 μg in about 0.30 mL to about 0.40 mL, about 75 μg to about 375 μg in about 0.30 mL to about 0.40 mL, about 75 μg to about 350 μg in about 0.30 mL to about 0.40 mL, about 75 μg to about 325 μg in about 0.30 mL to about 0.40 mL, about 75 μg to about 300 μg in about 0.30 mL to about 0.40 mL, about 75 μg to about 275 μg in about 0.30 mL to about 0.40 mL, about 75 μg to about 250 μg in about 0.30 mL to about 0.40 mL, about 75 μg to about 225 μg in about 0.30 mL to about 0.40 mL, about 75 μg to about 200 μg in about 0.30 mL to about 0.40 mL, about 75 μg to about 175 μg in about 0.30 mL to about 0.40 mL, about 75 μg to about 150 μg in about 0.30 mL to about 0.40 mL, about 75 μg to about 125 μg in about 0.30 mL to about 0.40 mL, about 75 μg to about 100 μg in about 0.30 mL to about 0.40 mL, about 100 μg to about 600 μg in about 0.30 mL to about 0.40 mL, about 100 μg to about 575 μg in about 0.30 mL to about 0.40 mL, about 100 μg to about 550 μg in about 0.30 mL to about 0.40 mL, about 100 μg to about 525 μg in about 0.30 mL to about 0.40 mL, about 100 μg to about 500 μg in about 0.30 mL to about 0.40 mL, about 100 μg to about 475 μg in about 0.30 mL to about 0.40 mL, about 100 μg to about 450 μg in about 0.30 mL to about 0.40 mL, about 100 μg to about 425 μg in about 0.30 mL to about 0.40 mL, about 100 μg to about 400 μg in about 0.30 mL to about 0.40 mL, about 100 μg to about 375 μg in about 0.30 mL to about 0.40 mL, about 100 μg to about 350 μg in about 0.30 mL to about 0.40 mL, about 100 μg to about 325 μg in about 0.30 mL to about 0.40 mL, about 100 μg to about 300 μg in about 0.30 mL to about 0.40 mL, about 100 μg to about 275 μg in about 0.30 mL to about 0.40 mL, about 100 μg to about 250 μg in about 0.30 mL to about 0.40 mL, about 100 μg to about 225 μg in about 0.30 mL to about 0.40 mL, about 100 μg to about 200 μg in about 0.30 mL to about 0.40 mL, about 100 μg to about 175 μg in about 0.30 mL to about 0.40 mL, about 100 μg to about 150 μg in about 0.30 mL to about 0.40 mL, about 100 μg to about 125 μg in about 0.30 mL to about 0.40 mL, about 125 μg to about 600 μg in about 0.30 mL to about 0.40 mL, about 125 μg to about 575 μg in about 0.30 mL to about 0.40 mL, about 125 μg to about 550 μg in about 0.30 mL to about 0.40 mL, about 125 μg to about 525 μg in about 0.30 mL to about 0.40 mL, about 125 μg to about 500 μg in about 0.30 mL to about 0.40 mL, about 125 μg to about 475 μg in about 0.30 mL to about 0.40 mL, about 125 μg to about 450 μg in about 0.30 mL to about 0.40 mL, about 125 μg to about 425 μg in about 0.30 mL to about 0.40 mL, about 125 μg to about 400 μg in about 0.30 mL to about 0.40 mL, about 125 μg to about 375 μg in about 0.30 mL to about 0.40 mL, about 125 μg to about 350 μg in about 0.30 mL to about 0.40 mL, about 125 μg to about 325 μg in about 0.30 mL to about 0.40 mL, about 125 μg to about 300 μg in about 0.30 mL to about 0.40 mL, about 125 μg to about 275 μg in about 0.30 mL to about 0.40 mL, about 125 μg to about 250 μg in about 0.30 mL to about 0.40 mL, about 125 μg to about 225 μg in about 0.30 mL to about 0.40 mL, about 125 μg to about 200 μg in about 0.30 mL to about 0.40 mL, about 125 μg to about 175 μg in about 0.30 mL to about 0.40 mL, about 125 μg to about 150 μg in about 0.30 mL to about 0.40 mL, about 150 μg to about 600 μg in about 0.30 mL to about 0.40 mL, about 150 μg to about 575 μg in about 0.30 mL to about 0.40 mL, about 150 μg to about 550 μg in about 0.30 mL to about 0.40 mL, about 150 μg to about 525 μg in about 0.30 mL to about 0.40 mL, about 150 μg to about 500 μg in about 0.30 mL to about 0.40 mL, about 150 μg to about 475 μg in about 0.30 mL to about 0.40 mL, about 150 μg to about 450 μg in about 0.30 mL to about 0.40 mL, about 150 μg to about 425 μg in about 0.30 mL to about 0.40 mL, about 150 μg to about 400 μg in about 0.30 mL to about 0.40 mL, about 150 μg to about 375 μg in about 0.30 mL to about 0.40 mL, about 150 μg to about 350 μg in about 0.30 mL to about 0.40 mL, about 150 μg to about 325 μg in about 0.30 mL to about 0.40 mL, about 150 μg to about 300 μg in about 0.30 mL to about 0.40 mL, about 150 μg to about 275 μg in about 0.30 mL to about 0.40 mL, about 150 μg to about 250 μg in about 0.30 mL to about 0.40 mL, about 150 μg to about 225 μg in about 0.30 mL to about 0.40 mL, about 150 μg to about 200 μg in about 0.30 mL to about 0.40 mL, about 150 μg to about 175 μg in about 0.30 mL to about 0.40 mL, about 175 μg to about 600 μg in about 0.30 mL to about 0.40 mL, about 175 μg to about 575 μg in about 0.30 mL to about 0.40 mL, about 175 μg to about 550 μg in about 0.30 mL to about 0.40 mL, about 175 μg to about 525 μg in about 0.30 mL to about 0.40 mL, about 175 μg to about 500 μg in about 0.30 mL to about 0.40 mL, about 175 μg to about 475 μg in about 0.30 mL to about 0.40 mL, about 175 μg to about 450 μg in about 0.30 mL to about 0.40 mL, about 175 μg to about 425 μg in about 0.30 mL to about 0.40 mL, about 175 μg to about 400 μg in about 0.30 mL to about 0.40 mL, about 175 μg to about 375 μg in about 0.30 mL to about 0.40 mL, about 175 μg to about 350 μg in about 0.30 mL to about 0.40 mL, about 175 μg to about 325 μg in about 0.30 mL to about 0.40 mL, about 175 μg to about 300 μg in about 0.30 mL to about 0.40 mL, about 175 μg to about 275 μg in about 0.30 mL to about 0.40 mL, about 175 μg to about 250 μg in about 0.30 mL to about 0.40 mL, about 175 μg to about 225 μg in about 0.30 mL to about 0.40 mL, about 175 μg to about 200 μg in about 0.30 mL to about 0.40 mL, about 200 μg to about 600 μg in about 0.30 mL to about 0.40 mL, about 200 μg to about 575 μg in about 0.30 mL to about 0.40 mL, about 200 μg to about 550 μg in about 0.30 mL to about 0.40 mL, about 200 μg to about 525 μg in about 0.30 mL to about 0.40 mL, about 200 μg to about 500 μg in about 0.30 mL to about 0.40 mL, about 200 μg to about 475 μg in about 0.30 mL to about 0.40 mL, about 200 μg to about 450 μg in about 0.30 mL to about 0.40 mL, about 200 μg to about 425 μg in about 0.30 mL to about 0.40 mL, about 200 μg to about 400 μg in about 0.30 mL to about 0.40 mL, about 200 μg to about 375 μg in about 0.30 mL to about 0.40 mL, about 200 μg to about 350 μg in about 0.30 mL to about 0.40 mL, about 200 μg to about 325 μg in about 0.30 mL to about 0.40 mL, about 200 μg to about 300 μg in about 0.30 mL to about 0.40 mL, about 200 μg to about 275 μg in about 0.30 mL to about 0.40 mL, about 200 μg to about 250 μg in about 0.30 mL to about 0.40 mL, about 200 μg to about 225 μg in about 0.30 mL to about 0.40 mL, about 225 μg to about 600 μg in about 0.30 mL to about 0.40 mL, about 225 μg to about 575 μg in about 0.30 mL to about 0.40 mL, about 225 μg to about 550 μg in about 0.30 mL to about 0.40 mL, about 225 μg to about 525 μg in about 0.30 mL to about 0.40 mL, about 225 μg to about 500 μg in about 0.30 mL to about 0.40 mL, about 225 μg to about 475 μg in about 0.30 mL to about 0.40 mL, about 225 μg to about 450 μg in about 0.30 mL to about 0.40 mL, about 225 μg to about 425 μg in about 0.30 mL to about 0.40 mL, about 225 μg to about 400 μg in about 0.30 mL to about 0.40 mL, about 225 μg to about 375 μg in about 0.30 mL to about 0.40 mL, about 225 μg to about 350 μg in about 0.30 mL to about 0.40 mL, about 225 μg to about 325 μg in about 0.30 mL to about 0.40 mL, about 225 μg to about 300 μg in about 0.30 mL to about 0.40 mL, about 225 μg to about 275 μg in about 0.30 mL to about 0.40 mL, or about 225 μg to about 250 μg in about 0.30 mL to about 0.40 mL. In some embodiments, each of the one or more ampules contains about 5 μg to about 600 μg prostaglandin, prostaglandin analogue, prostacyclin or prostacyclin analogue in about 0.40 mL to about 0.50 mL, about 5 μg to about 575 μg in about 0.40 mL to about 0.50 mL, about 5 μg to about 550 μg in about 0.40 mL to about 0.50 mL, about 5 μg to about 525 μg in about 0.40 mL to about 0.50 mL, about 5 μg to about 500 μg in about 0.40 mL to about 0.50 mL, about 5 μg to about 475 μg in about 0.40 mL to about 0.50 mL, about 5 μg to about 450 μg in about 0.40 mL to about 0.50 mL, about 5 μg to about 425 μg in about 0.40 mL to about 0.50 mL, about 5 μg to about 400 μg in about 0.40 mL to about 0.50 mL, about 5 μg to about 375 μg in about 0.40 mL to about 0.50 mL, about 5 μg to about 350 μg in about 0.40 mL to about 0.50 mL, about 5 μg to about 325 μg in about 0.40 mL to about 0.50 mL, about 5 μg to about 300 μg in about 0.50 mL, about 5 μg to about 275 μg in about 0.40 mL to about 0.50 mL, about 5 μg to about 250 μg in about 0.40 mL to about 0.50 mL, about 5 μg to about 225 μg in about 0.40 mL to about 0.50 mL, about 5 μg to about 200 μg in about 0.40 mL to about 0.50 mL, about 5 μg to about 175 μg in about 0.40 mL to about 0.50 mL, about 5 μg to about 150 μg in about 0.40 mL to about 0.50 mL, about 5 μg to about 125 μg in about 0.40 mL to about 0.50 mL, about 5 μg to about 100 μg in about 0.40 mL to about 0.50 mL, about 5 μg to about 75 μg in about 0.40 mL to about 0.50 mL, about 5 μg to about 50 μg in about 0.40 mL to about 0.50 mL, about 5 μg to about 25 μg in about 0.40 mL to about 0.50 mL, about 5 μg to about 20 μg in about 0.40 mL to about 0.50 mL, about 5 μg to about 15 μg in about 0.40 mL to about 0.50 mL, about 5 μg to about 10 μg in about 0.40 mL to about 0.50 mL, about 10 μg to about 600 μg in about 0.40 mL to about 0.50 mL, about 10 μg to about 575 μg in about 0.40 mL to about 0.50 mL, about 10 μg to about 550 μg in about 0.40 mL to about 0.50 mL, about 10 μg to about 525 μg in about 0.40 mL to about 0.50 mL, about 10 μg to about 500 μg in about 0.40 mL to about 0.50 mL, about 10 μg to about 475 μg in about 0.40 mL to about 0.50 mL, about 10 μg to about 450 μg in about 0.40 mL to about 0.50 mL, about 10 μg to about 425 μg in about 0.40 mL to about 0.50 mL, about 10 μg to about 400 μg in about 0.40 mL to about 0.50 mL, about 10 μg to about 375 μg in about 0.40 mL to about 0.50 mL, about 10 μg to about 350 μg in about 0.40 mL to about 0.50 mL, about 10 μg to about 325 μg in about 0.40 mL to about 0.50 mL, about 10 μg to about 300 μg in about 0.40 mL to about 0.50 mL, about 10 μg to about 275 μg in about 0.40 mL to about 0.50 mL, about 10 μg to about 250 μg in about 0.40 mL to about 0.50 mL, about 10 μg to about 225 μg in about 0.40 mL to about 0.50 mL, about 10 μg to about 200 μg in about 0.40 mL to about 0.50 mL, about 10 μg to about 175 μg in about 0.40 mL to about 0.50 mL, about 10 μg to about 150 μg in about 0.40 mL to about 0.50 mL, about 10 μg to about 125 μg in about 0.40 mL to about 0.50 mL, about 10 μg to about 100 μg in about 0.40 mL to about 0.50 mL, about 10 μg to about 75 μg in about 0.40 mL to about 0.50 mL, about 10 μg to about 50 μg in about 0.40 mL to about 0.50 mL, about 10 μg to about 25 μg in about 0.40 mL to about 0.50 mL, about 10 μg to about 20 μg in about 0.40 mL to about 0.50 mL, about 10 μg to about 15 μg in about 0.40 mL to about 0.50 mL, about 15 μg to about 600 μg in about 0.40 mL to about 0.50 mL, about 15 μg to about 575 μg in about 0.40 mL to about 0.50 mL, about 15 μg to about 550 μg in about 0.40 mL to about 0.50 mL, about 15 μg to about 525 μg in about 0.40 mL to about 0.50 mL, about 15 μg to about 500 μg in about 0.40 mL to about 0.50 mL, about 15 μg to about 475 μg in about 0.40 mL to about 0.50 mL, about 15 μg to about 450 μg in about 0.40 mL to about 0.50 mL, about 15 μg to about 425 μg in about 0.40 mL to about 0.50 mL, about 15 μg to about 400 μg in about 0.40 mL to about 0.50 mL, about 15 μg to about 375 μg in about 0.40 mL to about 0.50 mL, about 15 μg to about 350 μg in about 0.40 mL to about 0.50 mL, about 15 μg to about 325 μg in about 0.40 mL to about 0.50 mL, about 15 μg to about 300 μg in about 0.40 mL to about 0.50 mL, about 15 μg to about 275 μg in about 0.40 mL to about 0.50 mL, about 15 μg to about 250 μg in about 0.40 mL to about 0.50 mL, about 15 μg to about 225 μg in about 0.40 mL to about 0.50 mL, about 15 μg to about 200 μg in about 0.40 mL to about 0.50 mL, about 15 μg to about 175 μg in about 0.40 mL to about 0.50 mL, about 15 μg to about 150 μg in about 0.40 mL to about 0.50 mL, about 15 μg to about 125 μg in about 0.40 mL to about 0.50 mL, about 15 μg to about 100 μg in about 0.40 mL to about 0.50 mL, about 15 μg to about 75 μg in about 0.40 mL to about 0.50 mL, about 15 μg to about 50 μg in about 0.40 mL to about 0.50 mL, about 15 μg to about 25 μg in about 0.40 mL to about 0.50 mL, about 15 μg to about 20 μg in about 0.40 mL to about 0.50 mL, about 20 μg to about 600 μg in about 0.40 mL to about 0.50 mL, about 20 μg to about 575 μg in about 0.40 mL to about 0.50 mL, about 20 μg to about 550 μg in about 0.40 mL to about 0.50 mL, about 20 μg to about 525 μg in about 0.40 mL to about 0.50 mL, about 20 μg to about 500 μg in about 0.40 mL to about 0.50 mL, about 20 μg to about 475 μg in about 0.40 mL to about 0.50 mL, about 20 μg to about 450 μg in about 0.40 mL to about 0.50 mL, about 20 μg to about 425 μg in about 0.40 mL to about 0.50 mL, about 20 μg to about 400 μg in about 0.40 mL to about 0.50 mL, about 20 μg to about 375 μg in about 0.40 mL to about 0.50 mL, about 20 μg to about 350 μg in about 0.40 mL to about 0.50 mL, about 20 μg to about 325 μg in about 0.40 mL to about 0.50 mL, about 20 μg to about 300 μg in about 0.40 mL to about 0.50 mL, about 20 μg to about 275 μg in about 0.40 mL to about 0.50 mL, about 20 μg to about 250 μg in about 0.40 mL to about 0.50 mL, about 20 μg to about 225 μg in about 0.40 mL to about 0.50 mL, about 20 μg to about 200 μg in about 0.40 mL to about 0.50 mL, about 20 μg to about 175 μg in about 0.40 mL to about 0.50 mL, about 20 μg to about 150 μg in about 0.40 mL to about 0.50 mL, about 20 μg to about 125 μg in about 0.40 mL to about 0.50 mL, about 20 μg to about 100 μg in about 0.40 mL to about 0.50 mL, about 20 μg to about 75 μg in about 0.40 mL to about 0.50 mL, about 20 μg to about 50 μg in about 0.40 mL to about 0.50 mL, about 20 μg to about 25 μg in about 0.40 mL to about 0.50 mL, about 50 μg to about 600 μg in about 0.40 mL to about 0.50 mL, about 50 μg to about 575 μg in about 0.40 mL to about 0.50 mL, about 50 μg to about 550 μg in about 0.40 mL to about 0.50 mL, about 50 μg to about 525 μg in about 0.40 mL to about 0.50 mL, about 50 μg to about 500 μg in about 0.40 mL to about 0.50 mL, about 50 μg to about 475 μg in about 0.40 mL to about 0.50 mL, about 50 μg to about 450 μg in about 0.40 mL to about 0.50 mL, about 50 μg to about 425 μg in about 0.40 mL to about 0.50 mL, about 50 μg to about 400 μg in about 0.40 mL to about 0.50 mL, about 50 μg to about 375 μg in about 0.40 mL to about 0.50 mL, about 50 μg to about 350 μg in about 0.40 mL to about 0.50 mL, about 50 μg to about 325 μg in about 0.40 mL to about 0.50 mL, about 50 μg to about 300 μg in about 0.40 mL to about 0.50 mL, about 50 μg to about 275 μg in about 0.40 mL to about 0.50 mL, about 50 μg to about 250 μg in about 0.40 mL to about 0.50 mL, about 50 μg to about 225 μg in about 0.40 mL to about 0.50 mL, about 50 μg to about 200 μg in about 0.40 mL to about 0.50 mL, about 50 μg to about 175 μg in about 0.40 mL to about 0.50 mL, about 50 μg to about 150 μg in about 0.40 mL to about 0.50 mL, about 50 μg to about 125 μg in about 0.40 mL to about 0.50 mL, about 50 μg to about 100 μg in about 0.40 mL to about 0.50 mL, about 50 μg to about 75 μg in about 0.40 mL to about 0.50 mL, about 75 μg to about 600 μg in about 0.40 mL to about 0.50 mL, about 75 μg to about 575 μg in about 0.40 mL to about 0.50 mL, about 75 μg to about 550 μg in about 0.40 mL to about 0.50 mL, about 75 μg to about 525 μg in about 0.40 mL to about 0.50 mL, about 75 μg to about 500 μg in about 0.40 mL to about 0.50 mL, about 75 μg to about 475 μg in about 0.40 mL to about 0.50 mL, about 75 μg to about 450 μg in about 0.40 mL to about 0.50 mL, about 75 μg to about 425 μg in about 0.40 mL to about 0.50 mL, about 75 μg to about 400 μg in about 0.40 mL to about 0.50 mL, about 75 μg to about 375 μg in about 0.40 mL to about 0.50 mL, about 75 μg to about 350 μg in about 0.40 mL to about 0.50 mL, about 75 μg to about 325 μg in about 0.40 mL to about 0.50 mL, about 75 μg to about 300 μg in about 0.40 mL to about 0.50 mL, about 75 μg to about 275 μg in about 0.40 mL to about 0.50 mL, about 75 μg to about 250 μg in about 0.40 mL to about 0.50 mL, about 75 μg to about 225 μg in about 0.40 mL to about 0.50 mL, about 75 μg to about 200 μg in about 0.40 mL to about 0.50 mL, about 75 μg to about 175 μg in about 0.40 mL to about 0.50 mL, about 75 μg to about 150 μg in about 0.40 mL to about 0.50 mL, about 75 μg to about 125 μg in about 0.40 mL to about 0.50 mL, about 75 μg to about 100 μg in about 0.40 mL to about 0.50 mL, about 100 μg to about 600 μg in about 0.40 mL to about 0.50 mL, about 100 μg to about 575 μg in about 0.40 mL to about 0.50 mL, about 100 μg to about 550 μg in about 0.40 mL to about 0.50 mL, about 100 μg to about 525 μg in about 0.40 ml to about 0.50 mL, about 100 μg to about 500 μg in about 0.40 mL to about 0.50 mL, about 100 μg to about 475 μg in about 0.40 mL to about 0.50 mL, about 100 μg to about 450 μg in about 0.40 mL to about 0.50 mL, about 100 μg to about 425 μg in about 0.40 mL to about 0.50 mL, about 100 μg to about 400 μg in about 0.40 mL to about 0.50 mL, about 100 μg to about 375 μg in about 0.40 mL to about 0.50 mL, about 100 μg to about 350 μg in about 0.40 mL to about 0.50 mL, about 100 μg to about 325 μg in about 0.40 mL to about 0.50 mL, about 100 μg to about 300 μg in about 0.40 mL to about 0.50 mL, about 100 μg to about 275 μg in about 0.40 mL to about 0.50 mL, about 100 μg to about 250 μg in about 0.40 mL to about 0.50 mL, about 100 μg to about 225 μg in about 0.40 mL to about 0.50 mL, about 100 μg to about 200 μg in about 0.40 mL to about 0.50 mL, about 100 μg to about 175 μg in about 0.40 mL to about 0.50 mL, about 100 μg to about 150 μg in about 0.40 mL to about 0.50 mL, about 100 μg to about 125 μg in about 0.40 mL to about 0.50 mL, about 125 μg to about 600 μg in about 0.40 mL to about 0.50 mL, about 125 μg to about 575 μg in about 0.40 mL to about 0.50 mL, about 125 μg to about 550 μg in about 0.40 mL to about 0.50 mL, about 125 μg to about 525 μg in about 0.40 mL to about 0.50 mL, about 125 μg to about 500 μg in about 0.40 mL to about 0.50 mL, about 125 μg to about 475 μg in about 0.40 mL to about 0.50 mL, about 125 μg to about 450 μg in about 0.40 mL to about 0.50 mL, about 125 μg to about 425 μg in about 0.40 mL to about 0.50 mL, about 125 μg to about 400 μg in about 0.40 mL to about 0.50 mL, about 125 μg to about 375 μg in about 0.40 mL to about 0.50 mL, about 125 μg to about 350 μg in about 0.40 mL to about 0.50 mL, about 125 μg to about 325 μg in about 0.40 mL to about 0.50 mL, about 125 μg to about 300 μg in about 0.40 mL to about 0.50 mL, about 125 μg to about 275 μg in about 0.40 mL to about 0.50 mL, about 125 μg to about 250 μg in about 0.40 mL to about 0.50 mL, about 125 μg to about 225 μg in about 0.40 mL to about 0.50 mL, about 125 μg to about 200 μg in about 0.40 mL to about 0.50 mL, about 125 μg to about 175 μg in about 0.40 mL to about 0.50 mL, about 125 μg to about 150 μg in about 0.40 mL to about 0.50 mL, about 150 μg to about 600 μg in about 0.40 mL to about 0.50 mL, about 150 μg to about 575 μg in about 0.40 mL to about 0.50 mL, about 150 μg to about 550 μg in about 0.40 mL to about 0.50 mL, about 150 μg to about 525 μg in about 0.40 mL to about 0.50 mL, about 150 μg to about 500 μg in about 0.40 mL to about 0.50 mL, about 150 μg to about 475 μg in about 0.40 mL to about 0.50 mL, about 150 μg to about 450 μg in about 0.40 mL to about 0.50 mL, about 150 μg to about 425 μg in about 0.40 mL to about 0.50 mL, about 150 μg to about 400 μg in about 0.40 mL to about 0.50 mL, about 150 μg to about 375 μg in about 0.40 mL to about 0.50 mL, about 150 μg to about 350 μg in about 0.40 mL to about 0.50 mL, about 150 μg to about 325 μg in about 0.40 mL to about 0.50 mL, about 150 μg to about 300 μg in about 0.40 mL to about 0.50 mL, about 150 μg to about 275 μg in about 0.40 mL to about 0.50 mL, about 150 μg to about 250 μg in about 0.40 mL to about 0.50 mL, about 150 μg to about 225 μg in about 0.40 mL to about 0.50 mL, about 150 μg to about 200 μg in about 0.40 mL to about 0.50 mL, about 150 μg to about 175 μg in about 0.40 mL to about 0.50 mL, about 175 μg to about 600 μg in about 0.40 mL to about 0.50 mL, about 175 μg to about 575 μg in about 0.40 mL to about 0.50 mL, about 175 μg to about 550 μg in about 0.40 mL to about 0.50 mL, about 175 μg to about 525 μg in about 0.40 mL to about 0.50 mL, about 175 μg to about 500 μg in about 0.40 mL to about 0.50 mL, about 175 μg to about 475 μg in about 0.40 mL to about 0.50 mL, about 175 μg to about 450 μg in about 0.40 mL to about 0.50 mL, about 175 μg to about 425 μg in about 0.40 mL to about 0.50 mL, about 175 μg to about 400 μg in about 0.40 mL to about 0.50 mL, about 175 μg to about 375 μg in about 0.40 mL to about 0.50 mL, about 175 μg to about 350 μg in about 0.40 mL to about 0.50 mL, about 175 μg to about 325 μg in about 0.40 mL to about 0.50 mL, about 175 μg to about 300 μg in about 0.40 mL to about 0.50 mL, about 175 μg to about 275 μg in about 0.40 mL to about 0.50 mL, about 175 μg to about 250 μg in about 0.40 mL to about 0.50 mL, about 175 μg to about 225 μg in about 0.40 mL to about 0.50 mL, about 175 μg to about 200 μg in about 0.40 mL to about 0.50 mL, about 200 μg to about 600 μg in about 0.40 mL to about 0.50 mL, about 200 μg to about 575 μg in about 0.40 mL to about 0.50 mL, about 200 μg to about 550 μg in about 0.40 mL to about 0.50 mL, about 200 μg to about 525 μg in about 0.40 mL to about 0.50 mL, about 200 μg to about 500 μg in about 0.40 mL to about 0.50 mL, about 200 μg to about 475 μg in about 0.40 mL to about 0.50 mL, about 200 μg to about 450 μg in about 0.40 mL to about 0.50 mL, about 200 μg to about 425 μg in about 0.40 mL to about 0.50 mL, about 200 μg to about 400 μg in about 0.40 mL to about 0.50 mL, about 200 μg to about 375 μg in about 0.40 mL to about 0.50 mL, about 200 μg to about 350 μg in about 0.40 mL to about 0.50 mL, about 200 μg to about 325 μg in about 0.40 mL to about 0.50 mL, about 200 μg to about 300 μg in about 0.40 mL to about 0.50 mL, about 200 μg to about 275 μg in about 0.40 mL to about 0.50 mL, about 200 μg to about 250 μg in about 0.40 mL to about 0.50 mL, about 200 μg to about 225 μg in about 0.40 mL to about 0.50 mL, about 225 μg to about 600 μg in about 0.40 mL to about 0.50 mL, about 225 μg to about 575 μg in about 0.40 mL to about 0.50 mL, about 225 μg to about 550 μg in about 0.40 mL to about 0.50 mL, about 225 μg to about 525 μg in about 0.40 mL to about 0.50 mL, about 225 μg to about 500 μg in about 0.40 mL to about 0.50 mL, about 225 μg to about 475 μg in about 0.40 mL to about 0.50 mL, about 225 μg to about 450 μg in about 0.40 mL to about 0.50 mL, about 225 μg to about 425 μg in about 0.40 mL to about 0.50 mL, about 225 μg to about 400 μg in about 0.40 mL to about 0.50 mL, about 225 μg to about 375 μg in about 0.40 mL to about 0.50 mL, about 225 μg to about 350 μg in about 0.40 mL to about 0.50 mL, about 225 μg to about 325 μg in about 0.40 mL to about 0.50 mL, about 225 μg to about 300 μg in about 0.40 mL to about 0.50 mL, about 225 μg to about 275 μg in about 0.40 mL to about 0.50 mL, or about 225 μg to about 250 μg in about 0.40 mL to about 0.50 mL of the pharmaceutical composition.

In some embodiments, each of the one or more ampules or the reservoir of the nebulizer contains prostaglandin, prostaglandin analogue, prostacyclin or prostacyclin analogue in an amount of about 5 μg to about 600 μg in about 0.10 mL, about 5 μg to about 575 μg in about 0.10 mL, about 5 μg to about 550 μg in about 0.10 mL, about 5 μg to about 525 μg in about 0.10 mL, about 5 μg to about 500 μg in about 0.10 mL, about 5 μg to about 475 μg in about 0.10 mL, about 5 μg to about 450 μg in about 0.10 mL, about 5 μg to about 425 μg in about 0.10 mL, about 5 μg to about 400 μg in about 0.10 mL, about 5 μg to about 375 μg in about 0.10 mL, about 5 μg to about 350 μg in about 0.10 mL, about 5 μg to about 325 μg in about 0.10 mL, about 5 μg to about 300 μg in about 0.50 mL, about 5 μg to about 275 μg in about 0.10 mL, about 5 μg to about 250 μg in about 0.10 mL, about 5 μg to about 225 μg in about 0.10 mL, about 5 μg to about 200 μg in about 0.10 mL, about 5 μg to about 175 μg in about 0.10 mL, about 5 μg to about 150 μg in about 0.10 mL, about 5 μg to about 125 μg in about 0.10 mL, about 5 μg to about 100 μg in about 0.10 mL, about 5 μg to about 75 μg in about 0.10 mL, about 5 μg to about 50 μg in about 0.10 mL, about 5 μg to about 25 μg in about 0.10 mL, about 5 μg to about 20 μg in about 0.10 mL, about 5 μg to about 15 μg in about 0.10 mL, about 5 μg to about 10 μg in about 0.10 mL, about 10 μg to about 600 μg in about 0.10 mL, about 10 μg to about 575 μg in about 0.10 mL, about 10 μg to about 550 μg in about 0.10 mL, about 10 μg to about 525 μg in about 0.10 mL, about 10 μg to about 500 μg in about 0.10 mL, about 10 μg to about 475 μg in about 0.10 mL, about 10 μg to about 450 μg in about 0.10 mL, about 10 μg to about 425 μg in about 0.10 mL, about 10 μg to about 400 μg in about 0.10 mL, about 10 μg to about 375 μg in about 0.10 mL, about 10 μg to about 350 μg in about 0.10 mL, about 10 μg to about 325 μg in about 0.10 mL, about 10 μg to about 300 μg in about 0.10 mL, about 10 μg to about 275 μg in about 0.10 mL, about 10 μg to about 250 μg in about 0.10 mL, about 10 μg to about 225 μg in about 0.10 mL, about 10 μg to about 200 μg in about 0.10 mL, about 10 μg to about 175 μg in about 0.10 mL, about 10 μg to about 150 μg in about 0.10 mL, about 10 μg to about 125 μg in about 0.10 mL, about 10 μg to about 100 μg in about 0.10 mL, about 10 μg to about 75 μg in about 0.10 mL, about 10 μg to about 50 μg in about 0.10 mL, about 10 μg to about 25 μg in about 0.10 mL, about 10 μg to about 20 μg in about 0.10 mL, about 10 μg to about 15 μg in about 0.10 mL, about 15 μg to about 600 μg in about 0.10 mL, about 15 μg to about 575 μg in about 0.10 mL, about 15 μg to about 550 μg in about 0.10 mL, about 15 μg to about 525 μg in about 0.10 mL, about 15 μg to about 500 μg in about 0.10 mL, about 15 μg to about 475 μg in about 0.10 mL, about 15 μg to about 450 μg in about 0.10 mL, about 15 μg to about 425 μg in about 0.10 mL, about 15 μg to about 400 μg in about 0.10 mL, about 15 μg to about 375 μg in about 0.10 mL, about 15 μg to about 350 μg in about 0.10 mL, about 15 μg to about 325 μg in about 0.10 mL, about 15 μg to about 300 μg in about 0.10 mL, about 15 μg to about 275 μg in about 0.10 mL, about 15 μg to about 250 μg in about 0.10 mL, about 15 μg to about 225 μg in about 0.10 mL, about 15 μg to about 200 μg in about 0.10 mL, about 15 μg to about 175 μg in about 0.10 mL, about 15 μg to about 150 μg in about 0.10 mL, about 15 μg to about 125 μg in about 0.10 mL, about 15 μg to about 100 μg in about 0.10 mL, about 15 μg to about 75 μg in about 0.10 mL, about 15 μg to about 50 μg in about 0.10 mL, about 15 μg to about 25 μg in about 0.10 mL, about 15 μg to about 20 μg in about 0.10 mL, about 20 μg to about 600 μg in about 0.10 mL, about 20 μg to about 575 μg in about 0.10 mL, about 20 μg to about 550 μg in about 0.10 mL, about 20 μg to about 525 μg in about 0.10 mL, about 20 μg to about 500 μg in about 0.10 mL, about 20 μg to about 475 μg in about 0.10 mL, about 20 μg to about 450 μg in about 0.10 mL, about 20 μg to about 425 μg in about 0.10 mL, about 20 μg to about 400 μg in about 0.10 mL, about 20 μg to about 375 μg in about 0.10 mL, about 20 μg to about 350 μg in about 0.10 mL, about 20 μg to about 325 μg in about 0.10 mL, about 20 μg to about 300 μg in about 0.10 mL, about 20 μg to about 275 μg in about 0.10 mL, about 20 μg to about 250 μg in about 0.10 mL, about 20 μg to about 225 μg in about 0.10 mL, about 20 μg to about 200 μg in about 0.10 mL, about 20 μg to about 175 μg in about 0.10 mL, about 20 μg to about 150 μg in about 0.10 mL, about 20 μg to about 125 μg in about 0.10 mL, about 20 μg to about 100 μg in about 0.10 mL, about 20 μg to about 75 μg in about 0.10 mL, about 20 μg to about 50 μg in about 0.10 mL, about 20 μg to about 25 μg in about 0.10 mL, about 50 μg to about 600 μg in about 0.10 mL, about 50 μg to about 575 μg in about 0.10 mL, about 50 μg to about 550 μg in about 0.10 mL, about 50 μg to about 525 μg in about 0.10 mL, about 50 μg to about 500 μg in about 0.10 mL, about 50 μg to about 475 μg in about 0.10 mL, about 50 μg to about 450 μg in about 0.10 mL, about 50 μg to about 425 μg in about 0.10 mL, about 50 μg to about 400 μg in about 0.10 mL, about 50 μg to about 375 μg in about 0.10 mL, about 50 μg to about 350 μg in about 0.10 mL, about 50 μg to about 325 μg in about 0.10 mL, about 50 μg to about 300 μg in about 0.10 mL, about 50 μg to about 275 μg in about 0.10 mL, about 50 μg to about 250 μg in about 0.10 mL, about 50 μg to about 225 μg in about 0.10 mL, about 50 μg to about 200 μg in about 0.10 mL, about 50 μg to about 175 μg in about 0.10 mL, about 50 μg to about 150 μg in about 0.10 mL, about 50 μg to about 125 μg in about 0.10 mL, about 50 μg to about 100 μg in about 0.10 mL, about 50 μg to about 75 μg in about 0.10 mL, about 75 μg to about 600 μg in about 0.10 mL, about 75 μg to about 575 μg in about 0.10 mL, about 75 μg to about 550 μg in about 0.10 mL, about 75 μg to about 525 μg in about 0.10 mL, about 75 μg to about 500 μg in about 0.10 mL, about 75 μg to about 475 μg in about 0.10 mL, about 75 μg to about 450 μg in about 0.10 mL, about 75 μg to about 425 μg in about 0.10 mL, about 75 μg to about 400 μg in about 0.10 mL, about 75 μg to about 375 μg in about 0.10 mL, about 75 μg to about 350 μg in about 0.10 mL, about 75 μg to about 325 μg in about 0.10 mL, about 75 μg to about 300 μg in about 0.10 mL, about 75 μg to about 275 μg in about 0.10 mL, about 75 μg to about 250 μg in about 0.10 mL, about 75 μg to about 225 μg in about 0.10 mL, about 75 μg to about 200 μg in about 0.10 mL, about 75 μg to about 175 μg in about 0.10 mL, about 75 μg to about 150 μg in about 0.10 mL, about 75 μg to about 125 μg in about 0.10 mL, about 75 μg to about 100 μg in about 0.10 mL, about 100 μg to about 600 μg in about 0.10 mL, about 100 μg to about 575 μg in about 0.10 mL, about 100 μg to about 550 μg in about 0.10 mL, about 100 μg to about 525 μg in about 0.10 mL, about 100 μg to about 500 μg in about 0.10 mL, about 100 μg to about 475 μg in about 0.10 mL, about 100 μg to about 450 μg in about 0.10 mL, about 100 μg to about 425 μg in about 0.10 mL, about 100 μg to about 400 μg in about 0.10 mL, about 100 μg to about 375 μg in about 0.10 mL, about 100 μg to about 350 μg in about 0.10 mL, about 100 μg to about 325 μg in about 0.10 mL, about 100 μg to about 300 μg in about 0.10 mL, about 100 μg to about 275 μg in about 0.10 mL, about 100 μg to about 250 μg in about 0.10 mL, about 100 μg to about 225 μg in about 0.10 mL, about 100 μg to about 200 μg in about 0.10 mL, about 100 μg to about 175 μg in about 0.10 mL, about 100 μg to about 150 μg in about 0.10 mL, about 100 μg to about 125 μg in about 0.10 mL, about 125 μg to about 600 μg in about 0.10 mL, about 125 μg to about 575 μg in about 0.10 mL, about 125 μg to about 550 μg in about 0.10 mL, about 125 μg to about 525 μg in about 0.10 mL, about 125 μg to about 500 μg in about 0.10 mL, about 125 μg to about 475 μg in about 0.10 mL, about 125 μg to about 450 μg in about 0.10 mL, about 125 μg to about 425 μg in about 0.10 mL, about 125 μg to about 400 μg in about 0.10 mL, about 125 μg to about 375 μg in about 0.10 mL, about 125 μg to about 350 μg in about 0.10 mL, about 125 μg to about 325 μg in about 0.10 mL, about 125 μg to about 300 μg in about 0.10 mL, about 125 μg to about 275 μg in about 0.10 mL, about 125 μg to about 250 μg in about 0.10 mL, about 125 μg to about 225 μg in about 0.10 mL, about 125 μg to about 200 μg in about 0.10 mL, about 125 μg to about 175 μg in about 0.10 mL, about 125 μg to about 150 μg in about 0.10 mL, about 150 μg to about 600 μg in about 0.10 mL, about 150 μg to about 575 μg in about 0.10 mL, about 150 μg to about 550 μg in about 0.10 mL, about 150 μg to about 525 μg in about 0.10 mL, about 150 μg to about 500 μg in about 0.10 mL, about 150 μg to about 475 μg in about 0.10 mL, about 150 μg to about 450 μg in about 0.10 mL, about 150 μg to about 425 μg in about 0.10 mL, about 150 μg to about 400 μg in about 0.10 mL, about 150 μg to about 375 μg in about 0.10 mL, about 150 μg to about 350 μg in about 0.10 mL, about 150 μg to about 325 μg in about 0.10 mL, about 150 μg to about 300 μg in about 0.10 mL, about 150 μg to about 275 μg in about 0.10 mL, about 150 μg to about 250 μg in about 0.10 mL, about 150 μg to about 225 μg in about 0.10 mL, about 150 μg to about 200 μg in about 0.10 mL, about 150 μg to about 175 μg in about 0.10 mL, about 175 μg to about 600 μg in about 0.10 mL, about 175 μg to about 575 μg in about 0.10 mL, about 175 μg to about 550 μg in about 0.10 mL, about 175 μg to about 525 μg in about 0.10 mL, about 175 μg to about 500 μg in about 0.10 mL, about 175 μg to about 475 μg in about 0.10 mL, about 175 μg to about 450 μg in about 0.10 mL, about 175 μg to about 425 μg in about 0.10 mL, about 175 μg to about 400 μg in about 0.10 mL, about 175 μg to about 375 μg in about 0.10 mL, about 175 μg to about 350 μg in about 0.10 mL, about 175 μg to about 325 μg in about 0.10 mL, about 175 μg to about 300 μg in about 0.10 mL, about 175 μg to about 275 μg in about 0.10 mL, about 175 μg to about 250 μg in about 0.10 mL, about 175 μg to about 225 μg in about 0.10 mL, about 175 μg to about 200 μg in about 0.10 mL, about 200 μg to about 600 μg in about 0.10 mL, about 200 μg to about 575 μg in about 0.10 mL, about 200 μg to about 550 μg in about 0.10 mL, about 200 μg to about 525 μg in about 0.10 mL, about 200 μg to about 500 μg in about 0.10 mL, about 200 μg to about 475 μg in about 0.10 mL, about 200 μg to about 450 μg in about 0.10 mL, about 200 μg to about 425 μg in about 0.10 mL, about 200 μg to about 400 μg in about 0.10 mL, about 200 μg to about 375 μg in about 0.10 mL, about 200 μg to about 350 μg in about 0.10 mL, about 200 μg to about 325 μg in about 0.10 mL, about 200 μg to about 300 μg in about 0.10 mL, about 200 μg to about 275 μg in about 0.10 mL, about 200 μg to about 250 μg in about 0.10 mL, about 200 μg to about 225 μg in about 0.10 mL, about 225 μg to about 600 μg in about 0.10 mL, about 225 μg to about 575 μg in about 0.10 mL, about 225 μg to about 550 μg in about 0.10 mL, about 225 μg to about 525 μg in about 0.10 mL, about 225 μg to about 500 μg in about 0.10 mL, about 225 μg to about 475 μg in about 0.10 mL, about 225 μg to about 450 μg in about 0.10 mL, about 225 μg to about 425 μg in about 0.10 mL, about 225 μg to about 400 μg in about 0.10 mL, about 225 μg to about 375 μg in about 0.10 mL, about 225 μg to about 350 μg in about 0.10 mL, about 225 μg to about 325 μg in about 0.10 mL, about 225 μg to about 300 μg in about 0.10 mL, about 225 μg to about 275 μg in about 0.10 mL, or about 225 μg to about 250 μg in about 0.10 mL. In some embodiments, each of the one or more ampules contains about 5 μg to about 600 μg prostaglandin, prostaglandin analogue, prostacyclin or prostacyclin analogue in about 0.11 mL, about 5 μg to about 575 μg in about 0.11 mL, about 5 μg to about 550 μg in about 0.11 mL, about 5 μg to about 525 μg in about 0.11 mL, about 5 μg to about 500 μg in about 0.11 mL, about 5 μg to about 475 μg in about 0.11 mL, about 5 μg to about 450 μg in about 0.11 mL, about 5 μg to about 425 μg in about 0.11 mL, about 5 μg to about 400 μg in about 0.11 mL, about 5 μg to about 375 μg in about 0.11 mL, about 5 μg to about 350 μg in about 0.11 mL, about 5 μg to about 325 μg in about 0.11 mL, about 5 μg to about 300 μg in about 0.50 mL, about 5 μg to about 275 μg in about 0.11 mL, about 5 μg to about 250 μg in about 0.11 mL, about 5 μg to about 225 μg in about 0.11 mL, about 5 μg to about 200 μg in about 0.11 mL, about 5 μg to about 175 μg in about 0.11 mL, about 5 μg to about 150 μg in about 0.11 mL, about 5 μg to about 125 μg in about 0.11 mL, about 5 μg to about 100 μg in about 0.11 mL, about 5 μg to about 75 μg in about 0.11 mL, about 5 μg to about 50 μg in about 0.11 mL, about 5 μg to about 25 μg in about 0.11 mL, about 5 μg to about 20 μg in about 0.11 mL, about 5 μg to about 15 μg in about 0.11 mL, about 5 μg to about 10 μg in about 0.11 mL, about 10 μg to about 600 μg in about 0.11 mL, about 10 μg to about 575 μg in about 0.11 mL, about 10 μg to about 550 μg in about 0.11 mL, about 10 μg to about 525 μg in about 0.11 mL, about 10 μg to about 500 μg in about 0.11 mL, about 10 μg to about 475 μg in about 0.11 mL, about 10 μg to about 450 μg in about 0.11 mL, about 10 μg to about 425 μg in about 0.11 mL, about 10 μg to about 400 μg in about 0.11 mL, about 10 μg to about 375 μg in about 0.11 mL, about 10 μg to about 350 μg in about 0.11 mL, about 10 μg to about 325 μg in about 0.11 mL, about 10 μg to about 300 μg in about 0.11 mL, about 10 μg to about 275 μg in about 0.11 mL, about 10 μg to about 250 μg in about 0.11 mL, about 10 μg to about 225 μg in about 0.11 mL, about 10 μg to about 200 μg in about 0.11 mL, about 10 μg to about 175 μg in about 0.11 mL, about 10 μg to about 150 μg in about 0.11 mL, about 10 μg to about 125 μg in about 0.11 mL, about 10 μg to about 100 μg in about 0.11 mL, about 10 μg to about 75 μg in about 0.11 mL, about 10 μg to about 50 μg in about 0.11 mL, about 10 μg to about 25 μg in about 0.11 mL, about 10 μg to about 20 μg in about 0.11 mL, about 10 μg to about 15 μg in about 0.11 mL, about 15 μg to about 600 μg in about 0.11 mL, about 15 μg to about 575 μg in about 0.11 mL, about 15 μg to about 550 μg in about 0.11 mL, about 15 μg to about 525 μg in about 0.11 mL, about 15 μg to about 500 μg in about 0.11 mL, about 15 μg to about 475 μg in about 0.11 mL, about 15 μg to about 450 μg in about 0.11 mL, about 15 μg to about 425 μg in about 0.11 mL, about 15 μg to about 400 μg in about 0.11 mL, about 15 μg to about 375 μg in about 0.11 mL, about 15 μg to about 350 μg in about 0.11 mL, about 15 μg to about 325 μg in about 0.11 mL, about 15 μg to about 300 μg in about 0.11 mL, about 15 μg to about 275 μg in about 0.11 mL, about 15 μg to about 250 μg in about 0.11 mL, about 15 μg to about 225 μg in about 0.11 mL, about 15 μg to about 200 μg in about 0.11 mL, about 15 μg to about 175 μg in about 0.11 mL, about 15 μg to about 150 μg in about 0.11 mL, about 15 μg to about 125 μg in about 0.11 mL, about 15 μg to about 100 μg in about 0.11 mL, about 15 μg to about 75 μg in about 0.11 mL, about 15 μg to about 50 μg in about 0.11 mL, about 15 μg to about 25 μg in about 0.11 mL, about 15 μg to about 20 μg in about 0.11 mL, about 20 μg to about 600 μg in about 0.11 mL, about 20 μg to about 575 μg in about 0.11 mL, about 20 μg to about 550 μg in about 0.11 mL, about 20 μg to about 525 μg in about 0.11 mL, about 20 μg to about 500 μg in about 0.11 mL, about 20 μg to about 475 μg in about 0.11 mL, about 20 μg to about 450 μg in about 0.11 mL, about 20 μg to about 425 μg in about 0.11 mL, about 20 μg to about 400 μg in about 0.11 mL, about 20 μg to about 375 μg in about 0.11 mL, about 20 μg to about 350 μg in about 0.11 mL, about 20 μg to about 325 μg in about 0.11 mL, about 20 μg to about 300 μg in about 0.11 mL, about 20 μg to about 275 μg in about 0.11 mL, about 20 μg to about 250 μg in about 0.11 mL, about 20 μg to about 225 μg in about 0.11 mL, about 20 μg to about 200 μg in about 0.11 mL, about 20 μg to about 175 μg in about 0.11 mL, about 20 μg to about 150 μg in about 0.11 mL, about 20 μg to about 125 μg in about 0.11 mL, about 20 μg to about 100 μg in about 0.11 mL, about 20 μg to about 75 μg in about 0.11 mL, about 20 μg to about 50 μg in about 0.11 mL, about 20 μg to about 25 μg in about 0.11 mL, about 50 μg to about 600 μg in about 0.11 mL, about 50 μg to about 575 μg in about 0.11 mL, about 50 μg to about 550 μg in about 0.11 mL, about 50 μg to about 525 μg in about 0.11 mL, about 50 μg to about 500 μg in about 0.11 mL, about 50 μg to about 475 μg in about 0.11 mL, about 50 μg to about 450 μg in about 0.11 mL, about 50 μg to about 425 μg in about 0.11 mL, about 50 μg to about 400 μg in about 0.11 mL, about 50 μg to about 375 μg in about 0.11 mL, about 50 μg to about 350 μg in about 0.11 mL, about 50 μg to about 325 μg in about 0.11 mL, about 50 μg to about 300 μg in about 0.11 mL, about 50 μg to about 275 μg in about 0.11 mL, about 50 μg to about 250 μg in about 0.11 mL, about 50 μg to about 225 μg in about 0.11 mL, about 50 μg to about 200 μg in about 0.11 mL, about 50 μg to about 175 μg in about 0.11 mL, about 50 μg to about 150 μg in about 0.11 mL, about 50 μg to about 125 μg in about 0.11 mL, about 50 μg to about 100 μg in about 0.11 mL, about 50 μg to about 75 μg in about 0.11 mL, about 75 μg to about 600 μg in about 0.11 mL, about 75 μg to about 575 μg in about 0.11 mL, about 75 μg to about 550 μg in about 0.11 mL, about 75 μg to about 525 μg in about 0.11 mL, about 75 μg to about 500 μg in about 0.11 mL, about 75 μg to about 475 μg in about 0.11 mL, about 75 μg to about 450 μg in about 0.11 mL, about 75 μg to about 425 μg in about 0.11 mL, about 75 μg to about 400 μg in about 0.11 mL, about 75 μg to about 375 μg in about 0.11 mL, about 75 μg to about 350 μg in about 0.11 mL, about 75 μg to about 325 μg in about 0.11 mL, about 75 μg to about 300 μg in about 0.11 mL, about 75 μg to about 275 μg in about 0.11 mL, about 75 μg to about 250 μg in about 0.11 mL, about 75 μg to about 225 μg in about 0.11 mL, about 75 μg to about 200 μg in about 0.11 mL, about 75 μg to about 175 μg in about 0.11 mL, about 75 μg to about 150 μg in about 0.11 mL, about 75 μg to about 125 μg in about 0.11 mL, about 75 μg to about 100 μg in about 0.11 mL, about 100 μg to about 600 μg in about 0.11 mL, about 100 μg to about 575 μg in about 0.11 mL, about 100 μg to about 550 μg in about 0.11 mL, about 100 μg to about 525 μg in about 0.11 mL, about 100 μg to about 500 μg in about 0.11 mL, about 100 μg to about 475 μg in about 0.11 mL, about 100 μg to about 450 μg in about 0.11 mL, about 100 μg to about 425 μg in about 0.11 mL, about 100 μg to about 400 μg in about 0.11 mL, about 100 μg to about 375 μg in about 0.11 mL, about 100 μg to about 350 μg in about 0.11 mL, about 100 μg to about 325 μg in about 0.11 mL, about 100 μg to about 300 μg in about 0.11 mL, about 100 μg to about 275 μg in about 0.11 mL, about 100 μg to about 250 μg in about 0.11 mL, about 100 μg to about 225 μg in about 0.11 mL, about 100 μg to about 200 μg in about 0.11 mL, about 100 μg to about 175 μg in about 0.11 mL, about 100 μg to about 150 μg in about 0.11 mL, about 100 μg to about 125 μg in about 0.11 mL, about 125 μg to about 600 μg in about 0.11 mL, about 125 μg to about 575 μg in about 0.11 mL, about 125 μg to about 550 μg in about 0.11 mL, about 125 μg to about 525 μg in about 0.11 mL, about 125 μg to about 500 μg in about 0.11 mL, about 125 μg to about 475 μg in about 0.11 mL, about 125 μg to about 450 μg in about 0.11 mL, about 125 μg to about 425 μg in about 0.11 mL, about 125 μg to about 400 μg in about 0.11 mL, about 125 μg to about 375 μg in about 0.11 mL, about 125 μg to about 350 μg in about 0.11 mL, about 125 μg to about 325 μg in about 0.11 mL, about 125 μg to about 300 μg in about 0.11 mL, about 125 μg to about 275 μg in about 0.11 mL, about 125 μg to about 250 μg in about 0.11 mL, about 125 μg to about 225 μg in about 0.11 mL, about 125 μg to about 200 μg in about 0.11 mL, about 125 μg to about 175 μg in about 0.11 mL, about 125 μg to about 150 μg in about 0.11 mL, about 150 μg to about 600 μg in about 0.11 mL, about 150 μg to about 575 μg in about 0.11 mL, about 150 μg to about 550 μg in about 0.11 mL, about 150 μg to about 525 μg in about 0.11 mL, about 150 μg to about 500 μg in about 0.11 mL, about 150 μg to about 475 μg in about 0.11 mL, about 150 μg to about 450 μg in about 0.11 mL, about 150 μg to about 425 μg in about 0.11 mL, about 150 μg to about 400 μg in about 0.11 mL, about 150 μg to about 375 μg in about 0.11 mL, about 150 μg to about 350 μg in about 0.11 mL, about 150 μg to about 325 μg in about 0.11 mL, about 150 μg to about 300 μg in about 0.11 mL, about 150 μg to about 275 μg in about 0.11 mL, about 150 μg to about 250 μg in about 0.11 mL, about 150 μg to about 225 μg in about 0.11 mL, about 150 μg to about 200 μg in about 0.11 mL, about 150 μg to about 175 μg in about 0.11 mL, about 175 μg to about 600 μg in about 0.11 mL, about 175 μg to about 575 μg in about 0.11 mL, about 175 μg to about 550 μg in about 0.11 mL, about 175 μg to about 525 μg in about 0.11 mL, about 175 μg to about 500 μg in about 0.11 mL, about 175 μg to about 475 μg in about 0.11 mL, about 175 μg to about 450 μg in about 0.11 mL, about 175 μg to about 425 μg in about 0.11 mL, about 175 μg to about 400 μg in about 0.11 mL, about 175 μg to about 375 μg in about 0.11 mL, about 175 μg to about 350 μg in about 0.11 mL, about 175 μg to about 325 μg in about 0.11 mL, about 175 μg to about 300 μg in about 0.11 mL, about 175 μg to about 275 μg in about 0.11 mL, about 175 μg to about 250 μg in about 0.11 mL, about 175 μg to about 225 μg in about 0.11 mL, about 175 μg to about 200 μg in about 0.11 mL, about 200 μg to about 600 μg in about 0.11 mL, about 200 μg to about 575 μg in about 0.11 mL, about 200 μg to about 550 μg in about 0.11 mL, about 200 μg to about 525 μg in about 0.11 mL, about 200 μg to about 500 μg in about 0.11 mL, about 200 μg to about 475 μg in about 0.11 mL, about 200 μg to about 450 μg in about 0.11 mL, about 200 μg to about 425 μg in about 0.11 mL, about 200 μg to about 400 μg in about 0.11 mL, about 200 μg to about 375 μg in about 0.11 mL, about 200 μg to about 350 μg in about 0.11 mL, about 200 μg to about 325 μg in about 0.11 mL, about 200 μg to about 300 μg in about 0.11 mL, about 200 μg to about 275 μg in about 0.11 mL, about 200 μg to about 250 μg in about 0.11 mL, about 200 μg to about 225 μg in about 0.11 mL, about 225 μg to about 600 μg in about 0.11 mL, about 225 μg to about 575 μg in about 0.11 mL, about 225 μg to about 550 μg in about 0.11 mL, about 225 μg to about 525 μg in about 0.11 mL, about 225 μg to about 500 μg in about 0.11 mL, about 225 μg to about 475 μg in about 0.11 mL, about 225 μg to about 450 μg in about 0.11 mL, about 225 μg to about 425 μg in about 0.11 mL, about 225 μg to about 400 μg in about 0.11 mL, about 225 μg to about 375 μg in about 0.11 mL, about 225 μg to about 350 μg in about 0.11 mL, about 225 μg to about 325 μg in about 0.11 mL, about 225 μg to about 300 μg in about 0.11 mL, about 225 μg to about 275 μg in about 0.11 mL, or about 225 μg to about 250 μg in about 0.11 mL of the pharmaceutical composition.

In some embodiments, each of the one or more ampules or the reservoir of the nebulizer contains prostaglandin, prostaglandin analogue, prostacyclin or prostacyclin analogue in an amount of about 5 μg to about 600 μg in about 0.12 mL, about 5 μg to about 575 μg in about 0.12 mL, about 5 μg to about 550 μg in about 0.12 mL, about 5 μg to about 525 μg in about 0.12 mL, about 5 μg to about 500 μg in about 0.12 mL, about 5 μg to about 475 μg in about 0.12 mL, about 5 μg to about 450 μg in about 0.12 mL, about 5 μg to about 425 μg in about 0.12 mL, about 5 μg to about 400 μg in about 0.12 mL, about 5 μg to about 375 μg in about 0.12 mL, about 5 μg to about 350 μg in about 0.12 mL, about 5 μg to about 325 μg in about 0.12 mL, about 5 μg to about 300 μg in about 0.12 mL, about 5 μg to about 275 μg in about 0.12 mL, about 5 μg to about 250 μg in about 0.12 mL, about 5 μg to about 225 μg in about 0.12 mL, about 5 μg to about 200 μg in about 0.12 mL, about 5 μg to about 175 μg in about 0.12 mL, about 5 μg to about 150 μg in about 0.12 mL, about 5 μg to about 125 μg in about 0.12 mL, about 5 μg to about 100 μg in about 0.12 mL, about 5 μg to about 75 μg in about 0.12 mL, about 5 μg to about 50 μg in about 0.12 mL, about 5 μg to about 25 μg in about 0.12 mL, about 5 μg to about 20 μg in about 0.12 mL, about 5 μg to about 15 μg in about 0.12 mL, about 5 μg to about 10 μg in about 0.12 mL, about 10 μg to about 600 μg in about 0.12 mL, about 10 μg to about 575 μg in about 0.12 mL, about 10 μg to about 550 μg in about 0.12 mL, about 10 μg to about 525 μg in about 0.12 mL, about 10 μg to about 500 μg in about 0.12 mL, about 10 μg to about 475 μg in about 0.12 mL, about 10 μg to about 450 μg in about 0.12 mL, about 10 μg to about 425 μg in about 0.12 mL, about 10 μg to about 400 μg in about 0.12 mL, about 10 μg to about 375 μg in about 0.12 mL, about 10 μg to about 350 μg in about 0.12 mL, about 10 μg to about 325 μg in about 0.12 mL, about 10 μg to about 300 μg in about 0.12 mL, about 10 μg to about 275 μg in about 0.12 mL, about 10 μg to about 250 μg in about 0.12 mL, about 10 μg to about 225 μg in about 0.12 mL, about 10 μg to about 200 μg in about 0.12 mL, about 10 μg to about 175 μg in about 0.12 mL, about 10 μg to about 150 μg in about 0.12 mL, about 10 μg to about 125 μg in about 0.12 mL, about 10 μg to about 100 μg in about 0.12 mL, about 10 μg to about 75 μg in about 0.12 mL, about 10 μg to about 50 μg in about 0.12 mL, about 10 μg to about 25 μg in about 0.12 mL, about 10 μg to about 20 μg in about 0.12 mL, about 10 μg to about 15 μg in about 0.12 mL, about 15 μg to about 600 μg in about 0.12 mL, about 15 μg to about 575 μg in about 0.12 mL, about 15 μg to about 550 μg in about 0.12 mL, about 15 μg to about 525 μg in about 0.12 mL, about 15 μg to about 500 μg in about 0.12 mL, about 15 μg to about 475 μg in about 0.12 mL, about 15 μg to about 450 μg in about 0.12 mL, about 15 μg to about 425 μg in about 0.12 mL, about 15 μg to about 400 μg in about 0.12 mL, about 15 μg to about 375 μg in about 0.12 mL, about 15 μg to about 350 μg in about 0.12 mL, about 15 μg to about 325 μg in about 0.12 mL, about 15 μg to about 300 μg in about 0.12 mL, about 15 μg to about 275 μg in about 0.12 mL, about 15 μg to about 250 μg in about 0.12 mL, about 15 μg to about 225 μg in about 0.12 mL, about 15 μg to about 200 μg in about 0.12 mL, about 15 μg to about 175 μg in about 0.12 mL, about 15 μg to about 150 μg in about 0.12 mL, about 15 μg to about 125 μg in about 0.12 mL, about 15 μg to about 100 μg in about 0.12 mL, about 15 μg to about 75 μg in about 0.12 mL, about 15 μg to about 50 μg in about 0.12 mL, about 15 μg to about 25 μg in about 0.12 mL, about 15 μg to about 20 μg in about 0.12 mL, about 20 μg to about 600 μg in about 0.12 mL, about 20 μg to about 575 μg in about 0.12 mL, about 20 μg to about 550 μg in about 0.12 mL, about 20 μg to about 525 μg in about 0.12 mL, about 20 μg to about 500 μg in about 0.12 mL, about 20 μg to about 475 μg in about 0.12 mL, about 20 μg to about 450 μg in about 0.12 mL, about 20 μg to about 425 μg in about 0.12 mL, about 20 μg to about 400 μg in about 0.12 mL, about 20 μg to about 375 μg in about 0.12 mL, about 20 μg to about 350 μg in about 0.12 mL, about 20 μg to about 325 μg in about 0.12 mL, about 20 μg to about 300 μg in about 0.12 mL, about 20 μg to about 275 μg in about 0.12 mL, about 20 μg to about 250 μg in about 0.12 mL, about 20 μg to about 225 μg in about 0.12 mL, about 20 μg to about 200 μg in about 0.12 mL, about 20 μg to about 175 μg in about 0.12 mL, about 20 μg to about 150 μg in about 0.12 mL, about 20 μg to about 125 μg in about 0.12 mL, about 20 μg to about 100 μg in about 0.12 mL, about 20 μg to about 75 μg in about 0.12 mL, about 20 μg to about 50 μg in about 0.12 mL, about 20 μg to about 25 μg in about 0.12 mL, about 50 μg to about 600 μg in about 0.12 mL, about 50 μg to about 575 μg in about 0.12 mL, about 50 μg to about 550 μg in about 0.12 mL, about 50 μg to about 525 μg in about 0.12 mL, about 50 μg to about 500 μg in about 0.12 mL, about 50 μg to about 475 μg in about 0.12 mL, about 50 μg to about 450 μg in about 0.12 mL, about 50 μg to about 425 μg in about 0.12 mL, about 50 μg to about 400 μg in about 0.12 mL, about 50 μg to about 375 μg in about 0.12 mL, about 50 μg to about 350 μg in about 0.12 mL, about 50 μg to about 325 μg in about 0.12 mL, about 50 μg to about 300 μg in about 0.12 mL, about 50 μg to about 275 μg in about 0.12 mL, about 50 μg to about 250 μg in about 0.12 mL, about 50 μg to about 225 μg in about 0.12 mL, about 50 μg to about 200 μg in about 0.12 mL, about 50 μg to about 175 μg in about 0.12 mL, about 50 μg to about 150 μg in about 0.12 mL, about 50 μg to about 125 μg in about 0.12 mL, about 50 μg to about 100 μg in about 0.12 mL, about 50 μg to about 75 μg in about 0.12 mL, about 75 μg to about 600 μg in about 0.12 mL, about 75 μg to about 575 μg in about 0.12 mL, about 75 μg to about 550 μg in about 0.12 mL, about 75 μg to about 525 μg in about 0.12 mL, about 75 μg to about 500 μg in about 0.12 mL, about 75 μg to about 475 μg in about 0.12 mL, about 75 μg to about 450 μg in about 0.12 mL, about 75 μg to about 425 μg in about 0.12 mL, about 75 μg to about 400 μg in about 0.12 mL, about 75 μg to about 375 μg in about 0.12 mL, about 75 μg to about 350 μg in about 0.12 mL, about 75 μg to about 325 μg in about 0.12 mL, about 75 μg to about 300 μg in about 0.12 mL, about 75 μg to about 275 μg in about 0.12 mL, about 75 μg to about 250 μg in about 0.12 mL, about 75 μg to about 225 μg in about 0.12 mL, about 75 μg to about 200 μg in about 0.12 mL, about 75 μg to about 175 μg in about 0.12 mL, about 75 μg to about 150 μg in about 0.12 mL, about 75 μg to about 125 μg in about 0.12 mL, about 75 μg to about 100 μg in about 0.12 mL, about 100 μg to about 600 μg in about 0.12 mL, about 100 μg to about 575 μg in about 0.12 mL, about 100 μg to about 550 μg in about 0.12 mL, about 100 μg to about 525 μg in about 0.12 mL, about 100 μg to about 500 μg in about 0.12 mL, about 100 μg to about 475 μg in about 0.12 mL, about 100 μg to about 450 μg in about 0.12 mL, about 100 μg to about 425 μg in about 0.12 mL, about 100 μg to about 400 μg in about 0.12 mL, about 100 μg to about 375 μg in about 0.12 mL, about 100 μg to about 350 μg in about 0.12 mL, about 100 μg to about 325 μg in about 0.12 mL, about 100 μg to about 300 μg in about 0.12 mL, about 100 μg to about 275 μg in about 0.12 mL, about 100 μg to about 250 μg in about 0.12 mL, about 100 μg to about 225 μg in about 0.12 mL, about 100 μg to about 200 μg in about 0.12 mL, about 100 μg to about 175 μg in about 0.12 mL, about 100 μg to about 150 μg in about 0.12 mL, about 100 μg to about 125 μg in about 0.12 mL, about 125 μg to about 600 μg in about 0.12 mL, about 125 μg to about 575 μg in about 0.12 mL, about 125 μg to about 550 μg in about 0.12 mL, about 125 μg to about 525 μg in about 0.12 mL, about 125 μg to about 500 μg in about 0.12 mL, about 125 μg to about 475 μg in about 0.12 mL, about 125 μg to about 450 μg in about 0.12 mL, about 125 μg to about 425 μg in about 0.12 mL, about 125 μg to about 400 μg in about 0.12 mL, about 125 μg to about 375 μg in about 0.12 mL, about 125 μg to about 350 μg in about 0.12 mL, about 125 μg to about 325 μg in about 0.12 mL, about 125 μg to about 300 μg in about 0.12 mL, about 125 μg to about 275 μg in about 0.12 mL, about 125 μg to about 250 μg in about 0.12 mL, about 125 μg to about 225 μg in about 0.12 mL, about 125 μg to about 200 μg in about 0.12 mL, about 125 μg to about 175 μg in about 0.12 mL, about 125 μg to about 150 μg in about 0.12 mL, about 150 μg to about 600 μg in about 0.12 mL, about 150 μg to about 575 μg in about 0.12 mL, about 150 μg to about 550 μg in about 0.12 mL, about 150 μg to about 525 μg in about 0.12 mL, about 150 μg to about 500 μg in about 0.12 mL, about 150 μg to about 475 μg in about 0.12 mL, about 150 μg to about 450 μg in about 0.12 mL, about 150 μg to about 425 μg in about 0.12 mL, about 150 μg to about 400 μg in about 0.12 mL, about 150 μg to about 375 μg in about 0.12 mL, about 150 μg to about 350 μg in about 0.12 mL, about 150 μg to about 325 μg in about 0.12 mL, about 150 μg to about 300 μg in about 0.12 mL, about 150 μg to about 275 μg in about 0.12 mL, about 150 μg to about 250 μg in about 0.12 mL, about 150 μg to about 225 μg in about 0.12 mL, about 150 μg to about 200 μg in about 0.12 mL, about 150 μg to about 175 μg in about 0.12 mL, about 175 μg to about 600 μg in about 0.12 mL, about 175 μg to about 575 μg in about 0.12 mL, about 175 μg to about 550 μg in about 0.12 mL, about 175 μg to about 525 μg in about 0.12 mL, about 175 μg to about 500 μg in about 0.12 mL, about 175 μg to about 475 μg in about 0.12 mL, about 175 μg to about 450 μg in about 0.12 mL, about 175 μg to about 425 μg in about 0.12 mL, about 175 μg to about 400 μg in about 0.12 mL, about 175 μg to about 375 μg in about 0.12 mL, about 175 μg to about 350 μg in about 0.12 mL, about 175 μg to about 325 μg in about 0.12 mL, about 175 μg to about 300 μg in about 0.12 mL, about 175 μg to about 275 μg in about 0.12 mL, about 175 μg to about 250 μg in about 0.12 mL, about 175 μg to about 225 μg in about 0.12 mL, about 175 μg to about 200 μg in about 0.12 mL, about 200 μg to about 600 μg in about 0.12 mL, about 200 μg to about 575 μg in about 0.12 mL, about 200 μg to about 550 μg in about 0.12 mL, about 200 μg to about 525 μg in about 0.12 mL, about 200 μg to about 500 μg in about 0.12 mL, about 200 μg to about 475 μg in about 0.12 mL, about 200 μg to about 450 μg in about 0.12 mL, about 200 μg to about 425 μg in about 0.12 mL, about 200 μg to about 400 μg in about 0.12 mL, about 200 μg to about 375 μg in about 0.12 mL, about 200 μg to about 350 μg in about 0.12 mL, about 200 μg to about 325 μg in about 0.12 mL, about 200 μg to about 300 μg in about 0.12 mL, about 200 μg to about 275 μg in about 0.12 mL, about 200 μg to about 250 μg in about 0.12 mL, about 200 μg to about 225 μg in about 0.12 mL, about 225 μg to about 600 μg in about 0.12 mL, about 225 μg to about 575 μg in about 0.12 mL, about 225 μg to about 550 μg in about 0.12 mL, about 225 μg to about 525 μg in about 0.12 mL, about 225 μg to about 500 μg in about 0.12 mL, about 225 μg to about 475 μg in about 0.12 mL, about 225 μg to about 450 μg in about 0.12 mL, about 225 μg to about 425 μg in about 0.12 mL, about 225 μg to about 400 μg in about 0.12 mL, about 225 μg to about 375 μg in about 0.12 mL, about 225 μg to about 350 μg in about 0.12 mL, about 225 μg to about 325 μg in about 0.12 mL, about 225 μg to about 300 μg in about 0.12 mL, about 225 μg to about 275 μg in about 0.12 mL, or about 225 μg to about 250 μg in about 0.12 mL of the pharmaceutical composition.

In some embodiments, each of the one or more ampules or the reservoir of the nebulizer contains about 5 μg to about 600 μg prostaglandin, prostaglandin analogue, prostacyclin or prostacyclin analogue in about 0.13 mL, about 5 μg to about 575 μg in about 0.13 mL, about 5 μg to about 550 μg in about 0.13 mL, about 5 μg to about 525 μg in about 0.13 mL, about 5 μg to about 500 μg in about 0.13 mL, about 5 μg to about 475 μg in about 0.13 mL, about 5 μg to about 450 μg in about 0.13 mL, about 5 μg to about 425 μg in about 0.13 mL, about 5 μg to about 400 μg in about 0.13 mL, about 5 μg to about 375 μg in about 0.13 mL, about 5 μg to about 350 μg in about 0.13 mL, about 5 μg to about 325 μg in about 0.13 mL, about 5 μg to about 300 μg in about 0.13 mL, about 5 μg to about 275 μg in about 0.13 mL, about 5 μg to about 250 μg in about 0.13 mL, about 5 μg to about 225 μg in about 0.13 mL, about 5 μg to about 200 μg in about 0.13 mL, about 5 μg to about 175 μg in about 0.13 mL, about 5 μg to about 150 μg in about 0.13 mL, about 5 μg to about 125 μg in about 0.13 mL, about 5 μg to about 100 μg in about 0.13 mL, about 5 μg to about 75 μg in about 0.13 mL, about 5 μg to about 50 μg in about 0.13 mL, about 5 μg to about 25 μg in about 0.13 mL, about 5 μg to about 20 μg in about 0.13 mL, about 5 μg to about 15 μg in about 0.13 mL, about 5 μg to about 10 μg in about 0.13 mL, about 10 μg to about 600 μg in about 0.13 mL, about 10 μg to about 575 μg in about 0.13 mL, about 10 μg to about 550 μg in about 0.13 mL, about 10 μg to about 525 μg in about 0.13 mL, about 10 μg to about 500 μg in about 0.13 mL, about 10 μg to about 475 μg in about 0.13 mL, about 10 μg to about 450 μg in about 0.13 mL, about 10 μg to about 425 μg in about 0.13 mL, about 10 μg to about 400 μg in about 0.13 mL, about 10 μg to about 375 μg in about 0.13 mL, about 10 μg to about 350 μg in about 0.13 mL, about 10 μg to about 325 μg in about 0.13 mL, about 10 μg to about 300 μg in about 0.13 mL, about 10 μg to about 275 μg in about 0.13 mL, about 10 μg to about 250 μg in about 0.13 mL, about 10 μg to about 225 μg in about 0.13 mL, about 10 μg to about 200 μg in about 0.13 mL, about 10 μg to about 175 μg in about 0.13 mL, about 10 μg to about 150 μg in about 0.13 mL, about 10 μg to about 125 μg in about 0.13 mL, about 10 μg to about 100 μg in about 0.13 mL, about 10 μg to about 75 μg in about 0.13 mL, about 10 μg to about 50 μg in about 0.13 mL, about 10 μg to about 25 μg in about 0.13 mL, about 10 μg to about 20 μg in about 0.13 mL, about 10 μg to about 15 μg in about 0.13 mL, about 15 μg to about 600 μg in about 0.13 mL, about 15 μg to about 575 μg in about 0.13 mL, about 15 μg to about 550 μg in about 0.13 mL, about 15 μg to about 525 μg in about 0.13 mL, about 15 μg to about 500 μg in about 0.13 mL, about 15 μg to about 475 μg in about 0.13 mL, about 15 μg to about 450 μg in about 0.13 mL, about 15 μg to about 425 μg in about 0.13 mL, about 15 μg to about 400 μg in about 0.13 mL, about 15 μg to about 375 μg in about 0.13 mL, about 15 μg to about 350 μg in about 0.13 mL, about 15 μg to about 325 μg in about 0.13 mL, about 15 μg to about 300 μg in about 0.13 mL, about 15 μg to about 275 μg in about 0.13 mL, about 15 μg to about 250 μg in about 0.13 mL, about 15 μg to about 225 μg in about 0.13 mL, about 15 μg to about 200 μg in about 0.13 mL, about 15 μg to about 175 μg in about 0.13 mL, about 15 μg to about 150 μg in about 0.13 mL, about 15 μg to about 125 μg in about 0.13 mL, about 15 μg to about 100 μg in about 0.13 mL, about 15 μg to about 75 μg in about 0.13 mL, about 15 μg to about 50 μg in about 0.13 mL, about 15 μg to about 25 μg in about 0.13 mL, about 15 μg to about 20 μg in about 0.13 mL, about 20 μg to about 600 μg in about 0.13 mL, about 20 μg to about 575 μg in about 0.13 mL, about 20 μg to about 550 μg in about 0.13 mL, about 20 μg to about 525 μg in about 0.13 mL, about 20 μg to about 500 μg in about 0.13 mL, about 20 μg to about 475 μg in about 0.13 mL, about 20 μg to about 450 μg in about 0.13 mL, about 20 μg to about 425 μg in about 0.13 mL, about 20 μg to about 400 μg in about 0.13 mL, about 20 μg to about 375 μg in about 0.13 mL, about 20 μg to about 350 μg in about 0.13 mL, about 20 μg to about 325 μg in about 0.13 mL, about 20 μg to about 300 μg in about 0.13 mL, about 20 μg to about 275 μg in about 0.13 mL, about 20 μg to about 250 μg in about 0.13 mL, about 20 μg to about 225 μg in about 0.13 mL, about 20 μg to about 200 μg in about 0.13 mL, about 20 μg to about 175 μg in about 0.13 mL, about 20 μg to about 150 μg in about 0.13 mL, about 20 μg to about 125 μg in about 0.13 mL, about 20 μg to about 100 μg in about 0.13 mL, about 20 μg to about 75 μg in about 0.13 mL, about 20 μg to about 50 μg in about 0.13 mL, about 20 μg to about 25 μg in about 0.13 mL, about 50 μg to about 600 μg in about 0.13 mL, about 50 μg to about 575 μg in about 0.13 mL, about 50 μg to about 550 μg in about 0.13 mL, about 50 μg to about 525 μg in about 0.13 mL, about 50 μg to about 500 μg in about 0.13 mL, about 50 μg to about 475 μg in about 0.13 mL, about 50 μg to about 450 μg in about 0.13 mL, about 50 μg to about 425 μg in about 0.13 mL, about 50 μg to about 400 μg in about 0.13 mL, about 50 μg to about 375 μg in about 0.13 mL, about 50 μg to about 350 μg in about 0.13 mL, about 50 μg to about 325 μg in about 0.13 mL, about 50 μg to about 300 μg in about 0.13 mL, about 50 μg to about 275 μg in about 0.13 mL, about 50 μg to about 250 μg in about 0.13 mL, about 50 μg to about 225 μg in about 0.13 mL, about 50 μg to about 200 μg in about 0.13 mL, about 50 μg to about 175 μg in about 0.13 mL, about 50 μg to about 150 μg in about 0.13 mL, about 50 μg to about 125 μg in about 0.13 mL, about 50 μg to about 100 μg in about 0.13 mL, about 50 μg to about 75 μg in about 0.13 mL, about 75 μg to about 600 μg in about 0.13 mL, about 75 μg to about 575 μg in about 0.13 mL, about 75 μg to about 550 μg in about 0.13 mL, about 75 μg to about 525 μg in about 0.13 mL, about 75 μg to about 500 μg in about 0.13 mL, about 75 μg to about 475 μg in about 0.13 mL, about 75 μg to about 450 μg in about 0.13 mL, about 75 μg to about 425 μg in about 0.13 mL, about 75 μg to about 400 μg in about 0.13 mL, about 75 μg to about 375 μg in about 0.13 mL, about 75 μg to about 350 μg in about 0.13 mL, about 75 μg to about 325 μg in about 0.13 mL, about 75 μg to about 300 μg in about 0.13 mL, about 75 μg to about 275 μg in about 0.13 mL, about 75 μg to about 250 μg in about 0.13 mL, about 75 μg to about 225 μg in about 0.13 mL, about 75 μg to about 200 μg in about 0.13 mL, about 75 μg to about 175 μg in about 0.13 mL, about 75 μg to about 150 μg in about 0.13 mL, about 75 μg to about 125 μg in about 0.13 mL, about 75 μg to about 100 μg in about 0.13 mL, about 100 μg to about 600 μg in about 0.13 mL, about 100 μg to about 575 μg in about 0.13 mL, about 100 μg to about 550 μg in about 0.13 mL, about 100 μg to about 525 μg in about 0.13 mL, about 100 μg to about 500 μg in about 0.13 mL, about 100 μg to about 475 μg in about 0.13 mL, about 100 μg to about 450 μg in about 0.13 mL, about 100 μg to about 425 μg in about 0.13 mL, about 100 μg to about 400 μg in about 0.13 mL, about 100 μg to about 375 μg in about 0.13 mL, about 100 μg to about 350 μg in about 0.13 mL, about 100 μg to about 325 μg in about 0.13 mL, about 100 μg to about 300 μg in about 0.13 mL, about 100 μg to about 275 μg in about 0.13 mL, about 100 μg to about 250 μg in about 0.13 mL, about 100 μg to about 225 μg in about 0.13 mL, about 100 μg to about 200 μg in about 0.13 mL, about 100 μg to about 175 μg in about 0.13 mL, about 100 μg to about 150 μg in about 0.13 mL, about 100 μg to about 125 μg in about 0.13 mL, about 125 μg to about 600 μg in about 0.13 mL, about 125 μg to about 575 μg in about 0.13 mL, about 125 μg to about 550 μg in about 0.13 mL, about 125 μg to about 525 μg in about 0.13 mL, about 125 μg to about 500 μg in about 0.13 mL, about 125 μg to about 475 μg in about 0.13 mL, about 125 μg to about 450 μg in about 0.13 mL, about 125 μg to about 425 μg in about 0.13 mL, about 125 μg to about 400 μg in about 0.13 mL, about 125 μg to about 375 μg in about 0.13 mL, about 125 μg to about 350 μg in about 0.13 mL, about 125 μg to about 325 μg in about 0.13 mL, about 125 μg to about 300 μg in about 0.13 mL, about 125 μg to about 275 μg in about 0.13 mL, about 125 μg to about 250 μg in about 0.13 mL, about 125 μg to about 225 μg in about 0.13 mL, about 125 μg to about 200 μg in about 0.13 mL, about 125 μg to about 175 μg in about 0.13 mL, about 125 μg to about 150 μg in about 0.13 mL, about 150 μg to about 600 μg in about 0.13 mL, about 150 μg to about 575 μg in about 0.13 mL, about 150 μg to about 550 μg in about 0.13 mL, about 150 μg to about 525 μg in about 0.13 mL, about 150 μg to about 500 μg in about 0.13 mL, about 150 μg to about 475 μg in about 0.13 mL, about 150 μg to about 450 μg in about 0.13 mL, about 150 μg to about 425 μg in about 0.13 mL, about 150 μg to about 400 μg in about 0.13 mL, about 150 μg to about 375 μg in about 0.13 mL, about 150 μg to about 350 μg in about 0.13 mL, about 150 μg to about 325 μg in about 0.13 mL, about 150 μg to about 300 μg in about 0.13 mL, about 150 μg to about 275 μg in about 0.13 mL, about 150 μg to about 250 μg in about 0.13 mL, about 150 μg to about 225 μg in about 0.13 mL, about 150 μg to about 200 μg in about 0.13 mL, about 150 μg to about 175 μg in about 0.13 mL, about 175 μg to about 600 μg in about 0.13 mL, about 175 μg to about 575 μg in about 0.13 mL, about 175 μg to about 550 μg in about 0.13 mL, about 175 μg to about 525 μg in about 0.13 mL, about 175 μg to about 500 μg in about 0.13 mL, about 175 μg to about 475 μg in about 0.13 mL, about 175 μg to about 450 μg in about 0.13 mL, about 175 μg to about 425 μg in about 0.13 mL, about 175 μg to about 400 μg in about 0.13 mL, about 175 μg to about 375 μg in about 0.13 mL, about 175 μg to about 350 μg in about 0.13 mL, about 175 μg to about 325 μg in about 0.13 mL, about 175 μg to about 300 μg in about 0.13 mL, about 175 μg to about 275 μg in about 0.13 mL, about 175 μg to about 250 μg in about 0.13 mL, about 175 μg to about 225 μg in about 0.13 mL, about 175 μg to about 200 μg in about 0.13 mL, about 200 μg to about 600 μg in about 0.13 mL, about 200 μg to about 575 μg in about 0.13 mL, about 200 μg to about 550 μg in about 0.13 mL, about 200 μg to about 525 μg in about 0.13 mL, about 200 μg to about 500 μg in about 0.13 mL, about 200 μg to about 475 μg in about 0.13 mL, about 200 μg to about 450 μg in about 0.13 mL, about 200 μg to about 425 μg in about 0.13 mL, about 200 μg to about 400 μg in about 0.13 mL, about 200 μg to about 375 μg in about 0.13 mL, about 200 μg to about 350 μg in about 0.13 mL, about 200 μg to about 325 μg in about 0.13 mL, about 200 μg to about 300 μg in about 0.13 mL, about 200 μg to about 275 μg in about 0.13 mL, about 200 μg to about 250 μg in about 0.13 mL, about 200 μg to about 225 μg in about 0.13 mL, about 225 μg to about 600 μg in about 0.13 mL, about 225 μg to about 575 μg in about 0.13 mL, about 225 μg to about 550 μg in about 0.13 mL, about 225 μg to about 525 μg in about 0.13 mL, about 225 μg to about 500 μg in about 0.13 mL, about 225 μg to about 475 μg in about 0.13 mL, about 225 μg to about 450 μg in about 0.13 mL, about 225 μg to about 425 μg in about 0.13 mL, about 225 μg to about 400 μg in about 0.13 mL, about 225 μg to about 375 μg in about 0.13 mL, about 225 μg to about 350 μg in about 0.13 mL, about 225 μg to about 325 μg in about 0.13 mL, about 225 μg to about 300 μg in about 0.13 mL, about 225 μg to about 275 μg in about 0.13 mL, or about 225 μg to about 250 μg in about 0.13 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.14 mL, about 5 μg to about 575 μg in about 0.14 mL, about 5 μg to about 550 μg in about 0.14 mL, about 5 μg to about 525 μg in about 0.14 mL, about 5 μg to about 500 μg in about 0.14 mL, about 5 μg to about 475 μg in about 0.14 mL, about 5 μg to about 450 μg in about 0.14 mL, about 5 μg to about 425 μg in about 0.14 mL, about 5 μg to about 400 μg in about 0.14 mL, about 5 μg to about 375 μg in about 0.14 mL, about 5 μg to about 350 μg in about 0.14 mL, about 5 μg to about 325 μg in about 0.14 mL, about 5 μg to about 300 μg in about 0.14 mL, about 5 μg to about 275 μg in about 0.14 mL, about 5 μg to about 250 μg in about 0.14 mL, about 5 μg to about 225 μg in about 0.14 mL, about 5 μg to about 200 μg in about 0.14 mL, about 5 μg to about 175 μg in about 0.14 mL, about 5 μg to about 150 μg in about 0.14 mL, about 5 μg to about 125 μg in about 0.14 mL, about 5 μg to about 100 μg in about 0.14 mL, about 5 μg to about 75 μg in about 0.14 mL, about 5 μg to about 50 μg in about 0.14 mL, about 5 μg to about 25 μg in about 0.14 mL, about 5 μg to about 20 μg in about 0.14 mL, about 5 μg to about 15 μg in about 0.14 mL, about 5 μg to about 10 μg in about 0.14 mL, about 10 μg to about 600 μg in about 0.14 mL, about 10 μg to about 575 μg in about 0.14 mL, about 10 μg to about 550 μg in about 0.14 mL, about 10 μg to about 525 μg in about 0.14 mL, about 10 μg to about 500 μg in about 0.14 mL, about 10 μg to about 475 μg in about 0.14 mL, about 10 μg to about 450 μg in about 0.14 mL, about 10 μg to about 425 μg in about 0.14 mL, about 10 μg to about 400 μg in about 0.14 mL, about 10 μg to about 375 μg in about 0.14 mL, about 10 μg to about 350 μg in about 0.14 mL, about 10 μg to about 325 μg in about 0.14 mL, about 10 μg to about 300 μg in about 0.14 mL, about 10 μg to about 275 μg in about 0.14 mL, about 10 μg to about 250 μg in about 0.14 mL, about 10 μg to about 225 μg in about 0.14 mL, about 10 μg to about 200 μg in about 0.14 mL, about 10 μg to about 175 μg in about 0.14 mL, about 10 μg to about 150 μg in about 0.14 mL, about 10 μg to about 125 μg in about 0.14 mL, about 10 μg to about 100 μg in about 0.14 mL, about 10 μg to about 75 μg in about 0.14 mL, about 10 μg to about 50 μg in about 0.14 mL, about 10 μg to about 25 μg in about 0.14 mL, about 10 μg to about 20 μg in about 0.14 mL, about 10 μg to about 15 μg in about 0.14 mL, about 15 μg to about 600 μg in about 0.14 mL, about 15 μg to about 575 μg in about 0.14 mL, about 15 μg to about 550 μg in about 0.14 mL, about 15 μg to about 525 μg in about 0.14 mL, about 15 μg to about 500 μg in about 0.14 mL, about 15 μg to about 475 μg in about 0.14 mL, about 15 μg to about 450 μg in about 0.14 mL, about 15 μg to about 425 μg in about 0.14 mL, about 15 μg to about 400 μg in about 0.14 mL, about 15 μg to about 375 μg in about 0.14 mL, about 15 μg to about 350 μg in about 0.14 mL, about 15 μg to about 325 μg in about 0.14 mL, about 15 μg to about 300 μg in about 0.14 mL, about 15 μg to about 275 μg in about 0.14 mL, about 15 μg to about 250 μg in about 0.14 mL, about 15 μg to about 225 μg in about 0.14 mL, about 15 μg to about 200 μg in about 0.14 mL, about 15 μg to about 175 μg in about 0.14 mL, about 15 μg to about 150 μg in about 0.14 mL, about 15 μg to about 125 μg in about 0.14 mL, about 15 μg to about 100 μg in about 0.14 mL, about 15 μg to about 75 μg in about 0.14 mL, about 15 μg to about 50 μg in about 0.14 mL, about 15 μg to about 25 μg in about 0.14 mL, about 15 μg to about 20 μg in about 0.14 mL, about 20 μg to about 600 μg in about 0.14 mL, about 20 μg to about 575 μg in about 0.14 mL, about 20 μg to about 550 μg in about 0.14 mL, about 20 μg to about 525 μg in about 0.14 mL, about 20 μg to about 500 μg in about 0.14 mL, about 20 μg to about 475 μg in about 0.14 mL, about 20 μg to about 450 μg in about 0.14 mL, about 20 μg to about 425 μg in about 0.14 mL, about 20 μg to about 400 μg in about 0.14 mL, about 20 μg to about 375 μg in about 0.14 mL, about 20 μg to about 350 μg in about 0.14 mL, about 20 μg to about 325 μg in about 0.14 mL, about 20 μg to about 300 μg in about 0.14 mL, about 20 μg to about 275 μg in about 0.14 mL, about 20 μg to about 250 μg in about 0.14 mL, about 20 μg to about 225 μg in about 0.14 mL, about 20 μg to about 200 μg in about 0.14 mL, about 20 μg to about 175 μg in about 0.14 mL, about 20 μg to about 150 μg in about 0.14 mL, about 20 μg to about 125 μg in about 0.14 mL, about 20 μg to about 100 μg in about 0.14 mL, about 20 μg to about 75 μg in about 0.14 mL, about 20 μg to about 50 μg in about 0.14 mL, about 20 μg to about 25 μg in about 0.14 mL, about 50 μg to about 600 μg in about 0.14 mL, about 50 μg to about 575 μg in about 0.14 mL, about 50 μg to about 550 μg in about 0.14 mL, about 50 μg to about 525 μg in about 0.14 mL, about 50 μg to about 500 μg in about 0.14 mL, about 50 μg to about 475 μg in about 0.14 mL, about 50 μg to about 450 μg in about 0.14 mL, about 50 μg to about 425 μg in about 0.14 mL, about 50 μg to about 400 μg in about 0.14 mL, about 50 μg to about 375 μg in about 0.14 mL, about 50 μg to about 350 μg in about 0.14 mL, about 50 μg to about 325 μg in about 0.14 mL, about 50 μg to about 300 μg in about 0.14 mL, about 50 μg to about 275 μg in about 0.14 mL, about 50 μg to about 250 μg in about 0.14 mL, about 50 μg to about 225 μg in about 0.14 mL, about 50 μg to about 200 μg in about 0.14 mL, about 50 μg to about 175 μg in about 0.14 mL, about 50 μg to about 150 μg in about 0.14 mL, about 50 μg to about 125 μg in about 0.14 mL, about 50 μg to about 100 μg in about 0.14 mL, about 50 μg to about 75 μg in about 0.14 mL, about 75 μg to about 600 μg in about 0.14 mL, about 75 μg to about 575 μg in about 0.14 mL, about 75 μg to about 550 μg in about 0.14 mL, about 75 μg to about 525 μg in about 0.14 mL, about 75 μg to about 500 μg in about 0.14 mL, about 75 μg to about 475 μg in about 0.14 mL, about 75 μg to about 450 μg in about 0.14 mL, about 75 μg to about 425 μg in about 0.14 mL, about 75 μg to about 400 μg in about 0.14 mL, about 75 μg to about 375 μg in about 0.14 mL, about 75 μg to about 350 μg in about 0.14 mL, about 75 μg to about 325 μg in about 0.14 mL, about 75 μg to about 300 μg in about 0.14 mL, about 75 μg to about 275 μg in about 0.14 mL, about 75 μg to about 250 μg in about 0.14 mL, about 75 μg to about 225 μg in about 0.14 mL, about 75 μg to about 200 μg in about 0.14 mL, about 75 μg to about 175 μg in about 0.14 mL, about 75 μg to about 150 μg in about 0.14 mL, about 75 μg to about 125 μg in about 0.14 mL, about 75 μg to about 100 μg in about 0.14 mL, about 100 μg to about 600 μg in about 0.14 mL, about 100 μg to about 575 μg in about 0.14 mL, about 100 μg to about 550 μg in about 0.14 mL, about 100 μg to about 525 μg in about 0.14 mL, about 100 μg to about 500 μg in about 0.14 mL, about 100 μg to about 475 μg in about 0.14 mL, about 100 μg to about 450 μg in about 0.14 mL, about 100 μg to about 425 μg in about 0.14 mL, about 100 μg to about 400 μg in about 0.14 mL, about 100 μg to about 375 μg in about 0.14 mL, about 100 μg to about 350 μg in about 0.14 mL, about 100 μg to about 325 μg in about 0.14 mL, about 100 μg to about 300 μg in about 0.14 mL, about 100 μg to about 275 μg in about 0.14 mL, about 100 μg to about 250 μg in about 0.14 mL, about 100 μg to about 225 μg in about 0.14 mL, about 100 μg to about 200 μg in about 0.14 mL, about 100 μg to about 175 μg in about 0.14 mL, about 100 μg to about 150 μg in about 0.14 mL, about 100 μg to about 125 μg in about 0.14 mL, about 125 μg to about 600 μg in about 0.14 mL, about 125 μg to about 575 μg in about 0.14 mL, about 125 μg to about 550 μg in about 0.14 mL, about 125 μg to about 525 μg in about 0.14 mL, about 125 μg to about 500 μg in about 0.14 mL, about 125 μg to about 475 μg in about 0.14 mL, about 125 μg to about 450 μg in about 0.14 mL, about 125 μg to about 425 μg in about 0.14 mL, about 125 μg to about 400 μg in about 0.14 mL, about 125 μg to about 375 μg in about 0.14 mL, about 125 μg to about 350 μg in about 0.14 mL, about 125 μg to about 325 μg in about 0.14 mL, about 125 μg to about 300 μg in about 0.14 mL, about 125 μg to about 275 μg in about 0.14 mL, about 125 μg to about 250 μg in about 0.14 mL, about 125 μg to about 225 μg in about 0.14 mL, about 125 μg to about 200 μg in about 0.14 mL, about 125 μg to about 175 μg in about 0.14 mL, about 125 μg to about 150 μg in about 0.14 mL, about 150 μg to about 600 μg in about 0.14 mL, about 150 μg to about 575 μg in about 0.14 mL, about 150 μg to about 550 μg in about 0.14 mL, about 150 μg to about 525 μg in about 0.14 mL, about 150 μg to about 500 μg in about 0.14 mL, about 150 μg to about 475 μg in about 0.14 mL, about 150 μg to about 450 μg in about 0.14 mL, about 150 μg to about 425 μg in about 0.14 mL, about 150 μg to about 400 μg in about 0.14 mL, about 150 μg to about 375 μg in about 0.14 mL, about 150 μg to about 350 μg in about 0.14 mL, about 150 μg to about 325 μg in about 0.14 mL, about 150 μg to about 300 μg in about 0.14 mL, about 150 μg to about 275 μg in about 0.14 mL, about 150 μg to about 250 μg in about 0.14 mL, about 150 μg to about 225 μg in about 0.14 mL, about 150 μg to about 200 μg in about 0.14 mL, about 150 μg to about 175 μg in about 0.14 mL, about 175 μg to about 600 μg in about 0.14 mL, about 175 μg to about 575 μg in about 0.14 mL, about 175 μg to about 550 μg in about 0.14 mL, about 175 μg to about 525 μg in about 0.14 mL, about 175 μg to about 500 μg in about 0.14 mL, about 175 μg to about 475 μg in about 0.14 mL, about 175 μg to about 450 μg in about 0.14 mL, about 175 μg to about 425 μg in about 0.14 mL, about 175 μg to about 400 μg in about 0.14 mL, about 175 μg to about 375 μg in about 0.14 mL, about 175 μg to about 350 μg in about 0.14 mL, about 175 μg to about 325 μg in about 0.14 mL, about 175 μg to about 300 μg in about 0.14 mL, about 175 μg to about 275 μg in about 0.14 mL, about 175 μg to about 250 μg in about 0.14 mL, about 175 μg to about 225 μg in about 0.14 mL, about 175 μg to about 200 μg in about 0.14 mL, about 200 μg to about 600 μg in about 0.14 mL, about 200 μg to about 575 μg in about 0.14 mL, about 200 μg to about 550 μg in about 0.14 mL, about 200 μg to about 525 μg in about 0.14 mL, about 200 μg to about 500 μg in about 0.14 mL, about 200 μg to about 475 μg in about 0.14 mL, about 200 μg to about 450 μg in about 0.14 mL, about 200 μg to about 425 μg in about 0.14 mL, about 200 μg to about 400 μg in about 0.14 mL, about 200 μg to about 375 μg in about 0.14 mL, about 200 μg to about 350 μg in about 0.14 mL, about 200 μg to about 325 μg in about 0.14 mL, about 200 μg to about 300 μg in about 0.14 mL, about 200 μg to about 275 μg in about 0.14 mL, about 200 μg to about 250 μg in about 0.14 mL, about 200 μg to about 225 μg in about 0.14 mL, about 225 μg to about 600 μg in about 0.14 mL, about 225 μg to about 575 μg in about 0.14 mL, about 225 μg to about 550 μg in about 0.14 mL, about 225 μg to about 525 μg in about 0.14 mL, about 225 μg to about 500 μg in about 0.14 mL, about 225 μg to about 475 μg in about 0.14 mL, about 225 μg to about 450 μg in about 0.14 mL, about 225 μg to about 425 μg in about 0.14 mL, about 225 μg to about 400 μg in about 0.14 mL, about 225 μg to about 375 μg in about 0.14 mL, about 225 μg to about 350 μg in about 0.14 mL, about 225 μg to about 325 μg in about 0.14 mL, about 225 μg to about 300 μg in about 0.14 mL, about 225 μg to about 275 μg in about 0.14 mL, or about 225 μg to about 250 μg in about 0.14 mL of the pharmaceutical composition.

In some embodiments, each of the one or more ampules or the reservoir of the nebulizer contains prostaglandin, prostaglandin analogue, prostacyclin or prostacyclin analogue in an amount of about 5 μg to about 600 μg in about 0.15 mL, about 5 μg to about 575 μg in about 0.15 mL, about 5 μg to about 550 μg in about 0.15 mL, about 5 μg to about 525 μg in about 0.15 mL, about 5 μg to about 500 μg in about 0.15 mL, about 5 μg to about 475 μg in about 0.15 mL, about 5 μg to about 450 μg in about 0.15 mL, about 5 μg to about 425 μg in about 0.15 mL, about 5 μg to about 400 μg in about 0.15 mL, about 5 μg to about 375 μg in about 0.15 mL, about 5 μg to about 350 μg in about 0.15 mL, about 5 μg to about 325 μg in about 0.15 mL, about 5 μg to about 300 μg in about 0.15 mL, about 5 μg to about 275 μg in about 0.15 mL, about 5 μg to about 250 μg in about 0.15 mL, about 5 μg to about 225 μg in about 0.15 mL, about 5 μg to about 200 μg in about 0.15 mL, about 5 μg to about 175 μg in about 0.15 mL, about 5 μg to about 150 μg in about 0.15 mL, about 5 μg to about 125 μg in about 0.15 mL, about 5 μg to about 100 μg in about 0.15 mL, about 5 μg to about 75 μg in about 0.15 mL, about 5 μg to about 50 μg in about 0.15 mL, about 5 μg to about 25 μg in about 0.15 mL, about 5 μg to about 20 μg in about 0.15 mL, about 5 μg to about 15 μg in about 0.15 mL, about 5 μg to about 10 μg in about 0.15 mL, about 10 μg to about 600 μg in about 0.15 mL, about 10 μg to about 575 μg in about 0.15 mL, about 10 μg to about 550 μg in about 0.15 mL, about 10 μg to about 525 μg in about 0.15 mL, about 10 μg to about 500 μg in about 0.15 mL, about 10 μg to about 475 μg in about 0.15 mL, about 10 μg to about 450 μg in about 0.15 mL, about 10 μg to about 425 μg in about 0.15 mL, about 10 μg to about 400 μg in about 0.15 mL, about 10 μg to about 375 μg in about 0.15 mL, about 10 μg to about 350 μg in about 0.15 mL, about 10 μg to about 325 μg in about 0.15 mL, about 10 μg to about 300 μg in about 0.15 mL, about 10 μg to about 275 μg in about 0.15 mL, about 10 μg to about 250 μg in about 0.15 mL, about 10 μg to about 225 μg in about 0.15 mL, about 10 μg to about 200 μg in about 0.15 mL, about 10 μg to about 175 μg in about 0.15 mL, about 10 μg to about 150 μg in about 0.15 mL, about 10 μg to about 125 μg in about 0.15 mL, about 10 μg to about 100 μg in about 0.15 mL, about 10 μg to about 75 μg in about 0.15 mL, about 10 μg to about 50 μg in about 0.15 mL, about 10 μg to about 25 μg in about 0.15 mL, about 10 μg to about 20 μg in about 0.15 mL, about 10 μg to about 15 μg in about 0.15 mL, about 15 μg to about 600 μg in about 0.15 mL, about 15 μg to about 575 μg in about 0.15 mL, about 15 μg to about 550 μg in about 0.15 mL, about 15 μg to about 525 μg in about 0.15 mL, about 15 μg to about 500 μg in about 0.15 mL, about 15 μg to about 475 μg in about 0.15 mL, about 15 μg to about 450 μg in about 0.15 mL, about 15 μg to about 425 μg in about 0.15 mL, about 15 μg to about 400 μg in about 0.15 mL, about 15 μg to about 375 μg in about 0.15 mL, about 15 μg to about 350 μg in about 0.15 mL, about 15 μg to about 325 μg in about 0.15 mL, about 15 μg to about 300 μg in about 0.15 mL, about 15 μg to about 275 μg in about 0.15 mL, about 15 μg to about 250 μg in about 0.15 mL, about 15 μg to about 225 μg in about 0.15 mL, about 15 μg to about 200 μg in about 0.15 mL, about 15 μg to about 175 μg in about 0.15 mL, about 15 μg to about 150 μg in about 0.15 mL, about 15 μg to about 125 μg in about 0.15 mL, about 15 μg to about 100 μg in about 0.15 mL, about 15 μg to about 75 μg in about 0.15 mL, about 15 μg to about 50 μg in about 0.15 mL, about 15 μg to about 25 μg in about 0.15 mL, about 15 μg to about 20 μg in about 0.15 mL, about 20 μg to about 600 μg in about 0.15 mL, about 20 μg to about 575 μg in about 0.15 mL, about 20 μg to about 550 μg in about 0.15 mL, about 20 μg to about 525 μg in about 0.15 mL, about 20 μg to about 500 μg in about 0.15 mL, about 20 μg to about 475 μg in about 0.15 mL, about 20 μg to about 450 μg in about 0.15 mL, about 20 μg to about 425 μg in about 0.15 mL, about 20 μg to about 400 μg in about 0.15 mL, about 20 μg to about 375 μg in about 0.15 mL, about 20 μg to about 350 μg in about 0.15 mL, about 20 μg to about 325 μg in about 0.15 mL, about 20 μg to about 300 μg in about 0.15 mL, about 20 μg to about 275 μg in about 0.15 mL, about 20 μg to about 250 μg in about 0.15 mL, about 20 μg to about 225 μg in about 0.15 mL, about 20 μg to about 200 μg in about 0.15 mL, about 20 μg to about 175 μg in about 0.15 mL, about 20 μg to about 150 μg in about 0.15 mL, about 20 μg to about 125 μg in about 0.15 mL, about 20 μg to about 100 μg in about 0.15 mL, about 20 μg to about 75 μg in about 0.15 mL, about 20 μg to about 50 μg in about 0.15 mL, about 20 μg to about 25 μg in about 0.15 mL, about 50 μg to about 600 μg in about 0.15 mL, about 50 μg to about 575 μg in about 0.15 mL, about 50 μg to about 550 μg in about 0.15 mL, about 50 μg to about 525 μg in about 0.15 mL, about 50 μg to about 500 μg in about 0.15 mL, about 50 μg to about 475 μg in about 0.15 mL, about 50 μg to about 450 μg in about 0.15 mL, about 50 μg to about 425 μg in about 0.15 mL, about 50 μg to about 400 μg in about 0.15 mL, about 50 μg to about 375 μg in about 0.15 mL, about 50 μg to about 350 μg in about 0.15 mL, about 50 μg to about 325 μg in about 0.15 mL, about 50 μg to about 300 μg in about 0.15 mL, about 50 μg to about 275 μg in about 0.15 mL, about 50 μg to about 250 μg in about 0.15 mL, about 50 μg to about 225 μg in about 0.15 mL, about 50 μg to about 200 μg in about 0.15 mL, about 50 μg to about 175 μg in about 0.15 mL, about 50 μg to about 150 μg in about 0.15 mL, about 50 μg to about 125 μg in about 0.15 mL, about 50 μg to about 100 μg in about 0.15 mL, about 50 μg to about 75 μg in about 0.15 mL, about 75 μg to about 600 μg in about 0.15 mL, about 75 μg to about 575 μg in about 0.15 mL, about 75 μg to about 550 μg in about 0.15 mL, about 75 μg to about 525 μg in about 0.15 mL, about 75 μg to about 500 μg in about 0.15 mL, about 75 μg to about 475 μg in about 0.15 mL, about 75 μg to about 450 μg in about 0.15 mL, about 75 μg to about 425 μg in about 0.15 mL, about 75 μg to about 400 μg in about 0.15 mL, about 75 μg to about 375 μg in about 0.15 mL, about 75 μg to about 350 μg in about 0.15 mL, about 75 μg to about 325 μg in about 0.15 mL, about 75 μg to about 300 μg in about 0.15 mL, about 75 μg to about 275 μg in about 0.15 mL, about 75 μg to about 250 μg in about 0.15 mL, about 75 μg to about 225 μg in about 0.15 mL, about 75 μg to about 200 μg in about 0.15 mL, about 75 μg to about 175 μg in about 0.15 mL, about 75 μg to about 150 μg in about 0.15 mL, about 75 μg to about 125 μg in about 0.15 mL, about 75 μg to about 100 μg in about 0.15 mL, about 100 μg to about 600 μg in about 0.15 mL, about 100 μg to about 575 μg in about 0.15 mL, about 100 μg to about 550 μg in about 0.15 mL, about 100 μg to about 525 μg in about 0.15 mL, about 100 μg to about 500 μg in about 0.15 mL, about 100 μg to about 475 μg in about 0.15 mL, about 100 μg to about 450 μg in about 0.15 mL, about 100 μg to about 425 μg in about 0.15 mL, about 100 μg to about 400 μg in about 0.15 mL, about 100 μg to about 375 μg in about 0.15 mL, about 100 μg to about 350 μg in about 0.15 mL, about 100 μg to about 325 μg in about 0.15 mL, about 100 μg to about 300 μg in about 0.15 mL, about 100 μg to about 275 μg in about 0.15 mL, about 100 μg to about 250 μg in about 0.15 mL, about 100 μg to about 225 μg in about 0.15 mL, about 100 μg to about 200 μg in about 0.15 mL, about 100 μg to about 175 μg in about 0.15 mL, about 100 μg to about 150 μg in about 0.15 mL, about 100 μg to about 125 μg in about 0.15 mL, about 125 μg to about 600 μg in about 0.15 mL, about 125 μg to about 575 μg in about 0.15 mL, about 125 μg to about 550 μg in about 0.15 mL, about 125 μg to about 525 μg in about 0.15 mL, about 125 μg to about 500 μg in about 0.15 mL, about 125 μg to about 475 μg in about 0.15 mL, about 125 μg to about 450 μg in about 0.15 mL, about 125 μg to about 425 μg in about 0.15 mL, about 125 μg to about 400 μg in about 0.15 mL, about 125 μg to about 375 μg in about 0.15 mL, about 125 μg to about 350 μg in about 0.15 mL, about 125 μg to about 325 μg in about 0.15 mL, about 125 μg to about 300 μg in about 0.15 mL, about 125 μg to about 275 μg in about 0.15 mL, about 125 μg to about 250 μg in about 0.15 mL, about 125 μg to about 225 μg in about 0.15 mL, about 125 μg to about 200 μg in about 0.15 mL, about 125 μg to about 175 μg in about 0.15 mL, about 125 μg to about 150 μg in about 0.15 mL, about 150 μg to about 600 μg in about 0.15 mL, about 150 μg to about 575 μg in about 0.15 mL, about 150 μg to about 550 μg in about 0.15 mL, about 150 μg to about 525 μg in about 0.15 mL, about 150 μg to about 500 μg in about 0.15 mL, about 150 μg to about 475 μg in about 0.15 mL, about 150 μg to about 450 μg in about 0.15 mL, about 150 μg to about 425 μg in about 0.15 mL, about 150 μg to about 400 μg in about 0.15 mL, about 150 μg to about 375 μg in about 0.15 mL, about 150 μg to about 350 μg in about 0.15 mL, about 150 μg to about 325 μg in about 0.15 mL, about 150 μg to about 300 μg in about 0.15 mL, about 150 μg to about 275 μg in about 0.15 mL, about 150 μg to about 250 μg in about 0.15 mL, about 150 μg to about 225 μg in about 0.15 mL, about 150 μg to about 200 μg in about 0.15 mL, about 150 μg to about 175 μg in about 0.15 mL, about 175 μg to about 600 μg in about 0.15 mL, about 175 μg to about 575 μg in about 0.15 mL, about 175 μg to about 550 μg in about 0.15 mL, about 175 μg to about 525 μg in about 0.15 mL, about 175 μg to about 500 μg in about 0.15 mL, about 175 μg to about 475 μg in about 0.15 mL, about 175 μg to about 450 μg in about 0.15 mL, about 175 μg to about 425 μg in about 0.15 mL, about 175 μg to about 400 μg in about 0.15 mL, about 175 μg to about 375 μg in about 0.15 mL, about 175 μg to about 350 μg in about 0.15 mL, about 175 μg to about 325 μg in about 0.15 mL, about 175 μg to about 300 μg in about 0.15 mL, about 175 μg to about 275 μg in about 0.15 mL, about 175 μg to about 250 μg in about 0.15 mL, about 175 μg to about 225 μg in about 0.15 mL, about 175 μg to about 200 μg in about 0.15 mL, about 200 μg to about 600 μg in about 0.15 mL, about 200 μg to about 575 μg in about 0.15 mL, about 200 μg to about 550 μg in about 0.15 mL, about 200 μg to about 525 μg in about 0.15 mL, about 200 μg to about 500 μg in about 0.15 mL, about 200 μg to about 475 μg in about 0.15 mL, about 200 μg to about 450 μg in about 0.15 mL, about 200 μg to about 425 μg in about 0.15 mL, about 200 μg to about 400 μg in about 0.15 mL, about 200 μg to about 375 μg in about 0.15 mL, about 200 μg to about 350 μg in about 0.15 mL, about 200 μg to about 325 μg in about 0.15 mL, about 200 μg to about 300 μg in about 0.15 mL, about 200 μg to about 275 μg in about 0.15 mL, about 200 μg to about 250 μg in about 0.15 mL, about 200 μg to about 225 μg in about 0.15 mL, about 225 μg to about 600 μg in about 0.15 mL, about 225 μg to about 575 μg in about 0.15 mL, about 225 μg to about 550 μg in about 0.15 mL, about 225 μg to about 525 μg in about 0.15 mL, about 225 μg to about 500 μg in about 0.15 mL, about 225 μg to about 475 μg in about 0.15 mL, about 225 μg to about 450 μg in about 0.15 mL, about 225 μg to about 425 μg in about 0.15 mL, about 225 μg to about 400 μg in about 0.15 mL, about 225 μg to about 375 μg in about 0.15 mL, about 225 μg to about 350 μg in about 0.15 mL, about 225 μg to about 325 μg in about 0.15 mL, about 225 μg to about 300 μg in about 0.15 mL, about 225 μg to about 275 μg in about 0.15 mL, or about 225 μg to about 250 μg in about 0.15 mL of the pharmaceutical composition.

In some embodiments, each of the one or more ampules or the reservoir of the nebulizer contains prostaglandin, prostaglandin analogue, prostacyclin or prostacyclin analogue in an amount of about 5 μg to about 600 μg in about 0.16 mL, about 5 μg to about 575 μg in about 0.16 mL, about 5 μg to about 550 μg in about 0.16 mL, about 5 μg to about 525 μg in about 0.16 mL, about 5 μg to about 500 μg in about 0.16 mL, about 5 μg to about 475 μg in about 0.16 mL, about 5 μg to about 450 μg in about 0.16 mL, about 5 μg to about 425 μg in about 0.16 mL, about 5 μg to about 400 μg in about 0.16 mL, about 5 μg to about 375 μg in about 0.16 mL, about 5 μg to about 350 μg in about 0.16 mL, about 5 μg to about 325 μg in about 0.16 mL, about 5 μg to about 300 μg in about 0.16 mL, about 5 μg to about 275 μg in about 0.16 mL, about 5 μg to about 250 μg in about 0.16 mL, about 5 μg to about 225 μg in about 0.16 mL, about 5 μg to about 200 μg in about 0.16 mL, about 5 μg to about 175 μg in about 0.16 mL, about 5 μg to about 150 μg in about 0.16 mL, about 5 μg to about 125 μg in about 0.16 mL, about 5 μg to about 100 μg in about 0.16 mL, about 5 μg to about 75 μg in about 0.16 mL, about 5 μg to about 50 μg in about 0.16 mL, about 5 μg to about 25 μg in about 0.16 mL, about 5 μg to about 20 μg in about 0.16 mL, about 5 μg to about 15 μg in about 0.16 mL, about 5 μg to about 10 μg in about 0.16 mL, about 10 μg to about 600 μg in about 0.16 mL, about 10 μg to about 575 μg in about 0.16 mL, about 10 μg to about 550 μg in about 0.16 mL, about 10 μg to about 525 μg in about 0.16 mL, about 10 μg to about 500 μg in about 0.16 mL, about 10 μg to about 475 μg in about 0.16 mL, about 10 μg to about 450 μg in about 0.16 mL, about 10 μg to about 425 μg in about 0.16 mL, about 10 μg to about 400 μg in about 0.16 mL, about 10 μg to about 375 μg in about 0.16 mL, about 10 μg to about 350 μg in about 0.16 mL, about 10 μg to about 325 μg in about 0.16 mL, about 10 μg to about 300 μg in about 0.16 mL, about 10 μg to about 275 μg in about 0.16 mL, about 10 μg to about 250 μg in about 0.16 mL, about 10 μg to about 225 μg in about 0.16 mL, about 10 μg to about 200 μg in about 0.16 mL, about 10 μg to about 175 μg in about 0.16 mL, about 10 μg to about 150 μg in about 0.16 mL, about 10 μg to about 125 μg in about 0.16 mL, about 10 μg to about 100 μg in about 0.16 mL, about 10 μg to about 75 μg in about 0.16 mL, about 10 μg to about 50 μg in about 0.16 mL, about 10 μg to about 25 μg in about 0.16 mL, about 10 μg to about 20 μg in about 0.16 mL, about 10 μg to about 15 μg in about 0.16 mL, about 15 μg to about 600 μg in about 0.16 mL, about 15 μg to about 575 μg in about 0.16 mL, about 15 μg to about 550 μg in about 0.16 mL, about 15 μg to about 525 μg in about 0.16 mL, about 15 μg to about 500 μg in about 0.16 mL, about 15 μg to about 475 μg in about 0.16 mL, about 15 μg to about 450 μg in about 0.16 mL, about 15 μg to about 425 μg in about 0.16 mL, about 15 μg to about 400 μg in about 0.16 mL, about 15 μg to about 375 μg in about 0.16 mL, about 15 μg to about 350 μg in about 0.16 mL, about 15 μg to about 325 μg in about 0.16 mL, about 15 μg to about 300 μg in about 0.16 mL, about 15 μg to about 275 μg in about 0.16 mL, about 15 μg to about 250 μg in about 0.16 mL, about 15 μg to about 225 μg in about 0.16 mL, about 15 μg to about 200 μg in about 0.16 mL, about 15 μg to about 175 μg in about 0.16 mL, about 15 μg to about 150 μg in about 0.16 mL, about 15 μg to about 125 μg in about 0.16 mL, about 15 μg to about 100 μg in about 0.16 mL, about 15 μg to about 75 μg in about 0.16 mL, about 15 μg to about 50 μg in about 0.16 mL, about 15 μg to about 25 μg in about 0.16 mL, about 15 μg to about 20 μg in about 0.16 mL, about 20 μg to about 600 μg in about 0.16 mL, about 20 μg to about 575 μg in about 0.16 mL, about 20 μg to about 550 μg in about 0.16 mL, about 20 μg to about 525 μg in about 0.16 mL, about 20 μg to about 500 μg in about 0.16 mL, about 20 μg to about 475 μg in about 0.16 mL, about 20 μg to about 450 μg in about 0.16 mL, about 20 μg to about 425 μg in about 0.16 mL, about 20 μg to about 400 μg in about 0.16 mL, about 20 μg to about 375 μg in about 0.16 mL, about 20 μg to about 350 μg in about 0.16 mL, about 20 μg to about 325 μg in about 0.16 mL, about 20 μg to about 300 μg in about 0.16 mL, about 20 μg to about 275 μg in about 0.16 mL, about 20 μg to about 250 μg in about 0.16 mL, about 20 μg to about 225 μg in about 0.16 mL, about 20 μg to about 200 μg in about 0.16 mL, about 20 μg to about 175 μg in about 0.16 mL, about 20 μg to about 150 μg in about 0.16 mL, about 20 μg to about 125 μg in about 0.16 mL, about 20 μg to about 100 μg in about 0.16 mL, about 20 μg to about 75 μg in about 0.16 mL, about 20 μg to about 50 μg in about 0.16 mL, about 20 μg to about 25 μg in about 0.16 mL, about 50 μg to about 600 μg in about 0.16 mL, about 50 μg to about 575 μg in about 0.16 mL, about 50 μg to about 550 μg in about 0.16 mL, about 50 μg to about 525 μg in about 0.16 mL, about 50 μg to about 500 μg in about 0.16 mL, about 50 μg to about 475 μg in about 0.16 mL, about 50 μg to about 450 μg in about 0.16 mL, about 50 μg to about 425 μg in about 0.16 mL, about 50 μg to about 400 μg in about 0.16 mL, about 50 μg to about 375 μg in about 0.16 mL, about 50 μg to about 350 μg in about 0.16 mL, about 50 μg to about 325 μg in about 0.16 mL, about 50 μg to about 300 μg in about 0.16 mL, about 50 μg to about 275 μg in about 0.16 mL, about 50 μg to about 250 μg in about 0.16 mL, about 50 μg to about 225 μg in about 0.16 mL, about 50 μg to about 200 μg in about 0.16 mL, about 50 μg to about 175 μg in about 0.16 mL, about 50 μg to about 150 μg in about 0.16 mL, about 50 μg to about 125 μg in about 0.16 mL, about 50 μg to about 100 μg in about 0.16 mL, about 50 μg to about 75 μg in about 0.16 mL, about 75 μg to about 600 μg in about 0.16 mL, about 75 μg to about 575 μg in about 0.16 mL, about 75 μg to about 550 μg in about 0.16 mL, about 75 μg to about 525 μg in about 0.16 mL, about 75 μg to about 500 μg in about 0.16 mL, about 75 μg to about 475 μg in about 0.16 mL, about 75 μg to about 450 μg in about 0.16 mL, about 75 μg to about 425 μg in about 0.16 mL, about 75 μg to about 400 μg in about 0.16 mL, about 75 μg to about 375 μg in about 0.16 mL, about 75 μg to about 350 μg in about 0.16 mL, about 75 μg to about 325 μg in about 0.16 mL, about 75 μg to about 300 μg in about 0.16 mL, about 75 μg to about 275 μg in about 0.16 mL, about 75 μg to about 250 μg in about 0.16 mL, about 75 μg to about 225 μg in about 0.16 mL, about 75 μg to about 200 μg in about 0.16 mL, about 75 μg to about 175 μg in about 0.16 mL, about 75 μg to about 150 μg in about 0.16 mL, about 75 μg to about 125 μg in about 0.16 mL, about 75 μg to about 100 μg in about 0.16 mL, about 100 μg to about 600 μg in about 0.16 mL, about 100 μg to about 575 μg in about 0.16 mL, about 100 μg to about 550 μg in about 0.16 mL, about 100 μg to about 525 μg in about 0.16 mL, about 100 μg to about 500 μg in about 0.16 mL, about 100 μg to about 475 μg in about 0.16 mL, about 100 μg to about 450 μg in about 0.16 mL, about 100 μg to about 425 μg in about 0.16 mL, about 100 μg to about 400 μg in about 0.16 mL, about 100 μg to about 375 μg in about 0.16 mL, about 100 μg to about 350 μg in about 0.16 mL, about 100 μg to about 325 μg in about 0.16 mL, about 100 μg to about 300 μg in about 0.16 mL, about 100 μg to about 275 μg in about 0.16 mL, about 100 μg to about 250 μg in about 0.16 mL, about 100 μg to about 225 μg in about 0.16 mL, about 100 μg to about 200 μg in about 0.16 mL, about 100 μg to about 175 μg in about 0.16 mL, about 100 μg to about 150 μg in about 0.16 mL, about 100 μg to about 125 μg in about 0.16 mL, about 125 μg to about 600 μg in about 0.16 mL, about 125 μg to about 575 μg in about 0.16 mL, about 125 μg to about 550 μg in about 0.16 mL, about 125 μg to about 525 μg in about 0.16 mL, about 125 μg to about 500 μg in about 0.16 mL, about 125 μg to about 475 μg in about 0.16 mL, about 125 μg to about 450 μg in about 0.16 mL, about 125 μg to about 425 μg in about 0.16 mL, about 125 μg to about 400 μg in about 0.16 mL, about 125 μg to about 375 μg in about 0.16 mL, about 125 μg to about 350 μg in about 0.16 mL, about 125 μg to about 325 μg in about 0.16 mL, about 125 μg to about 300 μg in about 0.16 mL, about 125 μg to about 275 μg in about 0.16 mL, about 125 μg to about 250 μg in about 0.16 mL, about 125 μg to about 225 μg in about 0.16 mL, about 125 μg to about 200 μg in about 0.16 mL, about 125 μg to about 175 μg in about 0.16 mL, about 125 μg to about 150 μg in about 0.16 mL, about 150 μg to about 600 μg in about 0.16 mL, about 150 μg to about 575 μg in about 0.16 mL, about 150 μg to about 550 μg in about 0.16 mL, about 150 μg to about 525 μg in about 0.16 mL, about 150 μg to about 500 μg in about 0.16 mL, about 150 μg to about 475 μg in about 0.16 mL, about 150 μg to about 450 μg in about 0.16 mL, about 150 μg to about 425 μg in about 0.16 mL, about 150 μg to about 400 μg in about 0.16 mL, about 150 μg to about 375 μg in about 0.16 mL, about 150 μg to about 350 μg in about 0.16 mL, about 150 μg to about 325 μg in about 0.16 mL, about 150 μg to about 300 μg in about 0.16 mL, about 150 μg to about 275 μg in about 0.16 mL, about 150 μg to about 250 μg in about 0.16 mL, about 150 μg to about 225 μg in about 0.16 mL, about 150 μg to about 200 μg in about 0.16 mL, about 150 μg to about 175 μg in about 0.16 mL, about 175 μg to about 600 μg in about 0.16 mL, about 175 μg to about 575 μg in about 0.16 mL, about 175 μg to about 550 μg in about 0.16 mL, about 175 μg to about 525 μg in about 0.16 mL, about 175 μg to about 500 μg in about 0.16 mL, about 175 μg to about 475 μg in about 0.16 mL, about 175 μg to about 450 μg in about 0.16 mL, about 175 μg to about 425 μg in about 0.16 mL, about 175 μg to about 400 μg in about 0.16 mL, about 175 μg to about 375 μg in about 0.16 mL, about 175 μg to about 350 μg in about 0.16 mL, about 175 μg to about 325 μg in about 0.16 mL, about 175 μg to about 300 μg in about 0.16 mL, about 175 μg to about 275 μg in about 0.16 mL, about 175 μg to about 250 μg in about 0.16 mL, about 175 μg to about 225 μg in about 0.16 mL, about 175 μg to about 200 μg in about 0.16 mL, about 200 μg to about 600 μg in about 0.16 mL, about 200 μg to about 575 μg in about 0.16 mL, about 200 μg to about 550 μg in about 0.16 mL, about 200 μg to about 525 μg in about 0.16 mL, about 200 μg to about 500 μg in about 0.16 mL, about 200 μg to about 475 μg in about 0.16 mL, about 200 μg to about 450 μg in about 0.16 mL, about 200 μg to about 425 μg in about 0.16 mL, about 200 μg to about 400 μg in about 0.16 mL, about 200 μg to about 375 μg in about 0.16 mL, about 200 μg to about 350 μg in about 0.16 mL, about 200 μg to about 325 μg in about 0.16 mL, about 200 μg to about 300 μg in about 0.16 mL, about 200 μg to about 275 μg in about 0.16 mL, about 200 μg to about 250 μg in about 0.16 mL, about 200 μg to about 225 μg in about 0.16 mL, about 225 μg to about 600 μg in about 0.16 mL, about 225 μg to about 575 μg in about 0.16 mL, about 225 μg to about 550 μg in about 0.16 mL, about 225 μg to about 525 μg in about 0.16 mL, about 225 μg to about 500 μg in about 0.16 mL, about 225 μg to about 475 μg in about 0.16 mL, about 225 μg to about 450 μg in about 0.16 mL, about 225 μg to about 425 μg in about 0.16 mL, about 225 μg to about 400 μg in about 0.16 mL, about 225 μg to about 375 μg in about 0.16 mL, about 225 μg to about 350 μg in about 0.16 mL, about 225 μg to about 325 μg in about 0.16 mL, about 225 μg to about 300 μg in about 0.16 mL, about 225 μg to about 275 μg in about 0.16 mL, or about 225 μg to about 250 μg in about 0.16 mL of the pharmaceutical composition.

In some embodiments, each of the one or more ampules or the reservoir of the nebulizer contains prostaglandin, prostaglandin analogue, prostacyclin or prostacyclin analogue in an amount of about 5 μg to about 600 μg in about 0.17 mL, about 5 μg to about 575 μg in about 0.17 mL, about 5 μg to about 550 μg in about 0.17 mL, about 5 μg to about 525 μg in about 0.17 mL, about 5 μg to about 500 μg in about 0.17 mL, about 5 μg to about 475 μg in about 0.17 mL, about 5 μg to about 450 μg in about 0.17 mL, about 5 μg to about 425 μg in about 0.17 mL, about 5 μg to about 400 μg in about 0.17 mL, about 5 μg to about 375 μg in about 0.17 mL, about 5 μg to about 350 μg in about 0.17 mL, about 5 μg to about 325 μg in about 0.17 mL, about 5 μg to about 300 μg in about 0.17 mL, about 5 μg to about 275 μg in about 0.17 mL, about 5 μg to about 250 μg in about 0.17 mL, about 5 μg to about 225 μg in about 0.17 mL, about 5 μg to about 200 μg in about 0.17 mL, about 5 μg to about 175 μg in about 0.17 mL, about 5 μg to about 150 μg in about 0.17 mL, about 5 μg to about 125 μg in about 0.17 mL, about 5 μg to about 100 μg in about 0.17 mL, about 5 μg to about 75 μg in about 0.17 mL, about 5 μg to about 50 μg in about 0.17 mL, about 5 μg to about 25 μg in about 0.17 mL, about 5 μg to about 20 μg in about 0.17 mL, about 5 μg to about 15 μg in about 0.17 mL, about 5 μg to about 10 μg in about 0.17 mL, about 10 μg to about 600 μg in about 0.17 mL, about 10 μg to about 575 μg in about 0.17 mL, about 10 μg to about 550 μg in about 0.17 mL, about 10 μg to about 525 μg in about 0.17 mL, about 10 μg to about 500 μg in about 0.17 mL, about 10 μg to about 475 μg in about 0.17 mL, about 10 μg to about 450 μg in about 0.17 mL, about 10 μg to about 425 μg in about 0.17 mL, about 10 μg to about 400 μg in about 0.17 mL, about 10 μg to about 375 μg in about 0.17 mL, about 10 μg to about 350 μg in about 0.17 mL, about 10 μg to about 325 μg in about 0.17 mL, about 10 μg to about 300 μg in about 0.17 mL, about 10 μg to about 275 μg in about 0.17 mL, about 10 μg to about 250 μg in about 0.17 mL, about 10 μg to about 225 μg in about 0.17 mL, about 10 μg to about 200 μg in about 0.17 mL, about 10 μg to about 175 μg in about 0.17 mL, about 10 μg to about 150 μg in about 0.17 mL, about 10 μg to about 125 μg in about 0.17 mL, about 10 μg to about 100 μg in about 0.17 mL, about 10 μg to about 75 μg in about 0.17 mL, about 10 μg to about 50 μg in about 0.17 mL, about 10 μg to about 25 μg in about 0.17 mL, about 10 μg to about 20 μg in about 0.17 mL, about 10 μg to about 15 μg in about 0.17 mL, about 15 μg to about 600 μg in about 0.17 mL, about 15 μg to about 575 μg in about 0.17 mL, about 15 μg to about 550 μg in about 0.17 mL, about 15 μg to about 525 μg in about 0.17 mL, about 15 μg to about 500 μg in about 0.17 mL, about 15 μg to about 475 μg in about 0.17 mL, about 15 μg to about 450 μg in about 0.17 mL, about 15 μg to about 425 μg in about 0.17 mL, about 15 μg to about 400 μg in about 0.17 mL, about 15 μg to about 375 μg in about 0.17 mL, about 15 μg to about 350 μg in about 0.17 mL, about 15 μg to about 325 μg in about 0.17 mL, about 15 μg to about 300 μg in about 0.17 mL, about 15 μg to about 275 μg in about 0.17 mL, about 15 μg to about 250 μg in about 0.17 mL, about 15 μg to about 225 μg in about 0.17 mL, about 15 μg to about 200 μg in about 0.17 mL, about 15 μg to about 175 μg in about 0.17 mL, about 15 μg to about 150 μg in about 0.17 mL, about 15 μg to about 125 μg in about 0.17 mL, about 15 μg to about 100 μg in about 0.17 mL, about 15 μg to about 75 μg in about 0.17 mL, about 15 μg to about 50 μg in about 0.17 mL, about 15 μg to about 25 μg in about 0.17 mL, about 15 μg to about 20 μg in about 0.17 mL, about 20 μg to about 600 μg in about 0.17 mL, about 20 μg to about 575 μg in about 0.17 mL, about 20 μg to about 550 μg in about 0.17 mL, about 20 μg to about 525 μg in about 0.17 mL, about 20 μg to about 500 μg in about 0.17 mL, about 20 μg to about 475 μg in about 0.17 mL, about 20 μg to about 450 μg in about 0.17 mL, about 20 μg to about 425 μg in about 0.17 mL, about 20 μg to about 400 μg in about 0.17 mL, about 20 μg to about 375 μg in about 0.17 mL, about 20 μg to about 350 μg in about 0.17 mL, about 20 μg to about 325 μg in about 0.17 mL, about 20 μg to about 300 μg in about 0.17 mL, about 20 μg to about 275 μg in about 0.17 mL, about 20 μg to about 250 μg in about 0.17 mL, about 20 μg to about 225 μg in about 0.17 mL, about 20 μg to about 200 μg in about 0.17 mL, about 20 μg to about 175 μg in about 0.17 mL, about 20 μg to about 150 μg in about 0.17 mL, about 20 μg to about 125 μg in about 0.17 mL, about 20 μg to about 100 μg in about 0.17 mL, about 20 μg to about 75 μg in about 0.17 mL, about 20 μg to about 50 μg in about 0.17 mL, about 20 μg to about 25 μg in about 0.17 mL, about 50 μg to about 600 μg in about 0.17 mL, about 50 μg to about 575 μg in about 0.17 mL, about 50 μg to about 550 μg in about 0.17 mL, about 50 μg to about 525 μg in about 0.17 mL, about 50 μg to about 500 μg in about 0.17 mL, about 50 μg to about 475 μg in about 0.17 mL, about 50 μg to about 450 μg in about 0.17 mL, about 50 μg to about 425 μg in about 0.17 mL, about 50 μg to about 400 μg in about 0.17 mL, about 50 μg to about 375 μg in about 0.17 mL, about 50 μg to about 350 μg in about 0.17 mL, about 50 μg to about 325 μg in about 0.17 mL, about 50 μg to about 300 μg in about 0.17 mL, about 50 μg to about 275 μg in about 0.17 mL, about 50 μg to about 250 μg in about 0.17 mL, about 50 μg to about 225 μg in about 0.17 mL, about 50 μg to about 200 μg in about 0.17 mL, about 50 μg to about 175 μg in about 0.17 mL, about 50 μg to about 150 μg in about 0.17 mL, about 50 μg to about 125 μg in about 0.17 mL, about 50 μg to about 100 μg in about 0.17 mL, about 50 μg to about 75 μg in about 0.17 mL, about 75 μg to about 600 μg in about 0.17 mL, about 75 μg to about 575 μg in about 0.17 mL, about 75 μg to about 550 μg in about 0.17 mL, about 75 μg to about 525 μg in about 0.17 mL, about 75 μg to about 500 μg in about 0.17 mL, about 75 μg to about 475 μg in about 0.17 mL, about 75 μg to about 450 μg in about 0.17 mL, about 75 μg to about 425 μg in about 0.17 mL, about 75 μg to about 400 μg in about 0.17 mL, about 75 μg to about 375 μg in about 0.17 mL, about 75 μg to about 350 μg in about 0.17 mL, about 75 μg to about 325 μg in about 0.17 mL, about 75 μg to about 300 μg in about 0.17 mL, about 75 μg to about 275 μg in about 0.17 mL, about 75 μg to about 250 μg in about 0.17 mL, about 75 μg to about 225 μg in about 0.17 mL, about 75 μg to about 200 μg in about 0.17 mL, about 75 μg to about 175 μg in about 0.17 mL, about 75 μg to about 150 μg in about 0.17 mL, about 75 μg to about 125 μg in about 0.17 mL, about 75 μg to about 100 μg in about 0.17 mL, about 100 μg to about 600 μg in about 0.17 mL, about 100 μg to about 575 μg in about 0.17 mL, about 100 μg to about 550 μg in about 0.17 mL, about 100 μg to about 525 μg in about 0.17 mL, about 100 μg to about 500 μg in about 0.17 mL, about 100 μg to about 475 μg in about 0.17 mL, about 100 μg to about 450 μg in about 0.17 mL, about 100 μg to about 425 μg in about 0.17 mL, about 100 μg to about 400 μg in about 0.17 mL, about 100 μg to about 375 μg in about 0.17 mL, about 100 μg to about 350 μg in about 0.17 mL, about 100 μg to about 325 μg in about 0.17 mL, about 100 μg to about 300 μg in about 0.17 mL, about 100 μg to about 275 μg in about 0.17 mL, about 100 μg to about 250 μg in about 0.17 mL, about 100 μg to about 225 μg in about 0.17 mL, about 100 μg to about 200 μg in about 0.17 mL, about 100 μg to about 175 μg in about 0.17 mL, about 100 μg to about 150 μg in about 0.17 mL, about 100 μg to about 125 μg in about 0.17 mL, about 125 μg to about 600 μg in about 0.17 mL, about 125 μg to about 575 μg in about 0.17 mL, about 125 μg to about 550 μg in about 0.17 mL, about 125 μg to about 525 μg in about 0.17 mL, about 125 μg to about 500 μg in about 0.17 mL, about 125 μg to about 475 μg in about 0.17 mL, about 125 μg to about 450 μg in about 0.17 mL, about 125 μg to about 425 μg in about 0.17 mL, about 125 μg to about 400 μg in about 0.17 mL, about 125 μg to about 375 μg in about 0.17 mL, about 125 μg to about 350 μg in about 0.17 mL, about 125 μg to about 325 μg in about 0.17 mL, about 125 μg to about 300 μg in about 0.17 mL, about 125 μg to about 275 μg in about 0.17 mL, about 125 μg to about 250 μg in about 0.17 mL, about 125 μg to about 225 μg in about 0.17 mL, about 125 μg to about 200 μg in about 0.17 mL, about 125 μg to about 175 μg in about 0.17 mL, about 125 μg to about 150 μg in about 0.17 mL, about 150 μg to about 600 μg in about 0.17 mL, about 150 μg to about 575 μg in about 0.17 mL, about 150 μg to about 550 μg in about 0.17 mL, about 150 μg to about 525 μg in about 0.17 mL, about 150 μg to about 500 μg in about 0.17 mL, about 150 μg to about 475 μg in about 0.17 mL, about 150 μg to about 450 μg in about 0.17 mL, about 150 μg to about 425 μg in about 0.17 mL, about 150 μg to about 400 μg in about 0.17 mL, about 150 μg to about 375 μg in about 0.17 mL, about 150 μg to about 350 μg in about 0.17 mL, about 150 μg to about 325 μg in about 0.17 mL, about 150 μg to about 300 μg in about 0.17 mL, about 150 μg to about 275 μg in about 0.17 mL, about 150 μg to about 250 μg in about 0.17 mL, about 150 μg to about 225 μg in about 0.17 mL, about 150 μg to about 200 μg in about 0.17 mL, about 150 μg to about 175 μg in about 0.17 mL, about 175 μg to about 600 μg in about 0.17 mL, about 175 μg to about 575 μg in about 0.17 mL, about 175 μg to about 550 μg in about 0.17 mL, about 175 μg to about 525 μg in about 0.17 mL, about 175 μg to about 500 μg in about 0.17 mL, about 175 μg to about 475 μg in about 0.17 mL, about 175 μg to about 450 μg in about 0.17 mL, about 175 μg to about 425 μg in about 0.17 mL, about 175 μg to about 400 μg in about 0.17 mL, about 175 μg to about 375 μg in about 0.17 mL, about 175 μg to about 350 μg in about 0.17 mL, about 175 μg to about 325 μg in about 0.17 mL, about 175 μg to about 300 μg in about 0.17 mL, about 175 μg to about 275 μg in about 0.17 mL, about 175 μg to about 250 μg in about 0.17 mL, about 175 μg to about 225 μg in about 0.17 mL, about 175 μg to about 200 μg in about 0.17 mL, about 200 μg to about 600 μg in about 0.17 mL, about 200 μg to about 575 μg in about 0.17 mL, about 200 μg to about 550 μg in about 0.17 mL, about 200 μg to about 525 μg in about 0.17 mL, about 200 μg to about 500 μg in about 0.17 mL, about 200 μg to about 475 μg in about 0.17 mL, about 200 μg to about 450 μg in about 0.17 mL, about 200 μg to about 425 μg in about 0.17 mL, about 200 μg to about 400 μg in about 0.17 mL, about 200 μg to about 375 μg in about 0.17 mL, about 200 μg to about 350 μg in about 0.17 mL, about 200 μg to about 325 μg in about 0.17 mL, about 200 μg to about 300 μg in about 0.17 mL, about 200 μg to about 275 μg in about 0.17 mL, about 200 μg to about 250 μg in about 0.17 mL, about 200 μg to about 225 μg in about 0.17 mL, about 225 μg to about 600 μg in about 0.17 mL, about 225 μg to about 575 μg in about 0.17 mL, about 225 μg to about 550 μg in about 0.17 mL, about 225 μg to about 525 μg in about 0.17 mL, about 225 μg to about 500 μg in about 0.17 mL, about 225 μg to about 475 μg in about 0.17 mL, about 225 μg to about 450 μg in about 0.17 mL, about 225 μg to about 425 μg in about 0.17 mL, about 225 μg to about 400 μg in about 0.17 mL, about 225 μg to about 375 μg in about 0.17 mL, about 225 μg to about 350 μg in about 0.17 mL, about 225 μg to about 325 μg in about 0.17 mL, about 225 μg to about 300 μg in about 0.17 mL, about 225 μg to about 275 μg in about 0.17 mL, or about 225 μg to about 250 μg in about 0.17 mL of the pharmaceutical composition.

In some embodiments, each of the one or more ampules or the reservoir of the nebulizer contains prostaglandin, prostaglandin analogue, prostacyclin or prostacyclin analogue in an amount of about 5 μg to about 600 μg in about 0.18 mL, about 5 μg to about 575 μg in about 0.18 mL, about 5 μg to about 550 μg in about 0.18 mL, about 5 μg to about 525 μg in about 0.18 mL, about 5 μg to about 500 μg in about 0.18 mL, about 5 μg to about 475 μg in about 0.18 mL, about 5 μg to about 450 μg in about 0.18 mL, about 5 μg to about 425 μg in about 0.18 mL, about 5 μg to about 400 μg in about 0.18 mL, about 5 μg to about 375 μg in about 0.18 mL, about 5 μg to about 350 μg in about 0.18 mL, about 5 μg to about 325 μg in about 0.18 mL, about 5 μg to about 300 μg in about 0.18 mL, about 5 μg to about 275 μg in about 0.18 mL, about 5 μg to about 250 μg in about 0.18 mL, about 5 μg to about 225 μg in about 0.18 mL, about 5 μg to about 200 μg in about 0.18 mL, about 5 μg to about 175 μg in about 0.18 mL, about 5 μg to about 150 μg in about 0.18 mL, about 5 μg to about 125 μg in about 0.18 mL, about 5 μg to about 100 μg in about 0.18 mL, about 5 μg to about 75 μg in about 0.18 mL, about 5 μg to about 50 μg in about 0.18 mL, about 5 μg to about 25 μg in about 0.18 mL, about 5 μg to about 20 μg in about 0.18 mL, about 5 μg to about 15 μg in about 0.18 mL, about 5 μg to about 10 μg in about 0.18 mL, about 10 μg to about 600 μg in about 0.18 mL, about 10 μg to about 575 μg in about 0.18 mL, about 10 μg to about 550 μg in about 0.18 mL, about 10 μg to about 525 μg in about 0.18 mL, about 10 μg to about 500 μg in about 0.18 mL, about 10 μg to about 475 μg in about 0.18 mL, about 10 μg to about 450 μg in about 0.18 mL, about 10 μg to about 425 μg in about 0.18 mL, about 10 μg to about 400 μg in about 0.18 mL, about 10 μg to about 375 μg in about 0.18 mL, about 10 μg to about 350 μg in about 0.18 mL, about 10 μg to about 325 μg in about 0.18 mL, about 10 μg to about 300 μg in about 0.18 mL, about 10 μg to about 275 μg in about 0.18 mL, about 10 μg to about 250 μg in about 0.18 mL, about 10 μg to about 225 μg in about 0.18 mL, about 10 μg to about 200 μg in about 0.18 mL, about 10 μg to about 175 μg in about 0.18 mL, about 10 μg to about 150 μg in about 0.18 mL, about 10 μg to about 125 μg in about 0.18 mL, about 10 μg to about 100 μg in about 0.18 mL, about 10 μg to about 75 μg in about 0.18 mL, about 10 μg to about 50 μg in about 0.18 mL, about 10 μg to about 25 μg in about 0.18 mL, about 10 μg to about 20 μg in about 0.18 mL, about 10 μg to about 15 μg in about 0.18 mL, about 15 μg to about 600 μg in about 0.18 mL, about 15 μg to about 575 μg in about 0.18 mL, about 15 μg to about 550 μg in about 0.18 mL, about 15 μg to about 525 μg in about 0.18 mL, about 15 μg to about 500 μg in about 0.18 mL, about 15 μg to about 475 μg in about 0.18 mL, about 15 μg to about 450 μg in about 0.18 mL, about 15 μg to about 425 μg in about 0.18 mL, about 15 μg to about 400 μg in about 0.18 mL, about 15 μg to about 375 μg in about 0.18 mL, about 15 μg to about 350 μg in about 0.18 mL, about 15 μg to about 325 μg in about 0.18 mL, about 15 μg to about 300 μg in about 0.18 mL, about 15 μg to about 275 μg in about 0.18 mL, about 15 μg to about 250 μg in about 0.18 mL, about 15 μg to about 225 μg in about 0.18 mL, about 15 μg to about 200 μg in about 0.18 mL, about 15 μg to about 175 μg in about 0.18 mL, about 15 μg to about 150 μg in about 0.18 mL, about 15 μg to about 125 μg in about 0.18 mL, about 15 μg to about 100 μg in about 0.18 mL, about 15 μg to about 75 μg in about 0.18 mL, about 15 μg to about 50 μg in about 0.18 mL, about 15 μg to about 25 μg in about 0.18 mL, about 15 μg to about 20 μg in about 0.18 mL, about 20 μg to about 600 μg in about 0.18 mL, about 20 μg to about 575 μg in about 0.18 mL, about 20 μg to about 550 μg in about 0.18 mL, about 20 μg to about 525 μg in about 0.18 mL, about 20 μg to about 500 μg in about 0.18 mL, about 20 μg to about 475 μg in about 0.18 mL, about 20 μg to about 450 μg in about 0.18 mL, about 20 μg to about 425 μg in about 0.18 mL, about 20 μg to about 400 μg in about 0.18 mL, about 20 μg to about 375 μg in about 0.18 mL, about 20 μg to about 350 μg in about 0.18 mL, about 20 μg to about 325 μg in about 0.18 mL, about 20 μg to about 300 μg in about 0.18 mL, about 20 μg to about 275 μg in about 0.18 mL, about 20 μg to about 250 μg in about 0.18 mL, about 20 μg to about 225 μg in about 0.18 mL, about 20 μg to about 200 μg in about 0.18 mL, about 20 μg to about 175 μg in about 0.18 mL, about 20 μg to about 150 μg in about 0.18 mL, about 20 μg to about 125 μg in about 0.18 mL, about 20 μg to about 100 μg in about 0.18 mL, about 20 μg to about 75 μg in about 0.18 mL, about 20 μg to about 50 μg in about 0.18 mL, about 20 μg to about 25 μg in about 0.18 mL, about 50 μg to about 600 μg in about 0.18 mL, about 50 μg to about 575 μg in about 0.18 mL, about 50 μg to about 550 μg in about 0.18 mL, about 50 μg to about 525 μg in about 0.18 mL, about 50 μg to about 500 μg in about 0.18 mL, about 50 μg to about 475 μg in about 0.18 mL, about 50 μg to about 450 μg in about 0.18 mL, about 50 μg to about 425 μg in about 0.18 mL, about 50 μg to about 400 μg in about 0.18 mL, about 50 μg to about 375 μg in about 0.18 mL, about 50 μg to about 350 μg in about 0.18 mL, about 50 μg to about 325 μg in about 0.18 mL, about 50 μg to about 300 μg in about 0.18 mL, about 50 μg to about 275 μg in about 0.18 mL, about 50 μg to about 250 μg in about 0.18 mL, about 50 μg to about 225 μg in about 0.18 mL, about 50 μg to about 200 μg in about 0.18 mL, about 50 μg to about 175 μg in about 0.18 mL, about 50 μg to about 150 μg in about 0.18 mL, about 50 μg to about 125 μg in about 0.18 mL, about 50 μg to about 100 μg in about 0.18 mL, about 50 μg to about 75 μg in about 0.18 mL, about 75 μg to about 600 μg in about 0.18 mL, about 75 μg to about 575 μg in about 0.18 mL, about 75 μg to about 550 μg in about 0.18 mL, about 75 μg to about 525 μg in about 0.18 mL, about 75 μg to about 500 μg in about 0.18 mL, about 75 μg to about 475 μg in about 0.18 mL, about 75 μg to about 450 μg in about 0.18 mL, about 75 μg to about 425 μg in about 0.18 mL, about 75 μg to about 400 μg in about 0.18 mL, about 75 μg to about 375 μg in about 0.18 mL, about 75 μg to about 350 μg in about 0.18 mL, about 75 μg to about 325 μg in about 0.18 mL, about 75 μg to about 300 μg in about 0.18 mL, about 75 μg to about 275 μg in about 0.18 mL, about 75 μg to about 250 μg in about 0.18 mL, about 75 μg to about 225 μg in about 0.18 mL, about 75 μg to about 200 μg in about 0.18 mL, about 75 μg to about 175 μg in about 0.18 mL, about 75 μg to about 150 μg in about 0.18 mL, about 75 μg to about 125 μg in about 0.18 mL, about 75 μg to about 100 μg in about 0.18 mL, about 100 μg to about 600 μg in about 0.18 mL, about 100 μg to about 575 μg in about 0.18 mL, about 100 μg to about 550 μg in about 0.18 mL, about 100 μg to about 525 μg in about 0.18 mL, about 100 μg to about 500 μg in about 0.18 mL, about 100 μg to about 475 μg in about 0.18 mL, about 100 μg to about 450 μg in about 0.18 mL, about 100 μg to about 425 μg in about 0.18 mL, about 100 μg to about 400 μg in about 0.18 mL, about 100 μg to about 375 μg in about 0.18 mL, about 100 μg to about 350 μg in about 0.18 mL, about 100 μg to about 325 μg in about 0.18 mL, about 100 μg to about 300 μg in about 0.18 mL, about 100 μg to about 275 μg in about 0.18 mL, about 100 μg to about 250 μg in about 0.18 mL, about 100 μg to about 225 μg in about 0.18 mL, about 100 μg to about 200 μg in about 0.18 mL, about 100 μg to about 175 μg in about 0.18 mL, about 100 μg to about 150 μg in about 0.18 mL, about 100 μg to about 125 μg in about 0.18 mL, about 125 μg to about 600 μg in about 0.18 mL, about 125 μg to about 575 μg in about 0.18 mL, about 125 μg to about 550 μg in about 0.18 mL, about 125 μg to about 525 μg in about 0.18 mL, about 125 μg to about 500 μg in about 0.18 mL, about 125 μg to about 475 μg in about 0.18 mL, about 125 μg to about 450 μg in about 0.18 mL, about 125 μg to about 425 μg in about 0.18 mL, about 125 μg to about 400 μg in about 0.18 mL, about 125 μg to about 375 μg in about 0.18 mL, about 125 μg to about 350 μg in about 0.18 mL, about 125 μg to about 325 μg in about 0.18 mL, about 125 μg to about 300 μg in about 0.18 mL, about 125 μg to about 275 μg in about 0.18 mL, about 125 μg to about 250 μg in about 0.18 mL, about 125 μg to about 225 μg in about 0.18 mL, about 125 μg to about 200 μg in about 0.18 mL, about 125 μg to about 175 μg in about 0.18 mL, about 125 μg to about 150 μg in about 0.18 mL, about 150 μg to about 600 μg in about 0.18 mL, about 150 μg to about 575 μg in about 0.18 mL, about 150 μg to about 550 μg in about 0.18 mL, about 150 μg to about 525 μg in about 0.18 mL, about 150 μg to about 500 μg in about 0.18 mL, about 150 μg to about 475 μg in about 0.18 mL, about 150 μg to about 450 μg in about 0.18 mL, about 150 μg to about 425 μg in about 0.18 mL, about 150 μg to about 400 μg in about 0.18 mL, about 150 μg to about 375 μg in about 0.18 mL, about 150 μg to about 350 μg in about 0.18 mL, about 150 μg to about 325 μg in about 0.18 mL, about 150 μg to about 300 μg in about 0.18 mL, about 150 μg to about 275 μg in about 0.18 mL, about 150 μg to about 250 μg in about 0.18 mL, about 150 μg to about 225 μg in about 0.18 mL, about 150 μg to about 200 μg in about 0.18 mL, about 150 μg to about 175 μg in about 0.18 mL, about 175 μg to about 600 μg in about 0.18 mL, about 175 μg to about 575 μg in about 0.18 mL, about 175 μg to about 550 μg in about 0.18 mL, about 175 μg to about 525 μg in about 0.18 mL, about 175 μg to about 500 μg in about 0.18 mL, about 175 μg to about 475 μg in about 0.18 mL, about 175 μg to about 450 μg in about 0.18 mL, about 175 μg to about 425 μg in about 0.18 mL, about 175 μg to about 400 μg in about 0.18 mL, about 175 μg to about 375 μg in about 0.18 mL, about 175 μg to about 350 μg in about 0.18 mL, about 175 μg to about 325 μg in about 0.18 mL, about 175 μg to about 300 μg in about 0.18 mL, about 175 μg to about 275 μg in about 0.18 mL, about 175 μg to about 250 μg in about 0.18 mL, about 175 μg to about 225 μg in about 0.18 mL, about 175 μg to about 200 μg in about 0.18 mL, about 200 μg to about 600 μg in about 0.18 mL, about 200 μg to about 575 μg in about 0.18 mL, about 200 μg to about 550 μg in about 0.18 mL, about 200 μg to about 525 μg in about 0.18 mL, about 200 μg to about 500 μg in about 0.18 mL, about 200 μg to about 475 μg in about 0.18 mL, about 200 μg to about 450 μg in about 0.18 mL, about 200 μg to about 425 μg in about 0.18 mL, about 200 μg to about 400 μg in about 0.18 mL, about 200 μg to about 375 μg in about 0.18 mL, about 200 μg to about 350 μg in about 0.18 mL, about 200 μg to about 325 μg in about 0.18 mL, about 200 μg to about 300 μg in about 0.18 mL, about 200 μg to about 275 μg in about 0.18 mL, about 200 μg to about 250 μg in about 0.18 mL, about 200 μg to about 225 μg in about 0.18 mL, about 225 μg to about 600 μg in about 0.18 mL, about 225 μg to about 575 μg in about 0.18 mL, about 225 μg to about 550 μg in about 0.18 mL, about 225 μg to about 525 μg in about 0.18 mL, about 225 μg to about 500 μg in about 0.18 mL, about 225 μg to about 475 μg in about 0.18 mL, about 225 μg to about 450 μg in about 0.18 mL, about 225 μg to about 425 μg in about 0.18 mL, about 225 μg to about 400 μg in about 0.18 mL, about 225 μg to about 375 μg in about 0.18 mL, about 225 μg to about 350 μg in about 0.18 mL, about 225 μg to about 325 μg in about 0.18 mL, about 225 μg to about 300 μg in about 0.18 mL, about 225 μg to about 275 μg in about 0.18 mL, or about 225 μg to about 250 μg in about 0.18 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.19 mL, about 5 μg to about 575 μg in about 0.19 mL, about 5 μg to about 550 μg in about 0.19 mL, about 5 μg to about 525 μg in about 0.19 mL, about 5 μg to about 500 μg in about 0.19 mL, about 5 μg to about 475 μg in about 0.19 mL, about 5 μg to about 450 μg in about 0.19 mL, about 5 μg to about 425 μg in about 0.19 mL, about 5 μg to about 400 μg in about 0.19 mL, about 5 μg to about 375 μg in about 0.19 mL, about 5 μg to about 350 μg in about 0.19 mL, about 5 μg to about 325 μg in about 0.19 mL, about 5 μg to about 300 μg in about 0.19 mL, about 5 μg to about 275 μg in about 0.19 mL, about 5 μg to about 250 μg in about 0.19 mL, about 5 μg to about 225 μg in about 0.19 mL, about 5 μg to about 200 μg in about 0.19 mL, about 5 μg to about 175 μg in about 0.19 mL, about 5 μg to about 150 μg in about 0.19 mL, about 5 μg to about 125 μg in about 0.19 mL, about 5 μg to about 100 μg in about 0.19 mL, about 5 μg to about 75 μg in about 0.19 mL, about 5 μg to about 50 μg in about 0.19 mL, about 5 μg to about 25 μg in about 0.19 mL, about 5 μg to about 20 μg in about 0.19 mL, about 5 μg to about 15 μg in about 0.19 mL, about 5 μg to about 10 μg in about 0.19 mL, about 10 μg to about 600 μg in about 0.19 mL, about 10 μg to about 575 μg in about 0.19 mL, about 10 μg to about 550 μg in about 0.19 mL, about 10 μg to about 525 μg in about 0.19 mL, about 10 μg to about 500 μg in about 0.19 mL, about 10 μg to about 475 μg in about 0.19 mL, about 10 μg to about 450 μg in about 0.19 mL, about 10 μg to about 425 μg in about 0.19 mL, about 10 μg to about 400 μg in about 0.19 mL, about 10 μg to about 375 μg in about 0.19 mL, about 10 μg to about 350 μg in about 0.19 mL, about 10 μg to about 325 μg in about 0.19 mL, about 10 μg to about 300 μg in about 0.19 mL, about 10 μg to about 275 μg in about 0.19 mL, about 10 μg to about 250 μg in about 0.19 mL, about 10 μg to about 225 μg in about 0.19 mL, about 10 μg to about 200 μg in about 0.19 mL, about 10 μg to about 175 μg in about 0.19 mL, about 10 μg to about 150 μg in about 0.19 mL, about 10 μg to about 125 μg in about 0.19 mL, about 10 μg to about 100 μg in about 0.19 mL, about 10 μg to about 75 μg in about 0.19 mL, about 10 μg to about 50 μg in about 0.19 mL, about 10 μg to about 25 μg in about 0.19 mL, about 10 μg to about 20 μg in about 0.19 mL, about 10 μg to about 15 μg in about 0.19 mL, about 15 μg to about 600 μg in about 0.19 mL, about 15 μg to about 575 μg in about 0.19 mL, about 15 μg to about 550 μg in about 0.19 mL, about 15 μg to about 525 μg in about 0.19 mL, about 15 μg to about 500 μg in about 0.19 mL, about 15 μg to about 475 μg in about 0.19 mL, about 15 μg to about 450 μg in about 0.19 mL, about 15 μg to about 425 μg in about 0.19 mL, about 15 μg to about 400 μg in about 0.19 mL, about 15 μg to about 375 μg in about 0.19 mL, about 15 μg to about 350 μg in about 0.19 mL, about 15 μg to about 325 μg in about 0.19 mL, about 15 μg to about 300 μg in about 0.19 mL, about 15 μg to about 275 μg in about 0.19 mL, about 15 μg to about 250 μg in about 0.19 mL, about 15 μg to about 225 μg in about 0.19 mL, about 15 μg to about 200 μg in about 0.19 mL, about 15 μg to about 175 μg in about 0.19 mL, about 15 μg to about 150 μg in about 0.19 mL, about 15 μg to about 125 μg in about 0.19 mL, about 15 μg to about 100 μg in about 0.19 mL, about 15 μg to about 75 μg in about 0.19 mL, about 15 μg to about 50 μg in about 0.19 mL, about 15 μg to about 25 μg in about 0.19 mL, about 15 μg to about 20 μg in about 0.19 mL, about 20 μg to about 600 μg in about 0.19 mL, about 20 μg to about 575 μg in about 0.19 mL, about 20 μg to about 550 μg in about 0.19 mL, about 20 μg to about 525 μg in about 0.19 mL, about 20 μg to about 500 μg in about 0.19 mL, about 20 μg to about 475 μg in about 0.19 mL, about 20 μg to about 450 μg in about 0.19 mL, about 20 μg to about 425 μg in about 0.19 mL, about 20 μg to about 400 μg in about 0.19 mL, about 20 μg to about 375 μg in about 0.19 mL, about 20 μg to about 350 μg in about 0.19 mL, about 20 μg to about 325 μg in about 0.19 mL, about 20 μg to about 300 μg in about 0.19 mL, about 20 μg to about 275 μg in about 0.19 mL, about 20 μg to about 250 μg in about 0.19 mL, about 20 μg to about 225 μg in about 0.19 mL, about 20 μg to about 200 μg in about 0.19 mL, about 20 μg to about 175 μg in about 0.19 mL, about 20 μg to about 150 μg in about 0.19 mL, about 20 μg to about 125 μg in about 0.19 mL, about 20 μg to about 100 μg in about 0.19 mL, about 20 μg to about 75 μg in about 0.19 mL, about 20 μg to about 50 μg in about 0.19 mL, about 20 μg to about 25 μg in about 0.19 mL, about 50 μg to about 600 μg in about 0.19 mL, about 50 μg to about 575 μg in about 0.19 mL, about 50 μg to about 550 μg in about 0.19 mL, about 50 μg to about 525 μg in about 0.19 mL, about 50 μg to about 500 μg in about 0.19 mL, about 50 μg to about 475 μg in about 0.19 mL, about 50 μg to about 450 μg in about 0.19 mL, about 50 μg to about 425 μg in about 0.19 mL, about 50 μg to about 400 μg in about 0.19 mL, about 50 μg to about 375 μg in about 0.19 mL, about 50 μg to about 350 μg in about 0.19 mL, about 50 μg to about 325 μg in about 0.19 mL, about 50 μg to about 300 μg in about 0.19 mL, about 50 μg to about 275 μg in about 0.19 mL, about 50 μg to about 250 μg in about 0.19 mL, about 50 μg to about 225 μg in about 0.19 mL, about 50 μg to about 200 μg in about 0.19 mL, about 50 μg to about 175 μg in about 0.19 mL, about 50 μg to about 150 μg in about 0.19 mL, about 50 μg to about 125 μg in about 0.19 mL, about 50 μg to about 100 μg in about 0.19 mL, about 50 μg to about 75 μg in about 0.19 mL, about 75 μg to about 600 μg in about 0.19 mL, about 75 μg to about 575 μg in about 0.19 mL, about 75 μg to about 550 μg in about 0.19 mL, about 75 μg to about 525 μg in about 0.19 mL, about 75 μg to about 500 μg in about 0.19 mL, about 75 μg to about 475 μg in about 0.19 mL, about 75 μg to about 450 μg in about 0.19 mL, about 75 μg to about 425 μg in about 0.19 mL, about 75 μg to about 400 μg in about 0.19 mL, about 75 μg to about 375 μg in about 0.19 mL, about 75 μg to about 350 μg in about 0.19 mL, about 75 μg to about 325 μg in about 0.19 mL, about 75 μg to about 300 μg in about 0.19 mL, about 75 μg to about 275 μg in about 0.19 mL, about 75 μg to about 250 μg in about 0.19 mL, about 75 μg to about 225 μg in about 0.19 mL, about 75 μg to about 200 μg in about 0.19 mL, about 75 μg to about 175 μg in about 0.19 mL, about 75 μg to about 150 μg in about 0.19 mL, about 75 μg to about 125 μg in about 0.19 mL, about 75 μg to about 100 μg in about 0.19 mL, about 100 μg to about 600 μg in about 0.19 mL, about 100 μg to about 575 μg in about 0.19 mL, about 100 μg to about 550 μg in about 0.19 mL, about 100 μg to about 525 μg in about 0.19 mL, about 100 μg to about 500 μg in about 0.19 mL, about 100 μg to about 475 μg in about 0.19 mL, about 100 μg to about 450 μg in about 0.19 mL, about 100 μg to about 425 μg in about 0.19 mL, about 100 μg to about 400 μg in about 0.19 mL, about 100 μg to about 375 μg in about 0.19 mL, about 100 μg to about 350 μg in about 0.19 mL, about 100 μg to about 325 μg in about 0.19 mL, about 100 μg to about 300 μg in about 0.19 mL, about 100 μg to about 275 μg in about 0.19 mL, about 100 μg to about 250 μg in about 0.19 mL, about 100 μg to about 225 μg in about 0.19 mL, about 100 μg to about 200 μg in about 0.19 mL, about 100 μg to about 175 μg in about 0.19 mL, about 100 μg to about 150 μg in about 0.19 mL, about 100 μg to about 125 μg in about 0.19 mL, about 125 μg to about 600 μg in about 0.19 mL, about 125 μg to about 575 μg in about 0.19 mL, about 125 μg to about 550 μg in about 0.19 mL, about 125 μg to about 525 μg in about 0.19 mL, about 125 μg to about 500 μg in about 0.19 mL, about 125 μg to about 475 μg in about 0.19 mL, about 125 μg to about 450 μg in about 0.19 mL, about 125 μg to about 425 μg in about 0.19 mL, about 125 μg to about 400 μg in about 0.19 mL, about 125 μg to about 375 μg in about 0.19 mL, about 125 μg to about 350 μg in about 0.19 mL, about 125 μg to about 325 μg in about 0.19 mL, about 125 μg to about 300 μg in about 0.19 mL, about 125 μg to about 275 μg in about 0.19 mL, about 125 μg to about 250 μg in about 0.19 mL, about 125 μg to about 225 μg in about 0.19 mL, about 125 μg to about 200 μg in about 0.19 mL, about 125 μg to about 175 μg in about 0.19 mL, about 125 μg to about 150 μg in about 0.19 mL, about 150 μg to about 600 μg in about 0.19 mL, about 150 μg to about 575 μg in about 0.19 mL, about 150 μg to about 550 μg in about 0.19 mL, about 150 μg to about 525 μg in about 0.19 mL, about 150 μg to about 500 μg in about 0.19 mL, about 150 μg to about 475 μg in about 0.19 mL, about 150 μg to about 450 μg in about 0.19 mL, about 150 μg to about 425 μg in about 0.19 mL, about 150 μg to about 400 μg in about 0.19 mL, about 150 μg to about 375 μg in about 0.19 mL, about 150 μg to about 350 μg in about 0.19 mL, about 150 μg to about 325 μg in about 0.19 mL, about 150 μg to about 300 μg in about 0.19 mL, about 150 μg to about 275 μg in about 0.19 mL, about 150 μg to about 250 μg in about 0.19 mL, about 150 μg to about 225 μg in about 0.19 mL, about 150 μg to about 200 μg in about 0.19 mL, about 150 μg to about 175 μg in about 0.19 mL, about 175 μg to about 600 μg in about 0.19 mL, about 175 μg to about 575 μg in about 0.19 mL, about 175 μg to about 550 μg in about 0.19 mL, about 175 μg to about 525 μg in about 0.19 mL, about 175 μg to about 500 μg in about 0.19 mL, about 175 μg to about 475 μg in about 0.19 mL, about 175 μg to about 450 μg in about 0.19 mL, about 175 μg to about 425 μg in about 0.19 mL, about 175 μg to about 400 μg in about 0.19 mL, about 175 μg to about 375 μg in about 0.19 mL, about 175 μg to about 350 μg in about 0.19 mL, about 175 μg to about 325 μg in about 0.19 mL, about 175 μg to about 300 μg in about 0.19 mL, about 175 μg to about 275 μg in about 0.19 mL, about 175 μg to about 250 μg in about 0.19 mL, about 175 μg to about 225 μg in about 0.19 mL, about 175 μg to about 200 μg in about 0.19 mL, about 200 μg to about 600 μg in about 0.19 mL, about 200 μg to about 575 μg in about 0.19 mL, about 200 μg to about 550 μg in about 0.19 mL, about 200 μg to about 525 μg in about 0.19 mL, about 200 μg to about 500 μg in about 0.19 mL, about 200 μg to about 475 μg in about 0.19 mL, about 200 μg to about 450 μg in about 0.19 mL, about 200 μg to about 425 μg in about 0.19 mL, about 200 μg to about 400 μg in about 0.19 mL, about 200 μg to about 375 μg in about 0.19 mL, about 200 μg to about 350 μg in about 0.19 mL, about 200 μg to about 325 μg in about 0.19 mL, about 200 μg to about 300 μg in about 0.19 mL, about 200 μg to about 275 μg in about 0.19 mL, about 200 μg to about 250 μg in about 0.19 mL, about 200 μg to about 225 μg in about 0.19 mL, about 225 μg to about 600 μg in about 0.19 mL, about 225 μg to about 575 μg in about 0.19 mL, about 225 μg to about 550 μg in about 0.19 mL, about 225 μg to about 525 μg in about 0.19 mL, about 225 μg to about 500 μg in about 0.19 mL, about 225 μg to about 475 μg in about 0.19 mL, about 225 μg to about 450 μg in about 0.19 mL, about 225 μg to about 425 μg in about 0.19 mL, about 225 μg to about 400 μg in about 0.19 mL, about 225 μg to about 375 μg in about 0.19 mL, about 225 μg to about 350 μg in about 0.19 mL, about 225 μg to about 325 μg in about 0.19 mL, about 225 μg to about 300 μg in about 0.19 mL, about 225 μg to about 275 μg in about 0.19 mL, or about 225 μg to about 250 μg in about 0.19 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.20 mL, about 5 μg to about 575 μg in about 0.20 mL, about 5 μg to about 550 μg in about 0.20 mL, about 5 μg to about 525 μg in about 0.20 mL, about 5 μg to about 500 μg in about 0.20 mL, about 5 μg to about 475 μg in about 0.20 mL, about 5 μg to about 450 μg in about 0.20 mL, about 5 μg to about 425 μg in about 0.20 mL, about 5 μg to about 400 μg in about 0.20 mL, about 5 μg to about 375 μg in about 0.20 mL, about 5 μg to about 350 μg in about 0.20 mL, about 5 μg to about 325 μg in about 0.20 mL, about 5 μg to about 300 μg in about 0.20 mL, about 5 μg to about 275 μg in about 0.20 mL, about 5 μg to about 250 μg in about 0.20 mL, about 5 μg to about 225 μg in about 0.20 mL, about 5 μg to about 200 μg in about 0.20 mL, about 5 μg to about 175 μg in about 0.20 mL, about 5 μg to about 150 μg in about 0.20 mL, about 5 μg to about 125 μg in about 0.20 mL, about 5 μg to about 100 μg in about 0.20 mL, about 5 μg to about 75 μg in about 0.20 mL, about 5 μg to about 50 μg in about 0.20 mL, about 5 μg to about 25 μg in about 0.20 mL, about 5 μg to about 20 μg in about 0.20 mL, about 5 μg to about 15 μg in about 0.20 mL, about 5 μg to about 10 μg in about 0.20 mL, about 10 μg to about 600 μg in about 0.20 mL, about 10 μg to about 575 μg in about 0.20 mL, about 10 μg to about 550 μg in about 0.20 mL, about 10 μg to about 525 μg in about 0.20 mL, about 10 μg to about 500 μg in about 0.20 mL, about 10 μg to about 475 μg in about 0.20 mL, about 10 μg to about 450 μg in about 0.20 mL, about 10 μg to about 425 μg in about 0.20 mL, about 10 μg to about 400 μg in about 0.20 mL, about 10 μg to about 375 μg in about 0.20 mL, about 10 μg to about 350 μg in about 0.20 mL, about 10 μg to about 325 μg in about 0.20 mL, about 10 μg to about 300 μg in about 0.20 mL, about 10 μg to about 275 μg in about 0.20 mL, about 10 μg to about 250 μg in about 0.20 mL, about 10 μg to about 225 μg in about 0.20 mL, about 10 μg to about 200 μg in about 0.20 mL, about 10 μg to about 175 μg in about 0.20 mL, about 10 μg to about 150 μg in about 0.20 mL, about 10 μg to about 125 μg in about 0.20 mL, about 10 μg to about 100 μg in about 0.20 mL, about 10 μg to about 75 μg in about 0.20 mL, about 10 μg to about 50 μg in about 0.20 mL, about 10 μg to about 25 μg in about 0.20 mL, about 10 μg to about 20 μg in about 0.20 mL, about 10 μg to about 15 μg in about 0.20 mL, about 15 μg to about 600 μg in about 0.20 mL, about 15 μg to about 575 μg in about 0.20 mL, about 15 μg to about 550 μg in about 0.20 mL, about 15 μg to about 525 μg in about 0.20 mL, about 15 μg to about 500 μg in about 0.20 mL, about 15 μg to about 475 μg in about 0.20 mL, about 15 μg to about 450 μg in about 0.20 mL, about 15 μg to about 425 μg in about 0.20 mL, about 15 μg to about 400 μg in about 0.20 mL, about 15 μg to about 375 μg in about 0.20 mL, about 15 μg to about 350 μg in about 0.20 mL, about 15 μg to about 325 μg in about 0.20 mL, about 15 μg to about 300 μg in about 0.20 mL, about 15 μg to about 275 μg in about 0.20 mL, about 15 μg to about 250 μg in about 0.20 mL, about 15 μg to about 225 μg in about 0.20 mL, about 15 μg to about 200 μg in about 0.20 mL, about 15 μg to about 175 μg in about 0.20 mL, about 15 μg to about 150 μg in about 0.20 mL, about 15 μg to about 125 μg in about 0.20 mL, about 15 μg to about 100 μg in about 0.20 mL, about 15 μg to about 75 μg in about 0.20 mL, about 15 μg to about 50 μg in about 0.20 mL, about 15 μg to about 25 μg in about 0.20 mL, about 15 μg to about 20 μg in about 0.20 mL, about 20 μg to about 600 μg in about 0.20 mL, about 20 μg to about 575 μg in about 0.20 mL, about 20 μg to about 550 μg in about 0.20 mL, about 20 μg to about 525 μg in about 0.20 mL, about 20 μg to about 500 μg in about 0.20 mL, about 20 μg to about 475 μg in about 0.20 mL, about 20 μg to about 450 μg in about 0.20 mL, about 20 μg to about 425 μg in about 0.20 mL, about 20 μg to about 400 μg in about 0.20 mL, about 20 μg to about 375 μg in about 0.20 mL, about 20 μg to about 350 μg in about 0.20 mL, about 20 μg to about 325 μg in about 0.20 mL, about 20 μg to about 300 μg in about 0.20 mL, about 20 μg to about 275 μg in about 0.20 mL, about 20 μg to about 250 μg in about 0.20 mL, about 20 μg to about 225 μg in about 0.20 mL, about 20 μg to about 200 μg in about 0.20 mL, about 20 μg to about 175 μg in about 0.20 mL, about 20 μg to about 150 μg in about 0.20 mL, about 20 μg to about 125 μg in about 0.20 mL, about 20 μg to about 100 μg in about 0.20 mL, about 20 μg to about 75 μg in about 0.20 mL, about 20 μg to about 50 μg in about 0.20 mL, about 20 μg to about 25 μg in about 0.20 mL, about 50 μg to about 600 μg in about 0.20 mL, about 50 μg to about 575 μg in about 0.20 mL, about 50 μg to about 550 μg in about 0.20 mL, about 50 μg to about 525 μg in about 0.20 mL, about 50 μg to about 500 μg in about 0.20 mL, about 50 μg to about 475 μg in about 0.20 mL, about 50 μg to about 450 μg in about 0.20 mL, about 50 μg to about 425 μg in about 0.20 mL, about 50 μg to about 400 μg in about 0.20 mL, about 50 μg to about 375 μg in about 0.20 mL, about 50 μg to about 350 μg in about 0.20 mL, about 50 μg to about 325 μg in about 0.20 mL, about 50 μg to about 300 μg in about 0.20 mL, about 50 μg to about 275 μg in about 0.20 mL, about 50 μg to about 250 μg in about 0.20 mL, about 50 μg to about 225 μg in about 0.20 mL, about 50 μg to about 200 μg in about 0.20 mL, about 50 μg to about 175 μg in about 0.20 mL, about 50 μg to about 150 μg in about 0.20 mL, about 50 μg to about 125 μg in about 0.20 mL, about 50 μg to about 100 μg in about 0.20 mL, about 50 μg to about 75 μg in about 0.20 mL, about 75 μg to about 600 μg in about 0.20 mL, about 75 μg to about 575 μg in about 0.20 mL, about 75 μg to about 550 μg in about 0.20 mL, about 75 μg to about 525 μg in about 0.20 mL, about 75 μg to about 500 μg in about 0.20 mL, about 75 μg to about 475 μg in about 0.20 mL, about 75 μg to about 450 μg in about 0.20 mL, about 75 μg to about 425 μg in about 0.20 mL, about 75 μg to about 400 μg in about 0.20 mL, about 75 μg to about 375 μg in about 0.20 mL, about 75 μg to about 350 μg in about 0.20 mL, about 75 μg to about 325 μg in about 0.20 mL, about 75 μg to about 300 μg in about 0.20 mL, about 75 μg to about 275 μg in about 0.20 mL, about 75 μg to about 250 μg in about 0.20 mL, about 75 μg to about 225 μg in about 0.20 mL, about 75 μg to about 200 μg in about 0.20 mL, about 75 μg to about 175 μg in about 0.20 mL, about 75 μg to about 150 μg in about 0.20 mL, about 75 μg to about 125 μg in about 0.20 mL, about 75 μg to about 100 μg in about 0.20 mL, about 100 μg to about 600 μg in about 0.20 mL, about 100 μg to about 575 μg in about 0.20 mL, about 100 μg to about 550 μg in about 0.20 mL, about 100 μg to about 525 μg in about 0.20 mL, about 100 μg to about 500 μg in about 0.20 mL, about 100 μg to about 475 μg in about 0.20 mL, about 100 μg to about 450 μg in about 0.20 mL, about 100 μg to about 425 μg in about 0.20 mL, about 100 μg to about 400 μg in about 0.20 mL, about 100 μg to about 375 μg in about 0.20 mL, about 100 μg to about 350 μg in about 0.20 mL, about 100 μg to about 325 μg in about 0.20 mL, about 100 μg to about 300 μg in about 0.20 mL, about 100 μg to about 275 μg in about 0.20 mL, about 100 μg to about 250 μg in about 0.20 mL, about 100 μg to about 225 μg in about 0.20 mL, about 100 μg to about 200 μg in about 0.20 mL, about 100 μg to about 175 μg in about 0.20 mL, about 100 μg to about 150 μg in about 0.20 mL, about 100 μg to about 125 μg in about 0.20 mL, about 125 μg to about 600 μg in about 0.20 mL, about 125 μg to about 575 μg in about 0.20 mL, about 125 μg to about 550 μg in about 0.20 mL, about 125 μg to about 525 μg in about 0.20 mL, about 125 μg to about 500 μg in about 0.20 mL, about 125 μg to about 475 μg in about 0.20 mL, about 125 μg to about 450 μg in about 0.20 mL, about 125 μg to about 425 μg in about 0.20 mL, about 125 μg to about 400 μg in about 0.20 mL, about 125 μg to about 375 μg in about 0.20 mL, about 125 μg to about 350 μg in about 0.20 mL, about 125 μg to about 325 μg in about 0.20 mL, about 125 μg to about 300 μg in about 0.20 mL, about 125 μg to about 275 μg in about 0.20 mL, about 125 μg to about 250 μg in about 0.20 mL, about 125 μg to about 225 μg in about 0.20 mL, about 125 μg to about 200 μg in about 0.20 mL, about 125 μg to about 175 μg in about 0.20 mL, about 125 μg to about 150 μg in about 0.20 mL, about 150 μg to about 600 μg in about 0.20 mL, about 150 μg to about 575 μg in about 0.20 mL, about 150 μg to about 550 μg in about 0.20 mL, about 150 μg to about 525 μg in about 0.20 mL, about 150 μg to about 500 μg in about 0.20 mL, about 150 μg to about 475 μg in about 0.20 mL, about 150 μg to about 450 μg in about 0.20 mL, about 150 μg to about 425 μg in about 0.20 mL, about 150 μg to about 400 μg in about 0.20 mL, about 150 μg to about 375 μg in about 0.20 mL, about 150 μg to about 350 μg in about 0.20 mL, about 150 μg to about 325 μg in about 0.20 mL, about 150 μg to about 300 μg in about 0.20 mL, about 150 μg to about 275 μg in about 0.20 mL, about 150 μg to about 250 μg in about 0.20 mL, about 150 μg to about 225 μg in about 0.20 mL, about 150 μg to about 200 μg in about 0.20 mL, about 150 μg to about 175 μg in about 0.20 mL, about 175 μg to about 600 μg in about 0.20 mL, about 175 μg to about 575 μg in about 0.20 mL, about 175 μg to about 550 μg in about 0.20 mL, about 175 μg to about 525 μg in about 0.20 mL, about 175 μg to about 500 μg in about 0.20 mL, about 175 μg to about 475 μg in about 0.20 mL, about 175 μg to about 450 μg in about 0.20 mL, about 175 μg to about 425 μg in about 0.20 mL, about 175 μg to about 400 μg in about 0.20 mL, about 175 μg to about 375 μg in about 0.20 mL, about 175 μg to about 350 μg in about 0.20 mL, about 175 μg to about 325 μg in about 0.20 mL, about 175 μg to about 300 μg in about 0.20 mL, about 175 μg to about 275 μg in about 0.20 mL, about 175 μg to about 250 μg in about 0.20 mL, about 175 μg to about 225 μg in about 0.20 mL, about 175 μg to about 200 μg in about 0.20 mL, about 200 μg to about 600 μg in about 0.20 mL, about 200 μg to about 575 μg in about 0.20 mL, about 200 μg to about 550 μg in about 0.20 mL, about 200 μg to about 525 μg in about 0.20 mL, about 200 μg to about 500 μg in about 0.20 mL, about 200 μg to about 475 μg in about 0.20 mL, about 200 μg to about 450 μg in about 0.20 mL, about 200 μg to about 425 μg in about 0.20 mL, about 200 μg to about 400 μg in about 0.20 mL, about 200 μg to about 375 μg in about 0.20 mL, about 200 μg to about 350 μg in about 0.20 mL, about 200 μg to about 325 μg in about 0.20 mL, about 200 μg to about 300 μg in about 0.20 mL, about 200 μg to about 275 μg in about 0.20 mL, about 200 μg to about 250 μg in about 0.20 mL, about 200 μg to about 225 μg in about 0.20 mL, about 225 μg to about 600 μg in about 0.20 mL, about 225 μg to about 575 μg in about 0.20 mL, about 225 μg to about 550 μg in about 0.20 mL, about 225 μg to about 525 μg in about 0.20 mL, about 225 μg to about 500 μg in about 0.20 mL, about 225 μg to about 475 μg in about 0.20 mL, about 225 μg to about 450 μg in about 0.20 mL, about 225 μg to about 425 μg in about 0.20 mL, about 225 μg to about 400 μg in about 0.20 mL, about 225 μg to about 375 μg in about 0.20 mL, about 225 μg to about 350 μg in about 0.20 mL, about 225 μg to about 325 μg in about 0.20 mL, about 225 μg to about 300 μg in about 0.20 mL, about 225 μg to about 275 μg in about 0.20 mL, or about 225 μg to about 250 μg in about 0.20 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.21 mL, about 5 μg to about 575 μg in about 0.21 mL, about 5 μg to about 550 μg in about 0.21 mL, about 5 μg to about 525 μg in about 0.21 mL, about 5 μg to about 500 μg in about 0.21 mL, about 5 μg to about 475 μg in about 0.21 mL, about 5 μg to about 450 μg in about 0.21 mL, about 5 μg to about 425 μg in about 0.21 mL, about 5 μg to about 400 μg in about 0.21 mL, about 5 μg to about 375 μg in about 0.21 mL, about 5 μg to about 350 μg in about 0.21 mL, about 5 μg to about 325 μg in about 0.21 mL, about 5 μg to about 300 μg in about 0.21 mL, about 5 μg to about 275 μg in about 0.21 mL, about 5 μg to about 250 μg in about 0.21 mL, about 5 μg to about 225 μg in about 0.21 mL, about 5 μg to about 200 μg in about 0.21 mL, about 5 μg to about 175 μg in about 0.21 mL, about 5 μg to about 150 μg in about 0.21 mL, about 5 μg to about 125 μg in about 0.21 mL, about 5 μg to about 100 μg in about 0.21 mL, about 5 μg to about 75 μg in about 0.21 mL, about 5 μg to about 50 μg in about 0.21 mL, about 5 μg to about 25 μg in about 0.21 mL, about 5 μg to about 20 μg in about 0.21 mL, about 5 μg to about 15 μg in about 0.21 mL, about 5 μg to about 10 μg in about 0.21 mL, about 10 μg to about 600 μg in about 0.21 mL, about 10 μg to about 575 μg in about 0.21 mL, about 10 μg to about 550 μg in about 0.21 mL, about 10 μg to about 525 μg in about 0.21 mL, about 10 μg to about 500 μg in about 0.21 mL, about 10 μg to about 475 μg in about 0.21 mL, about 10 μg to about 450 μg in about 0.21 mL, about 10 μg to about 425 μg in about 0.21 mL, about 10 μg to about 400 μg in about 0.21 mL, about 10 μg to about 375 μg in about 0.21 mL, about 10 μg to about 350 μg in about 0.21 mL, about 10 μg to about 325 μg in about 0.21 mL, about 10 μg to about 300 μg in about 0.21 mL, about 10 μg to about 275 μg in about 0.21 mL, about 10 μg to about 250 μg in about 0.21 mL, about 10 μg to about 225 μg in about 0.21 mL, about 10 μg to about 200 μg in about 0.21 mL, about 10 μg to about 175 μg in about 0.21 mL, about 10 μg to about 150 μg in about 0.21 mL, about 10 μg to about 125 μg in about 0.21 mL, about 10 μg to about 100 μg in about 0.21 mL, about 10 μg to about 75 μg in about 0.21 mL, about 10 μg to about 50 μg in about 0.21 mL, about 10 μg to about 25 μg in about 0.21 mL, about 10 μg to about 20 μg in about 0.21 mL, about 10 μg to about 15 μg in about 0.21 mL, about 15 μg to about 600 μg in about 0.21 mL, about 15 μg to about 575 μg in about 0.21 mL, about 15 μg to about 550 μg in about 0.21 mL, about 15 μg to about 525 μg in about 0.21 mL, about 15 μg to about 500 μg in about 0.21 mL, about 15 μg to about 475 μg in about 0.21 mL, about 15 μg to about 450 μg in about 0.21 mL, about 15 μg to about 425 μg in about 0.21 mL, about 15 μg to about 400 μg in about 0.21 mL, about 15 μg to about 375 μg in about 0.21 mL, about 15 μg to about 350 μg in about 0.21 mL, about 15 μg to about 325 μg in about 0.21 mL, about 15 μg to about 300 μg in about 0.21 mL, about 15 μg to about 275 μg in about 0.21 mL, about 15 μg to about 250 μg in about 0.21 mL, about 15 μg to about 225 μg in about 0.21 mL, about 15 μg to about 200 μg in about 0.21 mL, about 15 μg to about 175 μg in about 0.21 mL, about 15 μg to about 150 μg in about 0.21 mL, about 15 μg to about 125 μg in about 0.21 mL, about 15 μg to about 100 μg in about 0.21 mL, about 15 μg to about 75 μg in about 0.21 mL, about 15 μg to about 50 μg in about 0.21 mL, about 15 μg to about 25 μg in about 0.21 mL, about 15 μg to about 20 μg in about 0.21 mL, about 20 μg to about 600 μg in about 0.21 mL, about 20 μg to about 575 μg in about 0.21 mL, about 20 μg to about 550 μg in about 0.21 mL, about 20 μg to about 525 μg in about 0.21 mL, about 20 μg to about 500 μg in about 0.21 mL, about 20 μg to about 475 μg in about 0.21 mL, about 20 μg to about 450 μg in about 0.21 mL, about 20 μg to about 425 μg in about 0.21 mL, about 20 μg to about 400 μg in about 0.21 mL, about 20 μg to about 375 μg in about 0.21 mL, about 20 μg to about 350 μg in about 0.21 mL, about 20 μg to about 325 μg in about 0.21 mL, about 20 μg to about 300 μg in about 0.21 mL, about 20 μg to about 275 μg in about 0.21 mL, about 20 μg to about 250 μg in about 0.21 mL, about 20 μg to about 225 μg in about 0.21 mL, about 20 μg to about 200 μg in about 0.21 mL, about 20 μg to about 175 μg in about 0.21 mL, about 20 μg to about 150 μg in about 0.21 mL, about 20 μg to about 125 μg in about 0.21 mL, about 20 μg to about 100 μg in about 0.21 mL, about 20 μg to about 75 μg in about 0.21 mL, about 20 μg to about 50 μg in about 0.21 mL, about 20 μg to about 25 μg in about 0.21 mL, about 50 μg to about 600 μg in about 0.21 mL, about 50 μg to about 575 μg in about 0.21 mL, about 50 μg to about 550 μg in about 0.21 mL, about 50 μg to about 525 μg in about 0.21 mL, about 50 μg to about 500 μg in about 0.21 mL, about 50 μg to about 475 μg in about 0.21 mL, about 50 μg to about 450 μg in about 0.21 mL, about 50 μg to about 425 μg in about 0.21 mL, about 50 μg to about 400 μg in about 0.21 mL, about 50 μg to about 375 μg in about 0.21 mL, about 50 μg to about 350 μg in about 0.21 mL, about 50 μg to about 325 μg in about 0.21 mL, about 50 μg to about 300 μg in about 0.21 mL, about 50 μg to about 275 μg in about 0.21 mL, about 50 μg to about 250 μg in about 0.21 mL, about 50 μg to about 225 μg in about 0.21 mL, about 50 μg to about 200 μg in about 0.21 mL, about 50 μg to about 175 μg in about 0.21 mL, about 50 μg to about 150 μg in about 0.21 mL, about 50 μg to about 125 μg in about 0.21 mL, about 50 μg to about 100 μg in about 0.21 mL, about 50 μg to about 75 μg in about 0.21 mL, about 75 μg to about 600 μg in about 0.21 mL, about 75 μg to about 575 μg in about 0.21 mL, about 75 μg to about 550 μg in about 0.21 mL, about 75 μg to about 525 μg in about 0.21 mL, about 75 μg to about 500 μg in about 0.21 mL, about 75 μg to about 475 μg in about 0.21 mL, about 75 μg to about 450 μg in about 0.21 mL, about 75 μg to about 425 μg in about 0.21 mL, about 75 μg to about 400 μg in about 0.21 mL, about 75 μg to about 375 μg in about 0.21 mL, about 75 μg to about 350 μg in about 0.21 mL, about 75 μg to about 325 μg in about 0.21 mL, about 75 μg to about 300 μg in about 0.21 mL, about 75 μg to about 275 μg in about 0.21 mL, about 75 μg to about 250 μg in about 0.21 mL, about 75 μg to about 225 μg in about 0.21 mL, about 75 μg to about 200 μg in about 0.21 mL, about 75 μg to about 175 μg in about 0.21 mL, about 75 μg to about 150 μg in about 0.21 mL, about 75 μg to about 125 μg in about 0.21 mL, about 75 μg to about 100 μg in about 0.21 mL, about 100 μg to about 600 μg in about 0.21 mL, about 100 μg to about 575 μg in about 0.21 mL, about 100 μg to about 550 μg in about 0.21 mL, about 100 μg to about 525 μg in about 0.21 mL, about 100 μg to about 500 μg in about 0.21 mL, about 100 μg to about 475 μg in about 0.21 mL, about 100 μg to about 450 μg in about 0.21 mL, about 100 μg to about 425 μg in about 0.21 mL, about 100 μg to about 400 μg in about 0.21 mL, about 100 μg to about 375 μg in about 0.21 mL, about 100 μg to about 350 μg in about 0.21 mL, about 100 μg to about 325 μg in about 0.21 mL, about 100 μg to about 300 μg in about 0.21 mL, about 100 μg to about 275 μg in about 0.21 mL, about 100 μg to about 250 μg in about 0.21 mL, about 100 μg to about 225 μg in about 0.21 mL, about 100 μg to about 200 μg in about 0.21 mL, about 100 μg to about 175 μg in about 0.21 mL, about 100 μg to about 150 μg in about 0.21 mL, about 100 μg to about 125 μg in about 0.21 mL, about 125 μg to about 600 μg in about 0.21 mL, about 125 μg to about 575 μg in about 0.21 mL, about 125 μg to about 550 μg in about 0.21 mL, about 125 μg to about 525 μg in about 0.21 mL, about 125 μg to about 500 μg in about 0.21 mL, about 125 μg to about 475 μg in about 0.21 mL, about 125 μg to about 450 μg in about 0.21 mL, about 125 μg to about 425 μg in about 0.21 mL, about 125 μg to about 400 μg in about 0.21 mL, about 125 μg to about 375 μg in about 0.21 mL, about 125 μg to about 350 μg in about 0.21 mL, about 125 μg to about 325 μg in about 0.21 mL, about 125 μg to about 300 μg in about 0.21 mL, about 125 μg to about 275 μg in about 0.21 mL, about 125 μg to about 250 μg in about 0.21 mL, about 125 μg to about 225 μg in about 0.21 mL, about 125 μg to about 200 μg in about 0.21 mL, about 125 μg to about 175 μg in about 0.21 mL, about 125 μg to about 150 μg in about 0.21 mL, about 150 μg to about 600 μg in about 0.21 mL, about 150 μg to about 575 μg in about 0.21 mL, about 150 μg to about 550 μg in about 0.21 mL, about 150 μg to about 525 μg in about 0.21 mL, about 150 μg to about 500 μg in about 0.21 mL, about 150 μg to about 475 μg in about 0.21 mL, about 150 μg to about 450 μg in about 0.21 mL, about 150 μg to about 425 μg in about 0.21 mL, about 150 μg to about 400 μg in about 0.21 mL, about 150 μg to about 375 μg in about 0.21 mL, about 150 μg to about 350 μg in about 0.21 mL, about 150 μg to about 325 μg in about 0.21 mL, about 150 μg to about 300 μg in about 0.21 mL, about 150 μg to about 275 μg in about 0.21 mL, about 150 μg to about 250 μg in about 0.21 mL, about 150 μg to about 225 μg in about 0.21 mL, about 150 μg to about 200 μg in about 0.21 mL, about 150 μg to about 175 μg in about 0.21 mL, about 175 μg to about 600 μg in about 0.21 mL, about 175 μg to about 575 μg in about 0.21 mL, about 175 μg to about 550 μg in about 0.21 mL, about 175 μg to about 525 μg in about 0.21 mL, about 175 μg to about 500 μg in about 0.21 mL, about 175 μg to about 475 μg in about 0.21 mL, about 175 μg to about 450 μg in about 0.21 mL, about 175 μg to about 425 μg in about 0.21 mL, about 175 μg to about 400 μg in about 0.21 mL, about 175 μg to about 375 μg in about 0.21 mL, about 175 μg to about 350 μg in about 0.21 mL, about 175 μg to about 325 μg in about 0.21 mL, about 175 μg to about 300 μg in about 0.21 mL, about 175 μg to about 275 μg in about 0.21 mL, about 175 μg to about 250 μg in about 0.21 mL, about 175 μg to about 225 μg in about 0.21 mL, about 175 μg to about 200 μg in about 0.21 mL, about 200 μg to about 600 μg in about 0.21 mL, about 200 μg to about 575 μg in about 0.21 mL, about 200 μg to about 550 μg in about 0.21 mL, about 200 μg to about 525 μg in about 0.21 mL, about 200 μg to about 500 μg in about 0.21 mL, about 200 μg to about 475 μg in about 0.21 mL, about 200 μg to about 450 μg in about 0.21 mL, about 200 μg to about 425 μg in about 0.21 mL, about 200 μg to about 400 μg in about 0.21 mL, about 200 μg to about 375 μg in about 0.21 mL, about 200 μg to about 350 μg in about 0.21 mL, about 200 μg to about 325 μg in about 0.21 mL, about 200 μg to about 300 μg in about 0.21 mL, about 200 μg to about 275 μg in about 0.21 mL, about 200 μg to about 250 μg in about 0.21 mL, about 200 μg to about 225 μg in about 0.21 mL, about 225 μg to about 600 μg in about 0.21 mL, about 225 μg to about 575 μg in about 0.21 mL, about 225 μg to about 550 μg in about 0.21 mL, about 225 μg to about 525 μg in about 0.21 mL, about 225 μg to about 500 μg in about 0.21 mL, about 225 μg to about 475 μg in about 0.21 mL, about 225 μg to about 450 μg in about 0.21 mL, about 225 μg to about 425 μg in about 0.21 mL, about 225 μg to about 400 μg in about 0.21 mL, about 225 μg to about 375 μg in about 0.21 mL, about 225 μg to about 350 μg in about 0.21 mL, about 225 μg to about 325 μg in about 0.21 mL, about 225 μg to about 300 μg in about 0.21 mL, about 225 μg to about 275 μg in about 0.21 mL, or about 225 μg to about 250 μg in about 0.21 mL of the pharmaceutical composition. In some embodiments, each of the one or more ampules contains prostaglandin, prostaglandin analogue, prostacyclin or prostacyclin analogue in an amount of about 5 μg to about 600 μg in about 0.22 mL, about 5 μg to about 575 μg in about 0.22 mL, about 5 μg to about 550 μg in about 0.22 mL, about 5 μg to about 525 μg in about 0.22 mL, about 5 μg to about 500 μg in about 0.22 mL, about 5 μg to about 475 μg in about 0.22 mL, about 5 μg to about 450 μg in about 0.22 mL, about 5 μg to about 425 μg in about 0.22 mL, about 5 μg to about 400 μg in about 0.22 mL, about 5 μg to about 375 μg in about 0.22 mL, about 5 μg to about 350 μg in about 0.22 mL, about 5 μg to about 325 μg in about 0.22 mL, about 5 μg to about 300 μg in about 0.22 mL, about 5 μg to about 275 μg in about 0.22 mL, about 5 μg to about 250 μg in about 0.22 mL, about 5 μg to about 225 μg in about 0.22 mL, about 5 μg to about 200 μg in about 0.22 mL, about 5 μg to about 175 μg in about 0.22 mL, about 5 μg to about 150 μg in about 0.22 mL, about 5 μg to about 125 μg in about 0.22 mL, about 5 μg to about 100 μg in about 0.22 mL, about 5 μg to about 75 μg in about 0.22 mL, about 5 μg to about 50 μg in about 0.22 mL, about 5 μg to about 25 μg in about 0.22 mL, about 5 μg to about 20 μg in about 0.22 mL, about 5 μg to about 15 μg in about 0.22 mL, about 5 μg to about 10 μg in about 0.22 mL, about 10 μg to about 600 μg in about 0.22 mL, about 10 μg to about 575 μg in about 0.22 mL, about 10 μg to about 550 μg in about 0.22 mL, about 10 μg to about 525 μg in about 0.22 mL, about 10 μg to about 500 μg in about 0.22 mL, about 10 μg to about 475 μg in about 0.22 mL, about 10 μg to about 450 μg in about 0.22 mL, about 10 μg to about 425 μg in about 0.22 mL, about 10 μg to about 400 μg in about 0.22 mL, about 10 μg to about 375 μg in about 0.22 mL, about 10 μg to about 350 μg in about 0.22 mL, about 10 μg to about 325 μg in about 0.22 mL, about 10 μg to about 300 μg in about 0.22 mL, about 10 μg to about 275 μg in about 0.22 mL, about 10 μg to about 250 μg in about 0.22 mL, about 10 μg to about 225 μg in about 0.22 mL, about 10 μg to about 200 μg in about 0.22 mL, about 10 μg to about 175 μg in about 0.22 mL, about 10 μg to about 150 μg in about 0.22 mL, about 10 μg to about 125 μg in about 0.22 mL, about 10 μg to about 100 μg in about 0.22 mL, about 10 μg to about 75 μg in about 0.22 mL, about 10 μg to about 50 μg in about 0.22 mL, about 10 μg to about 25 μg in about 0.22 mL, about 10 μg to about 20 μg in about 0.22 mL, about 10 μg to about 15 μg in about 0.22 mL, about 15 μg to about 600 μg in about 0.22 mL, about 15 μg to about 575 μg in about 0.22 mL, about 15 μg to about 550 μg in about 0.22 mL, about 15 μg to about 525 μg in about 0.22 mL, about 15 μg to about 500 μg in about 0.22 mL, about 15 μg to about 475 μg in about 0.22 mL, about 15 μg to about 450 μg in about 0.22 mL, about 15 μg to about 425 μg in about 0.22 mL, about 15 μg to about 400 μg in about 0.22 mL, about 15 μg to about 375 μg in about 0.22 mL, about 15 μg to about 350 μg in about 0.22 mL, about 15 μg to about 325 μg in about 0.22 mL, about 15 μg to about 300 μg in about 0.22 mL, about 15 μg to about 275 μg in about 0.22 mL, about 15 μg to about 250 μg in about 0.22 mL, about 15 μg to about 225 μg in about 0.22 mL, about 15 μg to about 200 μg in about 0.22 mL, about 15 μg to about 175 μg in about 0.22 mL, about 15 μg to about 150 μg in about 0.22 mL, about 15 μg to about 125 μg in about 0.22 mL, about 15 μg to about 100 μg in about 0.22 mL, about 15 μg to about 75 μg in about 0.22 mL, about 15 μg to about 50 μg in about 0.22 mL, about 15 μg to about 25 μg in about 0.22 mL, about 15 μg to about 20 μg in about 0.22 mL, about 20 μg to about 600 μg in about 0.22 mL, about 20 μg to about 575 μg in about 0.22 mL, about 20 μg to about 550 μg in about 0.22 mL, about 20 μg to about 525 μg in about 0.22 mL, about 20 μg to about 500 μg in about 0.22 mL, about 20 μg to about 475 μg in about 0.22 mL, about 20 μg to about 450 μg in about 0.22 mL, about 20 μg to about 425 μg in about 0.22 mL, about 20 μg to about 400 μg in about 0.22 mL, about 20 μg to about 375 μg in about 0.22 mL, about 20 μg to about 350 μg in about 0.22 mL, about 20 μg to about 325 μg in about 0.22 mL, about 20 μg to about 300 μg in about 0.22 mL, about 20 μg to about 275 μg in about 0.22 mL, about 20 μg to about 250 μg in about 0.22 mL, about 20 μg to about 225 μg in about 0.22 mL, about 20 μg to about 200 μg in about 0.22 mL, about 20 μg to about 175 μg in about 0.22 mL, about 20 μg to about 150 μg in about 0.22 mL, about 20 μg to about 125 μg in about 0.22 mL, about 20 μg to about 100 μg in about 0.22 mL, about 20 μg to about 75 μg in about 0.22 mL, about 20 μg to about 50 μg in about 0.22 mL, about 20 μg to about 25 μg in about 0.22 mL, about 50 μg to about 600 μg in about 0.22 mL, about 50 μg to about 575 μg in about 0.22 mL, about 50 μg to about 550 μg in about 0.22 mL, about 50 μg to about 525 μg in about 0.22 mL, about 50 μg to about 500 μg in about 0.22 mL, about 50 μg to about 475 μg in about 0.22 mL, about 50 μg to about 450 μg in about 0.22 mL, about 50 μg to about 425 μg in about 0.22 mL, about 50 μg to about 400 μg in about 0.22 mL, about 50 μg to about 375 μg in about 0.22 mL, about 50 μg to about 350 μg in about 0.22 mL, about 50 μg to about 325 μg in about 0.22 mL, about 50 μg to about 300 μg in about 0.22 mL, about 50 μg to about 275 μg in about 0.22 mL, about 50 μg to about 250 μg in about 0.22 mL, about 50 μg to about 225 μg in about 0.22 mL, about 50 μg to about 200 μg in about 0.22 mL, about 50 μg to about 175 μg in about 0.22 mL, about 50 μg to about 150 μg in about 0.22 mL, about 50 μg to about 125 μg in about 0.22 mL, about 50 μg to about 100 μg in about 0.22 mL, about 50 μg to about 75 μg in about 0.22 mL, about 75 μg to about 600 μg in about 0.22 mL, about 75 μg to about 575 μg in about 0.22 mL, about 75 μg to about 550 μg in about 0.22 mL, about 75 μg to about 525 μg in about 0.22 mL, about 75 μg to about 500 μg in about 0.22 mL, about 75 μg to about 475 μg in about 0.22 mL, about 75 μg to about 450 μg in about 0.22 mL, about 75 μg to about 425 μg in about 0.22 mL, about 75 μg to about 400 μg in about 0.22 mL, about 75 μg to about 375 μg in about 0.22 mL, about 75 μg to about 350 μg in about 0.22 mL, about 75 μg to about 325 μg in about 0.22 mL, about 75 μg to about 300 μg in about 0.22 mL, about 75 μg to about 275 μg in about 0.22 mL, about 75 μg to about 250 μg in about 0.22 mL, about 75 μg to about 225 μg in about 0.22 mL, about 75 μg to about 200 μg in about 0.22 mL, about 75 μg to about 175 μg in about 0.22 mL, about 75 μg to about 150 μg in about 0.22 mL, about 75 μg to about 125 μg in about 0.22 mL, about 75 μg to about 100 μg in about 0.22 mL, about 100 μg to about 600 μg in about 0.22 mL, about 100 μg to about 575 μg in about 0.22 mL, about 100 μg to about 550 μg in about 0.22 mL, about 100 μg to about 525 μg in about 0.22 mL, about 100 μg to about 500 μg in about 0.22 mL, about 100 μg to about 475 μg in about 0.22 mL, about 100 μg to about 450 μg in about 0.22 mL, about 100 μg to about 425 μg in about 0.22 mL, about 100 μg to about 400 μg in about 0.22 mL, about 100 μg to about 375 μg in about 0.22 mL, about 100 μg to about 350 μg in about 0.22 mL, about 100 μg to about 325 μg in about 0.22 mL, about 100 μg to about 300 μg in about 0.22 mL, about 100 μg to about 275 μg in about 0.22 mL, about 100 μg to about 250 μg in about 0.22 mL, about 100 μg to about 225 μg in about 0.22 mL, about 100 μg to about 200 μg in about 0.22 mL, about 100 μg to about 175 μg in about 0.22 mL, about 100 μg to about 150 μg in about 0.22 mL, about 100 μg to about 125 μg in about 0.22 mL, about 125 μg to about 600 μg in about 0.22 mL, about 125 μg to about 575 μg in about 0.22 mL, about 125 μg to about 550 μg in about 0.22 mL, about 125 μg to about 525 μg in about 0.22 mL, about 125 μg to about 500 μg in about 0.22 mL, about 125 μg to about 475 μg in about 0.22 mL, about 125 μg to about 450 μg in about 0.22 mL, about 125 μg to about 425 μg in about 0.22 mL, about 125 μg to about 400 μg in about 0.22 mL, about 125 μg to about 375 μg in about 0.22 mL, about 125 μg to about 350 μg in about 0.22 mL, about 125 μg to about 325 μg in about 0.22 mL, about 125 μg to about 300 μg in about 0.22 mL, about 125 μg to about 275 μg in about 0.22 mL, about 125 μg to about 250 μg in about 0.22 mL, about 125 μg to about 225 μg in about 0.22 mL, about 125 μg to about 200 μg in about 0.22 mL, about 125 μg to about 175 μg in about 0.22 mL, about 125 μg to about 150 μg in about 0.22 mL, about 150 μg to about 600 μg in about 0.22 mL, about 150 μg to about 575 μg in about 0.22 mL, about 150 μg to about 550 μg in about 0.22 mL, about 150 μg to about 525 μg in about 0.22 mL, about 150 μg to about 500 μg in about 0.22 mL, about 150 μg to about 475 μg in about 0.22 mL, about 150 μg to about 450 μg in about 0.22 mL, about 150 μg to about 425 μg in about 0.22 mL, about 150 μg to about 400 μg in about 0.22 mL, about 150 μg to about 375 μg in about 0.22 mL, about 150 μg to about 350 μg in about 0.22 mL, about 150 μg to about 325 μg in about 0.22 mL, about 150 μg to about 300 μg in about 0.22 mL, about 150 μg to about 275 μg in about 0.22 mL, about 150 μg to about 250 μg in about 0.22 mL, about 150 μg to about 225 μg in about 0.22 mL, about 150 μg to about 200 μg in about 0.22 mL, about 150 μg to about 175 μg in about 0.22 mL, about 175 μg to about 600 μg in about 0.22 mL, about 175 μg to about 575 μg in about 0.22 mL, about 175 μg to about 550 μg in about 0.22 mL, about 175 μg to about 525 μg in about 0.22 mL, about 175 μg to about 500 μg in about 0.22 mL, about 175 μg to about 475 μg in about 0.22 mL, about 175 μg to about 450 μg in about 0.22 mL, about 175 μg to about 425 μg in about 0.22 mL, about 175 μg to about 400 μg in about 0.22 mL, about 175 μg to about 375 μg in about 0.22 mL, about 175 μg to about 350 μg in about 0.22 mL, about 175 μg to about 325 μg in about 0.22 mL, about 175 μg to about 300 μg in about 0.22 mL, about 175 μg to about 275 μg in about 0.22 mL, about 175 μg to about 250 μg in about 0.22 mL, about 175 μg to about 225 μg in about 0.22 mL, about 175 μg to about 200 μg in about 0.22 mL, about 200 μg to about 600 μg in about 0.22 mL, about 200 μg to about 575 μg in about 0.22 mL, about 200 μg to about 550 μg in about 0.22 mL, about 200 μg to about 525 μg in about 0.22 mL, about 200 μg to about 500 μg in about 0.22 mL, about 200 μg to about 475 μg in about 0.22 mL, about 200 μg to about 450 μg in about 0.22 mL, about 200 μg to about 425 μg in about 0.22 mL, about 200 μg to about 400 μg in about 0.22 mL, about 200 μg to about 375 μg in about 0.22 mL, about 200 μg to about 350 μg in about 0.22 mL, about 200 μg to about 325 μg in about 0.22 mL, about 200 μg to about 300 μg in about 0.22 mL, about 200 μg to about 275 μg in about 0.22 mL, about 200 μg to about 250 μg in about 0.22 mL, about 200 μg to about 225 μg in about 0.22 mL, about 225 μg to about 600 μg in about 0.22 mL, about 225 μg to about 575 μg in about 0.22 mL, about 225 μg to about 550 μg in about 0.22 mL, about 225 μg to about 525 μg in about 0.22 mL, about 225 μg to about 500 μg in about 0.22 mL, about 225 μg to about 475 μg in about 0.22 mL, about 225 μg to about 450 μg in about 0.22 mL, about 225 μg to about 425 μg in about 0.22 mL, about 225 μg to about 400 μg in about 0.22 mL, about 225 μg to about 375 μg in about 0.22 mL, about 225 μg to about 350 μg in about 0.22 mL, about 225 μg to about 325 μg in about 0.22 mL, about 225 μg to about 300 μg in about 0.22 mL, about 225 μg to about 275 μg in about 0.22 mL, or about 225 μg to about 250 μg in about 0.22 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.23 mL, about 5 μg to about 575 μg in about 0.23 mL, about 5 μg to about 550 μg in about 0.23 mL, about 5 μg to about 525 μg in about 0.23 mL, about 5 μg to about 500 μg in about 0.23 mL, about 5 μg to about 475 μg in about 0.23 mL, about 5 μg to about 450 μg in about 0.23 mL, about 5 μg to about 425 μg in about 0.23 mL, about 5 μg to about 400 μg in about 0.23 mL, about 5 μg to about 375 μg in about 0.23 mL, about 5 μg to about 350 μg in about 0.23 mL, about 5 μg to about 325 μg in about 0.23 mL, about 5 μg to about 300 μg in about 0.23 mL, about 5 μg to about 275 μg in about 0.23 mL, about 5 μg to about 250 μg in about 0.23 mL, about 5 μg to about 225 μg in about 0.23 mL, about 5 μg to about 200 μg in about 0.23 mL, about 5 μg to about 175 μg in about 0.23 mL, about 5 μg to about 150 μg in about 0.23 mL, about 5 μg to about 125 μg in about 0.23 mL, about 5 μg to about 100 μg in about 0.23 mL, about 5 μg to about 75 μg in about 0.23 mL, about 5 μg to about 50 μg in about 0.23 mL, about 5 μg to about 25 μg in about 0.23 mL, about 5 μg to about 20 μg in about 0.23 mL, about 5 μg to about 15 μg in about 0.23 mL, about 5 μg to about 10 μg in about 0.23 mL, about 10 μg to about 600 μg in about 0.23 mL, about 10 μg to about 575 μg in about 0.23 mL, about 10 μg to about 550 μg in about 0.23 mL, about 10 μg to about 525 μg in about 0.23 mL, about 10 μg to about 500 μg in about 0.23 mL, about 10 μg to about 475 μg in about 0.23 mL, about 10 μg to about 450 μg in about 0.23 mL, about 10 μg to about 425 μg in about 0.23 mL, about 10 μg to about 400 μg in about 0.23 mL, about 10 μg to about 375 μg in about 0.23 mL, about 10 μg to about 350 μg in about 0.23 mL, about 10 μg to about 325 μg in about 0.23 mL, about 10 μg to about 300 μg in about 0.23 mL, about 10 μg to about 275 μg in about 0.23 mL, about 10 μg to about 250 μg in about 0.23 mL, about 10 μg to about 225 μg in about 0.23 mL, about 10 μg to about 200 μg in about 0.23 mL, about 10 μg to about 175 μg in about 0.23 mL, about 10 μg to about 150 μg in about 0.23 mL, about 10 μg to about 125 μg in about 0.23 mL, about 10 μg to about 100 μg in about 0.23 mL, about 10 μg to about 75 μg in about 0.23 mL, about 10 μg to about 50 μg in about 0.23 mL, about 10 μg to about 25 μg in about 0.23 mL, about 10 μg to about 20 μg in about 0.23 mL, about 10 μg to about 15 μg in about 0.23 mL, about 15 μg to about 600 μg in about 0.23 mL, about 15 μg to about 575 μg in about 0.23 mL, about 15 μg to about 550 μg in about 0.23 mL, about 15 μg to about 525 μg in about 0.23 mL, about 15 μg to about 500 μg in about 0.23 mL, about 15 μg to about 475 μg in about 0.23 mL, about 15 μg to about 450 μg in about 0.23 mL, about 15 μg to about 425 μg in about 0.23 mL, about 15 μg to about 400 μg in about 0.23 mL, about 15 μg to about 375 μg in about 0.23 mL, about 15 μg to about 350 μg in about 0.23 mL, about 15 μg to about 325 μg in about 0.23 mL, about 15 μg to about 300 μg in about 0.23 mL, about 15 μg to about 275 μg in about 0.23 mL, about 15 μg to about 250 μg in about 0.23 mL, about 15 μg to about 225 μg in about 0.23 mL, about 15 μg to about 200 μg in about 0.23 mL, about 15 μg to about 175 μg in about 0.23 mL, about 15 μg to about 150 μg in about 0.23 mL, about 15 μg to about 125 μg in about 0.23 mL, about 15 μg to about 100 μg in about 0.23 mL, about 15 μg to about 75 μg in about 0.23 mL, about 15 μg to about 50 μg in about 0.23 mL, about 15 μg to about 25 μg in about 0.23 mL, about 15 μg to about 20 μg in about 0.23 mL, about 20 μg to about 600 μg in about 0.23 mL, about 20 μg to about 575 μg in about 0.23 mL, about 20 μg to about 550 μg in about 0.23 mL, about 20 μg to about 525 μg in about 0.23 mL, about 20 μg to about 500 μg in about 0.23 mL, about 20 μg to about 475 μg in about 0.23 mL, about 20 μg to about 450 μg in about 0.23 mL, about 20 μg to about 425 μg in about 0.23 mL, about 20 μg to about 400 μg in about 0.23 mL, about 20 μg to about 375 μg in about 0.23 mL, about 20 μg to about 350 μg in about 0.23 mL, about 20 μg to about 325 μg in about 0.23 mL, about 20 μg to about 300 μg in about 0.23 mL, about 20 μg to about 275 μg in about 0.23 mL, about 20 μg to about 250 μg in about 0.23 mL, about 20 μg to about 225 μg in about 0.23 mL, about 20 μg to about 200 μg in about 0.23 mL, about 20 μg to about 175 μg in about 0.23 mL, about 20 μg to about 150 μg in about 0.23 mL, about 20 μg to about 125 μg in about 0.23 mL, about 20 μg to about 100 μg in about 0.23 mL, about 20 μg to about 75 μg in about 0.23 mL, about 20 μg to about 50 μg in about 0.23 mL, about 20 μg to about 25 μg in about 0.23 mL, about 50 μg to about 600 μg in about 0.23 mL, about 50 μg to about 575 μg in about 0.23 mL, about 50 μg to about 550 μg in about 0.23 mL, about 50 μg to about 525 μg in about 0.23 mL, about 50 μg to about 500 μg in about 0.23 mL, about 50 μg to about 475 μg in about 0.23 mL, about 50 μg to about 450 μg in about 0.23 mL, about 50 μg to about 425 μg in about 0.23 mL, about 50 μg to about 400 μg in about 0.23 mL, about 50 μg to about 375 μg in about 0.23 mL, about 50 μg to about 350 μg in about 0.23 mL, about 50 μg to about 325 μg in about 0.23 mL, about 50 μg to about 300 μg in about 0.23 mL, about 50 μg to about 275 μg in about 0.23 mL, about 50 μg to about 250 μg in about 0.23 mL, about 50 μg to about 225 μg in about 0.23 mL, about 50 μg to about 200 μg in about 0.23 mL, about 50 μg to about 175 μg in about 0.23 mL, about 50 μg to about 150 μg in about 0.23 mL, about 50 μg to about 125 μg in about 0.23 mL, about 50 μg to about 100 μg in about 0.23 mL, about 50 μg to about 75 μg in about 0.23 mL, about 75 μg to about 600 μg in about 0.23 mL, about 75 μg to about 575 μg in about 0.23 mL, about 75 μg to about 550 μg in about 0.23 mL, about 75 μg to about 525 μg in about 0.23 mL, about 75 μg to about 500 μg in about 0.23 mL, about 75 μg to about 475 μg in about 0.23 mL, about 75 μg to about 450 μg in about 0.23 mL, about 75 μg to about 425 μg in about 0.23 mL, about 75 μg to about 400 μg in about 0.23 mL, about 75 μg to about 375 μg in about 0.23 mL, about 75 μg to about 350 μg in about 0.23 mL, about 75 μg to about 325 μg in about 0.23 mL, about 75 μg to about 300 μg in about 0.23 mL, about 75 μg to about 275 μg in about 0.23 mL, about 75 μg to about 250 μg in about 0.23 mL, about 75 μg to about 225 μg in about 0.23 mL, about 75 μg to about 200 μg in about 0.23 mL, about 75 μg to about 175 μg in about 0.23 mL, about 75 μg to about 150 μg in about 0.23 mL, about 75 μg to about 125 μg in about 0.23 mL, about 75 μg to about 100 μg in about 0.23 mL, about 100 μg to about 600 μg in about 0.23 mL, about 100 μg to about 575 μg in about 0.23 mL, about 100 μg to about 550 μg in about 0.23 mL, about 100 μg to about 525 μg in about 0.23 mL, about 100 μg to about 500 μg in about 0.23 mL, about 100 μg to about 475 μg in about 0.23 mL, about 100 μg to about 450 μg in about 0.23 mL, about 100 μg to about 425 μg in about 0.23 mL, about 100 μg to about 400 μg in about 0.23 mL, about 100 μg to about 375 μg in about 0.23 mL, about 100 μg to about 350 μg in about 0.23 mL, about 100 μg to about 325 μg in about 0.23 mL, about 100 μg to about 300 μg in about 0.23 mL, about 100 μg to about 275 μg in about 0.23 mL, about 100 μg to about 250 μg in about 0.23 mL, about 100 μg to about 225 μg in about 0.23 mL, about 100 μg to about 200 μg in about 0.23 mL, about 100 μg to about 175 μg in about 0.23 mL, about 100 μg to about 150 μg in about 0.23 mL, about 100 μg to about 125 μg in about 0.23 mL, about 125 μg to about 600 μg in about 0.23 mL, about 125 μg to about 575 μg in about 0.23 mL, about 125 μg to about 550 μg in about 0.23 mL, about 125 μg to about 525 μg in about 0.23 mL, about 125 μg to about 500 μg in about 0.23 mL, about 125 μg to about 475 μg in about 0.23 mL, about 125 μg to about 450 μg in about 0.23 mL, about 125 μg to about 425 μg in about 0.23 mL, about 125 μg to about 400 μg in about 0.23 mL, about 125 μg to about 375 μg in about 0.23 mL, about 125 μg to about 350 μg in about 0.23 mL, about 125 μg to about 325 μg in about 0.23 mL, about 125 μg to about 300 μg in about 0.23 mL, about 125 μg to about 275 μg in about 0.23 mL, about 125 μg to about 250 μg in about 0.23 mL, about 125 μg to about 225 μg in about 0.23 mL, about 125 μg to about 200 μg in about 0.23 mL, about 125 μg to about 175 μg in about 0.23 mL, about 125 μg to about 150 μg in about 0.23 mL, about 150 μg to about 600 μg in about 0.23 mL, about 150 μg to about 575 μg in about 0.23 mL, about 150 μg to about 550 μg in about 0.23 mL, about 150 μg to about 525 μg in about 0.23 mL, about 150 μg to about 500 μg in about 0.23 mL, about 150 μg to about 475 μg in about 0.23 mL, about 150 μg to about 450 μg in about 0.23 mL, about 150 μg to about 425 μg in about 0.23 mL, about 150 μg to about 400 μg in about 0.23 mL, about 150 μg to about 375 μg in about 0.23 mL, about 150 μg to about 350 μg in about 0.23 mL, about 150 μg to about 325 μg in about 0.23 mL, about 150 μg to about 300 μg in about 0.23 mL, about 150 μg to about 275 μg in about 0.23 mL, about 150 μg to about 250 μg in about 0.23 mL, about 150 μg to about 225 μg in about 0.23 mL, about 150 μg to about 200 μg in about 0.23 mL, about 150 μg to about 175 μg in about 0.23 mL, about 175 μg to about 600 μg in about 0.23 mL, about 175 μg to about 575 μg in about 0.23 mL, about 175 μg to about 550 μg in about 0.23 mL, about 175 μg to about 525 μg in about 0.23 mL, about 175 μg to about 500 μg in about 0.23 mL, about 175 μg to about 475 μg in about 0.23 mL, about 175 μg to about 450 μg in about 0.23 mL, about 175 μg to about 425 μg in about 0.23 mL, about 175 μg to about 400 μg in about 0.23 mL, about 175 μg to about 375 μg in about 0.23 mL, about 175 μg to about 350 μg in about 0.23 mL, about 175 μg to about 325 μg in about 0.23 mL, about 175 μg to about 300 μg in about 0.23 mL, about 175 μg to about 275 μg in about 0.23 mL, about 175 μg to about 250 μg in about 0.23 mL, about 175 μg to about 225 μg in about 0.23 mL, about 175 μg to about 200 μg in about 0.23 mL, about 200 μg to about 600 μg in about 0.23 mL, about 200 μg to about 575 μg in about 0.23 mL, about 200 μg to about 550 μg in about 0.23 mL, about 200 μg to about 525 μg in about 0.23 mL, about 200 μg to about 500 μg in about 0.23 mL, about 200 μg to about 475 μg in about 0.23 mL, about 200 μg to about 450 μg in about 0.23 mL, about 200 μg to about 425 μg in about 0.23 mL, about 200 μg to about 400 μg in about 0.23 mL, about 200 μg to about 375 μg in about 0.23 mL, about 200 μg to about 350 μg in about 0.23 mL, about 200 μg to about 325 μg in about 0.23 mL, about 200 μg to about 300 μg in about 0.23 mL, about 200 μg to about 275 μg in about 0.23 mL, about 200 μg to about 250 μg in about 0.23 mL, about 200 μg to about 225 μg in about 0.23 mL, about 225 μg to about 600 μg in about 0.23 mL, about 225 μg to about 575 μg in about 0.23 mL, about 225 μg to about 550 μg in about 0.23 mL, about 225 μg to about 525 μg in about 0.23 mL, about 225 μg to about 500 μg in about 0.23 mL, about 225 μg to about 475 μg in about 0.23 mL, about 225 μg to about 450 μg in about 0.23 mL, about 225 μg to about 425 μg in about 0.23 mL, about 225 μg to about 400 μg in about 0.23 mL, about 225 μg to about 375 μg in about 0.23 mL, about 225 μg to about 350 μg in about 0.23 mL, about 225 μg to about 325 μg in about 0.23 mL, about 225 μg to about 300 μg in about 0.23 mL, about 225 μg to about 275 μg in about 0.23 mL, or about 225 μg to about 250 μg in about 0.23 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.24 mL, about 5 μg to about 575 μg in about 0.24 mL, about 5 μg to about 550 μg in about 0.24 mL, about 5 μg to about 525 μg in about 0.24 mL, about 5 μg to about 500 μg in about 0.24 mL, about 5 μg to about 475 μg in about 0.24 mL, about 5 μg to about 450 μg in about 0.24 mL, about 5 μg to about 425 μg in about 0.24 mL, about 5 μg to about 400 μg in about 0.24 mL, about 5 μg to about 375 μg in about 0.24 mL, about 5 μg to about 350 μg in about 0.24 mL, about 5 μg to about 325 μg in about 0.24 mL, about 5 μg to about 300 μg in about 0.24 mL, about 5 μg to about 275 μg in about 0.24 mL, about 5 μg to about 250 μg in about 0.24 mL, about 5 μg to about 225 μg in about 0.24 mL, about 5 μg to about 200 μg in about 0.24 mL, about 5 μg to about 175 μg in about 0.24 mL, about 5 μg to about 150 μg in about 0.24 mL, about 5 μg to about 125 μg in about 0.24 mL, about 5 μg to about 100 μg in about 0.24 mL, about 5 μg to about 75 μg in about 0.24 mL, about 5 μg to about 50 μg in about 0.24 mL, about 5 μg to about 25 μg in about 0.24 mL, about 5 μg to about 20 μg in about 0.24 mL, about 5 μg to about 15 μg in about 0.24 mL, about 5 μg to about 10 μg in about 0.24 mL, about 10 μg to about 600 μg in about 0.24 mL, about 10 μg to about 575 μg in about 0.24 mL, about 10 μg to about 550 μg in about 0.24 mL, about 10 μg to about 525 μg in about 0.24 mL, about 10 μg to about 500 μg in about 0.24 mL, about 10 μg to about 475 μg in about 0.24 mL, about 10 μg to about 450 μg in about 0.24 mL, about 10 μg to about 425 μg in about 0.24 mL, about 10 μg to about 400 μg in about 0.24 mL, about 10 μg to about 375 μg in about 0.24 mL, about 10 μg to about 350 μg in about 0.24 mL, about 10 μg to about 325 μg in about 0.24 mL, about 10 μg to about 300 μg in about 0.24 mL, about 10 μg to about 275 μg in about 0.24 mL, about 10 μg to about 250 μg in about 0.24 mL, about 10 μg to about 225 μg in about 0.24 mL, about 10 μg to about 200 μg in about 0.24 mL, about 10 μg to about 175 μg in about 0.24 mL, about 10 μg to about 150 μg in about 0.24 mL, about 10 μg to about 125 μg in about 0.24 mL, about 10 μg to about 100 μg in about 0.24 mL, about 10 μg to about 75 μg in about 0.24 mL, about 10 μg to about 50 μg in about 0.24 mL, about 10 μg to about 25 μg in about 0.24 mL, about 10 μg to about 20 μg in about 0.24 mL, about 10 μg to about 15 μg in about 0.24 mL, about 15 μg to about 600 μg in about 0.24 mL, about 15 μg to about 575 μg in about 0.24 mL, about 15 μg to about 550 μg in about 0.24 mL, about 15 μg to about 525 μg in about 0.24 mL, about 15 μg to about 500 μg in about 0.24 mL, about 15 μg to about 475 μg in about 0.24 mL, about 15 μg to about 450 μg in about 0.24 mL, about 15 μg to about 425 μg in about 0.24 mL, about 15 μg to about 400 μg in about 0.24 mL, about 15 μg to about 375 μg in about 0.24 mL, about 15 μg to about 350 μg in about 0.24 mL, about 15 μg to about 325 μg in about 0.24 mL, about 15 μg to about 300 μg in about 0.24 mL, about 15 μg to about 275 μg in about 0.24 mL, about 15 μg to about 250 μg in about 0.24 mL, about 15 μg to about 225 μg in about 0.24 mL, about 15 μg to about 200 μg in about 0.24 mL, about 15 μg to about 175 μg in about 0.24 mL, about 15 μg to about 150 μg in about 0.24 mL, about 15 μg to about 125 μg in about 0.24 mL, about 15 μg to about 100 μg in about 0.24 mL, about 15 μg to about 75 μg in about 0.24 mL, about 15 μg to about 50 μg in about 0.24 mL, about 15 μg to about 25 μg in about 0.24 mL, about 15 μg to about 20 μg in about 0.24 mL, about 20 μg to about 600 μg in about 0.24 mL, about 20 μg to about 575 μg in about 0.24 mL, about 20 μg to about 550 μg in about 0.24 mL, about 20 μg to about 525 μg in about 0.24 mL, about 20 μg to about 500 μg in about 0.24 mL, about 20 μg to about 475 μg in about 0.24 mL, about 20 μg to about 450 μg in about 0.24 mL, about 20 μg to about 425 μg in about 0.24 mL, about 20 μg to about 400 μg in about 0.24 mL, about 20 μg to about 375 μg in about 0.24 mL, about 20 μg to about 350 μg in about 0.24 mL, about 20 μg to about 325 μg in about 0.24 mL, about 20 μg to about 300 μg in about 0.24 mL, about 20 μg to about 275 μg in about 0.24 mL, about 20 μg to about 250 μg in about 0.24 mL, about 20 μg to about 225 μg in about 0.24 mL, about 20 μg to about 200 μg in about 0.24 mL, about 20 μg to about 175 μg in about 0.24 mL, about 20 μg to about 150 μg in about 0.24 mL, about 20 μg to about 125 μg in about 0.24 mL, about 20 μg to about 100 μg in about 0.24 mL, about 20 μg to about 75 μg in about 0.24 mL, about 20 μg to about 50 μg in about 0.24 mL, about 20 μg to about 25 μg in about 0.24 mL, about 50 μg to about 600 μg in about 0.24 mL, about 50 μg to about 575 μg in about 0.24 mL, about 50 μg to about 550 μg in about 0.24 mL, about 50 μg to about 525 μg in about 0.24 mL, about 50 μg to about 500 μg in about 0.24 mL, about 50 μg to about 475 μg in about 0.24 mL, about 50 μg to about 450 μg in about 0.24 mL, about 50 μg to about 425 μg in about 0.24 mL, about 50 μg to about 400 μg in about 0.24 mL, about 50 μg to about 375 μg in about 0.24 mL, about 50 μg to about 350 μg in about 0.24 mL, about 50 μg to about 325 μg in about 0.24 mL, about 50 μg to about 300 μg in about 0.24 mL, about 50 μg to about 275 μg in about 0.24 mL, about 50 μg to about 250 μg in about 0.24 mL, about 50 μg to about 225 μg in about 0.24 mL, about 50 μg to about 200 μg in about 0.24 mL, about 50 μg to about 175 μg in about 0.24 mL, about 50 μg to about 150 μg in about 0.24 mL, about 50 μg to about 125 μg in about 0.24 mL, about 50 μg to about 100 μg in about 0.24 mL, about 50 μg to about 75 μg in about 0.24 mL, about 75 μg to about 600 μg in about 0.24 mL, about 75 μg to about 575 μg in about 0.24 mL, about 75 μg to about 550 μg in about 0.24 mL, about 75 μg to about 525 μg in about 0.24 mL, about 75 μg to about 500 μg in about 0.24 mL, about 75 μg to about 475 μg in about 0.24 mL, about 75 μg to about 450 μg in about 0.24 mL, about 75 μg to about 425 μg in about 0.24 mL, about 75 μg to about 400 μg in about 0.24 mL, about 75 μg to about 375 μg in about 0.24 mL, about 75 μg to about 350 μg in about 0.24 mL, about 75 μg to about 325 μg in about 0.24 mL, about 75 μg to about 300 μg in about 0.24 mL, about 75 μg to about 275 μg in about 0.24 mL, about 75 μg to about 250 μg in about 0.24 mL, about 75 μg to about 225 μg in about 0.24 mL, about 75 μg to about 200 μg in about 0.24 mL, about 75 μg to about 175 μg in about 0.24 mL, about 75 μg to about 150 μg in about 0.24 mL, about 75 μg to about 125 μg in about 0.24 mL, about 75 μg to about 100 μg in about 0.24 mL, about 100 μg to about 600 μg in about 0.24 mL, about 100 μg to about 575 μg in about 0.24 mL, about 100 μg to about 550 μg in about 0.24 mL, about 100 μg to about 525 μg in about 0.24 mL, about 100 μg to about 500 μg in about 0.24 mL, about 100 μg to about 475 μg in about 0.24 mL, about 100 μg to about 450 μg in about 0.24 mL, about 100 μg to about 425 μg in about 0.24 mL, about 100 μg to about 400 μg in about 0.24 mL, about 100 μg to about 375 μg in about 0.24 mL, about 100 μg to about 350 μg in about 0.24 mL, about 100 μg to about 325 μg in about 0.24 mL, about 100 μg to about 300 μg in about 0.24 mL, about 100 μg to about 275 μg in about 0.24 mL, about 100 μg to about 250 μg in about 0.24 mL, about 100 μg to about 225 μg in about 0.24 mL, about 100 μg to about 200 μg in about 0.24 mL, about 100 μg to about 175 μg in about 0.24 mL, about 100 μg to about 150 μg in about 0.24 mL, about 100 μg to about 125 μg in about 0.24 mL, about 125 μg to about 600 μg in about 0.24 mL, about 125 μg to about 575 μg in about 0.24 mL, about 125 μg to about 550 μg in about 0.24 mL, about 125 μg to about 525 μg in about 0.24 mL, about 125 μg to about 500 μg in about 0.24 mL, about 125 μg to about 475 μg in about 0.24 mL, about 125 μg to about 450 μg in about 0.24 mL, about 125 μg to about 425 μg in about 0.24 mL, about 125 μg to about 400 μg in about 0.24 mL, about 125 μg to about 375 μg in about 0.24 mL, about 125 μg to about 350 μg in about 0.24 mL, about 125 μg to about 325 μg in about 0.24 mL, about 125 μg to about 300 μg in about 0.24 mL, about 125 μg to about 275 μg in about 0.24 mL, about 125 μg to about 250 μg in about 0.24 mL, about 125 μg to about 225 μg in about 0.24 mL, about 125 μg to about 200 μg in about 0.24 mL, about 125 μg to about 175 μg in about 0.24 mL, about 125 μg to about 150 μg in about 0.24 mL, about 150 μg to about 600 μg in about 0.24 mL, about 150 μg to about 575 μg in about 0.24 mL, about 150 μg to about 550 μg in about 0.24 mL, about 150 μg to about 525 μg in about 0.24 mL, about 150 μg to about 500 μg in about 0.24 mL, about 150 μg to about 475 μg in about 0.24 mL, about 150 μg to about 450 μg in about 0.24 mL, about 150 μg to about 425 μg in about 0.24 mL, about 150 μg to about 400 μg in about 0.24 mL, about 150 μg to about 375 μg in about 0.24 mL, about 150 μg to about 350 μg in about 0.24 mL, about 150 μg to about 325 μg in about 0.24 mL, about 150 μg to about 300 μg in about 0.24 mL, about 150 μg to about 275 μg in about 0.24 mL, about 150 μg to about 250 μg in about 0.24 mL, about 150 μg to about 225 μg in about 0.24 mL, about 150 μg to about 200 μg in about 0.24 mL, about 150 μg to about 175 μg in about 0.24 mL, about 175 μg to about 600 μg in about 0.24 mL, about 175 μg to about 575 μg in about 0.24 mL, about 175 μg to about 550 μg in about 0.24 mL, about 175 μg to about 525 μg in about 0.24 mL, about 175 μg to about 500 μg in about 0.24 mL, about 175 μg to about 475 μg in about 0.24 mL, about 175 μg to about 450 μg in about 0.24 mL, about 175 μg to about 425 μg in about 0.24 mL, about 175 μg to about 400 μg in about 0.24 mL, about 175 μg to about 375 μg in about 0.24 mL, about 175 μg to about 350 μg in about 0.24 mL, about 175 μg to about 325 μg in about 0.24 mL, about 175 μg to about 300 μg in about 0.24 mL, about 175 μg to about 275 μg in about 0.24 mL, about 175 μg to about 250 μg in about 0.24 mL, about 175 μg to about 225 μg in about 0.24 mL, about 175 μg to about 200 μg in about 0.24 mL, about 200 μg to about 600 μg in about 0.24 mL, about 200 μg to about 575 μg in about 0.24 mL, about 200 μg to about 550 μg in about 0.24 mL, about 200 μg to about 525 μg in about 0.24 mL, about 200 μg to about 500 μg in about 0.24 mL, about 200 μg to about 475 μg in about 0.24 mL, about 200 μg to about 450 μg in about 0.24 mL, about 200 μg to about 425 μg in about 0.24 mL, about 200 μg to about 400 μg in about 0.24 mL, about 200 μg to about 375 μg in about 0.24 mL, about 200 μg to about 350 μg in about 0.24 mL, about 200 μg to about 325 μg in about 0.24 mL, about 200 μg to about 300 μg in about 0.24 mL, about 200 μg to about 275 μg in about 0.24 mL, about 200 μg to about 250 μg in about 0.24 mL, about 200 μg to about 225 μg in about 0.24 mL, about 225 μg to about 600 μg in about 0.24 mL, about 225 μg to about 575 μg in about 0.24 mL, about 225 μg to about 550 μg in about 0.24 mL, about 225 μg to about 525 μg in about 0.24 mL, about 225 μg to about 500 μg in about 0.24 mL, about 225 μg to about 475 μg in about 0.24 mL, about 225 μg to about 450 μg in about 0.24 mL, about 225 μg to about 425 μg in about 0.24 mL, about 225 μg to about 400 μg in about 0.24 mL, about 225 μg to about 375 μg in about 0.24 mL, about 225 μg to about 350 μg in about 0.24 mL, about 225 μg to about 325 μg in about 0.24 mL, about 225 μg to about 300 μg in about 0.24 mL, about 225 μg to about 275 μg in about 0.24 mL, or about 225 μg to about 250 μg in about 0.24 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.25 mL, about 5 μg to about 575 μg in about 0.25 mL, about 5 μg to about 550 μg in about 0.25 mL, about 5 μg to about 525 μg in about 0.25 mL, about 5 μg to about 500 μg in about 0.25 mL, about 5 μg to about 475 μg in about 0.25 mL, about 5 μg to about 450 μg in about 0.25 mL, about 5 μg to about 425 μg in about 0.25 mL, about 5 μg to about 400 μg in about 0.25 mL, about 5 μg to about 375 μg in about 0.25 mL, about 5 μg to about 350 μg in about 0.25 mL, about 5 μg to about 325 μg in about 0.25 mL, about 5 μg to about 300 μg in about 0.25 mL, about 5 μg to about 275 μg in about 0.25 mL, about 5 μg to about 250 μg in about 0.25 mL, about 5 μg to about 225 μg in about 0.25 mL, about 5 μg to about 200 μg in about 0.25 mL, about 5 μg to about 175 μg in about 0.25 mL, about 5 μg to about 150 μg in about 0.25 mL, about 5 μg to about 125 μg in about 0.25 mL, about 5 μg to about 100 μg in about 0.25 mL, about 5 μg to about 75 μg in about 0.25 mL, about 5 μg to about 50 μg in about 0.25 mL, about 5 μg to about 25 μg in about 0.25 mL, about 5 μg to about 20 μg in about 0.25 mL, about 5 μg to about 15 μg in about 0.25 mL, about 5 μg to about 10 μg in about 0.25 mL, about 10 μg to about 600 μg in about 0.25 mL, about 10 μg to about 575 μg in about 0.25 mL, about 10 μg to about 550 μg in about 0.25 mL, about 10 μg to about 525 μg in about 0.25 mL, about 10 μg to about 500 μg in about 0.25 mL, about 10 μg to about 475 μg in about 0.25 mL, about 10 μg to about 450 μg in about 0.25 mL, about 10 μg to about 425 μg in about 0.25 mL, about 10 μg to about 400 μg in about 0.25 mL, about 10 μg to about 375 μg in about 0.25 mL, about 10 μg to about 350 μg in about 0.25 mL, about 10 μg to about 325 μg in about 0.25 mL, about 10 μg to about 300 μg in about 0.25 mL, about 10 μg to about 275 μg in about 0.25 mL, about 10 μg to about 250 μg in about 0.25 mL, about 10 μg to about 225 μg in about 0.25 mL, about 10 μg to about 200 μg in about 0.25 mL, about 10 μg to about 175 μg in about 0.25 mL, about 10 μg to about 150 μg in about 0.25 mL, about 10 μg to about 125 μg in about 0.25 mL, about 10 μg to about 100 μg in about 0.25 mL, about 10 μg to about 75 μg in about 0.25 mL, about 10 μg to about 50 μg in about 0.25 mL, about 10 μg to about 25 μg in about 0.25 mL, about 10 μg to about 20 μg in about 0.25 mL, about 10 μg to about 15 μg in about 0.25 mL, about 15 μg to about 600 μg in about 0.25 mL, about 15 μg to about 575 μg in about 0.25 mL, about 15 μg to about 550 μg in about 0.25 mL, about 15 μg to about 525 μg in about 0.25 mL, about 15 μg to about 500 μg in about 0.25 mL, about 15 μg to about 475 μg in about 0.25 mL, about 15 μg to about 450 μg in about 0.25 mL, about 15 μg to about 425 μg in about 0.25 mL, about 15 μg to about 400 μg in about 0.25 mL, about 15 μg to about 375 μg in about 0.25 mL, about 15 μg to about 350 μg in about 0.25 mL, about 15 μg to about 325 μg in about 0.25 mL, about 15 μg to about 300 μg in about 0.25 mL, about 15 μg to about 275 μg in about 0.25 mL, about 15 μg to about 250 μg in about 0.25 mL, about 15 μg to about 225 μg in about 0.25 mL, about 15 μg to about 200 μg in about 0.25 mL, about 15 μg to about 175 μg in about 0.25 mL, about 15 μg to about 150 μg in about 0.25 mL, about 15 μg to about 125 μg in about 0.25 mL, about 15 μg to about 100 μg in about 0.25 mL, about 15 μg to about 75 μg in about 0.25 mL, about 15 μg to about 50 μg in about 0.25 mL, about 15 μg to about 25 μg in about 0.25 mL, about 15 μg to about 20 μg in about 0.25 mL, about 20 μg to about 600 μg in about 0.25 mL, about 20 μg to about 575 μg in about 0.25 mL, about 20 μg to about 550 μg in about 0.25 mL, about 20 μg to about 525 μg in about 0.25 mL, about 20 μg to about 500 μg in about 0.25 mL, about 20 μg to about 475 μg in about 0.25 mL, about 20 μg to about 450 μg in about 0.25 mL, about 20 μg to about 425 μg in about 0.25 mL, about 20 μg to about 400 μg in about 0.25 mL, about 20 μg to about 375 μg in about 0.25 mL, about 20 μg to about 350 μg in about 0.25 mL, about 20 μg to about 325 μg in about 0.25 mL, about 20 μg to about 300 μg in about 0.25 mL, about 20 μg to about 275 μg in about 0.25 mL, about 20 μg to about 250 μg in about 0.25 mL, about 20 μg to about 225 μg in about 0.25 mL, about 20 μg to about 200 μg in about 0.25 mL, about 20 μg to about 175 μg in about 0.25 mL, about 20 μg to about 150 μg in about 0.25 mL, about 20 μg to about 125 μg in about 0.25 mL, about 20 μg to about 100 μg in about 0.25 mL, about 20 μg to about 75 μg in about 0.25 mL, about 20 μg to about 50 μg in about 0.25 mL, about 20 μg to about 25 μg in about 0.25 mL, about 50 μg to about 600 μg in about 0.25 mL, about 50 μg to about 575 μg in about 0.25 mL, about 50 μg to about 550 μg in about 0.25 mL, about 50 μg to about 525 μg in about 0.25 mL, about 50 μg to about 500 μg in about 0.25 mL, about 50 μg to about 475 μg in about 0.25 mL, about 50 μg to about 450 μg in about 0.25 mL, about 50 μg to about 425 μg in about 0.25 mL, about 50 μg to about 400 μg in about 0.25 mL, about 50 μg to about 375 μg in about 0.25 mL, about 50 μg to about 350 μg in about 0.25 mL, about 50 μg to about 325 μg in about 0.25 mL, about 50 μg to about 300 μg in about 0.25 mL, about 50 μg to about 275 μg in about 0.25 mL, about 50 μg to about 250 μg in about 0.25 mL, about 50 μg to about 225 μg in about 0.25 mL, about 50 μg to about 200 μg in about 0.25 mL, about 50 μg to about 175 μg in about 0.25 mL, about 50 μg to about 150 μg in about 0.25 mL, about 50 μg to about 125 μg in about 0.25 mL, about 50 μg to about 100 μg in about 0.25 mL, about 50 μg to about 75 μg in about 0.25 mL, about 75 μg to about 600 μg in about 0.25 mL, about 75 μg to about 575 μg in about 0.25 mL, about 75 μg to about 550 μg in about 0.25 mL, about 75 μg to about 525 μg in about 0.25 mL, about 75 μg to about 500 μg in about 0.25 mL, about 75 μg to about 475 μg in about 0.25 mL, about 75 μg to about 450 μg in about 0.25 mL, about 75 μg to about 425 μg in about 0.25 mL, about 75 μg to about 400 μg in about 0.25 mL, about 75 μg to about 375 μg in about 0.25 mL, about 75 μg to about 350 μg in about 0.25 mL, about 75 μg to about 325 μg in about 0.25 mL, about 75 μg to about 300 μg in about 0.25 mL, about 75 μg to about 275 μg in about 0.25 mL, about 75 μg to about 250 μg in about 0.25 mL, about 75 μg to about 225 μg in about 0.25 mL, about 75 μg to about 200 μg in about 0.25 mL, about 75 μg to about 175 μg in about 0.25 mL, about 75 μg to about 150 μg in about 0.25 mL, about 75 μg to about 125 μg in about 0.25 mL, about 75 μg to about 100 μg in about 0.25 mL, about 100 μg to about 600 μg in about 0.25 mL, about 100 μg to about 575 μg in about 0.25 mL, about 100 μg to about 550 μg in about 0.25 mL, about 100 μg to about 525 μg in about 0.25 mL, about 100 μg to about 500 μg in about 0.25 mL, about 100 μg to about 475 μg in about 0.25 mL, about 100 μg to about 450 μg in about 0.25 mL, about 100 μg to about 425 μg in about 0.25 mL, about 100 μg to about 400 μg in about 0.25 mL, about 100 μg to about 375 μg in about 0.25 mL, about 100 μg to about 350 μg in about 0.25 mL, about 100 μg to about 325 μg in about 0.25 mL, about 100 μg to about 300 μg in about 0.25 mL, about 100 μg to about 275 μg in about 0.25 mL, about 100 μg to about 250 μg in about 0.25 mL, about 100 μg to about 225 μg in about 0.25 mL, about 100 μg to about 200 μg in about 0.25 mL, about 100 μg to about 175 μg in about 0.25 mL, about 100 μg to about 150 μg in about 0.25 mL, about 100 μg to about 125 μg in about 0.25 mL, about 125 μg to about 600 μg in about 0.25 mL, about 125 μg to about 575 μg in about 0.25 mL, about 125 μg to about 550 μg in about 0.25 mL, about 125 μg to about 525 μg in about 0.25 mL, about 125 μg to about 500 μg in about 0.25 mL, about 125 μg to about 475 μg in about 0.25 mL, about 125 μg to about 450 μg in about 0.25 mL, about 125 μg to about 425 μg in about 0.25 mL, about 125 μg to about 400 μg in about 0.25 mL, about 125 μg to about 375 μg in about 0.25 mL, about 125 μg to about 350 μg in about 0.25 mL, about 125 μg to about 325 μg in about 0.25 mL, about 125 μg to about 300 μg in about 0.25 mL, about 125 μg to about 275 μg in about 0.25 mL, about 125 μg to about 250 μg in about 0.25 mL, about 125 μg to about 225 μg in about 0.25 mL, about 125 μg to about 200 μg in about 0.25 mL, about 125 μg to about 175 μg in about 0.25 mL, about 125 μg to about 150 μg in about 0.25 mL, about 150 μg to about 600 μg in about 0.25 mL, about 150 μg to about 575 μg in about 0.25 mL, about 150 μg to about 550 μg in about 0.25 mL, about 150 μg to about 525 μg in about 0.25 mL, about 150 μg to about 500 μg in about 0.25 mL, about 150 μg to about 475 μg in about 0.25 mL, about 150 μg to about 450 μg in about 0.25 mL, about 150 μg to about 425 μg in about 0.25 mL, about 150 μg to about 400 μg in about 0.25 mL, about 150 μg to about 375 μg in about 0.25 mL, about 150 μg to about 350 μg in about 0.25 mL, about 150 μg to about 325 μg in about 0.25 mL, about 150 μg to about 300 μg in about 0.25 mL, about 150 μg to about 275 μg in about 0.25 mL, about 150 μg to about 250 μg in about 0.25 mL, about 150 μg to about 225 μg in about 0.25 mL, about 150 μg to about 200 μg in about 0.25 mL, about 150 μg to about 175 μg in about 0.25 mL, about 175 μg to about 600 μg in about 0.25 mL, about 175 μg to about 575 μg in about 0.25 mL, about 175 μg to about 550 μg in about 0.25 mL, about 175 μg to about 525 μg in about 0.25 mL, about 175 μg to about 500 μg in about 0.25 mL, about 175 μg to about 475 μg in about 0.25 mL, about 175 μg to about 450 μg in about 0.25 mL, about 175 μg to about 425 μg in about 0.25 mL, about 175 μg to about 400 μg in about 0.25 mL, about 175 μg to about 375 μg in about 0.25 mL, about 175 μg to about 350 μg in about 0.25 mL, about 175 μg to about 325 μg in about 0.25 mL, about 175 μg to about 300 μg in about 0.25 mL, about 175 μg to about 275 μg in about 0.25 mL, about 175 μg to about 250 μg in about 0.25 mL, about 175 μg to about 225 μg in about 0.25 mL, about 175 μg to about 200 μg in about 0.25 mL, about 200 μg to about 600 μg in about 0.25 mL, about 200 μg to about 575 μg in about 0.25 mL, about 200 μg to about 550 μg in about 0.25 mL, about 200 μg to about 525 μg in about 0.25 mL, about 200 μg to about 500 μg in about 0.25 mL, about 200 μg to about 475 μg in about 0.25 mL, about 200 μg to about 450 μg in about 0.25 mL, about 200 μg to about 425 μg in about 0.25 mL, about 200 μg to about 400 μg in about 0.25 mL, about 200 μg to about 375 μg in about 0.25 mL, about 200 μg to about 350 μg in about 0.25 mL, about 200 μg to about 325 μg in about 0.25 mL, about 200 μg to about 300 μg in about 0.25 mL, about 200 μg to about 275 μg in about 0.25 mL, about 200 μg to about 250 μg in about 0.25 mL, about 200 μg to about 225 μg in about 0.25 mL, about 225 μg to about 600 μg in about 0.25 mL, about 225 μg to about 575 μg in about 0.25 mL, about 225 μg to about 550 μg in about 0.25 mL, about 225 μg to about 525 μg in about 0.25 mL, about 225 μg to about 500 μg in about 0.25 mL, about 225 μg to about 475 μg in about 0.25 mL, about 225 μg to about 450 μg in about 0.25 mL, about 225 μg to about 425 μg in about 0.25 mL, about 225 μg to about 400 μg in about 0.25 mL, about 225 μg to about 375 μg in about 0.25 mL, about 225 μg to about 350 μg in about 0.25 mL, about 225 μg to about 325 μg in about 0.25 mL, about 225 μg to about 300 μg in about 0.25 mL, about 225 μg to about 275 μg in about 0.25 mL, or about 225 μg to about 250 μg in about 0.25 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.26 mL, about 5 μg to about 575 μg in about 0.26 mL, about 5 μg to about 550 μg in about 0.26 mL, about 5 μg to about 525 μg in about 0.26 mL, about 5 μg to about 500 μg in about 0.26 mL, about 5 μg to about 475 μg in about 0.26 mL, about 5 μg to about 450 μg in about 0.26 mL, about 5 μg to about 425 μg in about 0.26 mL, about 5 μg to about 400 μg in about 0.26 mL, about 5 μg to about 375 μg in about 0.26 mL, about 5 μg to about 350 μg in about 0.26 mL, about 5 μg to about 325 μg in about 0.26 mL, about 5 μg to about 300 μg in about 0.26 mL, about 5 μg to about 275 μg in about 0.26 mL, about 5 μg to about 250 μg in about 0.26 mL, about 5 μg to about 225 μg in about 0.26 mL, about 5 μg to about 200 μg in about 0.26 mL, about 5 μg to about 175 μg in about 0.26 mL, about 5 μg to about 150 μg in about 0.26 mL, about 5 μg to about 125 μg in about 0.26 mL, about 5 μg to about 100 μg in about 0.26 mL, about 5 μg to about 75 μg in about 0.26 mL, about 5 μg to about 50 μg in about 0.26 mL, about 5 μg to about 25 μg in about 0.26 mL, about 5 μg to about 20 μg in about 0.26 mL, about 5 μg to about 15 μg in about 0.26 mL, about 5 μg to about 10 μg in about 0.26 mL, about 10 μg to about 600 μg in about 0.26 mL, about 10 μg to about 575 μg in about 0.26 mL, about 10 μg to about 550 μg in about 0.26 mL, about 10 μg to about 525 μg in about 0.26 mL, about 10 μg to about 500 μg in about 0.26 mL, about 10 μg to about 475 μg in about 0.26 mL, about 10 μg to about 450 μg in about 0.26 mL, about 10 μg to about 425 μg in about 0.26 mL, about 10 μg to about 400 μg in about 0.26 mL, about 10 μg to about 375 μg in about 0.26 mL, about 10 μg to about 350 μg in about 0.26 mL, about 10 μg to about 325 μg in about 0.26 mL, about 10 μg to about 300 μg in about 0.26 mL, about 10 μg to about 275 μg in about 0.26 mL, about 10 μg to about 250 μg in about 0.26 mL, about 10 μg to about 225 μg in about 0.26 mL, about 10 μg to about 200 μg in about 0.26 mL, about 10 μg to about 175 μg in about 0.26 mL, about 10 μg to about 150 μg in about 0.26 mL, about 10 μg to about 125 μg in about 0.26 mL, about 10 μg to about 100 μg in about 0.26 mL, about 10 μg to about 75 μg in about 0.26 mL, about 10 μg to about 50 μg in about 0.26 mL, about 10 μg to about 25 μg in about 0.26 mL, about 10 μg to about 20 μg in about 0.26 mL, about 10 μg to about 15 μg in about 0.26 mL, about 15 μg to about 600 μg in about 0.26 mL, about 15 μg to about 575 μg in about 0.26 mL, about 15 μg to about 550 μg in about 0.26 mL, about 15 μg to about 525 μg in about 0.26 mL, about 15 μg to about 500 μg in about 0.26 mL, about 15 μg to about 475 μg in about 0.26 mL, about 15 μg to about 450 μg in about 0.26 mL, about 15 μg to about 425 μg in about 0.26 mL, about 15 μg to about 400 μg in about 0.26 mL, about 15 μg to about 375 μg in about 0.26 mL, about 15 μg to about 350 μg in about 0.26 mL, about 15 μg to about 325 μg in about 0.26 mL, about 15 μg to about 300 μg in about 0.26 mL, about 15 μg to about 275 μg in about 0.26 mL, about 15 μg to about 250 μg in about 0.26 mL, about 15 μg to about 225 μg in about 0.26 mL, about 15 μg to about 200 μg in about 0.26 mL, about 15 μg to about 175 μg in about 0.26 mL, about 15 μg to about 150 μg in about 0.26 mL, about 15 μg to about 125 μg in about 0.26 mL, about 15 μg to about 100 μg in about 0.26 mL, about 15 μg to about 75 μg in about 0.26 mL, about 15 μg to about 50 μg in about 0.26 mL, about 15 μg to about 25 μg in about 0.26 mL, about 15 μg to about 20 μg in about 0.26 mL, about 20 μg to about 600 μg in about 0.26 mL, about 20 μg to about 575 μg in about 0.26 mL, about 20 μg to about 550 μg in about 0.26 mL, about 20 μg to about 525 μg in about 0.26 mL, about 20 μg to about 500 μg in about 0.26 mL, about 20 μg to about 475 μg in about 0.26 mL, about 20 μg to about 450 μg in about 0.26 mL, about 20 μg to about 425 μg in about 0.26 mL, about 20 μg to about 400 μg in about 0.26 mL, about 20 μg to about 375 μg in about 0.26 mL, about 20 μg to about 350 μg in about 0.26 mL, about 20 μg to about 325 μg in about 0.26 mL, about 20 μg to about 300 μg in about 0.26 mL, about 20 μg to about 275 μg in about 0.26 mL, about 20 μg to about 250 μg in about 0.26 mL, about 20 μg to about 225 μg in about 0.26 mL, about 20 μg to about 200 μg in about 0.26 mL, about 20 μg to about 175 μg in about 0.26 mL, about 20 μg to about 150 μg in about 0.26 mL, about 20 μg to about 125 μg in about 0.26 mL, about 20 μg to about 100 μg in about 0.26 mL, about 20 μg to about 75 μg in about 0.26 mL, about 20 μg to about 50 μg in about 0.26 mL, about 20 μg to about 25 μg in about 0.26 mL, about 50 μg to about 600 μg in about 0.26 mL, about 50 μg to about 575 μg in about 0.26 mL, about 50 μg to about 550 μg in about 0.26 mL, about 50 μg to about 525 μg in about 0.26 mL, about 50 μg to about 500 μg in about 0.26 mL, about 50 μg to about 475 μg in about 0.26 mL, about 50 μg to about 450 μg in about 0.26 mL, about 50 μg to about 425 μg in about 0.26 mL, about 50 μg to about 400 μg in about 0.26 mL, about 50 μg to about 375 μg in about 0.26 mL, about 50 μg to about 350 μg in about 0.26 mL, about 50 μg to about 325 μg in about 0.26 mL, about 50 μg to about 300 μg in about 0.26 mL, about 50 μg to about 275 μg in about 0.26 mL, about 50 μg to about 250 μg in about 0.26 mL, about 50 μg to about 225 μg in about 0.26 mL, about 50 μg to about 200 μg in about 0.26 mL, about 50 μg to about 175 μg in about 0.26 mL, about 50 μg to about 150 μg in about 0.26 mL, about 50 μg to about 125 μg in about 0.26 mL, about 50 μg to about 100 μg in about 0.26 mL, about 50 μg to about 75 μg in about 0.26 mL, about 75 μg to about 600 μg in about 0.26 mL, about 75 μg to about 575 μg in about 0.26 mL, about 75 μg to about 550 μg in about 0.26 mL, about 75 μg to about 525 μg in about 0.26 mL, about 75 μg to about 500 μg in about 0.26 mL, about 75 μg to about 475 μg in about 0.26 mL, about 75 μg to about 450 μg in about 0.26 mL, about 75 μg to about 425 μg in about 0.26 mL, about 75 μg to about 400 μg in about 0.26 mL, about 75 μg to about 375 μg in about 0.26 mL, about 75 μg to about 350 μg in about 0.26 mL, about 75 μg to about 325 μg in about 0.26 mL, about 75 μg to about 300 μg in about 0.26 mL, about 75 μg to about 275 μg in about 0.26 mL, about 75 μg to about 250 μg in about 0.26 mL, about 75 μg to about 225 μg in about 0.26 mL, about 75 μg to about 200 μg in about 0.26 mL, about 75 μg to about 175 μg in about 0.26 mL, about 75 μg to about 150 μg in about 0.26 mL, about 75 μg to about 125 μg in about 0.26 mL, about 75 μg to about 100 μg in about 0.26 mL, about 100 μg to about 600 μg in about 0.26 mL, about 100 μg to about 575 μg in about 0.26 mL, about 100 μg to about 550 μg in about 0.26 mL, about 100 μg to about 525 μg in about 0.26 mL, about 100 μg to about 500 μg in about 0.26 mL, about 100 μg to about 475 μg in about 0.26 mL, about 100 μg to about 450 μg in about 0.26 mL, about 100 μg to about 425 μg in about 0.26 mL, about 100 μg to about 400 μg in about 0.26 mL, about 100 μg to about 375 μg in about 0.26 mL, about 100 μg to about 350 μg in about 0.26 mL, about 100 μg to about 325 μg in about 0.26 mL, about 100 μg to about 300 μg in about 0.26 mL, about 100 μg to about 275 μg in about 0.26 mL, about 100 μg to about 250 μg in about 0.26 mL, about 100 μg to about 225 μg in about 0.26 mL, about 100 μg to about 200 μg in about 0.26 mL, about 100 μg to about 175 μg in about 0.26 mL, about 100 μg to about 150 μg in about 0.26 mL, about 100 μg to about 125 μg in about 0.26 mL, about 125 μg to about 600 μg in about 0.26 mL, about 125 μg to about 575 μg in about 0.26 mL, about 125 μg to about 550 μg in about 0.26 mL, about 125 μg to about 525 μg in about 0.26 mL, about 125 μg to about 500 μg in about 0.26 mL, about 125 μg to about 475 μg in about 0.26 mL, about 125 μg to about 450 μg in about 0.26 mL, about 125 μg to about 425 μg in about 0.26 mL, about 125 μg to about 400 μg in about 0.26 mL, about 125 μg to about 375 μg in about 0.26 mL, about 125 μg to about 350 μg in about 0.26 mL, about 125 μg to about 325 μg in about 0.26 mL, about 125 μg to about 300 μg in about 0.26 mL, about 125 μg to about 275 μg in about 0.26 mL, about 125 μg to about 250 μg in about 0.26 mL, about 125 μg to about 225 μg in about 0.26 mL, about 125 μg to about 200 μg in about 0.26 mL, about 125 μg to about 175 μg in about 0.26 mL, about 125 μg to about 150 μg in about 0.26 mL, about 150 μg to about 600 μg in about 0.26 mL, about 150 μg to about 575 μg in about 0.26 mL, about 150 μg to about 550 μg in about 0.26 mL, about 150 μg to about 525 μg in about 0.26 mL, about 150 μg to about 500 μg in about 0.26 mL, about 150 μg to about 475 μg in about 0.26 mL, about 150 μg to about 450 μg in about 0.26 mL, about 150 μg to about 425 μg in about 0.26 mL, about 150 μg to about 400 μg in about 0.26 mL, about 150 μg to about 375 μg in about 0.26 mL, about 150 μg to about 350 μg in about 0.26 mL, about 150 μg to about 325 μg in about 0.26 mL, about 150 μg to about 300 μg in about 0.26 mL, about 150 μg to about 275 μg in about 0.26 mL, about 150 μg to about 250 μg in about 0.26 mL, about 150 μg to about 225 μg in about 0.26 mL, about 150 μg to about 200 μg in about 0.26 mL, about 150 μg to about 175 μg in about 0.26 mL, about 175 μg to about 600 μg in about 0.26 mL, about 175 μg to about 575 μg in about 0.26 mL, about 175 μg to about 550 μg in about 0.26 mL, about 175 μg to about 525 μg in about 0.26 mL, about 175 μg to about 500 μg in about 0.26 mL, about 175 μg to about 475 μg in about 0.26 mL, about 175 μg to about 450 μg in about 0.26 mL, about 175 μg to about 425 μg in about 0.26 mL, about 175 μg to about 400 μg in about 0.26 mL, about 175 μg to about 375 μg in about 0.26 mL, about 175 μg to about 350 μg in about 0.26 mL, about 175 μg to about 325 μg in about 0.26 mL, about 175 μg to about 300 μg in about 0.26 mL, about 175 μg to about 275 μg in about 0.26 mL, about 175 μg to about 250 μg in about 0.26 mL, about 175 μg to about 225 μg in about 0.26 mL, about 175 μg to about 200 μg in about 0.26 mL, about 200 μg to about 600 μg in about 0.26 mL, about 200 μg to about 575 μg in about 0.26 mL, about 200 μg to about 550 μg in about 0.26 mL, about 200 μg to about 525 μg in about 0.26 mL, about 200 μg to about 500 μg in about 0.26 mL, about 200 μg to about 475 μg in about 0.26 mL, about 200 μg to about 450 μg in about 0.26 mL, about 200 μg to about 425 μg in about 0.26 mL, about 200 μg to about 400 μg in about 0.26 mL, about 200 μg to about 375 μg in about 0.26 mL, about 200 μg to about 350 μg in about 0.26 mL, about 200 μg to about 325 μg in about 0.26 mL, about 200 μg to about 300 μg in about 0.26 mL, about 200 μg to about 275 μg in about 0.26 mL, about 200 μg to about 250 μg in about 0.26 mL, about 200 μg to about 225 μg in about 0.26 mL, about 225 μg to about 600 μg in about 0.26 mL, about 225 μg to about 575 μg in about 0.26 mL, about 225 μg to about 550 μg in about 0.26 mL, about 225 μg to about 525 μg in about 0.26 mL, about 225 μg to about 500 μg in about 0.26 mL, about 225 μg to about 475 μg in about 0.26 mL, about 225 μg to about 450 μg in about 0.26 mL, about 225 μg to about 425 μg in about 0.26 mL, about 225 μg to about 400 μg in about 0.26 mL, about 225 μg to about 375 μg in about 0.26 mL, about 225 μg to about 350 μg in about 0.26 mL, about 225 μg to about 325 μg in about 0.26 mL, about 225 μg to about 300 μg in about 0.26 mL, about 225 μg to about 275 μg in about 0.26 mL, or about 225 μg to about 250 μg in about 0.26 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.27 mL, about 5 μg to about 575 μg in about 0.27 mL, about 5 μg to about 550 μg in about 0.27 mL, about 5 μg to about 525 μg in about 0.27 mL, about 5 μg to about 500 μg in about 0.27 mL, about 5 μg to about 475 μg in about 0.27 mL, about 5 μg to about 450 μg in about 0.27 mL, about 5 μg to about 425 μg in about 0.27 mL, about 5 μg to about 400 μg in about 0.27 mL, about 5 μg to about 375 μg in about 0.27 mL, about 5 μg to about 350 μg in about 0.27 mL, about 5 μg to about 325 μg in about 0.27 mL, about 5 μg to about 300 μg in about 0.27 mL, about 5 μg to about 275 μg in about 0.27 mL, about 5 μg to about 250 μg in about 0.27 mL, about 5 μg to about 225 μg in about 0.27 mL, about 5 μg to about 200 μg in about 0.27 mL, about 5 μg to about 175 μg in about 0.27 mL, about 5 μg to about 150 μg in about 0.27 mL, about 5 μg to about 125 μg in about 0.27 mL, about 5 μg to about 100 μg in about 0.27 mL, about 5 μg to about 75 μg in about 0.27 mL, about 5 μg to about 50 μg in about 0.27 mL, about 5 μg to about 25 μg in about 0.27 mL, about 5 μg to about 20 μg in about 0.27 mL, about 5 μg to about 15 μg in about 0.27 mL, about 5 μg to about 10 μg in about 0.27 mL, about 10 μg to about 600 μg in about 0.27 mL, about 10 μg to about 575 μg in about 0.27 mL, about 10 μg to about 550 μg in about 0.27 mL, about 10 μg to about 525 μg in about 0.27 mL, about 10 μg to about 500 μg in about 0.27 mL, about 10 μg to about 475 μg in about 0.27 mL, about 10 μg to about 450 μg in about 0.27 mL, about 10 μg to about 425 μg in about 0.27 mL, about 10 μg to about 400 μg in about 0.27 mL, about 10 μg to about 375 μg in about 0.27 mL, about 10 μg to about 350 μg in about 0.27 mL, about 10 μg to about 325 μg in about 0.27 mL, about 10 μg to about 300 μg in about 0.27 mL, about 10 μg to about 275 μg in about 0.27 mL, about 10 μg to about 250 μg in about 0.27 mL, about 10 μg to about 225 μg in about 0.27 mL, about 10 μg to about 200 μg in about 0.27 mL, about 10 μg to about 175 μg in about 0.27 mL, about 10 μg to about 150 μg in about 0.27 mL, about 10 μg to about 125 μg in about 0.27 mL, about 10 μg to about 100 μg in about 0.27 mL, about 10 μg to about 75 μg in about 0.27 mL, about 10 μg to about 50 μg in about 0.27 mL, about 10 μg to about 25 μg in about 0.27 mL, about 10 μg to about 20 μg in about 0.27 mL, about 10 μg to about 15 μg in about 0.27 mL, about 15 μg to about 600 μg in about 0.27 mL, about 15 μg to about 575 μg in about 0.27 mL, about 15 μg to about 550 μg in about 0.27 mL, about 15 μg to about 525 μg in about 0.27 mL, about 15 μg to about 500 μg in about 0.27 mL, about 15 μg to about 475 μg in about 0.27 mL, about 15 μg to about 450 μg in about 0.27 mL, about 15 μg to about 425 μg in about 0.27 mL, about 15 μg to about 400 μg in about 0.27 mL, about 15 μg to about 375 μg in about 0.27 mL, about 15 μg to about 350 μg in about 0.27 mL, about 15 μg to about 325 μg in about 0.27 mL, about 15 μg to about 300 μg in about 0.27 mL, about 15 μg to about 275 μg in about 0.27 mL, about 15 μg to about 250 μg in about 0.27 mL, about 15 μg to about 225 μg in about 0.27 mL, about 15 μg to about 200 μg in about 0.27 mL, about 15 μg to about 175 μg in about 0.27 mL, about 15 μg to about 150 μg in about 0.27 mL, about 15 μg to about 125 μg in about 0.27 mL, about 15 μg to about 100 μg in about 0.27 mL, about 15 μg to about 75 μg in about 0.27 mL, about 15 μg to about 50 μg in about 0.27 mL, about 15 μg to about 25 μg in about 0.27 mL, about 15 μg to about 20 μg in about 0.27 mL, about 20 μg to about 600 μg in about 0.27 mL, about 20 μg to about 575 μg in about 0.27 mL, about 20 μg to about 550 μg in about 0.27 mL, about 20 μg to about 525 μg in about 0.27 mL, about 20 μg to about 500 μg in about 0.27 mL, about 20 μg to about 475 μg in about 0.27 mL, about 20 μg to about 450 μg in about 0.27 mL, about 20 μg to about 425 μg in about 0.27 mL, about 20 μg to about 400 μg in about 0.27 mL, about 20 μg to about 375 μg in about 0.27 mL, about 20 μg to about 350 μg in about 0.27 mL, about 20 μg to about 325 μg in about 0.27 mL, about 20 μg to about 300 μg in about 0.27 mL, about 20 μg to about 275 μg in about 0.27 mL, about 20 μg to about 250 μg in about 0.27 mL, about 20 μg to about 225 μg in about 0.27 mL, about 20 μg to about 200 μg in about 0.27 mL, about 20 μg to about 175 μg in about 0.27 mL, about 20 μg to about 150 μg in about 0.27 mL, about 20 μg to about 125 μg in about 0.27 mL, about 20 μg to about 100 μg in about 0.27 mL, about 20 μg to about 75 μg in about 0.27 mL, about 20 μg to about 50 μg in about 0.27 mL, about 20 μg to about 25 μg in about 0.27 mL, about 50 μg to about 600 μg in about 0.27 mL, about 50 μg to about 575 μg in about 0.27 mL, about 50 μg to about 550 μg in about 0.27 mL, about 50 μg to about 525 μg in about 0.27 mL, about 50 μg to about 500 μg in about 0.27 mL, about 50 μg to about 475 μg in about 0.27 mL, about 50 μg to about 450 μg in about 0.27 mL, about 50 μg to about 425 μg in about 0.27 mL, about 50 μg to about 400 μg in about 0.27 mL, about 50 μg to about 375 μg in about 0.27 mL, about 50 μg to about 350 μg in about 0.27 mL, about 50 μg to about 325 μg in about 0.27 mL, about 50 μg to about 300 μg in about 0.27 mL, about 50 μg to about 275 μg in about 0.27 mL, about 50 μg to about 250 μg in about 0.27 mL, about 50 μg to about 225 μg in about 0.27 mL, about 50 μg to about 200 μg in about 0.27 mL, about 50 μg to about 175 μg in about 0.27 mL, about 50 μg to about 150 μg in about 0.27 mL, about 50 μg to about 125 μg in about 0.27 mL, about 50 μg to about 100 μg in about 0.27 mL, about 50 μg to about 75 μg in about 0.27 mL, about 75 μg to about 600 μg in about 0.27 mL, about 75 μg to about 575 μg in about 0.27 mL, about 75 μg to about 550 μg in about 0.27 mL, about 75 μg to about 525 μg in about 0.27 mL, about 75 μg to about 500 μg in about 0.27 mL, about 75 μg to about 475 μg in about 0.27 mL, about 75 μg to about 450 μg in about 0.27 mL, about 75 μg to about 425 μg in about 0.27 mL, about 75 μg to about 400 μg in about 0.27 mL, about 75 μg to about 375 μg in about 0.27 mL, about 75 μg to about 350 μg in about 0.27 mL, about 75 μg to about 325 μg in about 0.27 mL, about 75 μg to about 300 μg in about 0.27 mL, about 75 μg to about 275 μg in about 0.27 mL, about 75 μg to about 250 μg in about 0.27 mL, about 75 μg to about 225 μg in about 0.27 mL, about 75 μg to about 200 μg in about 0.27 mL, about 75 μg to about 175 μg in about 0.27 mL, about 75 μg to about 150 μg in about 0.27 mL, about 75 μg to about 125 μg in about 0.27 mL, about 75 μg to about 100 μg in about 0.27 mL, about 100 μg to about 600 μg in about 0.27 mL, about 100 μg to about 575 μg in about 0.27 mL, about 100 μg to about 550 μg in about 0.27 mL, about 100 μg to about 525 μg in about 0.27 mL, about 100 μg to about 500 μg in about 0.27 mL, about 100 μg to about 475 μg in about 0.27 mL, about 100 μg to about 450 μg in about 0.27 mL, about 100 μg to about 425 μg in about 0.27 mL, about 100 μg to about 400 μg in about 0.27 mL, about 100 μg to about 375 μg in about 0.27 mL, about 100 μg to about 350 μg in about 0.27 mL, about 100 μg to about 325 μg in about 0.27 mL, about 100 μg to about 300 μg in about 0.27 mL, about 100 μg to about 275 μg in about 0.27 mL, about 100 μg to about 250 μg in about 0.27 mL, about 100 μg to about 225 μg in about 0.27 mL, about 100 μg to about 200 μg in about 0.27 mL, about 100 μg to about 175 μg in about 0.27 mL, about 100 μg to about 150 μg in about 0.27 mL, about 100 μg to about 125 μg in about 0.27 mL, about 125 μg to about 600 μg in about 0.27 mL, about 125 μg to about 575 μg in about 0.27 mL, about 125 μg to about 550 μg in about 0.27 mL, about 125 μg to about 525 μg in about 0.27 mL, about 125 μg to about 500 μg in about 0.27 mL, about 125 μg to about 475 μg in about 0.27 mL, about 125 μg to about 450 μg in about 0.27 mL, about 125 μg to about 425 μg in about 0.27 mL, about 125 μg to about 400 μg in about 0.27 mL, about 125 μg to about 375 μg in about 0.27 mL, about 125 μg to about 350 μg in about 0.27 mL, about 125 μg to about 325 μg in about 0.27 mL, about 125 μg to about 300 μg in about 0.27 mL, about 125 μg to about 275 μg in about 0.27 mL, about 125 μg to about 250 μg in about 0.27 mL, about 125 μg to about 225 μg in about 0.27 mL, about 125 μg to about 200 μg in about 0.27 mL, about 125 μg to about 175 μg in about 0.27 mL, about 125 μg to about 150 μg in about 0.27 mL, about 150 μg to about 600 μg in about 0.27 mL, about 150 μg to about 575 μg in about 0.27 mL, about 150 μg to about 550 μg in about 0.27 mL, about 150 μg to about 525 μg in about 0.27 mL, about 150 μg to about 500 μg in about 0.27 mL, about 150 μg to about 475 μg in about 0.27 mL, about 150 μg to about 450 μg in about 0.27 mL, about 150 μg to about 425 μg in about 0.27 mL, about 150 μg to about 400 μg in about 0.27 mL, about 150 μg to about 375 μg in about 0.27 mL, about 150 μg to about 350 μg in about 0.27 mL, about 150 μg to about 325 μg in about 0.27 mL, about 150 μg to about 300 μg in about 0.27 mL, about 150 μg to about 275 μg in about 0.27 mL, about 150 μg to about 250 μg in about 0.27 mL, about 150 μg to about 225 μg in about 0.27 mL, about 150 μg to about 200 μg in about 0.27 mL, about 150 μg to about 175 μg in about 0.27 mL, about 175 μg to about 600 μg in about 0.27 mL, about 175 μg to about 575 μg in about 0.27 mL, about 175 μg to about 550 μg in about 0.27 mL, about 175 μg to about 525 μg in about 0.27 mL, about 175 μg to about 500 μg in about 0.27 mL, about 175 μg to about 475 μg in about 0.27 mL, about 175 μg to about 450 μg in about 0.27 mL, about 175 μg to about 425 μg in about 0.27 mL, about 175 μg to about 400 μg in about 0.27 mL, about 175 μg to about 375 μg in about 0.27 mL, about 175 μg to about 350 μg in about 0.27 mL, about 175 μg to about 325 μg in about 0.27 mL, about 175 μg to about 300 μg in about 0.27 mL, about 175 μg to about 275 μg in about 0.27 mL, about 175 μg to about 250 μg in about 0.27 mL, about 175 μg to about 225 μg in about 0.27 mL, about 175 μg to about 200 μg in about 0.27 mL, about 200 μg to about 600 μg in about 0.27 mL, about 200 μg to about 575 μg in about 0.27 mL, about 200 μg to about 550 μg in about 0.27 mL, about 200 μg to about 525 μg in about 0.27 mL, about 200 μg to about 500 μg in about 0.27 mL, about 200 μg to about 475 μg in about 0.27 mL, about 200 μg to about 450 μg in about 0.27 mL, about 200 μg to about 425 μg in about 0.27 mL, about 200 μg to about 400 μg in about 0.27 mL, about 200 μg to about 375 μg in about 0.27 mL, about 200 μg to about 350 μg in about 0.27 mL, about 200 μg to about 325 μg in about 0.27 mL, about 200 μg to about 300 μg in about 0.27 mL, about 200 μg to about 275 μg in about 0.27 mL, about 200 μg to about 250 μg in about 0.27 mL, about 200 μg to about 225 μg in about 0.27 mL, about 225 μg to about 600 μg in about 0.27 mL, about 225 μg to about 575 μg in about 0.27 mL, about 225 μg to about 550 μg in about 0.27 mL, about 225 μg to about 525 μg in about 0.27 mL, about 225 μg to about 500 μg in about 0.27 mL, about 225 μg to about 475 μg in about 0.27 mL, about 225 μg to about 450 μg in about 0.27 mL, about 225 μg to about 425 μg in about 0.27 mL, about 225 μg to about 400 μg in about 0.27 mL, about 225 μg to about 375 μg in about 0.27 mL, about 225 μg to about 350 μg in about 0.27 mL, about 225 μg to about 325 μg in about 0.27 mL, about 225 μg to about 300 μg in about 0.27 mL, about 225 μg to about 275 μg in about 0.27 mL, or about 225 μg to about 250 μg in about 0.27 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.28 mL, about 5 μg to about 575 μg in about 0.28 mL, about 5 μg to about 550 μg in about 0.28 mL, about 5 μg to about 525 μg in about 0.28 mL, about 5 μg to about 500 μg in about 0.28 mL, about 5 μg to about 475 μg in about 0.28 mL, about 5 μg to about 450 μg in about 0.28 mL, about 5 μg to about 425 μg in about 0.28 mL, about 5 μg to about 400 μg in about 0.28 mL, about 5 μg to about 375 μg in about 0.28 mL, about 5 μg to about 350 μg in about 0.28 mL, about 5 μg to about 325 μg in about 0.28 mL, about 5 μg to about 300 μg in about 0.28 mL, about 5 μg to about 275 μg in about 0.28 mL, about 5 μg to about 250 μg in about 0.28 mL, about 5 μg to about 225 μg in about 0.28 mL, about 5 μg to about 200 μg in about 0.28 mL, about 5 μg to about 175 μg in about 0.28 mL, about 5 μg to about 150 μg in about 0.28 mL, about 5 μg to about 125 μg in about 0.28 mL, about 5 μg to about 100 μg in about 0.28 mL, about 5 μg to about 75 μg in about 0.28 mL, about 5 μg to about 50 μg in about 0.28 mL, about 5 μg to about 25 μg in about 0.28 mL, about 5 μg to about 20 μg in about 0.28 mL, about 5 μg to about 15 μg in about 0.28 mL, about 5 μg to about 10 μg in about 0.28 mL, about 10 μg to about 600 μg in about 0.28 mL, about 10 μg to about 575 μg in about 0.28 mL, about 10 μg to about 550 μg in about 0.28 mL, about 10 μg to about 525 μg in about 0.28 mL, about 10 μg to about 500 μg in about 0.28 mL, about 10 μg to about 475 μg in about 0.28 mL, about 10 μg to about 450 μg in about 0.28 mL, about 10 μg to about 425 μg in about 0.28 mL, about 10 μg to about 400 μg in about 0.28 mL, about 10 μg to about 375 μg in about 0.28 mL, about 10 μg to about 350 μg in about 0.28 mL, about 10 μg to about 325 μg in about 0.28 mL, about 10 μg to about 300 μg in about 0.28 mL, about 10 μg to about 275 μg in about 0.28 mL, about 10 μg to about 250 μg in about 0.28 mL, about 10 μg to about 225 μg in about 0.28 mL, about 10 μg to about 200 μg in about 0.28 mL, about 10 μg to about 175 μg in about 0.28 mL, about 10 μg to about 150 μg in about 0.28 mL, about 10 μg to about 125 μg in about 0.28 mL, about 10 μg to about 100 μg in about 0.28 mL, about 10 μg to about 75 μg in about 0.28 mL, about 10 μg to about 50 μg in about 0.28 mL, about 10 μg to about 25 μg in about 0.28 mL, about 10 μg to about 20 μg in about 0.28 mL, about 10 μg to about 15 μg in about 0.28 mL, about 15 μg to about 600 μg in about 0.28 mL, about 15 μg to about 575 μg in about 0.28 mL, about 15 μg to about 550 μg in about 0.28 mL, about 15 μg to about 525 μg in about 0.28 mL, about 15 μg to about 500 μg in about 0.28 mL, about 15 μg to about 475 μg in about 0.28 mL, about 15 μg to about 450 μg in about 0.28 mL, about 15 μg to about 425 μg in about 0.28 mL, about 15 μg to about 400 μg in about 0.28 mL, about 15 μg to about 375 μg in about 0.28 mL, about 15 μg to about 350 μg in about 0.28 mL, about 15 μg to about 325 μg in about 0.28 mL, about 15 μg to about 300 μg in about 0.28 mL, about 15 μg to about 275 μg in about 0.28 mL, about 15 μg to about 250 μg in about 0.28 mL, about 15 μg to about 225 μg in about 0.28 mL, about 15 μg to about 200 μg in about 0.28 mL, about 15 μg to about 175 μg in about 0.28 mL, about 15 μg to about 150 μg in about 0.28 mL, about 15 μg to about 125 μg in about 0.28 mL, about 15 μg to about 100 μg in about 0.28 mL, about 15 μg to about 75 μg in about 0.28 mL, about 15 μg to about 50 μg in about 0.28 mL, about 15 μg to about 25 μg in about 0.28 mL, about 15 μg to about 20 μg in about 0.28 mL, about 20 μg to about 600 μg in about 0.28 mL, about 20 μg to about 575 μg in about 0.28 mL, about 20 μg to about 550 μg in about 0.28 mL, about 20 μg to about 525 μg in about 0.28 mL, about 20 μg to about 500 μg in about 0.28 mL, about 20 μg to about 475 μg in about 0.28 mL, about 20 μg to about 450 μg in about 0.28 mL, about 20 μg to about 425 μg in about 0.28 mL, about 20 μg to about 400 μg in about 0.28 mL, about 20 μg to about 375 μg in about 0.28 mL, about 20 μg to about 350 μg in about 0.28 mL, about 20 μg to about 325 μg in about 0.28 mL, about 20 μg to about 300 μg in about 0.28 mL, about 20 μg to about 275 μg in about 0.28 mL, about 20 μg to about 250 μg in about 0.28 mL, about 20 μg to about 225 μg in about 0.28 mL, about 20 μg to about 200 μg in about 0.28 mL, about 20 μg to about 175 μg in about 0.28 mL, about 20 μg to about 150 μg in about 0.28 mL, about 20 μg to about 125 μg in about 0.28 mL, about 20 μg to about 100 μg in about 0.28 mL, about 20 μg to about 75 μg in about 0.28 mL, about 20 μg to about 50 μg in about 0.28 mL, about 20 μg to about 25 μg in about 0.28 mL, about 50 μg to about 600 μg in about 0.28 mL, about 50 μg to about 575 μg in about 0.28 mL, about 50 μg to about 550 μg in about 0.28 mL, about 50 μg to about 525 μg in about 0.28 mL, about 50 μg to about 500 μg in about 0.28 mL, about 50 μg to about 475 μg in about 0.28 mL, about 50 μg to about 450 μg in about 0.28 mL, about 50 μg to about 425 μg in about 0.28 mL, about 50 μg to about 400 μg in about 0.28 mL, about 50 μg to about 375 μg in about 0.28 mL, about 50 μg to about 350 μg in about 0.28 mL, about 50 μg to about 325 μg in about 0.28 mL, about 50 μg to about 300 μg in about 0.28 mL, about 50 μg to about 275 μg in about 0.28 mL, about 50 μg to about 250 μg in about 0.28 mL, about 50 μg to about 225 μg in about 0.28 mL, about 50 μg to about 200 μg in about 0.28 mL, about 50 μg to about 175 μg in about 0.28 mL, about 50 μg to about 150 μg in about 0.28 mL, about 50 μg to about 125 μg in about 0.28 mL, about 50 μg to about 100 μg in about 0.28 mL, about 50 μg to about 75 μg in about 0.28 mL, about 75 μg to about 600 μg in about 0.28 mL, about 75 μg to about 575 μg in about 0.28 mL, about 75 μg to about 550 μg in about 0.28 mL, about 75 μg to about 525 μg in about 0.28 mL, about 75 μg to about 500 μg in about 0.28 mL, about 75 μg to about 475 μg in about 0.28 mL, about 75 μg to about 450 μg in about 0.28 mL, about 75 μg to about 425 μg in about 0.28 mL, about 75 μg to about 400 μg in about 0.28 mL, about 75 μg to about 375 μg in about 0.28 mL, about 75 μg to about 350 μg in about 0.28 mL, about 75 μg to about 325 μg in about 0.28 mL, about 75 μg to about 300 μg in about 0.28 mL, about 75 μg to about 275 μg in about 0.28 mL, about 75 μg to about 250 μg in about 0.28 mL, about 75 μg to about 225 μg in about 0.28 mL, about 75 μg to about 200 μg in about 0.28 mL, about 75 μg to about 175 μg in about 0.28 mL, about 75 μg to about 150 μg in about 0.28 mL, about 75 μg to about 125 μg in about 0.28 mL, about 75 μg to about 100 μg in about 0.28 mL, about 100 μg to about 600 μg in about 0.28 mL, about 100 μg to about 575 μg in about 0.28 mL, about 100 μg to about 550 μg in about 0.28 mL, about 100 μg to about 525 μg in about 0.28 mL, about 100 μg to about 500 μg in about 0.28 mL, about 100 μg to about 475 μg in about 0.28 mL, about 100 μg to about 450 μg in about 0.28 mL, about 100 μg to about 425 μg in about 0.28 mL, about 100 μg to about 400 μg in about 0.28 mL, about 100 μg to about 375 μg in about 0.28 mL, about 100 μg to about 350 μg in about 0.28 mL, about 100 μg to about 325 μg in about 0.28 mL, about 100 μg to about 300 μg in about 0.28 mL, about 100 μg to about 275 μg in about 0.28 mL, about 100 μg to about 250 μg in about 0.28 mL, about 100 μg to about 225 μg in about 0.28 mL, about 100 μg to about 200 μg in about 0.28 mL, about 100 μg to about 175 μg in about 0.28 mL, about 100 μg to about 150 μg in about 0.28 mL, about 100 μg to about 125 μg in about 0.28 mL, about 125 μg to about 600 μg in about 0.28 mL, about 125 μg to about 575 μg in about 0.28 mL, about 125 μg to about 550 μg in about 0.28 mL, about 125 μg to about 525 μg in about 0.28 mL, about 125 μg to about 500 μg in about 0.28 mL, about 125 μg to about 475 μg in about 0.28 mL, about 125 μg to about 450 μg in about 0.28 mL, about 125 μg to about 425 μg in about 0.28 mL, about 125 μg to about 400 μg in about 0.28 mL, about 125 μg to about 375 μg in about 0.28 mL, about 125 μg to about 350 μg in about 0.28 mL, about 125 μg to about 325 μg in about 0.28 mL, about 125 μg to about 300 μg in about 0.28 mL, about 125 μg to about 275 μg in about 0.28 mL, about 125 μg to about 250 μg in about 0.28 mL, about 125 μg to about 225 μg in about 0.28 mL, about 125 μg to about 200 μg in about 0.28 mL, about 125 μg to about 175 μg in about 0.28 mL, about 125 μg to about 150 μg in about 0.28 mL, about 150 μg to about 600 μg in about 0.28 mL, about 150 μg to about 575 μg in about 0.28 mL, about 150 μg to about 550 μg in about 0.28 mL, about 150 μg to about 525 μg in about 0.28 mL, about 150 μg to about 500 μg in about 0.28 mL, about 150 μg to about 475 μg in about 0.28 mL, about 150 μg to about 450 μg in about 0.28 mL, about 150 μg to about 425 μg in about 0.28 mL, about 150 μg to about 400 μg in about 0.28 mL, about 150 μg to about 375 μg in about 0.28 mL, about 150 μg to about 350 μg in about 0.28 mL, about 150 μg to about 325 μg in about 0.28 mL, about 150 μg to about 300 μg in about 0.28 mL, about 150 μg to about 275 μg in about 0.28 mL, about 150 μg to about 250 μg in about 0.28 mL, about 150 μg to about 225 μg in about 0.28 mL, about 150 μg to about 200 μg in about 0.28 mL, about 150 μg to about 175 μg in about 0.28 mL, about 175 μg to about 600 μg in about 0.28 mL, about 175 μg to about 575 μg in about 0.28 mL, about 175 μg to about 550 μg in about 0.28 mL, about 175 μg to about 525 μg in about 0.28 mL, about 175 μg to about 500 μg in about 0.28 mL, about 175 μg to about 475 μg in about 0.28 mL, about 175 μg to about 450 μg in about 0.28 mL, about 175 μg to about 425 μg in about 0.28 mL, about 175 μg to about 400 μg in about 0.28 mL, about 175 μg to about 375 μg in about 0.28 mL, about 175 μg to about 350 μg in about 0.28 mL, about 175 μg to about 325 μg in about 0.28 mL, about 175 μg to about 300 μg in about 0.28 mL, about 175 μg to about 275 μg in about 0.28 mL, about 175 μg to about 250 μg in about 0.28 mL, about 175 μg to about 225 μg in about 0.28 mL, about 175 μg to about 200 μg in about 0.28 mL, about 200 μg to about 600 μg in about 0.28 mL, about 200 μg to about 575 μg in about 0.28 mL, about 200 μg to about 550 μg in about 0.28 mL, about 200 μg to about 525 μg in about 0.28 mL, about 200 μg to about 500 μg in about 0.28 mL, about 200 μg to about 475 μg in about 0.28 mL, about 200 μg to about 450 μg in about 0.28 mL, about 200 μg to about 425 μg in about 0.28 mL, about 200 μg to about 400 μg in about 0.28 mL, about 200 μg to about 375 μg in about 0.28 mL, about 200 μg to about 350 μg in about 0.28 mL, about 200 μg to about 325 μg in about 0.28 mL, about 200 μg to about 300 μg in about 0.28 mL, about 200 μg to about 275 μg in about 0.28 mL, about 200 μg to about 250 μg in about 0.28 mL, about 200 μg to about 225 μg in about 0.28 mL, about 225 μg to about 600 μg in about 0.28 mL, about 225 μg to about 575 μg in about 0.28 mL, about 225 μg to about 550 μg in about 0.28 mL, about 225 μg to about 525 μg in about 0.28 mL, about 225 μg to about 500 μg in about 0.28 mL, about 225 μg to about 475 μg in about 0.28 mL, about 225 μg to about 450 μg in about 0.28 mL, about 225 μg to about 425 μg in about 0.28 mL, about 225 μg to about 400 μg in about 0.28 mL, about 225 μg to about 375 μg in about 0.28 mL, about 225 μg to about 350 μg in about 0.28 mL, about 225 μg to about 325 μg in about 0.28 mL, about 225 μg to about 300 μg in about 0.28 mL, about 225 μg to about 275 μg in about 0.28 mL, or about 225 μg to about 250 μg in about 0.28 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.29 mL, about 5 μg to about 575 μg in about 0.29 mL, about 5 μg to about 550 μg in about 0.29 mL, about 5 μg to about 525 μg in about 0.29 mL, about 5 μg to about 500 μg in about 0.29 mL, about 5 μg to about 475 μg in about 0.29 mL, about 5 μg to about 450 μg in about 0.29 mL, about 5 μg to about 425 μg in about 0.29 mL, about 5 μg to about 400 μg in about 0.29 mL, about 5 μg to about 375 μg in about 0.29 mL, about 5 μg to about 350 μg in about 0.29 mL, about 5 μg to about 325 μg in about 0.29 mL, about 5 μg to about 300 μg in about 0.29 mL, about 5 μg to about 275 μg in about 0.29 mL, about 5 μg to about 250 μg in about 0.29 mL, about 5 μg to about 225 μg in about 0.29 mL, about 5 μg to about 200 μg in about 0.29 mL, about 5 μg to about 175 μg in about 0.29 mL, about 5 μg to about 150 μg in about 0.29 mL, about 5 μg to about 125 μg in about 0.29 mL, about 5 μg to about 100 μg in about 0.29 mL, about 5 μg to about 75 μg in about 0.29 mL, about 5 μg to about 50 μg in about 0.29 mL, about 5 μg to about 25 μg in about 0.29 mL, about 5 μg to about 20 μg in about 0.29 mL, about 5 μg to about 15 μg in about 0.29 mL, about 5 μg to about 10 μg in about 0.29 mL, about 10 μg to about 600 μg in about 0.29 mL, about 10 μg to about 575 μg in about 0.29 mL, about 10 μg to about 550 μg in about 0.29 mL, about 10 μg to about 525 μg in about 0.29 mL, about 10 μg to about 500 μg in about 0.29 mL, about 10 μg to about 475 μg in about 0.29 mL, about 10 μg to about 450 μg in about 0.29 mL, about 10 μg to about 425 μg in about 0.29 mL, about 10 μg to about 400 μg in about 0.29 mL, about 10 μg to about 375 μg in about 0.29 mL, about 10 μg to about 350 μg in about 0.29 mL, about 10 μg to about 325 μg in about 0.29 mL, about 10 μg to about 300 μg in about 0.29 mL, about 10 μg to about 275 μg in about 0.29 mL, about 10 μg to about 250 μg in about 0.29 mL, about 10 μg to about 225 μg in about 0.29 mL, about 10 μg to about 200 μg in about 0.29 mL, about 10 μg to about 175 μg in about 0.29 mL, about 10 μg to about 150 μg in about 0.29 mL, about 10 μg to about 125 μg in about 0.29 mL, about 10 μg to about 100 μg in about 0.29 mL, about 10 μg to about 75 μg in about 0.29 mL, about 10 μg to about 50 μg in about 0.29 mL, about 10 μg to about 25 μg in about 0.29 mL, about 10 μg to about 20 μg in about 0.29 mL, about 10 μg to about 15 μg in about 0.29 mL, about 15 μg to about 600 μg in about 0.29 mL, about 15 μg to about 575 μg in about 0.29 mL, about 15 μg to about 550 μg in about 0.29 mL, about 15 μg to about 525 μg in about 0.29 mL, about 15 μg to about 500 μg in about 0.29 mL, about 15 μg to about 475 μg in about 0.29 mL, about 15 μg to about 450 μg in about 0.29 mL, about 15 μg to about 425 μg in about 0.29 mL, about 15 μg to about 400 μg in about 0.29 mL, about 15 μg to about 375 μg in about 0.29 mL, about 15 μg to about 350 μg in about 0.29 mL, about 15 μg to about 325 μg in about 0.29 mL, about 15 μg to about 300 μg in about 0.29 mL, about 15 μg to about 275 μg in about 0.29 mL, about 15 μg to about 250 μg in about 0.29 mL, about 15 μg to about 225 μg in about 0.29 mL, about 15 μg to about 200 μg in about 0.29 mL, about 15 μg to about 175 μg in about 0.29 mL, about 15 μg to about 150 μg in about 0.29 mL, about 15 μg to about 125 μg in about 0.29 mL, about 15 μg to about 100 μg in about 0.29 mL, about 15 μg to about 75 μg in about 0.29 mL, about 15 μg to about 50 μg in about 0.29 mL, about 15 μg to about 25 μg in about 0.29 mL, about 15 μg to about 20 μg in about 0.29 mL, about 20 μg to about 600 μg in about 0.29 mL, about 20 μg to about 575 μg in about 0.29 mL, about 20 μg to about 550 μg in about 0.29 mL, about 20 μg to about 525 μg in about 0.29 mL, about 20 μg to about 500 μg in about 0.29 mL, about 20 μg to about 475 μg in about 0.29 mL, about 20 μg to about 450 μg in about 0.29 mL, about 20 μg to about 425 μg in about 0.29 mL, about 20 μg to about 400 μg in about 0.29 mL, about 20 μg to about 375 μg in about 0.29 mL, about 20 μg to about 350 μg in about 0.29 mL, about 20 μg to about 325 μg in about 0.29 mL, about 20 μg to about 300 μg in about 0.29 mL, about 20 μg to about 275 μg in about 0.29 mL, about 20 μg to about 250 μg in about 0.29 mL, about 20 μg to about 225 μg in about 0.29 mL, about 20 μg to about 200 μg in about 0.29 mL, about 20 μg to about 175 μg in about 0.29 mL, about 20 μg to about 150 μg in about 0.29 mL, about 20 μg to about 125 μg in about 0.29 mL, about 20 μg to about 100 μg in about 0.29 mL, about 20 μg to about 75 μg in about 0.29 mL, about 20 μg to about 50 μg in about 0.29 mL, about 20 μg to about 25 μg in about 0.29 mL, about 50 μg to about 600 μg in about 0.29 mL, about 50 μg to about 575 μg in about 0.29 mL, about 50 μg to about 550 μg in about 0.29 mL, about 50 μg to about 525 μg in about 0.29 mL, about 50 μg to about 500 μg in about 0.29 mL, about 50 μg to about 475 μg in about 0.29 mL, about 50 μg to about 450 μg in about 0.29 mL, about 50 μg to about 425 μg in about 0.29 mL, about 50 μg to about 400 μg in about 0.29 mL, about 50 μg to about 375 μg in about 0.29 mL, about 50 μg to about 350 μg in about 0.29 mL, about 50 μg to about 325 μg in about 0.29 mL, about 50 μg to about 300 μg in about 0.29 mL, about 50 μg to about 275 μg in about 0.29 mL, about 50 μg to about 250 μg in about 0.29 mL, about 50 μg to about 225 μg in about 0.29 mL, about 50 μg to about 200 μg in about 0.29 mL, about 50 μg to about 175 μg in about 0.29 mL, about 50 μg to about 150 μg in about 0.29 mL, about 50 μg to about 125 μg in about 0.29 mL, about 50 μg to about 100 μg in about 0.29 mL, about 50 μg to about 75 μg in about 0.29 mL, about 75 μg to about 600 μg in about 0.29 mL, about 75 μg to about 575 μg in about 0.29 mL, about 75 μg to about 550 μg in about 0.29 mL, about 75 μg to about 525 μg in about 0.29 mL, about 75 μg to about 500 μg in about 0.29 mL, about 75 μg to about 475 μg in about 0.29 mL, about 75 μg to about 450 μg in about 0.29 mL, about 75 μg to about 425 μg in about 0.29 mL, about 75 μg to about 400 μg in about 0.29 mL, about 75 μg to about 375 μg in about 0.29 mL, about 75 μg to about 350 μg in about 0.29 mL, about 75 μg to about 325 μg in about 0.29 mL, about 75 μg to about 300 μg in about 0.29 mL, about 75 μg to about 275 μg in about 0.29 mL, about 75 μg to about 250 μg in about 0.29 mL, about 75 μg to about 225 μg in about 0.29 mL, about 75 μg to about 200 μg in about 0.29 mL, about 75 μg to about 175 μg in about 0.29 mL, about 75 μg to about 150 μg in about 0.29 mL, about 75 μg to about 125 μg in about 0.29 mL, about 75 μg to about 100 μg in about 0.29 mL, about 100 μg to about 600 μg in about 0.29 mL, about 100 μg to about 575 μg in about 0.29 mL, about 100 μg to about 550 μg in about 0.29 mL, about 100 μg to about 525 μg in about 0.29 mL, about 100 μg to about 500 μg in about 0.29 mL, about 100 μg to about 475 μg in about 0.29 mL, about 100 μg to about 450 μg in about 0.29 mL, about 100 μg to about 425 μg in about 0.29 mL, about 100 μg to about 400 μg in about 0.29 mL, about 100 μg to about 375 μg in about 0.29 mL, about 100 μg to about 350 μg in about 0.29 mL, about 100 μg to about 325 μg in about 0.29 mL, about 100 μg to about 300 μg in about 0.29 mL, about 100 μg to about 275 μg in about 0.29 mL, about 100 μg to about 250 μg in about 0.29 mL, about 100 μg to about 225 μg in about 0.29 mL, about 100 μg to about 200 μg in about 0.29 mL, about 100 μg to about 175 μg in about 0.29 mL, about 100 μg to about 150 μg in about 0.29 mL, about 100 μg to about 125 μg in about 0.29 mL, about 125 μg to about 600 μg in about 0.29 mL, about 125 μg to about 575 μg in about 0.29 mL, about 125 μg to about 550 μg in about 0.29 mL, about 125 μg to about 525 μg in about 0.29 mL, about 125 μg to about 500 μg in about 0.29 mL, about 125 μg to about 475 μg in about 0.29 mL, about 125 μg to about 450 μg in about 0.29 mL, about 125 μg to about 425 μg in about 0.29 mL, about 125 μg to about 400 μg in about 0.29 mL, about 125 μg to about 375 μg in about 0.29 mL, about 125 μg to about 350 μg in about 0.29 mL, about 125 μg to about 325 μg in about 0.29 mL, about 125 μg to about 300 μg in about 0.29 mL, about 125 μg to about 275 μg in about 0.29 mL, about 125 μg to about 250 μg in about 0.29 mL, about 125 μg to about 225 μg in about 0.29 mL, about 125 μg to about 200 μg in about 0.29 mL, about 125 μg to about 175 μg in about 0.29 mL, about 125 μg to about 150 μg in about 0.29 mL, about 150 μg to about 600 μg in about 0.29 mL, about 150 μg to about 575 μg in about 0.29 mL, about 150 μg to about 550 μg in about 0.29 mL, about 150 μg to about 525 μg in about 0.29 mL, about 150 μg to about 500 μg in about 0.29 mL, about 150 μg to about 475 μg in about 0.29 mL, about 150 μg to about 450 μg in about 0.29 mL, about 150 μg to about 425 μg in about 0.29 mL, about 150 μg to about 400 μg in about 0.29 mL, about 150 μg to about 375 μg in about 0.29 mL, about 150 μg to about 350 μg in about 0.29 mL, about 150 μg to about 325 μg in about 0.29 mL, about 150 μg to about 300 μg in about 0.29 mL, about 150 μg to about 275 μg in about 0.29 mL, about 150 μg to about 250 μg in about 0.29 mL, about 150 μg to about 225 μg in about 0.29 mL, about 150 μg to about 200 μg in about 0.29 mL, about 150 μg to about 175 μg in about 0.29 mL, about 175 μg to about 600 μg in about 0.29 mL, about 175 μg to about 575 μg in about 0.29 mL, about 175 μg to about 550 μg in about 0.29 mL, about 175 μg to about 525 μg in about 0.29 mL, about 175 μg to about 500 μg in about 0.29 mL, about 175 μg to about 475 μg in about 0.29 mL, about 175 μg to about 450 μg in about 0.29 mL, about 175 μg to about 425 μg in about 0.29 mL, about 175 μg to about 400 μg in about 0.29 mL, about 175 μg to about 375 μg in about 0.29 mL, about 175 μg to about 350 μg in about 0.29 mL, about 175 μg to about 325 μg in about 0.29 mL, about 175 μg to about 300 μg in about 0.29 mL, about 175 μg to about 275 μg in about 0.29 mL, about 175 μg to about 250 μg in about 0.29 mL, about 175 μg to about 225 μg in about 0.29 mL, about 175 μg to about 200 μg in about 0.29 mL, about 200 μg to about 600 μg in about 0.29 mL, about 200 μg to about 575 μg in about 0.29 mL, about 200 μg to about 550 μg in about 0.29 mL, about 200 μg to about 525 μg in about 0.29 mL, about 200 μg to about 500 μg in about 0.29 mL, about 200 μg to about 475 μg in about 0.29 mL, about 200 μg to about 450 μg in about 0.29 mL, about 200 μg to about 425 μg in about 0.29 mL, about 200 μg to about 400 μg in about 0.29 mL, about 200 μg to about 375 μg in about 0.29 mL, about 200 μg to about 350 μg in about 0.29 mL, about 200 μg to about 325 μg in about 0.29 mL, about 200 μg to about 300 μg in about 0.29 mL, about 200 μg to about 275 μg in about 0.29 mL, about 200 μg to about 250 μg in about 0.29 mL, about 200 μg to about 225 μg in about 0.29 mL, about 225 μg to about 600 μg in about 0.29 mL, about 225 μg to about 575 μg in about 0.29 mL, about 225 μg to about 550 μg in about 0.29 mL, about 225 μg to about 525 μg in about 0.29 mL, about 225 μg to about 500 μg in about 0.29 mL, about 225 μg to about 475 μg in about 0.29 mL, about 225 μg to about 450 μg in about 0.29 mL, about 225 μg to about 425 μg in about 0.29 mL, about 225 μg to about 400 μg in about 0.29 mL, about 225 μg to about 375 μg in about 0.29 mL, about 225 μg to about 350 μg in about 0.29 mL, about 225 μg to about 325 μg in about 0.29 mL, about 225 μg to about 300 μg in about 0.29 mL, about 225 μg to about 275 μg in about 0.29 mL, or about 225 μg to about 250 μg in about 0.29 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.30 mL, about 5 μg to about 575 μg in about 0.30 mL, about 5 μg to about 550 μg in about 0.30 mL, about 5 μg to about 525 μg in about 0.30 mL, about 5 μg to about 500 μg in about 0.30 mL, about 5 μg to about 475 μg in about 0.30 mL, about 5 μg to about 450 μg in about 0.30 mL, about 5 μg to about 425 μg in about 0.30 mL, about 5 μg to about 400 μg in about 0.30 mL, about 5 μg to about 375 μg in about 0.30 mL, about 5 μg to about 350 μg in about 0.30 mL, about 5 μg to about 325 μg in about 0.30 mL, about 5 μg to about 300 μg in about 0.50 mL, about 5 μg to about 275 μg in about 0.30 mL, about 5 μg to about 250 μg in about 0.30 mL, about 5 μg to about 225 μg in about 0.30 mL, about 5 μg to about 200 μg in about 0.30 mL, about 5 μg to about 175 μg in about 0.30 mL, about 5 μg to about 150 μg in about 0.30 mL, about 5 μg to about 125 μg in about 0.30 mL, about 5 μg to about 100 μg in about 0.30 mL, about 5 μg to about 75 μg in about 0.30 mL, about 5 μg to about 50 μg in about 0.30 mL, about 5 μg to about 25 μg in about 0.30 mL, about 5 μg to about 20 μg in about 0.30 mL, about 5 μg to about 15 μg in about 0.30 mL, about 5 μg to about 10 μg in about 0.30 mL, about 10 μg to about 600 μg in about 0.30 mL, about 10 μg to about 575 μg in about 0.30 mL, about 10 μg to about 550 μg in about 0.30 mL, about 10 μg to about 525 μg in about 0.30 mL, about 10 μg to about 500 μg in about 0.30 mL, about 10 μg to about 475 μg in about 0.30 mL, about 10 μg to about 450 μg in about 0.30 mL, about 10 μg to about 425 μg in about 0.30 mL, about 10 μg to about 400 μg in about 0.30 mL, about 10 μg to about 375 μg in about 0.30 mL, about 10 μg to about 350 μg in about 0.30 mL, about 10 μg to about 325 μg in about 0.30 mL, about 10 μg to about 300 μg in about 0.30 mL, about 10 μg to about 275 μg in about 0.30 mL, about 10 μg to about 250 μg in about 0.30 mL, about 10 μg to about 225 μg in about 0.30 mL, about 10 μg to about 200 μg in about 0.30 mL, about 10 μg to about 175 μg in about 0.30 mL, about 10 μg to about 150 μg in about 0.30 mL, about 10 μg to about 125 μg in about 0.30 mL, about 10 μg to about 100 μg in about 0.30 mL, about 10 μg to about 75 μg in about 0.30 mL, about 10 μg to about 50 μg in about 0.30 mL, about 10 μg to about 25 μg in about 0.30 mL, about 10 μg to about 20 μg in about 0.30 mL, about 10 μg to about 15 μg in about 0.30 mL, about 15 μg to about 600 μg in about 0.30 mL, about 15 μg to about 575 μg in about 0.30 mL, about 15 μg to about 550 μg in about 0.30 mL, about 15 μg to about 525 μg in about 0.30 mL, about 15 μg to about 500 μg in about 0.30 mL, about 15 μg to about 475 μg in about 0.30 mL, about 15 μg to about 450 μg in about 0.30 mL, about 15 μg to about 425 μg in about 0.30 mL, about 15 μg to about 400 μg in about 0.30 mL, about 15 μg to about 375 μg in about 0.30 mL, about 15 μg to about 350 μg in about 0.30 mL, about 15 μg to about 325 μg in about 0.30 mL, about 15 μg to about 300 μg in about 0.30 mL, about 15 μg to about 275 μg in about 0.30 mL, about 15 μg to about 250 μg in about 0.30 mL, about 15 μg to about 225 μg in about 0.30 mL, about 15 μg to about 200 μg in about 0.30 mL, about 15 μg to about 175 μg in about 0.30 mL, about 15 μg to about 150 μg in about 0.30 mL, about 15 μg to about 125 μg in about 0.30 mL, about 15 μg to about 100 μg in about 0.30 mL, about 15 μg to about 75 μg in about 0.30 mL, about 15 μg to about 50 μg in about 0.30 mL, about 15 μg to about 25 μg in about 0.30 mL, about 15 μg to about 20 μg in about 0.30 mL, about 20 μg to about 600 μg in about 0.30 mL, about 20 μg to about 575 μg in about 0.30 mL, about 20 μg to about 550 μg in about 0.30 mL, about 20 μg to about 525 μg in about 0.30 mL, about 20 μg to about 500 μg in about 0.30 mL, about 20 μg to about 475 μg in about 0.30 mL, about 20 μg to about 450 μg in about 0.30 mL, about 20 μg to about 425 μg in about 0.30 mL, about 20 μg to about 400 μg in about 0.30 mL, about 20 μg to about 375 μg in about 0.30 mL, about 20 μg to about 350 μg in about 0.30 mL, about 20 μg to about 325 μg in about 0.30 mL, about 20 μg to about 300 μg in about 0.30 mL, about 20 μg to about 275 μg in about 0.30 mL, about 20 μg to about 250 μg in about 0.30 mL, about 20 μg to about 225 μg in about 0.30 mL, about 20 μg to about 200 μg in about 0.30 mL, about 20 μg to about 175 μg in about 0.30 mL, about 20 μg to about 150 μg in about 0.30 mL, about 20 μg to about 125 μg in about 0.30 mL, about 20 μg to about 100 μg in about 0.30 mL, about 20 μg to about 75 μg in about 0.30 mL, about 20 μg to about 50 μg in about 0.30 mL, about 20 μg to about 25 μg in about 0.30 mL, about 50 μg to about 600 μg in about 0.30 mL, about 50 μg to about 575 μg in about 0.30 mL, about 50 μg to about 550 μg in about 0.30 mL, about 50 μg to about 525 μg in about 0.30 mL, about 50 μg to about 500 μg in about 0.30 mL, about 50 μg to about 475 μg in about 0.30 mL, about 50 μg to about 450 μg in about 0.30 mL, about 50 μg to about 425 μg in about 0.30 mL, about 50 μg to about 400 μg in about 0.30 mL, about 50 μg to about 375 μg in about 0.30 mL, about 50 μg to about 350 μg in about 0.30 mL, about 50 μg to about 325 μg in about 0.30 mL, about 50 μg to about 300 μg in about 0.30 mL, about 50 μg to about 275 μg in about 0.30 mL, about 50 μg to about 250 μg in about 0.30 mL, about 50 μg to about 225 μg in about 0.30 mL, about 50 μg to about 200 μg in about 0.30 mL, about 50 μg to about 175 μg in about 0.30 mL, about 50 μg to about 150 μg in about 0.30 mL, about 50 μg to about 125 μg in about 0.30 mL, about 50 μg to about 100 μg in about 0.30 mL, about 50 μg to about 75 μg in about 0.30 mL, about 75 μg to about 600 μg in about 0.30 mL, about 75 μg to about 575 μg in about 0.30 mL, about 75 μg to about 550 μg in about 0.30 mL, about 75 μg to about 525 μg in about 0.30 mL, about 75 μg to about 500 μg in about 0.30 mL, about 75 μg to about 475 μg in about 0.30 mL, about 75 μg to about 450 μg in about 0.30 mL, about 75 μg to about 425 μg in about 0.30 mL, about 75 μg to about 400 μg in about 0.30 mL, about 75 μg to about 375 μg in about 0.30 mL, about 75 μg to about 350 μg in about 0.30 mL, about 75 μg to about 325 μg in about 0.30 mL, about 75 μg to about 300 μg in about 0.30 mL, about 75 μg to about 275 μg in about 0.30 mL, about 75 μg to about 250 μg in about 0.30 mL, about 75 μg to about 225 μg in about 0.30 mL, about 75 μg to about 200 μg in about 0.30 mL, about 75 μg to about 175 μg in about 0.30 mL, about 75 μg to about 150 μg in about 0.30 mL, about 75 μg to about 125 μg in about 0.30 mL, about 75 μg to about 100 μg in about 0.30 mL, about 100 μg to about 600 μg in about 0.30 mL, about 100 μg to about 575 μg in about 0.30 mL, about 100 μg to about 550 μg in about 0.30 mL, about 100 μg to about 525 μg in about 0.30 mL, about 100 μg to about 500 μg in about 0.30 mL, about 100 μg to about 475 μg in about 0.30 mL, about 100 μg to about 450 μg in about 0.30 mL, about 100 μg to about 425 μg in about 0.30 mL, about 100 μg to about 400 μg in about 0.30 mL, about 100 μg to about 375 μg in about 0.30 mL, about 100 μg to about 350 μg in about 0.30 mL, about 100 μg to about 325 μg in about 0.30 mL, about 100 μg to about 300 μg in about 0.30 mL, about 100 μg to about 275 μg in about 0.30 mL, about 100 μg to about 250 μg in about 0.30 mL, about 100 μg to about 225 μg in about 0.30 mL, about 100 μg to about 200 μg in about 0.30 mL, about 100 μg to about 175 μg in about 0.30 mL, about 100 μg to about 150 μg in about 0.30 mL, about 100 μg to about 125 μg in about 0.30 mL, about 125 μg to about 600 μg in about 0.30 mL, about 125 μg to about 575 μg in about 0.30 mL, about 125 μg to about 550 μg in about 0.30 mL, about 125 μg to about 525 μg in about 0.30 mL, about 125 μg to about 500 μg in about 0.30 mL, about 125 μg to about 475 μg in about 0.30 mL, about 125 μg to about 450 μg in about 0.30 mL, about 125 μg to about 425 μg in about 0.30 mL, about 125 μg to about 400 μg in about 0.30 mL, about 125 μg to about 375 μg in about 0.30 mL, about 125 μg to about 350 μg in about 0.30 mL, about 125 μg to about 325 μg in about 0.30 mL, about 125 μg to about 300 μg in about 0.30 mL, about 125 μg to about 275 μg in about 0.30 mL, about 125 μg to about 250 μg in about 0.30 mL, about 125 μg to about 225 μg in about 0.30 mL, about 125 μg to about 200 μg in about 0.30 mL, about 125 μg to about 175 μg in about 0.30 mL, about 125 μg to about 150 μg in about 0.30 mL, about 150 μg to about 600 μg in about 0.30 mL, about 150 μg to about 575 μg in about 0.30 mL, about 150 μg to about 550 μg in about 0.30 mL, about 150 μg to about 525 μg in about 0.30 mL, about 150 μg to about 500 μg in about 0.30 mL, about 150 μg to about 475 μg in about 0.30 mL, about 150 μg to about 450 μg in about 0.30 mL, about 150 μg to about 425 μg in about 0.30 mL, about 150 μg to about 400 μg in about 0.30 mL, about 150 μg to about 375 μg in about 0.30 mL, about 150 μg to about 350 μg in about 0.30 mL, about 150 μg to about 325 μg in about 0.30 mL, about 150 μg to about 300 μg in about 0.30 mL, about 150 μg to about 275 μg in about 0.30 mL, about 150 μg to about 250 μg in about 0.30 mL, about 150 μg to about 225 μg in about 0.30 mL, about 150 μg to about 200 μg in about 0.30 mL, about 150 μg to about 175 μg in about 0.30 mL, about 175 μg to about 600 μg in about 0.30 mL, about 175 μg to about 575 μg in about 0.30 mL, about 175 μg to about 550 μg in about 0.30 mL, about 175 μg to about 525 μg in about 0.30 mL, about 175 μg to about 500 μg in about 0.30 mL, about 175 μg to about 475 μg in about 0.30 mL, about 175 μg to about 450 μg in about 0.30 mL, about 175 μg to about 425 μg in about 0.30 mL, about 175 μg to about 400 μg in about 0.30 mL, about 175 μg to about 375 μg in about 0.30 mL, about 175 μg to about 350 μg in about 0.30 mL, about 175 μg to about 325 μg in about 0.30 mL, about 175 μg to about 300 μg in about 0.30 mL, about 175 μg to about 275 μg in about 0.30 mL, about 175 μg to about 250 μg in about 0.30 mL, about 175 μg to about 225 μg in about 0.30 mL, about 175 μg to about 200 μg in about 0.30 mL, about 200 μg to about 600 μg in about 0.30 mL, about 200 μg to about 575 μg in about 0.30 mL, about 200 μg to about 550 μg in about 0.30 mL, about 200 μg to about 525 μg in about 0.30 mL, about 200 μg to about 500 μg in about 0.30 mL, about 200 μg to about 475 μg in about 0.30 mL, about 200 μg to about 450 μg in about 0.30 mL, about 200 μg to about 425 μg in about 0.30 mL, about 200 μg to about 400 μg in about 0.30 mL, about 200 μg to about 375 μg in about 0.30 mL, about 200 μg to about 350 μg in about 0.30 mL, about 200μ g to about 325 μg in about 0.30 mL, about 200 g to about 300 μg in about 0.30 ml, about 200 μg to about 275 μg in about 0.30 mL, about 200 μg to about 250 μg in about 0.30 mL, about 200 μg to about 225 μg in about 0.30 mL, about 225 μg to about 600 μg in about 0.30 mL, about 225 μg to about 575 μg in about 0.30 mL, about 225 μg to about 550 μg in about 0.30 mL, about 225 μg to about 525 μg in about 0.30 mL, about 225 μg to about 500 μg in about 0.30 mL, about 225 μg to about 475 μg in about 0.30 mL, about 225 μg to about 450 μg in about 0.30 mL, about 225 μg to about 425 μg in about 0.30 mL, about 225 μg to about 400 μg in about 0.30 mL, about 225 μg to about 375 μg in about 0.30 mL, about 225 μg to about 350 μg in about 0.30 mL, about 225 μg to about 325 μg in about 0.30 mL, about 225 μg to about 300 μg in about 0.30 mL, about 225 μg to about 275 μg in about 0.30 mL, or about 225 μg to about 250 μg in about 0.30 mL of the pharmaceutical composition.

In some embodiments, each of the one or more ampules contains prostaglandin, prostaglandin analogue, prostacyclin or prostacyclin analogue in an amount of about 5 μg to about 600 μg in about 0.50 mL, about 5 μg to about 575 μg in about 0.50 mL, about 5 μg to about 550 μg in about 0.50 mL, about 5 μg to about 525 μg in about 0.50 mL, about 5 μg to about 500 μg in about 0.50 mL, about 5 μg to about 475 μg in about 0.50 mL, about 5 μg to about 450 μg in about 0.50 mL, about 5 μg to about 425 μg in about 0.50 mL, about 5 μg to about 400 μg in about 0.50 mL, about 5 μg to about 375 μg in about 0.50 mL, about 5 μg to about 350 μg in about 0.50 mL, about 5 μg to about 325 μg in about 0.50 mL, about 5 μg to about 300 μg in about 0.50 mL, about 5 μg to about 275 μg in about 0.50 mL, about 5 μg to about 250 μg in about 0.50 mL, about 5 μg to about 225 μg in about 0.50 mL, about 5 μg to about 200 μg in about 0.50 mL, about 5 μg to about 175 μg in about 0.50 mL, about 5 μg to about 150 μg in about 0.50 mL, about 5 μg to about 125 μg in about 0.50 mL, about 5 μg to about 100 μg in about 0.50 mL, about 5 μg to about 75 μg in about 0.50 mL, about 5 μg to about 50 μg in about 0.50 mL, about 5 μg to about 25 μg in about 0.50 mL, about 5 μg to about 20 μg in about 0.50 mL, about 5 μg to about 15 μg in about 0.50 mL, about 5 μg to about 10 μg in about 0.50 mL, about 10 μg to about 600 μg in about 0.50 mL, about 10 μg to about 575 μg in about 0.50 mL, about 10 μg to about 550 μg in about 0.50 mL, about 10 μg to about 525 μg in about 0.50 mL, about 10 μg to about 500 μg in about 0.50 mL, about 10 μg to about 475 μg in about 0.50 mL, about 10 μg to about 450 μg in about 0.50 mL, about 10 μg to about 425 μg in about 0.50 mL, about 10 μg to about 400 μg in about 0.50 mL, about 10 μg to about 375 μg in about 0.50 mL, about 10 μg to about 350 μg in about 0.50 mL, about 10 μg to about 325 μg in about 0.50 mL, about 10 μg to about 300 μg in about 0.50 mL, about 10 μg to about 275 μg in about 0.50 mL, about 10 μg to about 250 μg in about 0.50 mL, about 10 μg to about 225 μg in about 0.50 mL, about 10 μg to about 200 μg in about 0.50 mL, about 10 μg to about 175 μg in about 0.50 mL, about 10 μg to about 150 μg in about 0.50 mL, about 10 μg to about 125 μg in about 0.50 mL, about 10 μg to about 100 μg in about 0.50 mL, about 10 μg to about 75 μg in about 0.50 mL, about 10 μg to about 50 μg in about 0.50 mL, about 10 μg to about 25 μg in about 0.50 mL, about 10 μg to about 20 μg in about 0.50 mL, about 10 μg to about 15 μg in about 0.50 mL, about 15 μg to about 600 μg in about 0.50 mL, about 15 μg to about 575 μg in about 0.50 mL, about 15 μg to about 550 μg in about 0.50 mL, about 15 μg to about 525 μg in about 0.50 mL, about 15 μg to about 500 μg in about 0.50 mL, about 15 μg to about 475 μg in about 0.50 mL, about 15 μg to about 450 μg in about 0.50 mL, about 15 μg to about 425 μg in about 0.50 mL, about 15 μg to about 400 μg in about 0.50 mL, about 15 μg to about 375 μg in about 0.50 mL, about 15 μg to about 350 μg in about 0.50 mL, about 15 μg to about 325 μg in about 0.50 mL, about 15 μg to about 300 μg in about 0.50 mL, about 15 μg to about 275 μg in about 0.50 mL, about 15 μg to about 250 μg in about 0.50 mL, about 15 μg to about 225 μg in about 0.50 mL, about 15 μg to about 200 μg in about 0.50 mL, about 15 μg to about 175 μg in about 0.50 mL, about 15 μg to about 150 μg in about 0.50 mL, about 15 μg to about 125 μg in about 0.50 mL, about 15 μg to about 100 μg in about 0.50 mL, about 15 μg to about 75 μg in about 0.50 mL, about 15 μg to about 50 μg in about 0.50 mL, about 15 μg to about 25 μg in about 0.50 mL, about 15 μg to about 20 μg in about 0.50 mL, about 20 μg to about 600 μg in about 0.50 mL, about 20 μg to about 575 μg in about 0.50 mL, about 20 μg to about 550 μg in about 0.50 mL, about 20 μg to about 525 μg in about 0.50 mL, about 20 μg to about 500 μg in about 0.50 mL, about 20 μg to about 475 μg in about 0.50 mL, about 20 μg to about 450 μg in about 0.50 mL, about 20 μg to about 425 μg in about 0.50 mL, about 20 μg to about 400 μg in about 0.50 mL, about 20 μg to about 375 μg in about 0.50 mL, about 20 μg to about 350 μg in about 0.50 mL, about 20 μg to about 325 μg in about 0.50 mL, about 20 μg to about 300 μg in about 0.50 mL, about 20 μg to about 275 μg in about 0.50 mL, about 20 μg to about 250 μg in about 0.50 mL, about 20 μg to about 225 μg in about 0.50 mL, about 20 μg to about 200 μg in about 0.50 mL, about 20 μg to about 175 μg in about 0.50 mL, about 20 μg to about 150 μg in about 0.50 mL, about 20 μg to about 125 μg in about 0.50 mL, about 20 μg to about 100 μg in about 0.50 mL, about 20 μg to about 75 μg in about 0.50 mL, about 20 μg to about 50 μg in about 0.50 mL, about 20 μg to about 25 μg in about 0.50 mL, about 50 μg to about 600 μg in about 0.50 mL, about 50 μg to about 575 μg in about 0.50 mL, about 50 μg to about 550 μg in about 0.50 mL, about 50 μg to about 525 μg in about 0.50 mL, about 50 μg to about 500 μg in about 0.50 mL, about 50 μg to about 475 μg in about 0.50 mL, about 50 μg to about 450 μg in about 0.50 mL, about 50 μg to about 425 μg in about 0.50 mL, about 50 μg to about 400 μg in about 0.50 mL, about 50 μg to about 375 μg in about 0.50 mL, about 50 μg to about 350 μg in about 0.50 mL, about 50 μg to about 325 μg in about 0.50 mL, about 50 μg to about 300 μg in about 0.50 mL, about 50 μg to about 275 μg in about 0.50 mL, about 50 μg to about 250 μg in about 0.50 mL, about 50 μg to about 225 μg in about 0.50 mL, about 50 μg to about 200 μg in about 0.50 mL, about 50 μg to about 175 μg in about 0.50 mL, about 50 μg to about 150 μg in about 0.50 mL, about 50 μg to about 125 μg in about 0.50 mL, about 50 μg to about 100 μg in about 0.50 mL, about 50 μg to about 75 μg in about 0.50 mL, about 75 μg to about 600 μg in about 0.50 mL, about 75 μg to about 575 μg in about 0.50 mL, about 75 μg to about 550 μg in about 0.50 mL, about 75 μg to about 525 μg in about 0.50 mL, about 75 μg to about 500 μg in about 0.50 mL, about 75 μg to about 475 μg in about 0.50 mL, about 75 μg to about 450 μg in about 0.50 mL, about 75 μg to about 425 μg in about 0.50 mL, about 75 μg to about 400 μg in about 0.50 mL, about 75 μg to about 375 μg in about 0.50 mL, about 75 μg to about 350 μg in about 0.50 mL, about 75 μg to about 325 μg in about 0.50 mL, about 75 μg to about 300 μg in about 0.50 mL, about 75 μg to about 275 μg in about 0.50 mL, about 75 μg to about 250 μg in about 0.50 mL, about 75 μg to about 225 μg in about 0.50 mL, about 75 μg to about 200 μg in about 0.50 mL, about 75 μg to about 175 μg in about 0.50 mL, about 75 μg to about 150 μg in about 0.50 mL, about 75 μg to about 125 μg in about 0.50 mL, about 75 μg to about 100 μg in about 0.50 mL, about 100 μg to about 600 μg in about 0.50 mL, about 100 μg to about 575 μg in about 0.50 mL, about 100 μg to about 550 μg in about 0.50 mL, about 100 μg to about 525 μg in about 0.50 mL, about 100 μg to about 500 μg in about 0.50 mL, about 100 μg to about 475 μg in about 0.50 mL, about 100 μg to about 450 μg in about 0.50 mL, about 100 μg to about 425 μg in about 0.50 mL, about 100 μg to about 400 μg in about 0.50 mL, about 100 μg to about 375 μg in about 0.50 mL, about 100 μg to about 350 μg in about 0.50 mL, about 100 μg to about 325 μg in about 0.50 mL, about 100 μg to about 300 μg in about 0.50 mL, about 100 μg to about 275 μg in about 0.50 mL, about 100 μg to about 250 μg in about 0.50 mL, about 100 μg to about 225 μg in about 0.50 mL, about 100 μg to about 200 μg in about 0.50 mL, about 100 μg to about 175 μg in about 0.50 mL, about 100 μg to about 150 μg in about 0.50 mL, about 100 μg to about 125 μg in about 0.50 mL, about 125 μg to about 600 μg in about 0.50 mL, about 125 μg to about 575 μg in about 0.50 mL, about 125 μg to about 550 μg in about 0.50 mL, about 125 μg to about 525 μg in about 0.50 mL, about 125 μg to about 500 μg in about 0.50 mL, about 125 μg to about 475 μg in about 0.50 mL, about 125 μg to about 450 μg in about 0.50 mL, about 125 μg to about 425 μg in about 0.50 mL, about 125 μg to about 400 μg in about 0.50 mL, about 125 μg to about 375 μg in about 0.50 mL, about 125 μg to about 350 μg in about 0.50 mL, about 125 μg to about 325 μg in about 0.50 mL, about 125 μg to about 300 μg in about 0.50 mL, about 125 μg to about 275 μg in about 0.50 mL, about 125 μg to about 250 μg in about 0.50 mL, about 125 μg to about 225 μg in about 0.50 mL, about 125 μg to about 200 μg in about 0.50 mL, about 125 μg to about 175 μg in about 0.50 mL, about 125 μg to about 150 μg in about 0.50 mL, about 150 μg to about 600 μg in about 0.50 mL, about 150 μg to about 575 μg in about 0.50 mL, about 150 μg to about 550 μg in about 0.50 mL, about 150 μg to about 525 μg in about 0.50 mL, about 150 μg to about 500 μg in about 0.50 mL, about 150 μg to about 475 μg in about 0.50 mL, about 150 μg to about 450 μg in about 0.50 mL, about 150 μg to about 425 μg in about 0.50 mL, about 150 μg to about 400 μg in about 0.50 mL, about 150 μg to about 375 μg in about 0.50 mL, about 150 μg to about 350 μg in about 0.50 mL, about 150 μg to about 325 μg in about 0.50 mL, about 150 μg to about 300 μg in about 0.50 mL, about 150 μg to about 275 μg in about 0.50 mL, about 150 μg to about 250 μg in about 0.50 mL, about 150 μg to about 225 μg in about 0.50 mL, about 150 μg to about 200 μg in about 0.50 mL, about 150 μg to about 175 μg in about 0.50 mL, about 175 μg to about 600 μg in about 0.50 mL, about 175 μg to about 575 μg in about 0.50 mL, about 175 μg to about 550 μg in about 0.50 mL, about 175 μg to about 525 μg in about 0.50 mL, about 175 μg to about 500 μg in about 0.50 mL, about 175 μg to about 475 μg in about 0.50 mL, about 175 μg to about 450 μg in about 0.50 mL, about 175 μg to about 425 μg in about 0.50 mL, about 175 μg to about 400 μg in about 0.50 mL, about 175 μg to about 375 μg in about 0.50 mL, about 175 μg to about 350 μg in about 0.50 mL, about 175 μg to about 325 μg in about 0.50 mL, about 175 μg to about 300 μg in about 0.50 mL, about 175 μg to about 275 μg in about 0.50 mL, about 175 μg to about 250 μg in about 0.50 mL, about 175 μg to about 225 μg in about 0.50 mL, about 175 μg to about 200 μg in about 0.50 mL, about 200 μg to about 600 μg in about 0.50 mL, about 200 μg to about 575 μg in about 0.50 mL, about 200 μg to about 550 μg in about 0.50 mL, about 200 μg to about 525 μg in about 0.50 mL, about 200 μg to about 500 μg in about 0.50 mL, about 200 μg to about 475 μg in about 0.50 mL, about 200 μg to about 450 μg in about 0.50 mL, about 200 μg to about 425 μg in about 0.50 mL, about 200 μg to about 400 μg in about 0.50 mL, about 200 μg to about 375 μg in about 0.50 mL, about 200 μg to about 350 μg in about 0.50 mL, about 200 μg to about 325 μg in about 0.50 mL, about 200 μg to about 300 μg in about 0.50 mL, about 200 μg to about 275 μg in about 0.50 mL, about 200 μg to about 250 μg in about 0.50 mL, about 200 μg to about 225 μg in about 0.50 mL, about 225 μg to about 600 μg in about 0.50 mL, about 225 μg to about 575 μg in about 0.50 mL, about 225 μg to about 550 μg in about 0.50 mL, about 225 μg to about 525 μg in about 0.50 mL, about 225 μg to about 500 μg in about 0.50 mL, about 225 μg to about 475 μg in about 0.50 mL, about 225 μg to about 450 μg in about 0.50 mL, about 225 μg to about 425 μg in about 0.50 mL, about 225 μg to about 400 μg in about 0.50 mL, about 225 μg to about 375 μg in about 0.50 mL, about 225 μg to about 350 μg in about 0.50 mL, about 225 μg to about 325 μg in about 0.50 mL, about 225 μg to about 300 μg in about 0.50 mL, about 225 μg to about 275 μg in about 0.50 mL, or about 225 μg to about 250 μg in about 0.50 mL of the pharmaceutical composition.

In some embodiments, each of the one or more ampules contains about 20 μg to about 25 μg prostaglandin, prostaglandin analogue, prostacyclin or prostacyclin analogue in about 0.13 mL of the pharmaceutical composition. In some embodiments, each of the one or more ampules contains about 65 μg to about 75 μg prostaglandin, prostaglandin analogue, prostacyclin or prostacyclin analogue in about 0.13 mL of the pharmaceutical composition. In some embodiments, each of the one or more ampules contains about 50 μg to about 60 μg prostaglandin, prostaglandin analogue, prostacyclin or prostacyclin analogue in about 0.11 mL of the pharmaceutical composition. In some embodiments, each of the one or more ampules contains about 160 μg to about 170 μg prostaglandin, prostaglandin analogue, prostacyclin or prostacyclin analogue in about 0.11 mL of the pharmaceutical composition. In some embodiments, each of the one or more ampules contains about 25 μg to about 30 μg prostaglandin, prostaglandin analogue, prostacyclin or prostacyclin analogue in about 0.11 mL of the pharmaceutical composition. In some embodiments, each of the one or more ampules contains about 95 μg to about 100 μg prostaglandin, prostaglandin analogue, prostacyclin or prostacyclin analogue in about 0.2 mL of the pharmaceutical composition. In some embodiments, each of the one or more ampules contains about 205 μg to about 215 μg prostaglandin, prostaglandin analogue, prostacyclin or prostacyclin analogue in about 0.14 mL of the pharmaceutical composition. In some embodiments, each of the one or more ampules contains about 70 μg to about 80 μg prostaglandin, prostaglandin analogue, prostacyclin or prostacyclin analogue in about 0.15 mL of the pharmaceutical composition. In some embodiments, each of the one or more ampules contains about 115 μg to about 125 μg prostaglandin, prostaglandin analogue, prostacyclin or prostacyclin analogue in about 0.24 mL of the pharmaceutical composition.

In some embodiments, each of the one or more ampules contains about 23.4 μg prostaglandin, prostaglandin analogue, prostacyclin or prostacyclin analogue in about 0.13 mL of the pharmaceutical composition. In some embodiments, each of the one or more ampules contains about 70.2 μg prostaglandin, prostaglandin analogue, prostacyclin or prostacyclin analogue in about 0.13 mL of the pharmaceutical composition. In some embodiments, each of the one or more ampules contains about 55 μg prostaglandin, prostaglandin analogue, prostacyclin or prostacyclin analogue in about 0.11 mL of the pharmaceutical composition. In some embodiments, each of the one or more ampules contains about 165 μg prostaglandin, prostaglandin analogue, prostacyclin or prostacyclin analogue in about 0.11 mL of the pharmaceutical composition. In some embodiments, each of the one or more ampules contains about 27.3 prostaglandin, prostaglandin analogue, prostacyclin or prostacyclin analogue in about 0.11 mL of the pharmaceutical composition. In some embodiments, each of the one or more ampules contains about 100 μg prostaglandin, prostaglandin analogue, prostacyclin or prostacyclin analogue in about 0.2 mL of the pharmaceutical composition. In some embodiments, each of the one or more ampules contains about 210 μg prostaglandin, prostaglandin analogue, prostacyclin or prostacyclin analogue in about 0.14 mL of the pharmaceutical composition. In some embodiments, each of the one or more ampules contains about 75 μg prostaglandin, prostaglandin analogue, prostacyclin or prostacyclin analogue in about 0.15 mL of the pharmaceutical composition. In some embodiments, each of the one or more ampules contains about 120 μg prostaglandin, prostaglandin analogue, prostacyclin or prostacyclin analogue in about 0.24 mL of the pharmaceutical composition.

In one embodiment, prostaglandin, prostaglandin analogue, prostacyclin or prostacyclin analogue is encapsulated in a plurality of liposomes. The term “liposome” as used herein refers to microscopic vesicles or particles made up of one or more lipid bilayers enclosing an internal aqueous medium. To form liposomes, the presence of at least one “vesicle-forming lipid” is needed, which is an amphipathic lipid capable of either forming or being incorporated into a lipid bilayer. Any suitable vesicle-forming lipid may be used to form the lipid bilayer constituting the liposomes.

The lipid bilayer of the liposome includes at least one vesicle-forming lipid and a sterol, which is selected from the group consisting of cholesterol, cholesteryl hemisuccinate, ergosterol, lanosterol, and any combination thereof, but is not limited thereto. In an exemplary embodiment, the sterol is cholesterol.

In some embodiments, the vesicle-forming lipid is a mixture of a first phospholipid and a second phospholipid. In certain embodiments, the first phospholipid is phosphatidylcholine (PC), which is selected from the group consisting of hydrogenated egg phosphatidylcholine (HEPC), hydrogenated soy phosphatidylcholine (HSPC), dipalmitoyl phosphatidylcholine (DPPC), distearyloyl phosphatidylcholine (DSPC), diarachidoyl phosphatidylcholine, dimyristoyl phosphatidylcholine (DMPC), egg phosphatidylcholine (EPC), soy phosphatidylcholine (SPC), oleoyl palmitoyl phosphatidylcholine, dioleoyl phosphatidylcholine (DOPC), dipetroselinoyl phosphatidylcholine, palmitoylclaidoyl phosphatidylcholine, palmitoyloleoyl phosphatidylcholine, dilauroyl phosphatidylcholine (DLPC), diundecanoyl phosphatidylcholine PC (11:0/11:0), didecanoyl phosphatidylcholine PC (10:0/10:0), dinonanoyl phosphatidylcholine PC (9:0/9:0), and any combination thereof. In another embodiments, the second phospholipid is a polyethylene glycol modified phospholipid, containing a polyethylene glycol having a molecular weight of about 500 to about 10,000 daltons, such as 1,2-distearoyl-sn-glycero-3-phosphoethanolamine-N-[methoxy (polyethylene glycol)-2000] (DSPE-PEG2000), a negatively charged phospholipid, such as distearyloyl phosphatidylglycerol (DSPG), Dipalmitoylphosphatidylglycerol (DPPG) or dimyristoylphosphatidylglycerol (DMPG) or 1,2-dioleoyl-sn-glycero-3-phosphoglycerol (DOPG).

In other embodiments, the vesicle-forming lipid is a mixture of a first phospholipid and a charged lipid. The charged lipid, includes stearylamine, 1,2-dioleoyl-3-trimethylammonium-propane (DOTAP), 3ß-[N—(N′,N′-dimethylaminoethane)-carbamoyl]cholesterol (DC-Cholesterol), N⁴-Cholesteryl-Spermine (GL67), dimethyldioctadecylammonium (DDAB), 1,2-di-O-octadecenyl-3-trimethylammonium propane (DOTMA), ethylphosphocholine (ethyl PC) or combination thereof.

In an exemplary embodiment, the first phospholipid is phosphatidylcholine (PC), phosphatidylglycerol (PG), phosphatidylinositol (PI), phosphatidic acid (PA), phosphatidylethanolamine (PE), phosphatidylserine (PS) or any combination thereof, the second phospholipid is a PEG modified phospholipid, a positively charged or a negatively charged phospholipid, and the charged lipid is a positively charged lipid or a negatively charged lipid.

In another exemplary embodiment, the mole percent of the first phospholipid: cholesterol: second phospholipid or charged lipid is 50-70:20-45:0.1-10, 50-70:15-40:0.1-10 or 55-65:25-40:0.1-5. In yet another exemplary embodiment, the mole percent of the first phospholipid: cholesterol: second phospholipid or charged lipid is 75-99:0-14.9:0.1-25 or 75-99:0-13:0.1-25.

In an embodiment, the mole % of HSPC, cholesterol, and DSPG in the lipid bilayer is 50-70:20-45:0.1-10, 50-70:15-40:0.1-10, 55-65:25-40:0.1-5, 75-99:0-14.9:0.1-25 or 75-99:0-13:0.1-25. In another embodiment, the mole % of HSPC, cholesterol and DSPE-PEG2000 in the lipid bilayer is 50-70:20-45:0.1-10, 50-70:15-40:0.1-10, 55-65:25-40:0.1-5 or 75-99:0-14.9:0.1-25 or 75-99:0-13:0.1-25.

In one embodiment, the lipid bilayer of the liposomes may also include at least one vesicle-forming lipid and a surfactant, which can be a non-ionic surfactant, a cationic surfactant or a zwitterionic surfactant. A non-ionic surfactant has no formally charged groups in its head. A cationic surfactant carries a net positive charge in its head. A zwitterion surfactant is electrically neutral but carries formal positive and negative charges on different atoms.

Non limiting examples of non-ionic surfactant include non-ionic water soluble mono-, di-, and tri-glycerides; non-ionic water soluble mono- and di-fatty acid esters of polyethyelene glycol; non-ionic water soluble sorbitan fatty acid esters (e.g. sorbitan monooleates such as TWEEN 20® (polyoxyethylene 20 sorbitan monooleate), SPAN 80®); non-ionic water soluble triblock copolymers (e.g., poly(ethyleneoxide)/poly-(propyleneoxide)/poly (ethyleneoxide) triblock copolymers such as POLOXAMER 406 (PLURONIC® F-127), or derivatives thereof.

Non-limiting examples of cationic surfactants include dimethyldialkylammonium bromide or dodecyltrimethylammonium bromide.

Non limiting examples of zwitterionic surfactant include 3-(N,N-dimethyl palmitylammonio)-propanesulfonate.

A solvent for dissolving a vesicle-forming lipid for the preparation of liposomes can be, for example, methanol, ethanol, ether, and combinations thereof. Optionally, the solvent can be removed by a supercritical fluid later and is preferably used in a minimum amount so as to decrease the time for performing an organic solvent removing step.

According to the present invention, liposomes are prepared in a medium containing a weak acid salt or an amino acid. Non limiting examples of weak acid salt include carboxylic acid salt and bicarbonate salt.

“Bicarbonate salt” as used herein, refers to a pharmaceutically acceptable salt compound including a bicarbonate anion and a cationic component. In one embodiment, the cationic component of the salt compound is metal. Non-limiting examples of the metal include a Group IA or IIA metal, such as potassium (K), sodium (Na), calcium (Ca), magnesium (Mg), cesium (Cs), and lithium (Li) or a metal other than Group IA or IIA metal, such as ferrous iron (Fe) and nickel (Ni). Examples of bicarbonate salt include, but not limited to, potassium bicarbonate, sodium bicarbonate, calcium bicarbonate, magnesium bicarbonate, cesium bicarbonate, lithium bicarbonate, nickel bicarbonate, ferrous iron bicarbonate or any combination thereof.

In some embodiments, the concentration of bicarbonate salt in in the internal aqueous medium of the liposome is 50 mM or above, 100 mM or above, 150 mM or above, 200 mM or above, 250 mM or above, 300 mM or above, 350 mM or above, 400 mM or above, 450 mM or above, 500 mM or above, 600 mM or above, 700 M or above, 800 mM or above and at most less than 1000 mM. In some embodiments, the concentration of bicarbonate salt in the aqueous buffer solution is from 50 mM to less than 1000 mM, from 50 mM to 800 mM, from 200 mM to less than 1000 mM, from 200 mM to 800 mM, or from 200 mM to 600 mM, from 250 mM to less than 1000 mM, from 250 mM to 800 mM, or from 250 mM to 600 mM, from 300 mM to 600 mM. In an exemplary embodiment, the bicarbonate salt concentration in the internal aqueous medium of the liposome can be measured by any method known or will be known in the art, or by the following steps: (a) separate the bicarbonate salt in the external medium from that of the internal aqueous medium by dialysis or size exclusion chromatography; (b) the liposome suspension without the bicarbonate salt in the external medium is dissolved by methanol and the concentration of the bicarbonate salt in the internal aqueous medium is determined by ion chromatography. In some embodiments, the external medium is substantially free of bicarbonate salt.

“Carboxylic acid salt” as used herein includes, but not limited to, formate, acetate, propionate, butyrate, isobutyrate, valerate, isovalerate or a combination thereof. In one exemplary embodiment, the acetate is sodium acetate, calcium acetate, or a combination thereof.

In one embodiment, the amino acid is a polar amino acid, including a neutral polar amino acid (Tyrosine, Asparagine, Glutamine, Cysteine, Serine, Threonine), a basic polar amino acid (Arginine, Lysine, Histidine), an acidic polar amino acid (Aspartate, Glutamate) or a combination thereof.

Table. 1 shows the classification of 20 amino acids. The 20 amino acids are classified according to page 6 of the biochemistry lecture slides dated 2007, by Professor R H Juang of National Taiwan University (downloaded from http://juang.bst.ntu.edu.tw/files % 20BC/BC2007/Amino (1) % 202007B.pdf).

TABLE 1

| Non-polar | Glycine | Isoleucine | Phenylalanine |
|---|---|---|---|
| | Alanine | Valine | Methionine |
| | Leucine | Proline | Tryptophan |

| | | | |
|---|---|---|---|
| Polar | Acidic | Asparate | Glutamate |

| | | | |
|---|---|---|---|
| Neutral | Tyrosine | Serine | Asparagine |
| | Glutamine | Threonine | Cysteine |
| Basic | Histidine | Lysine | Arginine |

The liposomes in the suspension are subjected to size reduction. The size of a liposome is typically referred to its diameter. Liposome size reduction can be accomplished by a number of methods, such as extrusion, sonication, homogenization techniques or milling techniques, which are well known and can be performed by persons skilled in this art. Extrusion includes passing liposomes, under pressure, one or more times through filters having defined pore sizes. The filters are generally made of polycarbonate, but can also be made of any durable material which does not interact with the liposomes and which is sufficiently strong to allow extrusion under sufficient pressure. The size of the liposomes can be reduced by sonication, which employs sonic energy to disrupt or shear liposomes that will spontaneously reform into smaller liposomes. For example, sonication can be conducted by immersing a glass tube containing the liposome suspension into the sonic epicenter produced in a bath-type sonicator, or a probe type sonicator may be used in which the sonic energy is generated by vibration of a titanium probe in direct contact with the liposome suspension. In the present invention, the liposomes generally have a diameter of about 50 nm to 500 nm, such as about 500 nm or less, about 400 nm or less, about 300 nm or less, about 200 nm or less or about 100 nm or less.

After sizing, the concentration of the weak acid salt or the amino acid in the external medium is adjusted to provide a pH gradient between the internal aqueous medium and the external medium, which can be carried out by a number of ways, for example, by exchanging the external medium with a suitable buffer, such as citric acid buffer (H₃C₆H₅O), L-Histidine Buffer or phosphoric acid buffer (H₃PO₄), by methods such as diafiltration, dialysis, ultrafiltration, or tangential flow filtration.

In one embodiment, the pH of the internal aqueous medium is at least 0.1 unit, at least 0.5 unit or at least 1 unit higher than the pH of the external medium. In yet another embodiment, the pH of the internal aqueous medium is about 7, 8, 9 or 10 to 14, about 7-14 and the pH of the external medium is less than 7, less than 6, less than 5, less than 4, less than 3, about 3-7, about 3.5-6.5, or about 4-6. In yet another exemplary embodiment, the pH of the external medium is above the pKₐ of prostaglandin, prostaglandin analogue, prostacyclin or prostacyclin analogue listed in Table 2.

The prepared liposome can be stored for substantial periods of time prior to drug loading and administration to a subject. For example, liposomes can be stored at refrigerated conditions for substantial periods of time prior to drug loading. Alternatively, liposomes can be dehydrated, stored, and subsequently rehydrated and loaded with one or more active agents as needed, prior to administration. Liposomes may also be dehydrated after being loaded with one or more prostaglandin, prostaglandin analogue, prostacyclin or prostacyclin analogue. Dehydration can be performed by a number of methods available and known in the art. In some embodiments, liposomes are dehydrated using standard freeze-drying apparatus i.e. dehydration under low pressure conditions. Also, liposomes can be frozen e.g. using liquid nitrogen. Saccharides can be added to the liposomal environment, e.g., to the buffer containing the liposomes, prior to dehydration, to ensure stability and integrity of the liposome during dehydration. Examples of saccharides include but are not limited to maltose, lactose, sucrose, trehalose, dextrose, sorbitol, mannitol, xylitol, or a combination thereof.

A liposome suspension having a lower outside/higher inside pH gradient as described above are ready for drug loading. Typically, prostaglandin, prostaglandin analogue, prostacyclin or prostacyclin analogue is added to the external medium of the liposome and the resultant suspension is incubated under a condition to effect drug loading into the internal aqueous medium of the liposome, allowing diffusion of the weak acid drug into the internal aqueous medium of the liposome and until a desired loading concentration and encapsulation efficiency (the percentage of the internal/encapsulated amount of the drug relative to the total amount of the drug in the composition) is achieved.

Table 2 shows the non-limiting examples of prostaglandin, prostacyclin and the analogue thereof of the present invention.

TABLE 2

| Drug category | Drug species |
|---|---|
| Prostaglandin or its | Prostaglandin E1 (PGE1) |
| analogues | Prostaglandin E2 (PGE2) e.g., |
| | dinoprostone |
| | Beraprost |
| | Alprostadil |
| Prostacyclin or its | Epoprostenol |
| analogues | Iloprost |
| | Beraprost |
| | Treprostinil |
| | MRE-269 or ACT-333679 |
| | (prostacyclin receptor agonist) |
| | Ralinepag (prostacyclin receptor |
| | agonist) |

The term “receptor agonist” is considered an analogue, as the receptor agonist is a synthetic molecule designed to mimic the action of a naturally occurring ligand/molecule (such as prostaglandin) by binding to the same receptor and activating it, essentially acting as an “analogue” of the naturally occurring ligand/molecule.

In certain embodiments, the pharmaceutical composition comprises a weak acid drug, such as an endothelin (ET) receptor antagonist (e.g., Ambrisentan, Bosentan and Macitentan), a glucocorticoid (e.g., Hydrocortisone, Methylprednisolone Hemisuccinate (MPSS)), a non-steroid anti-inflammatory drug (NSAID, e.g., Ketorolac, Diflofenac, Ibuprofen), an antibiotic (e.g., Cephalexin) or an anti-coagulant (e.g. warfarin).

As used herein, the terms “encapsulation”, “loaded” and “entrapped” can be used interchangeably, and refer to the incorporation or association of the prostaglandin, prostaglandin analogue, prostacyclin or prostacyclin analogue (e.g., iloprost or treprostinil) in the internal aqueous medium of a liposome.

The liposomes are prepared by dissolving at least one vesicle-forming lipid in an organic solvent to form a lipid solution, which is mixed with an aqueous buffer solution of a weak acid salt or an amino acid to form a suspension which comprises an external medium and at least one liposome, wherein the liposome is suspended in the external medium. The concentration of the weak acid salt or amino acid in the external medium is adjusted by replacing the external medium with a suitable buffer solution lacking the weak acid salt or the amino acid to produce a lower outside/higher inside pH gradient between the external medium and the internal phase of the liposomes. The prostaglandin, prostaglandin analogue, prostacyclin or prostacyclin analogue is added to the external medium of the suspension and the resulting suspension is incubated under a predetermined condition to effect drug loading into the internal phase of the liposomes.

In some embodiments, the pH of the external medium is at least one unit, at least 0.5 unit or at least 0.1 unit above the pKa of the prostaglandin, prostacyclin or the analogues thereof.

In an embodiment, the pharmaceutical composition of the present invention has a drug encapsulation efficiency of at least 50%, at least 55%, at least 60%, at least 65%, at least 70%, at least 75%, at least 80%, at least 85% or at least 90%.

The pharmaceutical composition of the present invention can be administrated at a therapeutically effective amount for treating a respiratory disease or heart failure to a subject in need thereof. Examples of respiratory disease include, but are not limited to, pulmonary hypertension, pulmonary fibrotic disease, interstitial lung disease, chronic obstructive pulmonary disease (COPD), asthma, respiratory complications caused by cystic fibrosis, lower respiratory tract infection, bronchiectasis, bronchitis, bronchiolitis or croup.

In general, the term “treating” as used herein refers to the application or administration of a pharmaceutical composition described herein to a subject afflicted with a respiratory disease or heart failure or with symptoms or signs of the respiratory diseases, with the purpose to cure, heal, alleviate, relieve, alter, remedy, ameliorate, improve, or affect the respiratory disease or heart failure, the symptoms or conditions of the respiratory disease or heart failure, the disabilities induced by the respiratory disease or heart failure, or the progression of the respiratory disease or heart failure. The term “therapeutically effective amount” used herein refers to the amount of prostaglandin, prostaglandin analogue, prostacyclin or prostacyclin analogue to confer a therapeutic effect in a subject. The therapeutically effective amount may change depending on various factors, such as administration route and frequency, body weight and age. Persons skilled in the art may determine the dosage in each case based on the disclosure herein, established methods, and their own experience.

Aerosol administration methods include inhalation, intrathecal, intranasal and pulmonary administration.

### Nebulizer

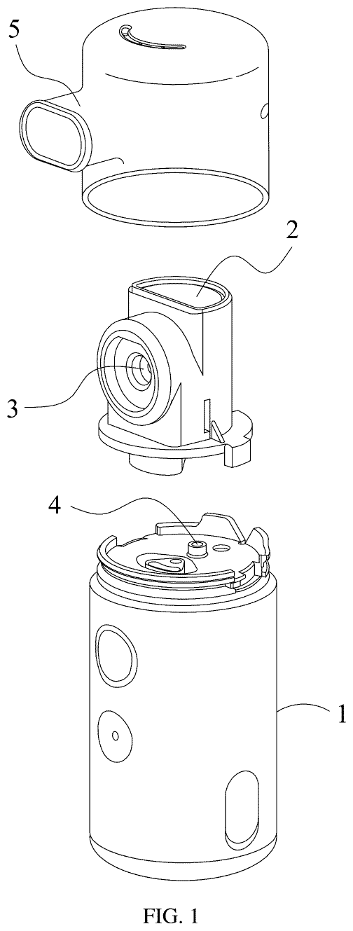

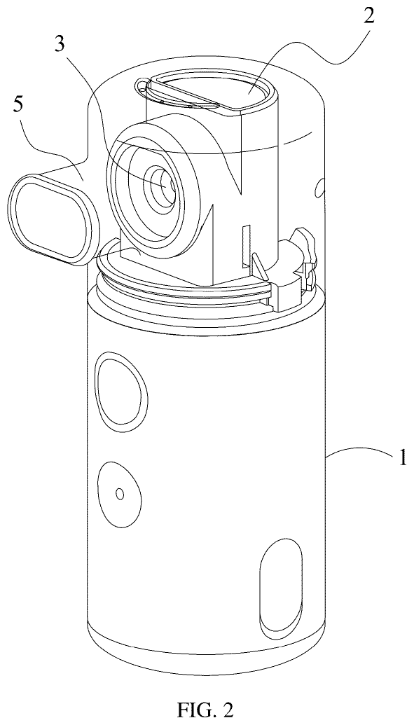

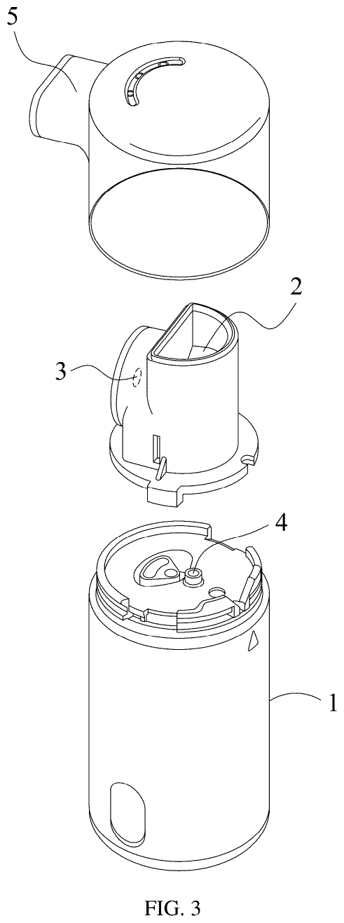

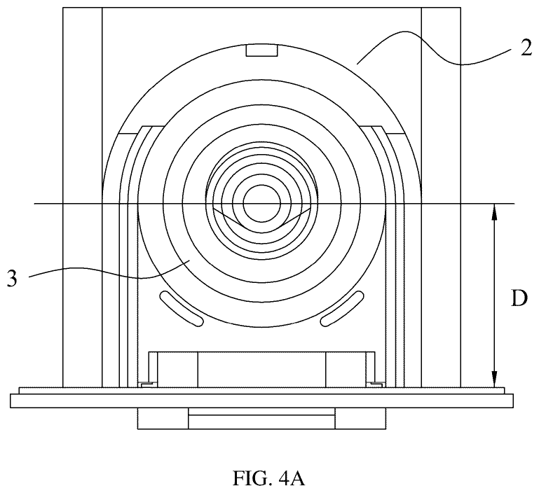

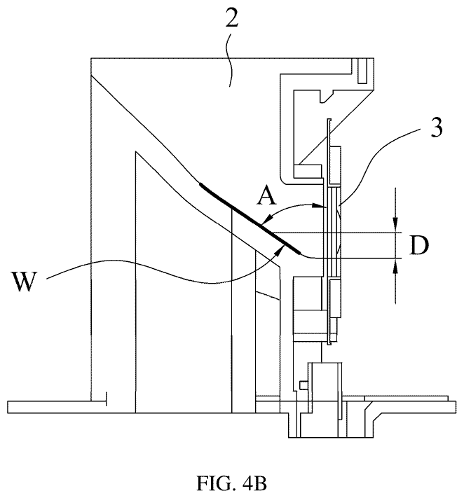

The nebulizer comprises a mesh, a breath-activated sensor and a reservoir or a medication cup. In one embodiment, the nebulizer is an ultrasonic nebulizer. In another embodiment, the nebulizer is a non-ultrasonic nebulizer. FIG. 1 to FIG. 3 illustrate one embodiment of the non-ultrasonic nebulizer, comprises a main body 1, a reservoir or a medication cup 2, a mesh 3, a breath-activated sensor 4 inside the main body and a mouthpiece 5. FIG. 4A and FIG. 4B show the depth of the pharmaceutical composition D, is in contact with the mesh 3 and is at least 1.0 mm, at least 1.1 mm, at least 1.2 mm, at least 1.3 mm, at least 1.4 mm, at least 1.5 mm, at least 1.6 mm, at least 1.7 mm, at least 1.8 mm, or at least 1.9 mm. In an exemplary embodiment, the maximum depth of the pharmaceutical composition is 1 cm. The angle A between the inclined wall of the reservoir and the mesh is about 40 to about 70 degrees, about 45 to about 65 degrees, about 50 to about 60 degrees or about 55 degrees. The combination of the depth of the pharmaceutical composition and the specific angle between the inclined wall of the reservoir and the mesh minimizes the residual volume.

The reservoir or the medication cup is configured to receive the pharmaceutical composition in one or more disposable ampules.

In an exemplary embodiment, the loading volume of the pharmaceutical composition in the reservoir is about 0.01 mL to about 2 mL, about 0.01 mL to less than about 2 mL, about 0.01 mL to about 1.5 mL, about 0.01 mL to about 1.4 mL, about 0.01 mL to about 1.3 mL, about 0.01 mL to about 1.2 mL, about 0.01 mL to about 1.1 mL, about 0.01 mL to about 1.0 mL, about 0.01 mL to less than about 1.0 mL, about 0.01 mL to about 0.9 mL, about 0.01 mL to about 0.8 mL, about 0.01 mL to about 0.7 mL, about 0.01 mL to about 0.6 mL, about 0.01 mL to about 0.5 mL, about 0.05 mL to about 2 mL, about 0.05 mL to about 2 mL, about 0.05 mL to less than about 2 mL, about 0.05 mL to about 1.5 mL, about 0.05 mL to about 1.4 mL, about 0.05 mL to about 1.3 mL, about 0.05 mL to about 1.2 mL, about 0.05 mL to about 1.1 mL, about 0.05 mL to about 1.0 mL, about 0.05 mL to less than about 1.0 mL, about 0.05 mL to about 0.9 mL, about 0.05 mL to about 0.8 mL, about 0.05 mL to about 0.7 mL, about 0.05 mL to about 0.6 mL, about 0.05 mL to about 0.5 mL, or about 0.08 mL to about 2 mL, about 0.08 mL to less than about 2 mL, about 0.08 mL to about 1.5 mL, about 0.08 mL to about 1.4 mL, about 0.08 mL to about 1.3 mL, about 0.08 mL to about 1.2 mL, about 0.08 mL to about 1.1 mL, about 0.08 mL to about 1.0 mL, about 0.08 mL to less than about 1.0 mL, about 0.08 mL to about 0.9 mL, about 0.08 mL to about 0.8 mL, about 0.08 mL to about 0.7 mL, about 0.08 mL to about 0.6 mL, about 0.08 mL to about 0.5 mL, or any range of volumes therebetween (e.g., 0.08 to 0.5 mL, 0.0.5 mL to 1.75 mL, 0.05 to 1.85 mL, 0.05 mL to 1.85 mL, 0.05 mL to 1.75 mL). In one embodiment, the reservoir/medication cup or the mouth piece does not include a piston or a cap with an integrated piston to push the correct amount of pharmaceutical composition into the reservoir or medication cup. In another embodiment, the reservoir/medication cup does not have a cap with an integrated piston.

The nebulizer is configured to synchronize the delivery of the pharmaceutical composition with a breathing pattern of the subject and releases aerosols only during the inhalation phase of the breathing cycle. By synchronizing nebulization with the patient's breathing, the nebulized pharmaceutical composition is effectively deposited in the subject's respiratory tract during inhalation.

The breath-activated sensor detects the subject's inhalation or a change in pressure signaling either the end of an exhalation cycle or commencement of an inhalation cycle. The breath-activated sensor detects inhalation of the subject by detecting a change in pressure that meets or exceeds a predetermined threshold value. The breath-activated sensor transmits an electric current to the mesh and the vibration of the mesh at high frequency (about 100-130 KHz) causes the nebulization of the liquid pharmaceutical composition and produces aerosol particles from the pharmaceutical composition having diameters in the range of about 1 to 5 micrometers.

The mesh is a fine membrane with a plurality of microscopic holes. The mesh vibrates at high frequency (about 100-130 KHz) and converts the liquid pharmaceutical composition into fine aerosols (about 1 to 5 micrometers) for optimal deposition of the aerosols at the targeted site. The vibrating mesh reduces shear forces and heat, preserves the integrity of therapeutic agents (for example, delicate biologics and liposomes) and reduces nebulization time.

The nebulizer delivers about 5 μg to about 2000 μg, about 5 μg to about 1500 μg, about 5 μg to about 1000 μg, about 5 μg to about 900 μg, about 5 μg to about 800 μg, about 5 μg to about 700 μg, 5 μg to about 600 μg, over 5 μg to about 600 μg, about 10 μg to about 600 μg, about 20 μg to about 600 μg, about μg to about 600 μg, about 72 μg to about 600μ, over 72μ g to about 600μ, about 5μ g to about 500μ, over 5μ g to about 500μ g, about 10 μg to about 500 μg, about 20 μg to about 500 μg, about 72 μg to about 500 μg, over 72 μg to about 500 μg, about 5 μg to about 400 μg, over 5 μg to about 400 μg, about 10 μg to about 400 μg, about 20 μg to about 400 μg, about 72 μg to about 400 μg, over 72 μg to about 600 μg, about 20 μg to about 400 μg, about 15 μg to about 75 μg, about 18 μg to about 72 μg, about 42 μg to about 378 μg, about 40 μg to about 380 μg, about 72 μg to about 378 μg, about 73 μg to about 400 μg, about 80 μg to about 400 μg, about 5 μg to about 100 μg, about 10 μg to about 80 μg, about 10 μg to about 70 μg, about 10 μg to about 60 μg, about 20 μg to about 80 μg, about 20 μg to about 70 μg, about 20 μg to about 60 μg, of prostaglandin, prostaglandin analogue, prostacyclin, prostacyclin analogue or other weak acid drugs during each nebulization or treatment session or any range of dose therebetween in 1 μg increment (e.g., 73-400 μg). In some embodiments, the nebulizer delivers the prostaglandin, prostaglandin analogue, prostacyclin, prostacyclin analogue or other weak acid drug from one or more ampules, such as one, two, three or four ampules. In one embodiment, about 15 μg to about 75 μg, about 18 μg to about 72 μg, about 42 μg to about 378 μg, about 40 μg to about 380 μg, about 72 μg to about 378 μg, about 73 μg to about 400 μg, about 80 μg to about 400 μg of treprostinil is delivered during each treatment session in less than three or two. In some embodiments, the nebulizer delivers the treprostinil from one or more ampules, such as one, two, three or four ampules. In another embodiment, about 5 μg to about 100 μg, about 5 μg to about 80 μg, about 10 μg to about 100 μg, about 5 μg to about 70 μg, about 5 μg to about 60 μg, about 10 μg to about 100 μg, about 10 μg to about 80 μg, about 10 μg to about 70 μg, about 10 μg to about 60 μg, about 20 μg to about 80 μg, about 20 μg to about 70 μg, about 20 μg to about 60 μg of iloprost is delivered during each treatment session. In some embodiments, the nebulizer delivers the iloprost from one or more ampules, such as one, two, three or four ampules.

Each nebulization or treatment session is about 5 seconds to less than about 300 seconds and a drug delivery efficiency higher than 50, 55, 60, 65 or 70%.

In some embodiments, nebulization or treatment session is about 5 seconds to about 285 seconds, about 5 seconds to about 270 seconds, about 5 seconds to about 255 seconds, about 5 seconds to about 240 seconds, about 5 seconds to about 225 seconds, about 5 seconds to about 210 seconds, about 5 seconds to about 195 seconds, about 5 seconds to about 180 seconds, about 5 seconds to about 170 seconds, about 5 seconds to about 160 seconds, about 5 seconds to about 150 seconds, about 5 seconds to about 140 seconds, about 5 seconds to about 130 seconds, about 5 seconds to about 120 seconds, about 5 seconds to about 110 seconds, about 5 seconds to about 100 seconds, about 5 seconds to about 90 seconds, about 5 seconds to about 80 seconds, about 5 seconds to about 70 seconds, about 5 seconds to about 60 seconds, about 5 seconds to about 55 seconds, about 5 seconds to about 50 seconds, about 5 seconds to about 45 seconds, about 5 seconds to about 40 seconds, about 5 seconds to about 35 seconds, about 5 seconds to about 30 seconds, about 5 seconds to about 25 seconds, about 5 seconds to about 20 seconds, about 5 seconds to about 15 seconds, about 5 seconds to about 10 seconds, or any range of time therebetween in 1 second increment (e.g., 10 seconds to 3 minutes 45 seconds, 10 seconds to 1 minute 30 seconds). In some embodiments, the nebulization or treatment session is 5 seconds to less than 5 minutes, 5 seconds to less than 4 minutes or 5 seconds to less than 3 minutes.

In some embodiments, each nebulization or treatment session is about 10 seconds to less than about 300 seconds, about 10 seconds to about 285 seconds, about 10 seconds to about 270 seconds, about 10 seconds to about 255 seconds, about 10 seconds to about 240 seconds, about 10 seconds to about 225 seconds, about 10 seconds to about 210 seconds, about 10 seconds to about 195 seconds, about 10 seconds to about 180 seconds, about 10 seconds to about 170 seconds, about 10 seconds to about 160 seconds, about 10 seconds to about 150 seconds, about 10 seconds to about 140 seconds, about 10 seconds to about 130 seconds, about 10 seconds to about 120 seconds, about 10 seconds to about 110 seconds, about 10 seconds to about 100 seconds, about 10 seconds to about 90 seconds, about 10 seconds to about 80 seconds, about 10 seconds to about 70 seconds, about 10 seconds to about 60 seconds, about 10 seconds to about 55 seconds, about 10 seconds to about 50 seconds, about 10 seconds to about 45 seconds, about 10 seconds to about 40 seconds, about 10 seconds to about 35 seconds, about 10 seconds to about 30 seconds, about 10 seconds to about 25 seconds, about 10 seconds to about 20 seconds, or about 10 seconds to about 15 seconds.

In some embodiments, each nebulization or treatment session is about 20 seconds to less than about 300 seconds, about 20 seconds to about 285 seconds, about 20 seconds to about 270 seconds, about 20 seconds to about 255 seconds, about 20 seconds to about 240 seconds, about 20 seconds to about 225 seconds, about 20 seconds to about 210 seconds, about 20 seconds to about 195 seconds, about 20 seconds to about 180 seconds, about 20 seconds to about 170 seconds, about 20 seconds to about 160 seconds, about 20 seconds to about 150 seconds, about 20 seconds to about 140 seconds, about 20 seconds to about 130 seconds, about 20 seconds to about 120 seconds, about 20 seconds to about 110 seconds, about 20 seconds to about 100 seconds, about 20 seconds to about 90 seconds, about 20 seconds to about 80 seconds, about 20 seconds to about 70 seconds, about 20 seconds to about 60 seconds, about 20 seconds to about 55 seconds, about 20 seconds to about 50 seconds, about 20 seconds to about 45 seconds, about 20 seconds to about 40 seconds, about 20 seconds to about 35 seconds, about 20 seconds to about 30 seconds, or about 20 seconds to about 25 seconds.

In some embodiments, each nebulization or treatment session is about 30 seconds to less than about 300 seconds, about 30 seconds to about 285 seconds, about 30 seconds to about 270 seconds, about 30 seconds to about 255 seconds, about 30 seconds to about 240 seconds, about 30 seconds to about 225 seconds, about 30 seconds to about 210 seconds, about 30 seconds to about 195 seconds, about 30 seconds to about 180 seconds, about 30 seconds to about 170 seconds, about 30 seconds to about 160 seconds, about 30 seconds to about 150 seconds, about 30 seconds to about 140 seconds, about 30 seconds to about 130 seconds, about 30 seconds to about 120 seconds, about 30 seconds to about 110 seconds, about 30 seconds to about 100 seconds, about 30 seconds to about 90 seconds, about 30 seconds to about 80 seconds, about 30 seconds to about 70 seconds, about 30 seconds to about 60 seconds, about 30 seconds to about 55 seconds, about 30 seconds to about 50 seconds, about 30 seconds to about 45 seconds, about 30 seconds to about 40 seconds, or about 30 seconds to about 35 seconds.

In some embodiments, each nebulization or treatment session is about 40 seconds to less than about 300 seconds, about 40 seconds to about 285 seconds, about 40 seconds to about 270 seconds, about 40 seconds to about 255 seconds, about 40 seconds to about 240 seconds, about 40 seconds to about 225 seconds, about 40 seconds to about 210 seconds, about 40 seconds to about 195 seconds, about 40 seconds to about 180 seconds, about 40 seconds to about 170 seconds, about 40 seconds to about 160 seconds, about 40 seconds to about 150 seconds, about 40 seconds to about 140 seconds, about 40 seconds to about 130 seconds, about 40 seconds to about 120 seconds, about 40 seconds to about 110 seconds, about 40 seconds to about 100 seconds, about 40 seconds to about 90 seconds, about 40 seconds to about 80 seconds, about 40 seconds to about 70 seconds, about 40 seconds to about 60 seconds, about 40 seconds to about 55 seconds, about 40 seconds to about 50 seconds, or about 40 seconds to about 45 seconds.

In some embodiments, each nebulization or treatment session is about 50 seconds to less than about 300 seconds, about 50 seconds to about 285 seconds, about 50 seconds to about 270 seconds, about 50 seconds to about 255 seconds, about 50 seconds to about 240 seconds, about 50 seconds to about 225 seconds, about 50 seconds to about 210 seconds, about 50 seconds to about 195 seconds, about 50 seconds to about 180 seconds, about 50 seconds to about 170 seconds, about 50 seconds to about 160 seconds, about 50 seconds to about 150 seconds, about 50 seconds to about 140 seconds, about 50 seconds to about 130 seconds, about 50 seconds to about 120 seconds, about 50 seconds to about 110 seconds, about 50 seconds to about 100 seconds, about 50 seconds to about 90 seconds, about 50 seconds to about 80 seconds, about 50 seconds to about 70 seconds, about 50 seconds to about 60 seconds, or about 50 seconds to about 55 seconds.

In some embodiments, each nebulization or treatment session is about 60 seconds to less than about 300 seconds, about 60 seconds to about 285 seconds, about 60 seconds to about 270 seconds, about 60 seconds to about 255 seconds, about 60 seconds to about 240 seconds, about 60 seconds to about 225 seconds, about 60 seconds to about 210 seconds, about 60 seconds to about 195 seconds, about 60 seconds to about 180 seconds, about 60 seconds to about 170 seconds, about 60 seconds to about 160 seconds, about 60 seconds to about 150 seconds, about 60 seconds to about 140 seconds, about 60 seconds to about 130 seconds, about 60 seconds to about 120 seconds, about 60 seconds to about 110 seconds, about 60 seconds to about 100 seconds, about 60 seconds to about 90 seconds, about 60 seconds to about 80 seconds, or about 60 seconds to about 70 seconds.

The nebulizer is configured to minimize the residual volume of the pharmaceutical composition after each nebulization session. The low residual volume of the pharmaceutical composition reduces medication waste, maximizes therapeutic efficacy and reduces cost. In one embodiment, the residual volume of the pharmaceutical composition in the reservoir after each nebulization session (or the volume the nebulizer is configured to retain in the reservoir after the drug delivery) is about 0.005 to about 0.01 mL, about 0.01 mL to about 1 mL, about 0.01 mL to about 0.9 mL, about 0.01 mL to about 0.8 mL, about 0.01 mL to about 0.7 mL, about 0.01 mL to about 0.6 mL, about 0.01 mL to about 0.5 mL, about 0.01 mL to about 0.4 mL, about 0.01 mL to about 0.3 mL, about 0.01 mL to about 0.2 mL, about 0.01 mL to about 0.1 mL, or about 0.01 mL to about 0.05 mL, or any range of volumes therebetween in 0.01 mL increments (e.g., 0.02 mL to 0.45 mL, 0.05 mL to 0.5 mL). In another embodiment, the residual volume is about 0.01 mL to about 0.05 mL, about 0.01 mL to about 0.1 mL, or about 0.01 mL to about 0.5 mL, about 0.02 mL to about 0.05 mL, about 0.02 mL to about 0.1 mL, or about 0.02 mL to about 0.5 mL.

The drug delivery efficiency of the nebulizer kit disclosed here (calculated as follows) is higher than about 60%, higher than about 70%, about 60% to about 95%, about 70% to about 95%.

$$Drug delivery efficiency ( \% ) = Measured delivered dose \text{} ( μg ) / [ sample concentration ( \frac{μg}{mL} ) \times sample loaded volume ( mL ) ] \times 100 \%$$

TABLE 3
Drug delivery Efficiency (% of Loading Dose) of
various embodiments of the present invention.

| | 0.1 mL | 0.2 mL | 0.3 mL |
|---|---|---|---|
| | (Loading volume) | (Loading volume) | (Loading volume) |
| Treprostinil Liposomal Composition* | | | |
| 0.5 mg/mL | 78.3% (39.15 μg) | 81.1% (81.1 μg) | 87.4% (131.1 μg) |
| 1.5 mg/mL | 77.9% (116.85 μg) | 82.4% (247.2 μg) | 86.3% (388.35 μg) |
| Iloprost Liposomal Composition** | | | |
| 0.25 mg/mL | 74.3% (18.58 μg) | 80.3% (40.15 μg) | 85.2% (63.9 μg) |

* *HSPC:CHOL:DSPG = 3:2:0.075 (mole ratio)
**HSPC:DSPE-mPEG2K = 98:2 (mole ratio)

Details of Tyvaso® Inhalation System, Ventavis® and Breelib™ nebulizer and various embodiments of the nebulizer kit of the present invention are listed in Tables 4-6 below.

### Treprostinil Composition

Details of the FDA approved Tyvaso® Inhalation System
To be used with an ultrasonic, pulsed delivery device
2.9 mL ampule containing 1.74 mg of treprostinil (0.6 mg per mL)
Initial Dose: 3 breaths (18 μg of treprostinil per breath*3) per treatment session, 4 times a day.
Treatment session will take 2 to 3 minutes.
Titration: Increase by 3 breaths per session every 1-2 weeks to a maximum of 9 breaths (54 μg) per session.
Administer in 4 separate treatment sessions each day approximately 4 hours apart, during waking hours.
Drug Delivery Efficiency:

$$( 72 ) / ( 1740 ) = 4.14 \% ( 2.9 mL solution per each treatment session ) ,$$

$$( 72 \times 4 ) / ( 1740 ) = 16.56 \% ( 2.9 mL solution for 4 treatment sessions daily )$$

One embodiment of the nebulizer kit disclosed herein
To be used with the nebulizer disclosed herein.
Two exemplary treprostinil liposomal compositions (HSPC:CHOL:DSPG=3:2:0.075)
23.4 μg/0.13 mL and 2. 70.2 μg/0.13 mL.
Initial Dose: 18 μg per treatment session, 4 times a day.
Titration: Increase by 18 μg per session every 1-2 weeks until they achieve their maximum clinically tolerated dose.
Administer in 4 separate treatment sessions each day approximately 4 hours apart, during waking hours
Drug Delivery Efficiency: >70%

TABLE 4

| | | Loading | | | | |
|---|---|---|---|---|---|---|
| | | volume | | | | |
| | Ampules | (considering | | | | |
| | to | 0.01 mL | | | Sample | Drug |
| | Achieve | residual | Measured | | Volume | delivery |
| Delivered | Nominal | vol. in | Delivered | Sample | Loading | efficiency¹ |
| Dose | Dose | ampoule) | Dose | Concentration | dose | (%) |
| 18 μg | 23.4 μg/ | 0.12 mL | 16.3 μg | 0.18 mg/mL | 21.6 μg | 75.5 |
| | 0.13 mL | | | | | 16.3 μg/(180 μg/ |
| | | | | | | mL × 0.12 |
| | | | | | | mL) × 100% |
| 36 μg | 23.4 μg/ | 0.24 mL | 38.5 μg | 0.18 mg/mL | 43.2 μg | 89.1 |
| | 0.13 mL + | | | | | 38.5 μg/(240 μg/ |
| | 23.4 μg/ | | | | | mL × 0.24 |
| | 0.13 mL | | | | | mL) × 100% |
| 54 μg | 70.2 μg/ | 0.12 mL | 47.2 μg | 0.54 mg/mL | 64.8 μg | 72.9 |
| | 0.13 mL | | | | | 47.2 μg/(540 μg/ |
| | | | | | | mL × 0.12 |
| | | | | | | mL) × 100% |
| 72 μg | 23.4 μg/ | 0.24 mL | 77.3 μg | 0.36 mg/mL | 86.4 μg | 89.5 |
| | 0.13 mL + | | | | | 77.3 μg/(360 μg/ |
| | 70.2 μg/ | | | | | mL × 0.24 |
| | 0.13 mL | | | | | mL) × 100% |

¹Drug delivery efficiency (%) = (Measured delivered dose (μg))/[sample concentration (μg/mL) × sample volume (mL)] × 100%

Second embodiment of the nebulizer kit disclosed herein.
To be used with the nebulizer disclosed herein.
Six exemplary treprostinil liposomal compositions (HSPC:CHOL:DSPG=3:2:0.075):
55 μg/0.11 mL, 75 μg/0.15 mL, 100 μg/0.20 mL and 120 μg/0.24 mL in 0.5 mg/mL, 165 μg/0.11 mL and 210 μg/0.14 mL in 1.5 mg/mL
Initial Dose: 42 μg per treatment session, 12 hours apart at approximately the same time each day.
Titration: Increase by 42 μg per session every 1-2 weeks until maximum clinically tolerated dose is achieved.
Drug Delivery Efficiency: >70%

TABLE 5

| | | Loading | | | | |
|---|---|---|---|---|---|---|
| | | volume | | | | |
| | | (considering | | | | |
| | Ampules | 0.009-0.010 | | | | Drug |
| | to Achieve | mL residual | Measured | | | delivery |
| Delivered | Nominal | vol. in | Delivered | Sample | Loading | efficiency¹ |
| Dose | Dose | ampoule) | Dose | Concentration | dose | (%) |
| 42 μg | 55 μg/ | 0.100 mL | 36.3 μg | 0.50 mg/mL | 50.0 μg | 72.5 |
| | 0.11 mL | | | | | 36.3 μg/(500 |
| | | | | | | μg/mL × 0.10 |
| | | | | | | mL) × 100% |
| 84 μg | 100 μg/ | 0.190 mL | 85.0 μg | 0.50 mg/mL | 95.0 μg | 89.5 |
| | 0.20 mL | | | | | 85.0 μg/(500 |
| | | | | | | μg/mL × 0.19 |
| | | | | | | mL) × 100% |
| 126 μg | 165 μg/ | 0.101 mL | 110.4 μg | 1.50 mg/mL | 151.5 μg | 72.9 |
| | 0.11 mL | | | | | 110.4 μg/ |
| | | | | | | (1500 μg/mL × |
| | | | | | | 0.101 mL) × |
| | | | | | | 100% |
| 169 μg | 210 μg/ | 0.131 mL | 145.8 μg | 1.50 mg/mL | 196.5 μg | 74.2 |
| | 0.14 mL | | | | | 145.8 μg/ |
| | | | | | | (1500 μg/mL × |
| | | | | | | 0.131 mL) × |
| | | | | | | 100% |
| 207 μg | 75 μg/ | 0.241 mL | 200.7 μg | 0.92 mg/mL | 221.7 μg | 90.5 |
| | 0.15 mL + | | | | | 200.7 μg/ |
| | 165 μg/ | | | | | (920 μg/mL × |
| | 0.11 mL | | | | | 0.241 mL) × |
| | | | | | | 100% |
| 251 μg | 120 μg/ | 0.331 mL | 246.9 μg | 0.81 mg/mL | 268.1 μg | 92.1 |
| | 0.24 mL + | | | | | 246.9 μg/ |
| | 165 μg/ | | | | | (810 μg/mL × |
| | 0.11 mL | | | | | 0.331 mL) × |
| | | | | | | 100% |
| 292 μg | 120 μg/ | 0.361 mL | 288.4 μg | 0.86 mg/mL | 310.5 μg | 92.9 |
| | 0.24 mL + | | | | | 288.4 μg/ |
| | 210 μg/ | | | | | (860 μg/mL × |
| | 0.14 mL | | | | | 0.361 mL) × |
| | | | | | | 100% |
| 333 μg | 120 μg/ | 0.690 mL | 324.6 μg | 0.50 mg/mL | 345.0 μg | 94.1 |
| | 0.24 mL + | | | | | 324.6 μg/ |
| | 120 μg/ | | | | | (500 μg/mL × |
| | 0.24 mL + | | | | | 0.690 mL) × |
| | 120 μg/ | | | | | 100% |
| | 0.24 mL | | | | | |
| 378 μg | 100 μg/ | 0.392 mL | 377.4 μg | 1.02 mg/mL | 399.8 μg | 94.4 |
| | 0.20 mL + | | | | | 377.4 μg/ |
| | 165 μg/ | | | | | (1020 μg/mL × |
| | 0.11 mL + | | | | | 0.392 mL) × |
| | 165 μg/ | | | | | 100% |
| | 0.11 mL | | | | | |

¹Drug delivery efficiency (%) = (Measured delivered dose (μg))/[sample concentration (μg/mL) × sample volume (mL)] × 100%

### Iloprost Compositions

Details of the Ventavis® and Breelib™ Nebulizer
Initial Dosage: 2.5 μg iloprost per treatment session. 6-9 times daily, at least 2 hours between doses.
Titration: increase to 5 μg per treatment session if tolerated.
Drug Delivery Efficiency:

$$5 / ( 20 \times 1 ) = 25 \% for single treatment session$$

One embodiment of the nebulizer kit
To be used with the nebulizer disclosed herein.
Two exemplary iloprost liposomal compositions (HSPC: DSPE-mPEG2K=98:2): 27.5 μg/0.11 mL and 40 μg/0.15 mL in 0.25 mg/mL
Initial Dose: 20 μg per treatment session, 12 hours apart at approximately the same time each day.
Titration: Start at 20 μg and increase to higher dose as tolerated.
Drug Delivery Efficiency: >70%

TABLE 6

| | | Loading | | | | |
|---|---|---|---|---|---|---|
| | | volume | | | | |
| | | (considering | | | | |
| | Ampules to | 0.009-0.010 | | | | Drug |
| | Achieve | mL residual | Measured | | | delivery |
| Delivered | Nominal | vol. in | Delivered | Sample | Loading | efficiency¹ |
| Dose | Dose | ampoule) | Dose | Concentration | dose | (%) |
| 20 μg | 27.5 μg/ | 0.10 mL | 17.9 μg | 0.25 mg/mL | 25.0 μg | 71.5 |
| | 0.11 mL | | | | | 17.9 μg/ |
| | | | | | | (250 μg/mL × |
| | | | | | | 0.10 mL) × |
| | | | | | | 100% |
| 30 μg | 37.5 μg/ | 0.14 mL | 28.3 μg | 0.25 mg/mL | 35.0 μg | 75.4 |
| | 0.15 mL | | | | | 28.3 μg/ |
| | | | | | | (250 μg/mL × |
| | | | | | | 0.14 mL) × |
| | | | | | | 100% |
| 40 μg | 27.5 μg/ | 0.20 mL | 38.2 μg | 0.25 mg/mL | 50.0 μg | 76.3 |
| | 0.11 mL + | | | | | 38.2 μg/ |
| | 27.5 μg/ | | | | | (250 μg/mL × |
| | 0.11 mL | | | | | 0.20 mL) × |
| | | | | | | 100% |
| 50 μg | 27.5 μg/ | 0.24 mL | 44.4 μg | 0.25 mg/mL | 60.0 μg | 74.0 |
| | 0.11 mL + | | | | | 44.4 μg/ |
| | 37.5 μg/ | | | | | (250 μg/mL × |
| | 0.15 mL | | | | | 0.24 mL) × |
| | | | | | | 100% |
| 60 μg | 37.5 μg/ | 0.28 mL | 57.4 μg | 0.25 mg/mL | 70.0 μg | 82.0 |
| | 0.15 mL + | | | | | 57.4 μg/ |
| | 37.5 μg/ | | | | | (250 μg/mL × |
| | 0.15 mL | | | | | 0.28 mL) × |
| | | | | | | 100% |

¹Drug delivery efficiency (%) = (Measured delivered dose (μg))/[sample concentration (μg/mL) × sample volume (mL)] × 100%

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Example 1

### 1. Materials and Methods

### 1.1 Liposome Treprostinil Composition

A Liposomal colloidal suspension was prepared by ethanol injection technique. Briefly, 469.8 mg of HSPC, 154.65 mg of cholesterol and 12.02 mg DSPG were dissolved in 2.86 mL of ethanol. The lipid solution in ethanol was injected into 17.4 mL of sodium bicarbonate (400 mM) with vigorous stirring at 60° C. Liposomes were formed as soon as the lipid solution was in contact with sodium acetate. This was followed by extruding the liposome suspension through a 200 nm and a 100 nm polycarbonate membrane (six times, respectively). The liposomes with a diameter around 100 nm to 200 nm and a polydispersity index (PdI) of <0.2 were obtained and diafiltrated against a citrate buffer (pH 5.5) to form a higher inside/lower outside pH gradient between the external medium and the internal aqueous medium of the liposomes. The empty liposome suspension was then loaded with treprostinil (5 mg/mL) at 37° C. over 30 minutes to achieve a final target concentration (e.g., 100 to 2000 μg/mL), compared to a 10 mM lipid concentration in the liposome suspension.

### 1.2 Liposomal Iloprost Composition

A liposomal colloidal suspension was prepared using ethanol injection technique. All of the lipid ingredients, including a first phospholipid (HSPC) and a second phospholipid (DSPE-mPEG2K) at a molar ratio of 98:2, were dissolved in 2.86 mL of ethanol solution at approximately 60° C. The resultant lipid solution was injected into 20 mL of sodium bicarbonate solution (100 mM; pH 8.5) and (2-Hydroxypropyl)-β-cyclodextrin (110 mM) and mixed with vigorous stirring at 60° C. for liposome hydration. The mixture was extruded 6 to 10 times through polycarbonate membranes with pore size of 0.2 or 0.1 μm to obtain a suspension of liposomes having a mean particle size within a range of around 100 nm to 200 nm and a polydispersity index (PdI) of <0.2. The suspension of liposomes was dialyzed with a tangential flow filtration system against L-Histidine Buffer (pH 6.5) to form a transmembrane pH gradient between the internal aqueous medium of the liposome and the external medium (i.e., a higher inside and lower outside pH gradient). The suspension of liposomes having such pH gradient was then loaded with iloprost (500 μg/mL) (purchased from Cayman Chemical, USA) at 37° C. over 30 minutes to achieve a final target concentration (e.g., 100 to 2000 μg/mL), compared to a 10 mM lipid concentration in the liposome suspension.

### 1.3 Nebulization

The nebulizer illustrated in FIG. 1, the Tyvaso Inhalation system (TD-100, an ultrasonic pulsed device with a minimum nebulizing solution loading volume of 2.9 mL, United Therapeutics Corporation, USA) and Breelib® FOX-001 nebulizer (Vectura, UK) were securely connected to breath simulators (BRS 2000, commercially available from Copley, UK) using appropriate tubing. The adult breathing pattern outlined in <1601> of the United States Pharmacopeia was programmed to the breath simulator, followed by calibration of the flow rate, volume, and breath frequency. The reservoir of the nebulizer was weighed before and after pharmaceutical composition loading. The following pharmaceutical compositions were used in this experiment:

TABLE 7

| | lipid | | |
|---|---|---|---|
| Pharmaceutical | composition | Internal | External |
| composition | (molar ratio) | buffer | buffer |
| liposomal | HSPC:CHOL:DSPG = | 400 mM | Citrate |
| treprostinil | 3:2:0.075 | Bicarbonate | Buffer |
| solution | | | pH 5.5 |
| liposomal | HSPC:DSPE-mPEG2K = | 100 mM | L-Histidine |
| iloprost | 98:2 | Bicarbonate + | Buffer |
| solution | | 110 mM | pH 6.5 |
| | | HP-β-CD | |
| Free | — | — | — |
| treprostinil | | | |
| (Tyvaso ®) | | | |
| solution | | | |
| Free | — | — | — |
| Iloprost | | | |
| solution | | | |
| (Ventavis ®) | | | |

The nebulizer and the breath simulator were turned on simultaneously and the total inhalation time to complete the drug delivery/nebulization was recorded. The treatment session time is calculated by multiplying the inhalation time by 2. The reservoir was weighed post-nebulization to determine the residual volume.

### 1.3 Quantitative Determination of Drug Delivery

The amount of the drug delivered by the nebulizer was measured by the following method:

A filter is placed between the nebulizer and the breath simulator during each nebulization/treatment session. The amount of drug delivered is the amount of drug on the filter at the end of each nebulization/treatment session, measured by HPLC.

### 2. Results

### 2.1 Treprostinil Solution

TABLE 8

| | | | | Tyvaso Inhalation |
|---|---|---|---|---|
| | | | Nebulizer of FIG. 1 | system ® |
| | | | Loading Volume/ | Loading Volume/ |
| | | | Treatment Session/ | Treatment Session/ |
| Pharmaceutical | | Delivered | Drug delivery | Drug delivery |
| composition | Strength | dose | efficiency | efficiency |
| Liposomal | 1.5 mg/mL | 384 μg | 0.26 mL / | 2.9 mL / |
| treprostinil | | | 1.48 minutes/ | 6.4 minutes / |
| | | | 339.9 μg/(1500 μg/mL × | 384 μg / 1500 μg/mL × |
| | | | 0.26 mL) × | 2.9 mL) × |
| | | | 100% = 88.52% | 100% = 8.83% |
| Free/Liposomal | 0.6 mg/mL | 72 μg | 0.12 mL liposomal | 2.9 mL free |
| Treprostinil | | | treprostinil | treprostinil |
| | | | 0.68 minutes/ | 3 minutes/ |
| | | | 53.9 μg/72 μg = | 72 μg/(600 μg/mL × |
| | | | 74.8% | 2.9 mL) × 100% = 4.14% |
| Treprostinil | 0.6 mg/mL | 72 μg | Free treprostinil | Liposomal treprostinil |
| | | | loading volume is | loading volume is |
| | | | 2.9 mL, higher than | 0.12 mL, less than |
| | | | the max loading | Min. working vol. |
| | | | volume of the | of the Tyvaso Inhalation |
| | | | claimed nebulizer. | System (2.9 mL) |

### Iloprost Solution

TABLE 9

| | | | | Nebulizer of FIG. 1 | Breelib ® nebulizer |
|---|---|---|---|---|---|
| | | Actual | | Treatment Session/ | Treatment Session/ |
| Pharma | | loading | Delivered | Drug delivery | Drug delivery |
| Composition | Strength | vol. | dose | efficiency | efficiency |
| Liposomal | 0.10 mg/mL | 0.05 mL | 5 μg | 0.31 min / 76.0% | — |
| Iloprost | | | | (3.8 μg / 5 μg × | |
| | | | | 100%) | |
| Liposomal | 0.02 mg/mL | 0.25 mL | 5 μg | 2.84 min / 84.3% | — |
| Iloprost | | | | (4.2 μg / 5 μg × | |
| | | | | 100%) | |
| Liposomal | 0.25 mg/mL | 0.16 mL | 40 μg | 0.92 min/ >71.5% | — |
| Iloprost | | | | (28.6 μg / 40 μg × | |
| | | | | 100%) | |
| Ven-tavis ® | 0.02 mg/mL | 1.0 mL | 5 μg | — | 5 minutes / |
| | | | | | 5 μg/(20 μg/mL × |
| | | | | | 1 mL) × 100% = 25% |
| Ven-tavis ® | 0.16 mg/mL* | 1.0 mL | 40 μg | — | 5 minutes / |
| | | | | | 40 μg/(160 μg/mL × |
| | | | | | 1 mL) × 100% = 25% |

*Solubility at 0.16 mg/mL in buffered aqueous solution may lead to precipitation during long-term storage due to potential pH shifts.

The results in Table 8 and Table 9 show the combination of the claimed pharmaceutical compositions and the claimed nebulizer increases the drug delivery efficiency and shortens the nebulization/treatment time.

1. A kit, comprising:
(i) about 0.01 mL to about 2 mL of a pharmaceutical composition, said pharmaceutical composition comprising about 100 to about 2000 μg/mL of a prostaglandin, prostaglandin analogue, prostacyclin or prostacyclin analogue;
(ii) a nebulizer, comprising a mesh, a breath-activated sensor and a reservoir, said reservoir is configured to hold about 0.01 mL to about 2 mL of the pharmaceutical composition;
wherein the nebulizer is configured to deliver about 5 to about 600 μg of the prostaglandin, prostaglandin analogue, prostacyclin or prostacyclin analogue in less than 5 minutes.

2. The kit of claim 1, wherein the pharmaceutical composition is contained in one or more ampules.

3. The kit of claim 2, wherein the volume of the pharmaceutical composition in each of the one or more ampules is about 0.01 mL to about 2 mL.

4. The kit of claim 1, wherein the nebulizer is configured to deliver the prostaglandin, prostaglandin analogue, prostacyclin or prostacyclin analogue with at least 60% drug delivery efficiency.

5. The kit of claim 1, wherein the prostaglandin, prostaglandin analogue, prostacyclin or prostacyclin analogue is encapsulated in a plurality of liposomes.

6. The kit of claim 5, wherein the liposomes comprise:
(a) a lipid bilayer, comprising a vesicle-forming lipid that is a mixture of a first phospholipid and a second phospholipid or a mixture of a first phospholipid and a charged lipid; and
(b) an internal aqueous medium inside the lipid bilayer, comprising (i) a weak acid salt or an amino acid and (ii) the prostaglandin, prostaglandin analogue, prostacyclin or prostacyclin analogue,
wherein the liposomes are suspended in an external medium and the pH of the external medium is above the pKₐ of the prostaglandin, prostaglandin analogue, prostacyclin or prostacyclin analogue.

7. The kit of claim 6, wherein the weak acid salt is a carboxylic acid salt or bicarbonate salt.

8. The kit of claim 7, wherein the carboxylic acid salt is selected from the group consisting of formate, acetate, propionate, butyrate, isobutyrate, valerate, isovalerate, benzoate and a combination thereof.

9. The kit of claim 7, wherein the concentration of the bicarbonate salt is about 50 mM to less than about 1000 mM.

10. The kit of claim 6, wherein the amino acid is a neutral polar amino acid or basic polar amino acid.

11. The kit of claim 6, wherein the first phospholipid is phosphatidylcholine (PC), phosphatidylglycerol (PG), phosphatidylinositol (PI), phosphatidic acid (PA), phosphatidylethanolamine (PE), phosphatidylserine (PS) or any combination thereof, the second phospholipid is a PEG modified phospholipid, a positively charged or a negatively charged phospholipid, and the charged lipid is a positively charged lipid or a negatively charged lipid.

12. The kit of claim 6, wherein the vesicle-forming lipid further comprises a sterol that is cholesterol, cholesteryl hemisuccinate, ergosterol, lanosterol, or any combination thereof.

13. The kit of claim 12, wherein the first phospholipid is hydrogenated egg phosphatidylcholine (HEPC), hydrogenated soy phosphatidylcholine (HSPC), dipalmitoyl phosphatidylcholine (DPPC), distearyloyl phosphatidylcholine (DSPC), diarachidoyl phosphatidylcholine, dimyristoyl phosphatidylcholine (DMPC), egg phosphatidylcholine (EPC), soy phosphatidylcholine (SPC), oleoyl palmitoyl phosphatidylcholine, dioleoyl phosphatidylcholine (DOPC), dipetroselinoyl phosphatidylcholine, palmitoylelaidoyl phosphatidylcholine, palmitoyloleoyl phosphatidylcholine, dilauroyl phosphatidylcholine (DLPC), diundecanoyl phosphatidylcholine PC (11:0/11:0), didecanoyl phosphatidylcholine PC (10:0/10:0), dinonanoyl phosphatidylcholine PC (9:0/9:0) or any combination thereof, the second phospholipid is 1,2-distearoly-sn-glycero-3-phosphoethanolamine-N-[methoxy (polyethylene glycol)-2000] (DSPE-PEG2000), distearyloyl phosphatidylglycerol (DSPG), Dipalmitoylphosphatidylglycerol (DPPG), dimyristoylphosphatidylglycerol (DMPG), 1,2-dioleoyl-sn-glycero-3-phosphoglycerol (DOPG) or any combination thereof, the charged lipid is stearylamine, 1,2-dioleoyl-3-trimethylammonium-propane (DOTAP), 3ß-[N—(N′,N′-dimethylaminoethane)-carbamoyl]cholesterol (DC-Cholesterol), N⁴-Cholesteryl-Spermine (GL67), dimethyldioctadecylammonium (DDAB), 1,2-di-O-octadecenyl-3-trimethylammonium propane (DOTMA), ethylphosphocholine (ethyl PC) or any combination thereof and the sterol is cholesterol.

14. The kit of claim 13, wherein the mole percent of the first phospholipid: sterol: the second phospholipid or the charged lipid is 50-70:20-45:0.1-10 or 75-99:0-14.9:0.1-10.

15. The kit of claim 1, wherein the prostaglandin, prostaglandin analogue, prostacyclin or prostacyclin analogue is iloprost or treprostinil.

16. The kit of claim 15, wherein the prostaglandin, prostaglandin analogue, prostacyclin or prostacyclin analogue is treprostinil, and wherein the nebulizer is configured to deliver about 40 to about 400 μg of treprostinil in less than 3 minutes.

17. The kit of claim 15, wherein the prostaglandin, prostaglandin analogue, prostacyclin or prostacyclin analogue is iloprost, and wherein the nebulizer is configured to deliver about 5 to 100 μg of iloprost in less than 3 minutes.

18. The kit of claim 1, wherein the nebulizer is configured to retain about 0.01 to 1 mL of the pharmaceutical composition remains in the reservoir after the delivery.

19. The use of the kit of claim 1 for treating a respiratory disease and heart failure.

20. The use of claim 19, wherein the respiratory disease is pulmonary hypertension, pulmonary fibrotic disease, interstitial lung disease, asthma, chronic obstructive airway disease and respiratory complications caused by cystic fibrosis.
